(19) United States
(12) Patent Application Publication
(10) Pub. No.: US 2015/0038359 A1
(43) Pub. Date: Feb. 5, 2015

(54) METHOD OF PREDICTING OUTCOME IN CANCER PATIENTS

(71) Applicant: McMaster University, Hamilton (CA)

(72) Inventors: Robin Hallett, Hamilton (CA); John Hassell, Dundas (CA); Anna Dvorkin, Hamilton (CA); Anita Bane, Hamilton (CA)

(21) Appl. No.: 14/366,543

(22) PCT Filed: Dec. 21, 2012
(86) PCT No.: PCT/CA2012/050937

§ 371 Date: Jun. 18, 2014

Related U.S. Application Data
(60) Provisional application No. 61/578,927, filed on Dec. 22, 2011.

(51) Int. Cl. C12Q 1/68

(52) U.S. Cl. CPC C12Q 1/6886; C12Q 2600/158; C12Q 2600/118
USPC 506/9; 506/16

(57) ABSTRACT

A method of prognosis for a mammal with cancer is provided. The method includes the steps of determining in a biological sample obtained from the mammal the expression level of each biomarker of the group DSTN, TDRD3, RGS4, MYO1E, RPL3, Hypothetical FLJ13769, ANP32C, MC2R, DKFZp434L092, GPR27, HPS and LCP1; comparing the expression level of each biomarker with the expression level of a housekeeping gene; and rendering a prognosis for the mammal of a greater than 50% survival for an extended period of time when the expression level of DSTN, TDRD3, RGS4, MYO1E, RPL3(1), RPL3(2), RPL3(3), Hypothetical FLJ13769 and ANP32C is decreased in comparison to the expression of the housekeeping gene, and the expression level of MC2R, DKFZp434L092, GPR27, HPS5 and LCP1 is increased in comparison to the expression of the housekeeping gene.

### FIELD OF INVENTION

The present invention relates to a prognostic method in mammals with cancer, and more particularly, relates to method of predicting prognosis based on a novel set of cancer-related biomarkers.

### BACKGROUND OF THE INVENTION

Traditionally a number of tumor characteristics have been used to determine the prognosis of breast cancer patients. Such factors include tumor size, grade, hormone receptor status, HER2 status, lympho-vascular space invasion and lymph node involvement. More recently, whole genome analysis technology (gene expression profiling) has been added to the armamentarium of experimental techniques, thus providing a new molecular classification for breast cancer and contributing to the development of a number of prognostic multi-gene assays including a 21-gene, 70-gene, 76-gene, 77-gene genomic grade profile, wound response signature and others. Oncotype DX™, for example, a 21-gene quantitative (q)RT-PCR assay, evaluates expression of 16 genes identified to be of prognostic importance as well as 5 house-keeping genes. Oncotype DX™ predicts the risk of distant recurrence in Estrogen Receptor (ER) positive breast cancers and their responsiveness to CMF (Cyclophosphamide, Methotrexate and 5-Fluorouracil) chemotherapy. MammaPrint™, a commercially available microarray, evaluates the expression of 70 genes using RNA extracted from fresh frozen tumor samples. This assay distinguishes patients that have a good prognosis (no relapse within 5 years) from those that have a poor prognosis (relapse within 5 years). Trials, TAILORx [Trial Assigning Individualized Options for Treatment] and MINDACT [Microarray In Node Negative and 1-3 positive lymph node Disease may Avoid Chemotherapy] are ongoing to evaluate how to incorporate both Oncotype DX™ and MammaPrint® into clinical practice.

The term basal-like breast cancer (BLBC) originated in 2000 from gene expression profiling experiments conducted on invasive breast cancers. Using hierarchical clustering, a new molecular taxonomy for breast cancer based on the relative expression of the ˜500 genes was identified, known as the ‘intrinsic’ gene set. It was discovered that breast cancers could be classified into five molecular subgroups. Two of these are ER positive, while three are ER negative. The ER positive subgroups, termed Luminal A and Luminal B, were identified based on their relative expression of the ER gene, ER regulated genes and other genes expressed by normal breast ‘luminal’ cells. The ER negative subgroups are referred to as HER2-overexpressing (ERBB2+), normal breast-like and BLBC. The HER2-overexpressing subgroup was characterized by the overexpression of HER-2 and other genes on the 17 q amplicon, such as GRB7. The normal breast-like subgroup expresses genes characteristic of adipose tissue suggesting that this subgroup may be a technical artifact resulting from low tumor cellularity. Lastly, the basal-like subgroup represents a distinct and novel class of tumors characterized by the lack of expression of ER, PR and HER2 and the high expression of cytokeratins (CK)5, and/or CK 17 (amongst other genes), characteristic of the basal/myoepithelial cell layer of the normal breast epithelium. As gene expression studies continued to evolve, new molecular subtypes of breast cancer continued to be discovered, for example, the claudin-low subtype.

The initial gene expression profiling experiments demonstrated that BLBCs together with the HER2-overexpressing subtype were associated with a particularly poor prognosis. By comparison, patients with Luminal A type tumors displayed an excellent prognosis. However, on closer examination these studies additionally demonstrated that the prognosis of patients with BLBCs is highly time dependent. Some patients with BLBCs experience particularly poor survival in the first 3-5 years following diagnosis, but others experience better survival than those with luminal-type (ER+) tumors. This suggests that patients with BLBCs can be separated into two clinically distinct groups: those likely to experience a recurrence and succumb to their disease in the first 3-5 years after diagnosis, and those expected to show excellent long term survival.

While several multi-gene signatures exist to predict breast cancer patient prognosis, their prognostic values appear to be, in large part, derived from their capacity to measure expression of genes associated with proliferation. Because BLBCs are generally highly proliferative, the existing prognostic signatures fail to identify a subset of BLBC with good prognosis. Some recent work has focused on identifying multi-gene predictors of outcome in triple negative (ER−, PR−, HER2−) and hormone receptor negative breast cancer. However, a robust method of distinguishing between BLBCs with good and poor outcome has yet to be developed.

### SUMMARY OF THE INVENTION

A method of accurately predicting outcome in mammals with basal-like breast cancer (BLBC) and molecularly similar cancers, has now been developed and is based on a 14-member biomarker signature.

Thus, in one aspect of the invention a method of prognosis in a mammal with BLBC and molecularly similar cancers is provided comprising: determining in a biological sample obtained from the mammal the level of each biomarker of the group DSTN, TDRD3, RGS4, MYO1E, RPL3, Hypothetical FLJ13769, ANP32C, MC2R, DKFZp434L092, GPR27, HPS5 and LCP1; comparing the expression level of each biomarker with the expression of one or more housekeeping genes; and rendering a prognosis for the mammal of a greater than 50% survival for an extended period of time when the expression level of DSTN, TDRD3, RGS4, MYO1E, RPL3, Hypothetical FLJ13769 and ANP32C is decreased in comparison to housekeeping gene expression levels, and the expression level of MC2R, DKFZp434L092, GPR27, HPS5 and LCP1 is increased in comparison to housekeeping gene expression levels.

In another aspect, an article of manufacture for use in a method of prognosis in a mammal with BLBC and molecularly similar cancers is provided. The article comprises packaging and a biomarker-specific reactant for one or more biomarker or nucleic acid encoding the biomarker of the group, DSTN, TDRD3, RGS4, MYO1E, RPL3, Hypothetical FLJ13769, ANP32C, MC2R, DKFZp434L092, GPR27, HPS5 and LCP1, wherein the reactant is suitable to determine the level of expression of the biomarker in a biological sample from the mammal, and wherein the packaging indicates that a determination in the sample of a decreased level of DSTN, TDRD3, RGS4, MYO1E, RPL3, Hypothetical FLJ13769 and ANP32C and an increased level of MC2R, DKFZp434L092, GPR27, HPS5 and LCP1 in comparison to the level of expression of a housekeeping gene is indicative of a prognosis for the mammal of greater than 50% survival for an extended period of time.

These and other aspects are described in the detailed description that follows by reference to the following figures.

### BRIEF DESCRIPTION OF THE FIGURES

FIG. 1 graphically illustrates a comparison of the relative risk of gene signatures of various signature lengths in which a 14 protein signature was identified as optimal;

FIG. 2 graphically illustrates the probability of increasing proportions of patients that experience disease relapse as predicted by Basal 14 signature (A), the sensitivity and specificity (and therefore accuracy) of the Basal 14 signature in the validation cohort (B), a Kaplan-Meier survival analysis of the validation cohort (C), and Kaplan-Meier survival analysis with chemotherapy naïve patients (D);

FIG. 3 graphically illustrates survival within groups having poor and good predicted outcome using various gene signatures: A) Basal 14, B) Genomic Grade Index, C) NKI-70, D) Recurrence Score, E) CSR/Wound response, F) Triple Negative and G) MS-14 signatures;

FIG. 4 is a graphical evaluation of the Basal-14 signature different breast cancer subtypes including: A) luminal A, B) luminal B, C) claudin low, D) Normal, and F) ERBB2 cancer;

FIG. 5 is a graphical comparison of various prognostic signatures including A) Basal 14, B) Genomic Grade Index, C) NKI-70, D) Recurrence Score, E) CSR/Wound response, F) Triple Negative and G) MS-14 signatures;

FIG. 6 graphically illustrates survival analysis of training data set (A) and validation set (B) using 50 prognostic genes in basal-like breast cancer patients in a microarray-based BLBC patient series, and in a BLBC patient cohort using the NanoString nCounter Gene Expression System (C-E);

FIG. 7 illustrates the amino acid sequence of isoform 1 (A) and isoform 2 (B) of the Destrin protein, as well as the transcript sequences thereof (C/D);

FIG. 8 illustrates the amino acid sequence of isoform 1 (A) and isoform 3 (B) of the Tudor domain containing protein 3, as well as the transcript sequences thereof (C/D);

FIG. 9 illustrates the amino acid sequence of isoforms of the Regulator of G-protein signaling (RGS4) protein, as well as the transcript sequences thereof (C-E);

FIG. 10 illustrates the amino acid sequence of myosin 1E (A) and the transcript sequence thereof (B);

FIG. 11 illustrates the amino acid sequence Hypothetical protein FLJ13769 (A), and the transcript sequence thereof (B);

FIG. 12 illustrates the amino acid sequence of human ribosomal protein L3 (60 s subunit) (A), mouse RPL3 (B) and the transcript sequence for the human form (C);

FIG. 13 illustrates the amino acid sequence of Acidic (leucine-rich) nuclear phosphoprotein 32 family, member C (ANP32C), and the transcript sequence thereof (B);

FIG. 14 illustrates the amino acid sequence of human (A) and (B) mouse melanocortin 2 receptor, and the transcript sequence of the human form (C);

FIG. 15 illustrates the amino acid sequence of DKFZp434L092;

FIG. 16 illustrates the amino acid sequence of human (A) and mouse (B) G protein-receptor 27, and the transcript sequence of the human form (C);

FIG. 17 illustrates the amino acid sequence of human (A) and mouse (B) of Hermansky-Pudlak syndrome 5 protein, and the transcript sequences of human isoforms A/B (C/D);

FIG. 18 illustrates the amino acid sequence of human (A) and mouse (B) of Lymphocyte cytosolic protein 1, and the transcript sequence of the human form (C);

FIG. 19 illustrates the amino acid sequence of human ADAM22 (A) and transcript sequences for isoforms 1-4 (B-E);

FIG. 20 illustrates the transcript sequence of ANXA2P1;

FIG. 21 illustrates the amino acid sequence of human APBB2 (A), as well as transcript sequences for isoforms A-D (B-E);

FIG. 22 illustrates the amino acid sequence of ATP10B (A), as well as the transcript sequence thereof (B);

FIG. 23 illustrates the amino acid sequence of BCL2L14 (A), as well as the transcript sequences for isoforms 1 and 2 (B and C);

FIG. 24 illustrates the amino acid sequence of CBWD1 (A), as well as to transcript sequences for isoforms 1-3 (B-D);

FIG. 25 illustrates the amino acid sequence of CBWD1 (A), as well as the transcript sequence for CNIH3 (B);

FIG. 26 illustrates the amino acid sequence of CR2 (A), as well as the transcript sequences for isoforms 1 and 2 (B and C);

FIG. 27 illustrates the amino acid sequence of DENND3 (A), as well as the transcript sequence for DENND3 (B);

FIG. 28 illustrates the amino acid sequence of DHX15 (A), as well as the transcript sequence thereof (B);

FIG. 29 illustrates the amino acid sequence of EIF3H (A), as well as the transcript sequence thereof (B);

FIG. 30 illustrates the amino acid sequence of EPHA5 (A), as well as the transcript sequences for isoforms A and B (B and C);

FIG. 31 illustrates the amino acid sequence of GADD45B (A), as well as the transcript sequence thereof (B);

FIG. 32 illustrates the amino acid sequence of GLP1R (A), as well as the transcript sequence thereof (B);

FIG. 33 illustrates the amino acid sequence of GLRA3 (A), as well as the transcript sequences for GLRA3 isoforms A and B (B and C);

FIG. 34 illustrates the amino acid sequence of HEXIM1 (A), as well as the transcript sequence thereof (B);

FIG. 35 illustrates the amino acid sequence of HIST1H3J (A), as well as the transcript sequence thereof (B);

FIG. 36 illustrates the amino acid sequence of IL17B (A), as well as the transcript sequence thereof (B);

FIG. 37 illustrates the amino acid sequence of IQCA1 (A), as well as the transcript sequence thereof (B);

FIG. 38 illustrates the amino acid sequence of KLHDC2 (A), as well as the transcript sequence thereof (B);

FIG. 39 illustrates the amino acid sequence of LMAN1L (A), as well as the transcript sequence thereof (B);

FIG. 40 illustrates the amino acid sequence of LOC642131 (A), as well as the transcript sequence thereof (B);

FIG. 41 illustrates the amino acid sequence of LRRC37A4 (A), as well as the transcript sequence thereof (B);

FIG. 42 illustrates the amino acid sequence of MRPL46 (A), as well as the transcript sequence; thereof (B);

FIG. 43 illustrates the amino acid sequence of MYCNOS (A), as well as the transcript sequence thereof (B);

FIG. 44 illustrates the amino acid sequence of NDUFAF1 (A), as well as the transcript sequence thereof (B);

FIG. 45 illustrates the amino acid sequence of PARP4 (A), as well as the transcript sequence thereof (B);

FIG. 46 illustrates the amino acid sequence of PDE4B (A), as well as the mRNA transcript sequences for isoforms 1 and 2 (B and C);

FIG. 47 illustrates the transcript sequence of PDZRN3;

FIG. 48 illustrates the mRNA transcript sequence for PFDN5, alpha isoform (A), and isoform gamma (B);

FIG. 49 illustrates the mRNA transcript sequence for PSG11, isoform 1 (A), and isoform 2 (B);

FIG. 50 illustrates the mRNA transcript sequence for isoforms A-C of RFPL3S (A-C);

FIG. 51 illustrates the mRNA transcript sequence for isoforms A-C of RHBG (A-C);

FIG. 52 illustrates the transcript sequence of RPL13A;

FIG. 53 illustrates the transcript sequence of RTF1;

FIG. 54 illustrates the transcript sequence of SHOX2;

FIG. 55 illustrates the transcript sequence of SLC11A1;

FIG. 56 illustrates the transcript sequence of SLC5A6;

FIG. 57 illustrates the transcript sequence of Steroid sulphatase;

FIG. 58 illustrates the mRNA transcript sequence for SYNC1, isoform 1 (A), and isoform 2 (B);

FIG. 59 illustrates the mRNA transcript sequence for TBC1D1, isoform 1 (A), and isoform 2 (B);

FIG. 60 illustrates the mRNA transcript sequence for TCEA2, isoform 1 (A), and isoform 2 (B); and

FIG. 61 illustrates a block diagram for a processor.

### DETAILED DESCRIPTION

A method of prognosis in a mammal with BLBC or a molecularly similar cancer is provided comprising: determining in a biological sample obtained from the mammal the level of each biomarker of the group DSTN, TDRD3, RGS4, MYO1E, RPL3, Hypothetical FLJ13769, ANP32C, MC2R, DKFZp434L092, GPR27, HPS5 and LCP1; comparing the expression level of each biomarker with the expression of one or more housekeeping genes; and rendering a prognosis for the mammal of a greater than 50% survival for an extended period of time when the expression level of DSTN, TDRD3, RGS4, MYO1E, RPL3, Hypothetical FLJ13769 and ANP32C is decreased in comparison to housekeeping gene expression levels, and the expression level of MC2R, DKFZp434L092, GPR27, HPS5 and LCP1 is increased in comparison to housekeeping gene expression levels.

The biomarker signature comprises the following biomarkers, Destrin; Tudor domain containing protein 3; Regulator of G-protein signaling; Myosin IE; Hypothetical protein FLJ13769; Ribosomal protein L3 (60 s subunit); Ribosomal protein L3, Acidic (leucine-rich) nuclear phosphoprotein 32 family, member C; Melanocortin 2 receptor; DKFZp434L092; G protein-receptor 27; Hermansky-Pudlak syndrome 5; and Lymphocyte cytosolic protein 1.

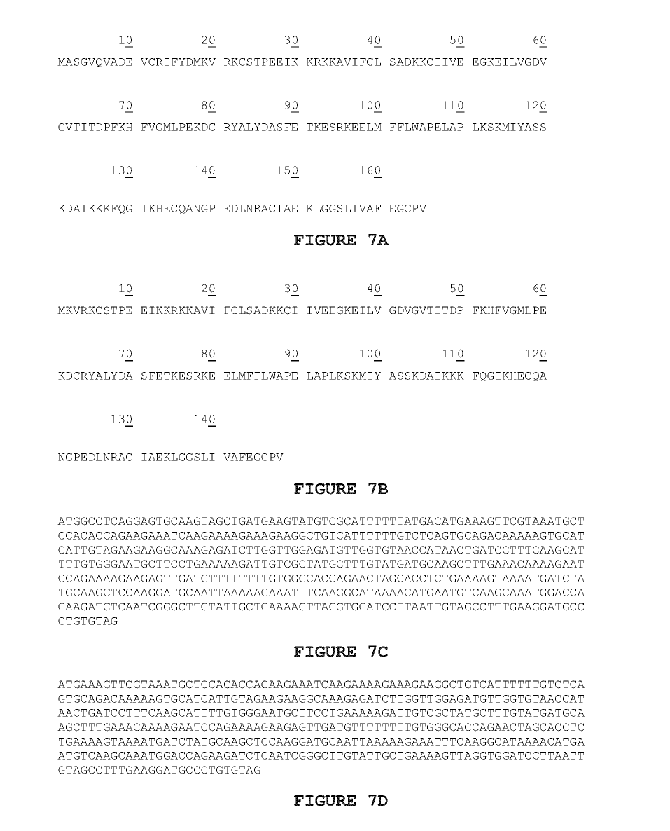

Destrin (DSTN) is a mammalian actin depolymerisation factor, and as used herein is meant to encompass both human destrin as depicted by Uniprot P60981, including all isoforms thereof, such as isoform 1 which is a 165 amino acid protein, and isoform 2 which is a 148 amino acid protein, as shown in FIG. 7A/B, as well as functionally equivalent variants thereof, such as other mammalian forms thereof. Transcript sequences for DSTN isoforms A and B are shown in FIG. 7C/D. The term “functionally equivalent” is used herein with respect to other forms of a biomarker protein or nucleic acid that may be used in the present method to generate a signature that is useful in the prognosis of a mammal with BLBC or a molecularly similar cancer.

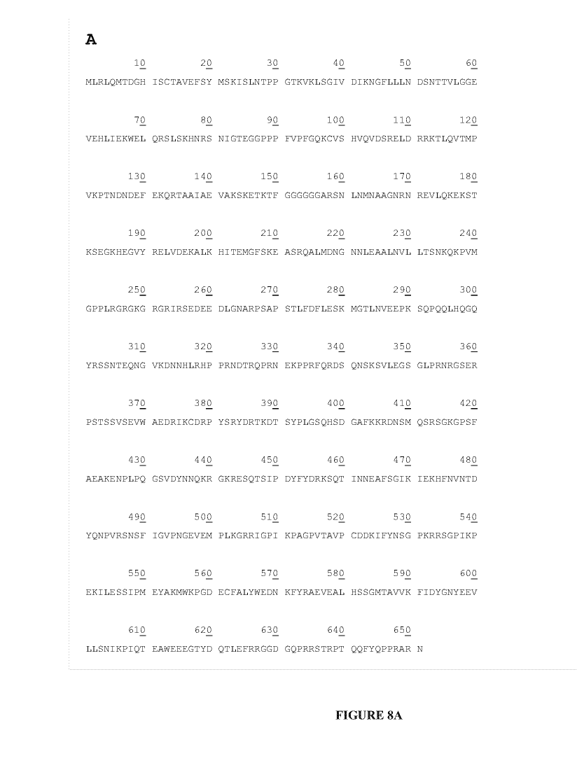

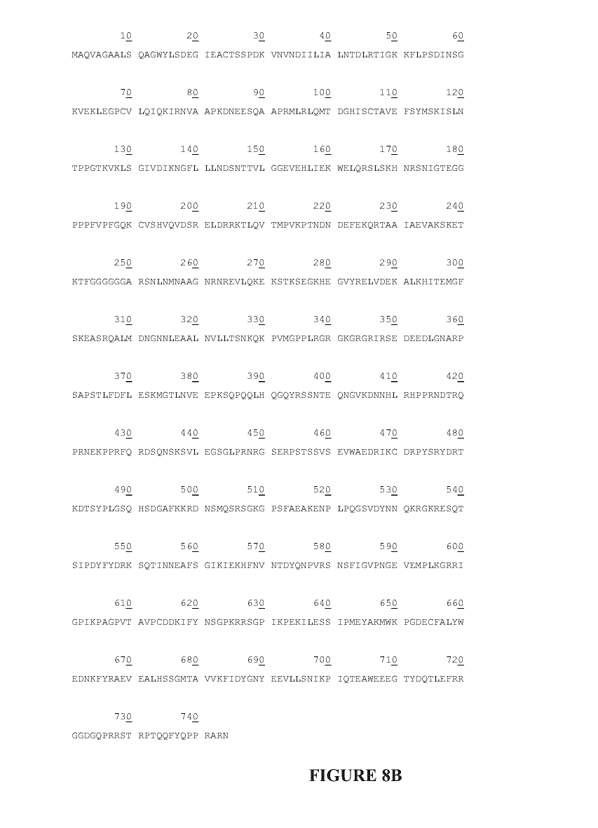

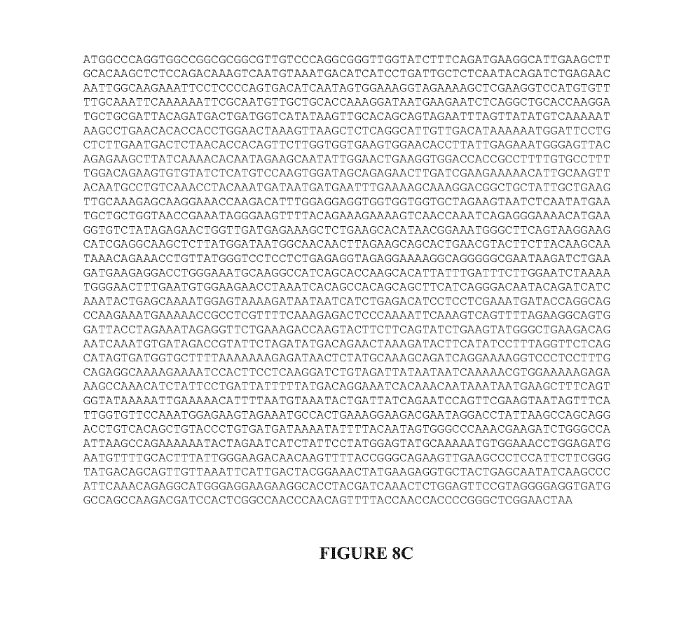

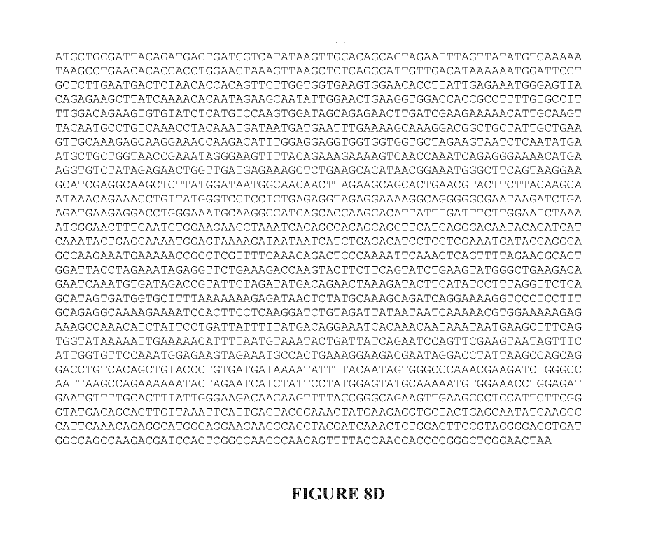

Tudor domain containing protein 3 (TDRD3) comprises a 50 amino acid structural motif known as a tudor domain, and interact with arginine-methylated polypeptides. As used herein, TDRD3 is meant to encompass both human TDRD3 as depicted by Uniprot Q9H7E2, including all isoforms thereof, such as isoform 1 which is a 65 amino acid protein, and isoform 2 which is a 650 amino acid protein that differs from isoform 1 by omission of the lysine at position 97, and isoform 3 which is a 744 amino acid protein, as shown in FIG. 8A/B, as well as functionally equivalent variants thereof, such as other mammalian forms thereof, e.g. mouse TDRD3 depicted by the 743 amino acid sequence of Uniprot Q91W18, and isoforms thereof. Transcript sequence for TDRD3 isoforms 1 and 2 are shown in FIG. 8C/D.

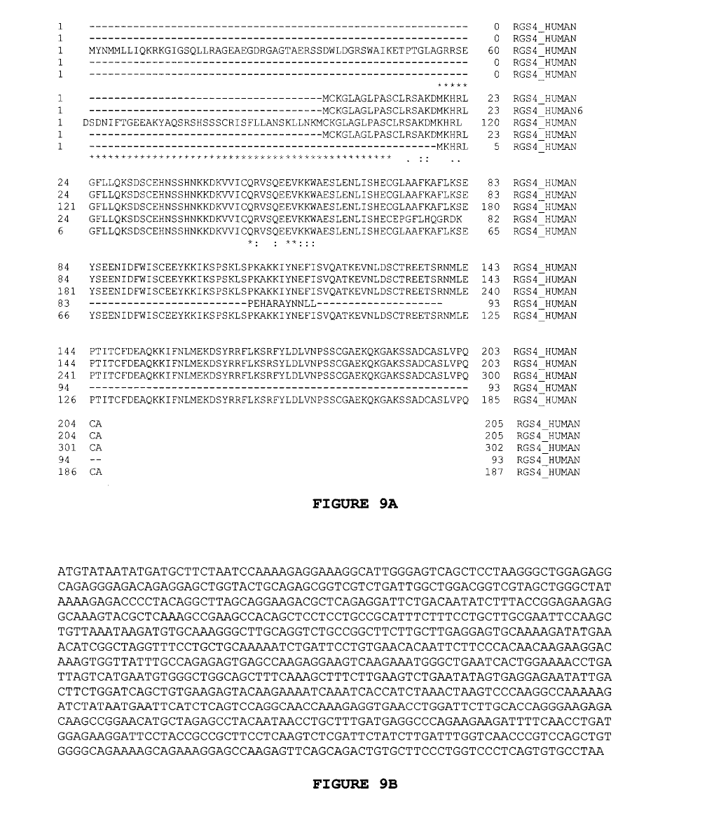

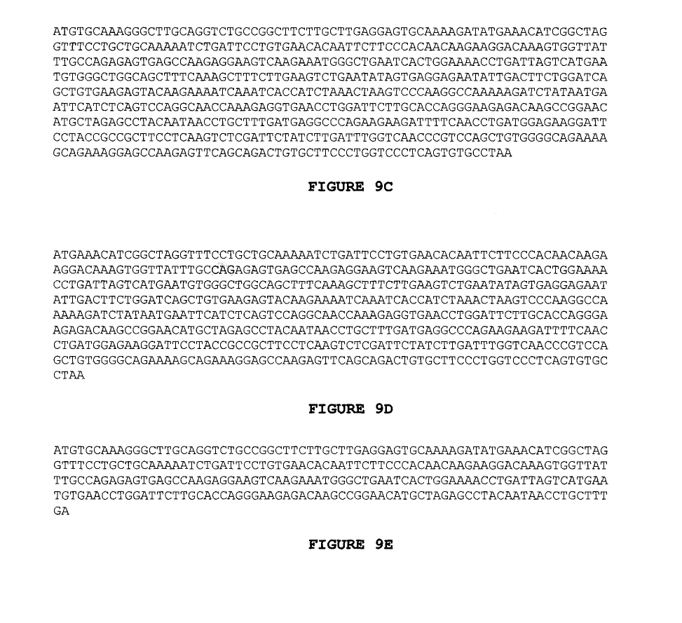

Regulator of G-protein signaling (RGS4) protein is a regulatory molecule that acts as a GTPase activating protein for G alpha subunits of heterotrimeric G proteins. RGS4 is used herein to encompass both human RGS4 as depicted by Uniprot P49798, including all isoforms thereof, such as isoforms 1-5 as shown in FIG. 9A, as well as functionally equivalent variants thereof, such as other mammalian forms thereof. Transcript sequences for RGS4 isoforms 1-4 are shown in FIG. 9B-E.

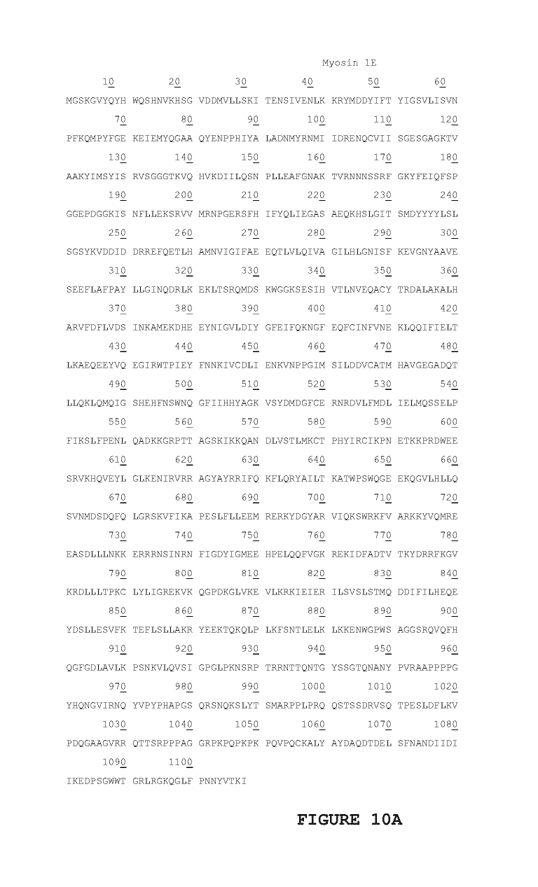

Myosin IE (MYO1E) is an unconventional myosin also referred to as myosin 1C. As used herein, the term “myosin 1E” is meant to encompass both human MYO1E as depicted by Uniprot Q12965 and FIG. 10A, including all isoforms and functionally equivalent variants thereof, such as the variant in which residue 159 is proline, the variant in which residue 185 is glycine, the variant in which residue 221 is valine, the variant in which residue 795 is arginine and the variant in which residue 1049 is histidine, as well as other mammalian forms thereof such as mouse myosin 1E as depicted by Uniprot E9Q634. Transcript sequence for MYO1E is shown in FIG. 10B.

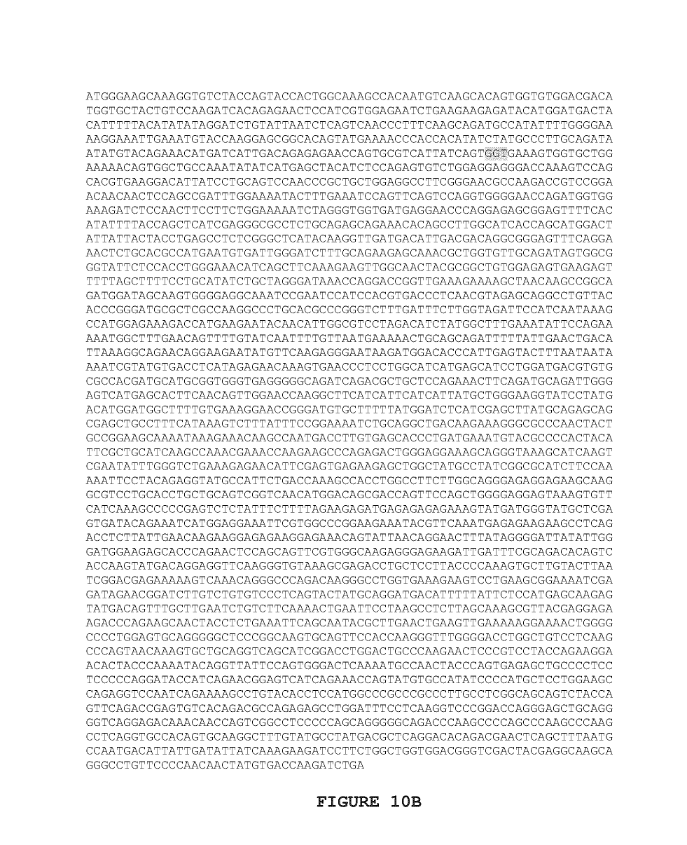

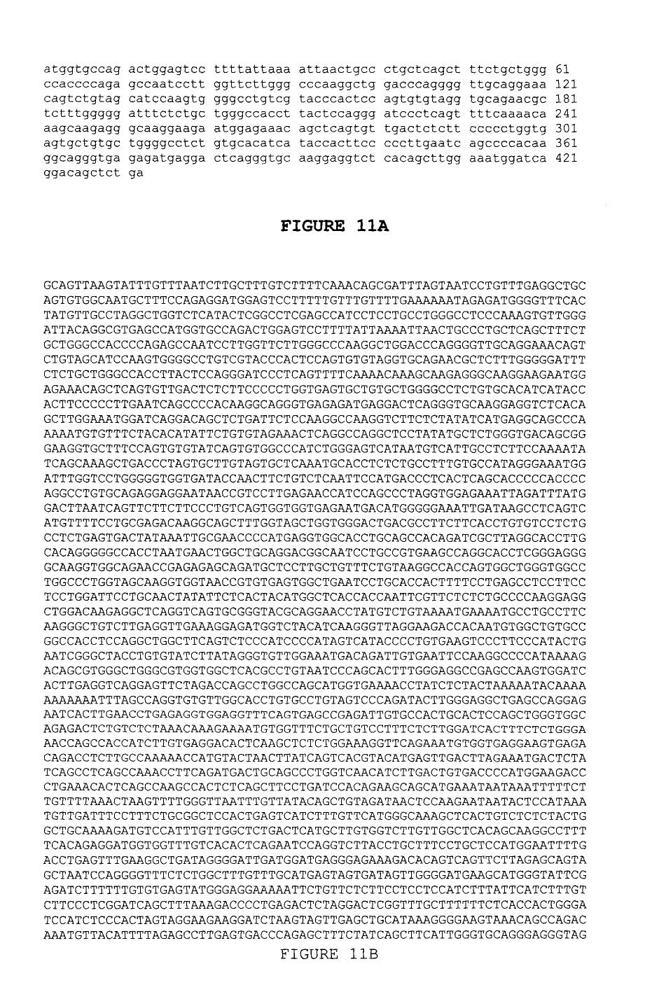

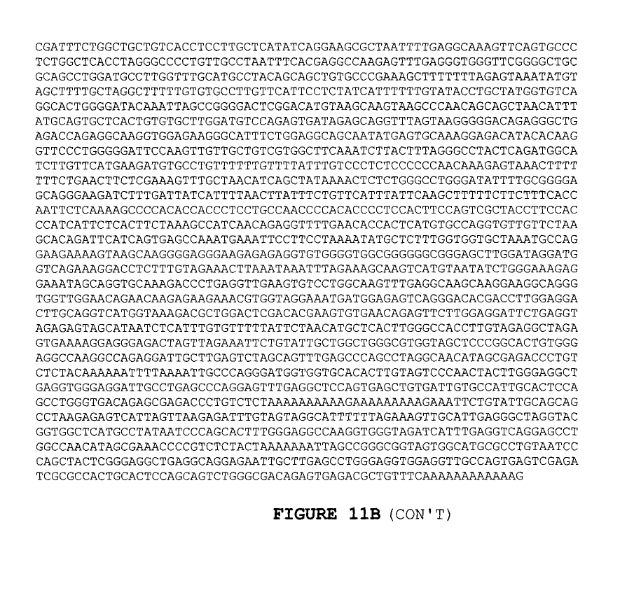

Hypothetical protein FLJ13769 is encoded by the gene, FLJ13769, having the DNA sequence as shown in FIG. 11A, and functional variants thereof as a result of degeneracy in the genetic code. Transcript sequence for EIF3H is shown in FIG. 11B.

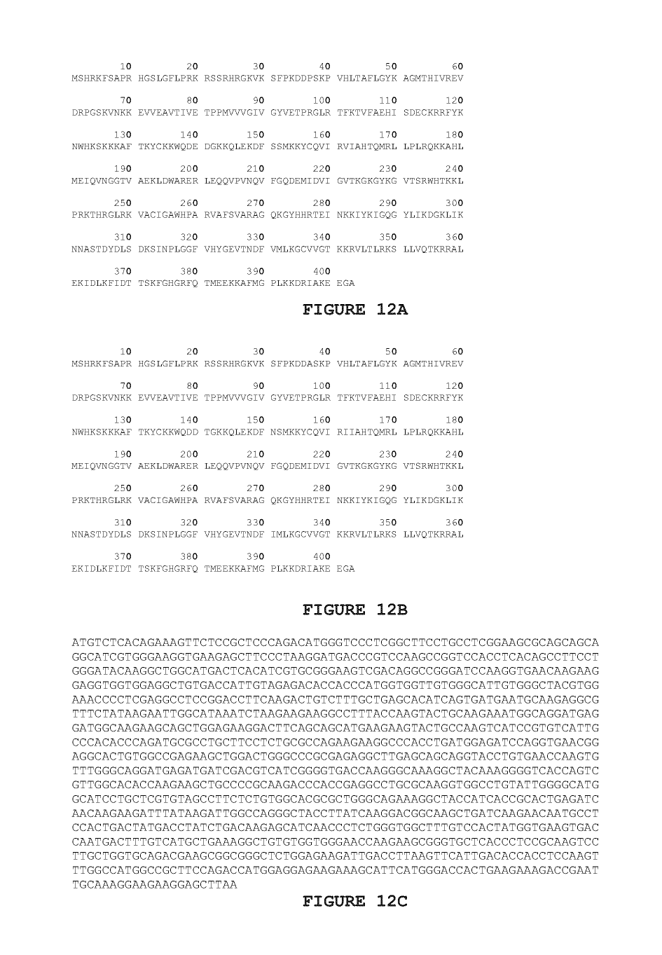

Ribosomal protein L3 (RPL3) is the 60 s subunit of the ribosomal protein encoded by the RPL3 gene. As used herein, the term “ribosomal protein L3” is meant to encompass both human RPL3 as depicted by Uniprot P39023 and FIG. 12A, including all isoforms and functionally equivalent variants thereof, such as the variant in which residue 78 is thymine, as well as other mammalian forms thereof such as mouse RPL3 as depicted by Uniprot P27659 and FIG. 12B. Transcript sequence for RPL3 is shown in FIG. 12C.

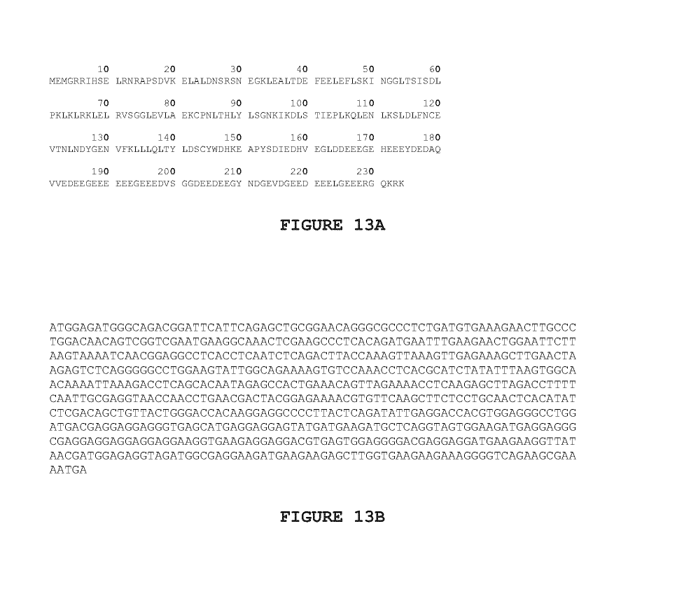

Acidic (leucine-rich) nuclear phosphoprotein 32 family, member C, also referred to as “ANP32C” is a protein encoded by the gene, ANP32C. As used herein, the term “ANP32C” is meant to encompass both human ANP32C as depicted by Uniprot 043423 and FIG. 13A, including all isoforms and functionally equivalent variants thereof, such as the variant in which residue 23 is valine, the variant in which residue 71 is lysine, the variant in which residue 105 is proline, the variant in which residue 140 is histidine and the variant in which residue 204 is glycine, as well as other mammalian forms thereof. Transcript sequence for ANP32C is shown in FIG. 13B.

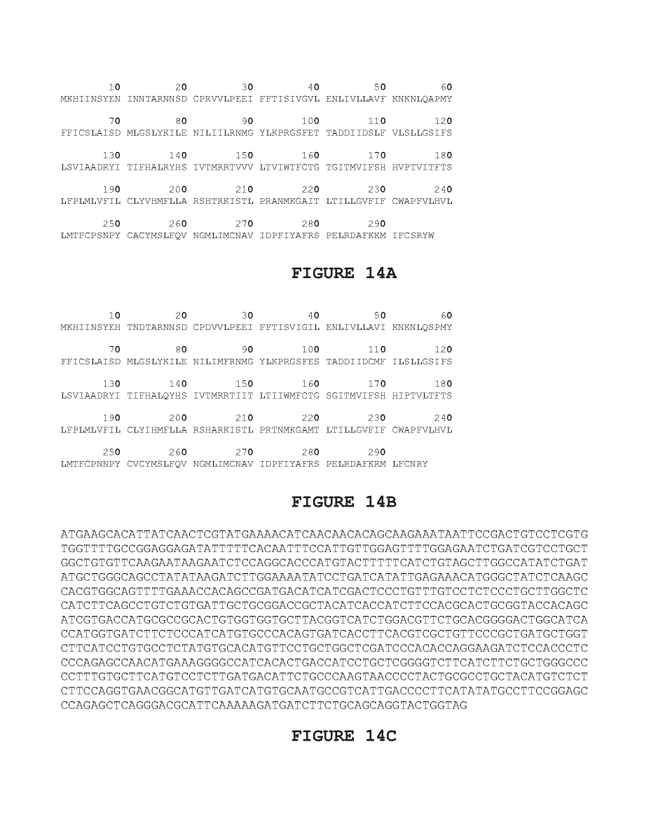

Melanocortin 2 receptor (MC2R), also referred to as adrenocorticotropic hormone receptor (ACTHR), is a melanocortin receptor that is specific for adrenocorticotropic hormone. As used herein, the term “MC2R” is meant to encompass both human MC2R as depicted by Uniprot Q01718 and FIG. 14A, including all isoforms and functionally equivalent variants thereof, such as the variant in which residue 27 is arginine, the variant in which residue 103 is asparagine, the variant in which residue 107 is asparagine, the variant in which residue 120 is arginine, the variant in which residue 128 is cysteine, the variant in which residue 146 is histidine, and the variant in which residue 251 is phenylalanine, as well as other mammalian forms thereof such as mouse MC2R as depicted by Uniprot Q64326 and FIG. 14B. Transcript sequence for MC2R is shown in FIG. 14C.

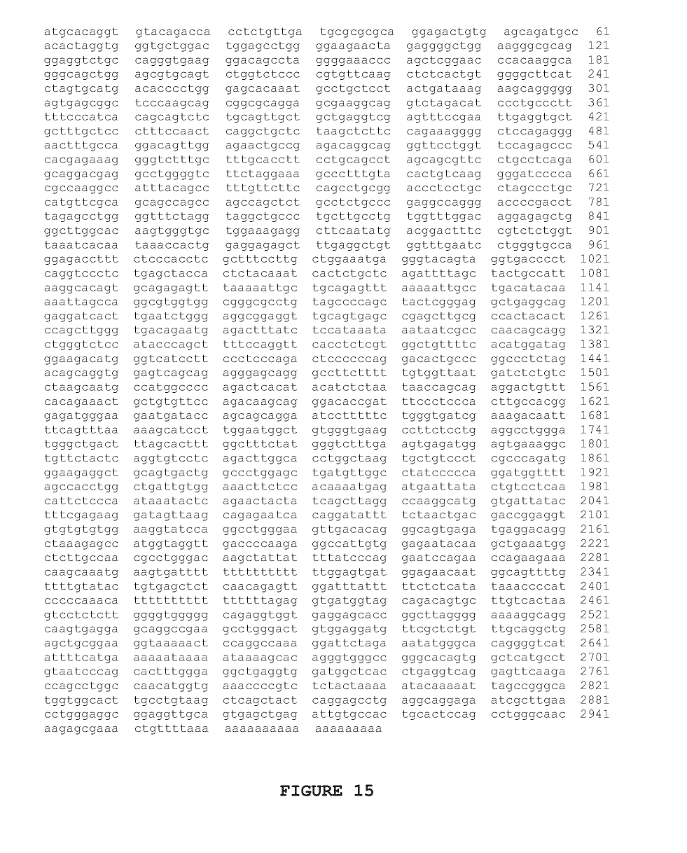

DKFZp434L092 (from clone DKFZp434L092) has the DNA sequence as shown in FIG. 15.

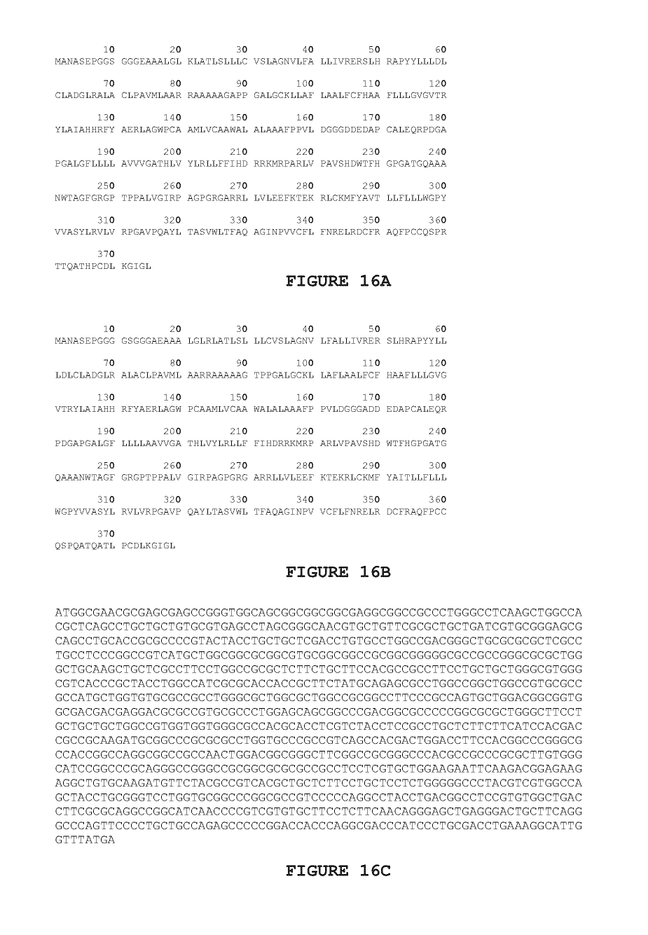

G protein-receptor 27 (GPR27) is a protein encoded by the GPR27 gene. As used herein, the term “GPR27” is meant to encompass both human GPR27 as depicted by Uniprot Q9NS67 and FIG. 16A, including all isoforms and functionally equivalent variants thereof, as well as non-mammalian forms thereof such as mouse GPR27 as depicted by Uniprot 054897 and FIG. 16B. Transcript sequence for GPR27 is shown in FIG. 16C.

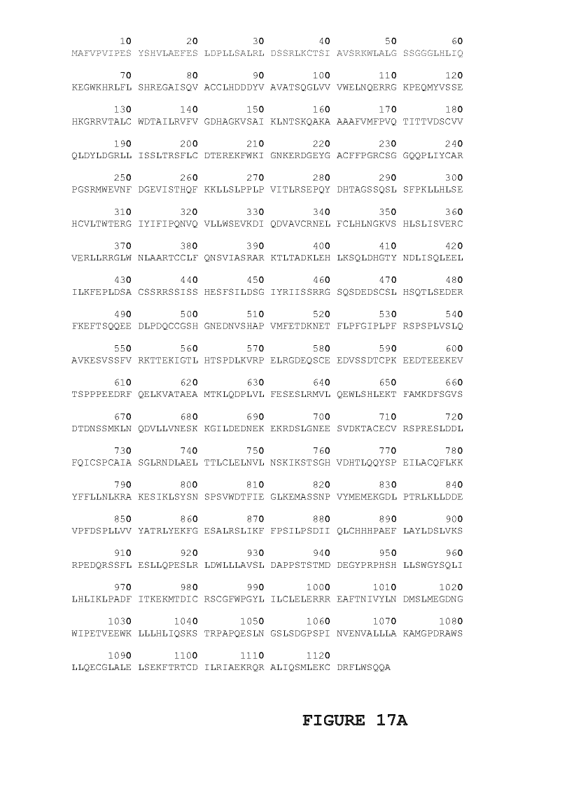

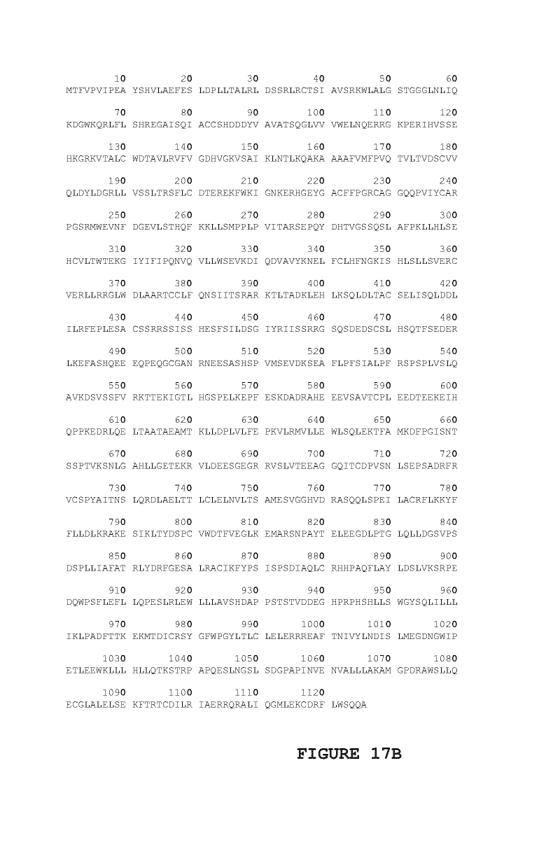

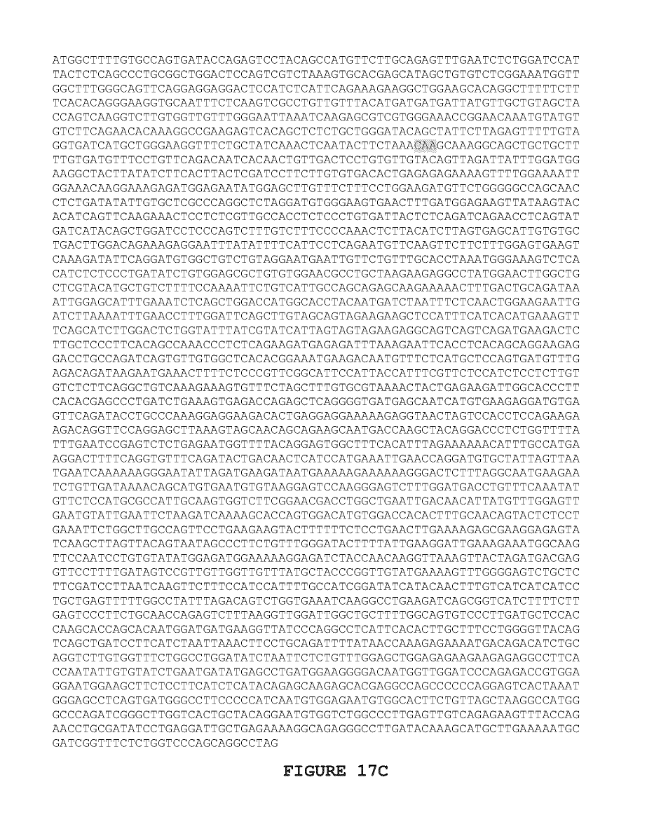

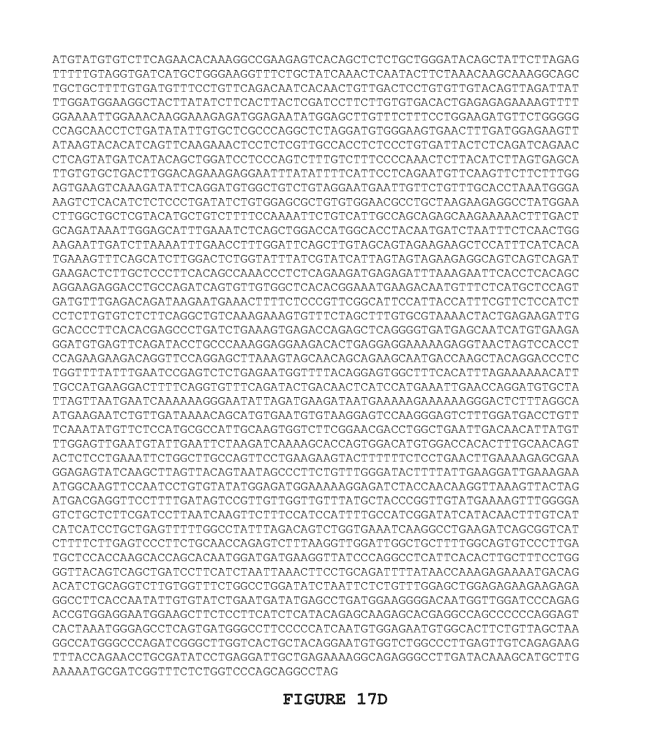

Hermansky-Pudlak syndrome 5 (HPS5) is a protein encoded by the HPS57 gene. As used herein, the term “HPS5” is used to encompass both human HPS5 as depicted by Uniprot Q9UPZ3 and FIG. 17A, including all isoforms, such as isoform 2 which is missing residues 1-114 of the sequence of FIG. 17A, and functionally equivalent variants such as the variant in which residue 417 is methionine, the variant in which residue 624 is arginine, the variant in which residue 1098 is isocleucine, as well as non-human forms thereof such as mouse H as depicted by Uniprot P59438 and FIG. 17B, and isoforms thereof such as isoform 2 in which residues 1-165 is missing from the sequence shown in FIG. 17B. Transcript sequences for isoforms A and B are shown in FIGS. 17C and D.

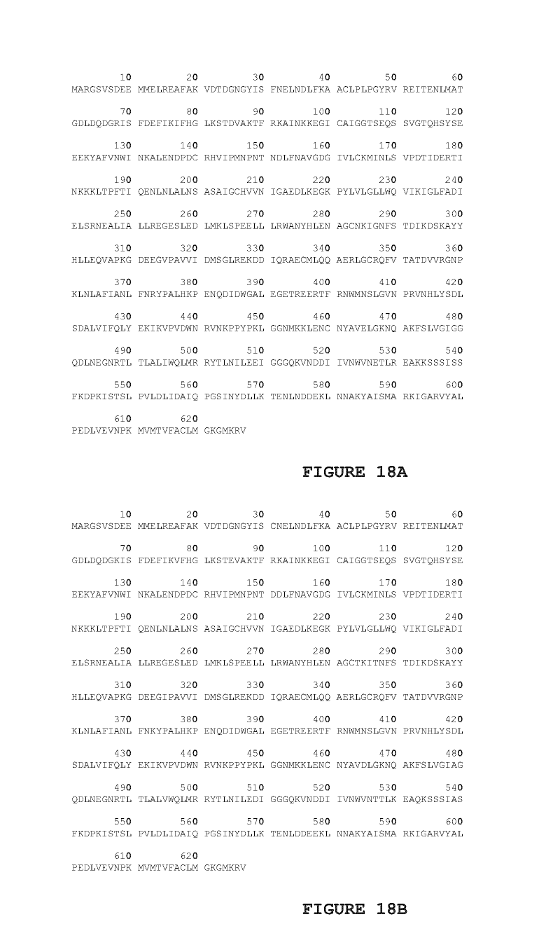

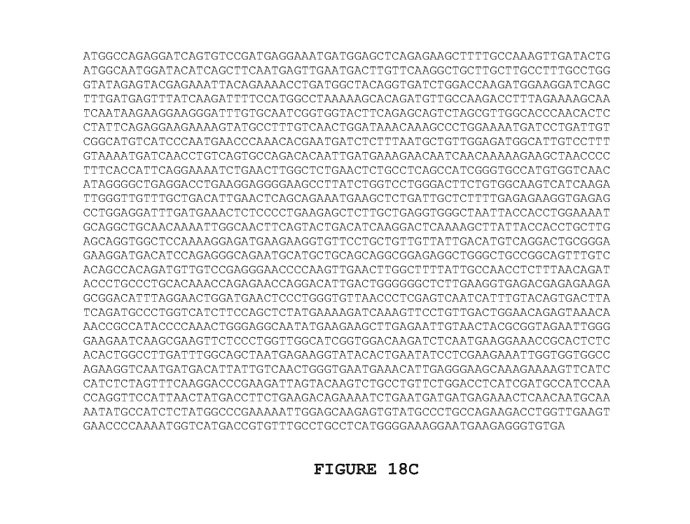

Lymphocyte cytosolic protein 1, also referred to as L-plastin or LCP1, is used herein to encompass both human LCP1 as depicted by Uniprot P13796 and FIG. 18A, including all isoforms and functionally equivalent variants thereof, such as the variant in which residue 24 is glutamic acid, the variant in which residue 533 is glutamic acid, and the variant in which residue 544 is alanine, as well as non-human forms thereof such as mouse LCP1 as depicted by Uniprot Q61233 and FIG. 18B. Transcript sequence LCP1 is shown in FIG. 18C.

In embodiments of the invention, the biomarker signature may additionally comprise one or more of the following biomarkers, or transcript encoding the biomarker: ADAM22, ANP32C, ANXA2P1, APBB2, ATP10B, BCL2L14, CBWD1, CNIH3, CR2, DENND3, DHX15, DSTN, EIF3H, EPHA5, GADD45B, GLP1R, GLRA3, GPR27, HEXIM1, HIST1H3J, HPS5, IL17B, IQCA1, KLHDC2, LCP1, LMAN1L, LOC642131, LRRC37A4, MC2R, MRPL46, MYCNOS, MYO1E, NDUFAF1, PARP4, PDE4B, PDZRN3, PFDN5, PSG11, RFPL3S, RGS4, RHBG, RPL13A, RTF1, SHOX2, SLC11A1, SLC5A6, STS, SYNC, TBC1D1, and TCEA2.

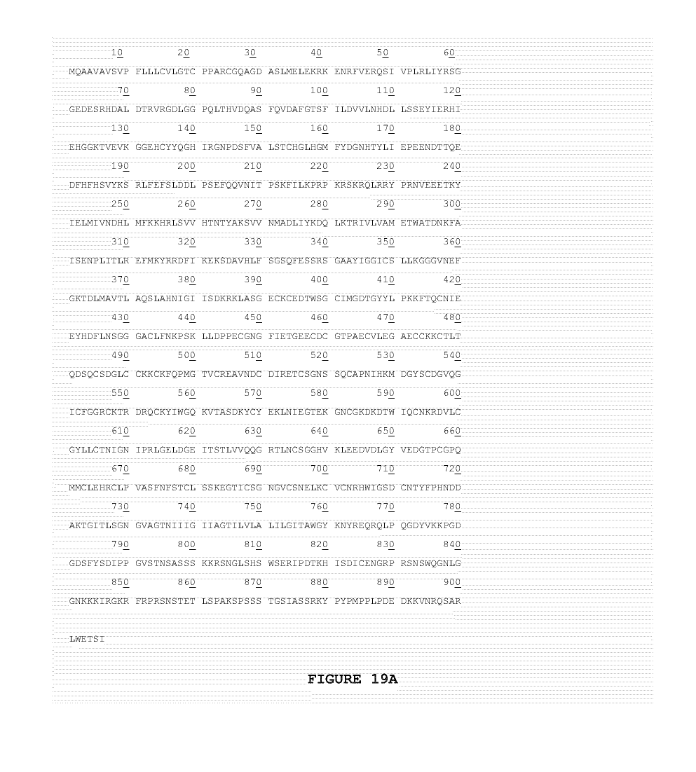

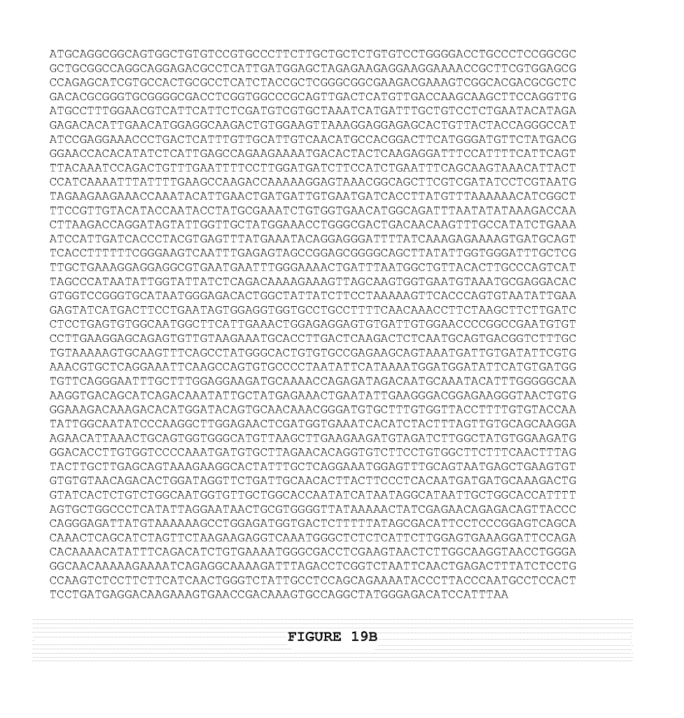

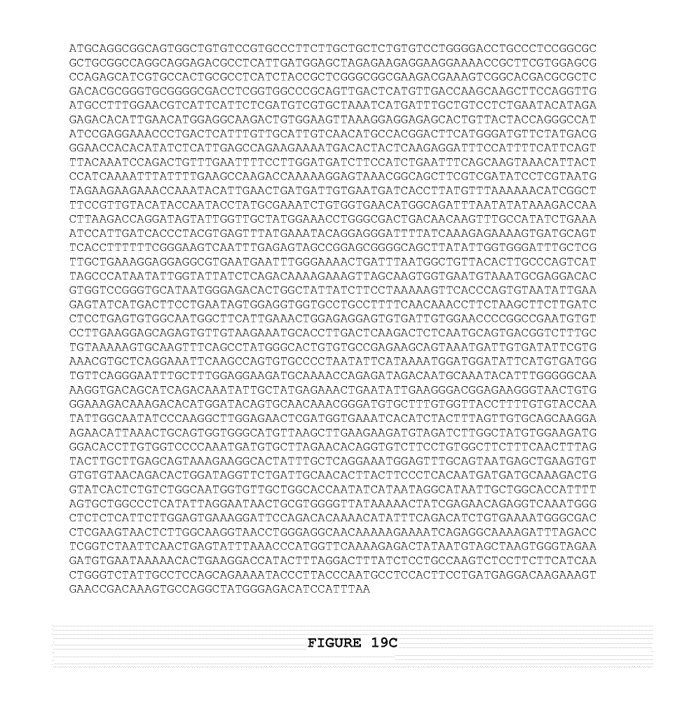

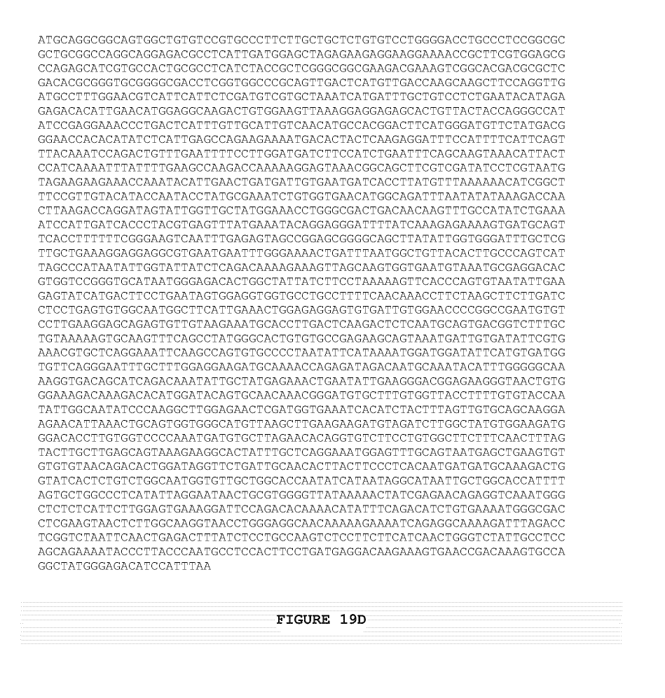

Disintegrin and metalloproteinase domain-containing protein 22, also known as ADAM22, is used herein to encompass both human ADAM22 as shown in FIG. 19A, including all isoforms, such as isoform 2 including an insert at position 859 and in which residues 768-803 are missing, isoform 3 in which residues 860-906 are missing, isoform 4 in which residues 768-803 and residues 860-906 are missing and isoform 5 in which residues 768-803 are missing; and functionally equivalent variants thereof, such as the variant in which residue 81 is arginine, the variant in which residue 119 is tryptophan, and the variant in which residue 207 is isoleucine, as well as non-human forms thereof. Transcript sequences for isoforms 1-4 are shown in FIG. 19B-E.

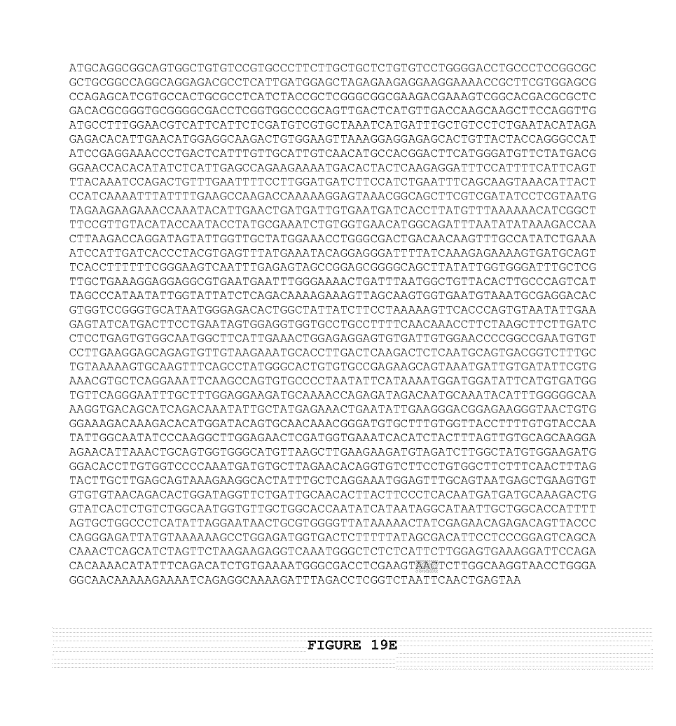

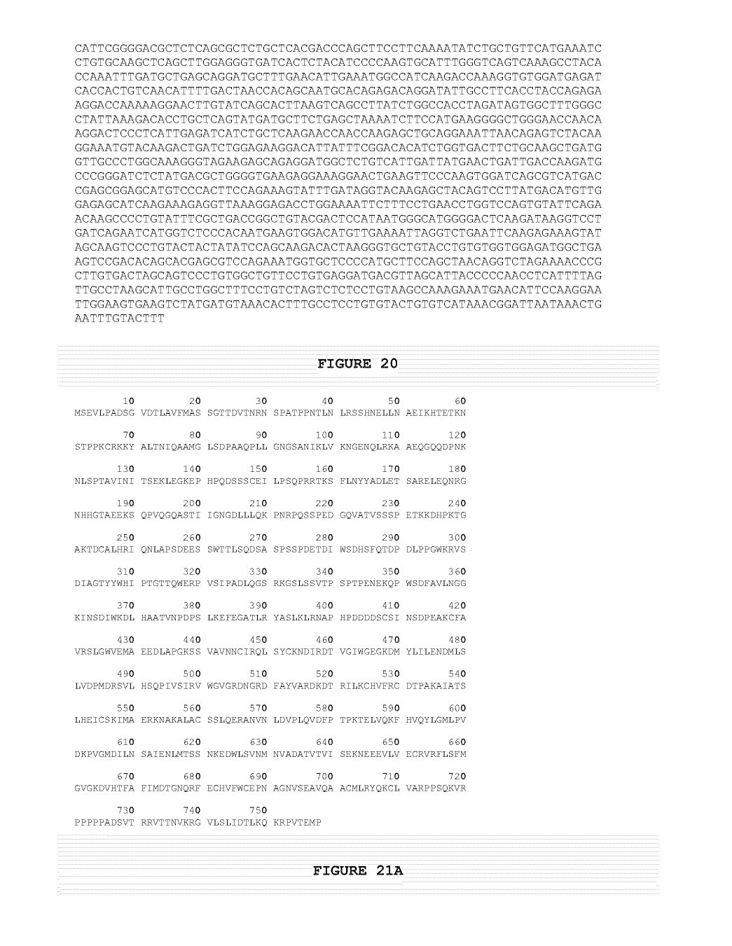

Annexin A2 pseudogene 1, also referred to as ANXA2P1, does not encode a protein, and its in vivo function is currently unknown. The gene sequence of ANXA2P1 is shown in FIG. 20.

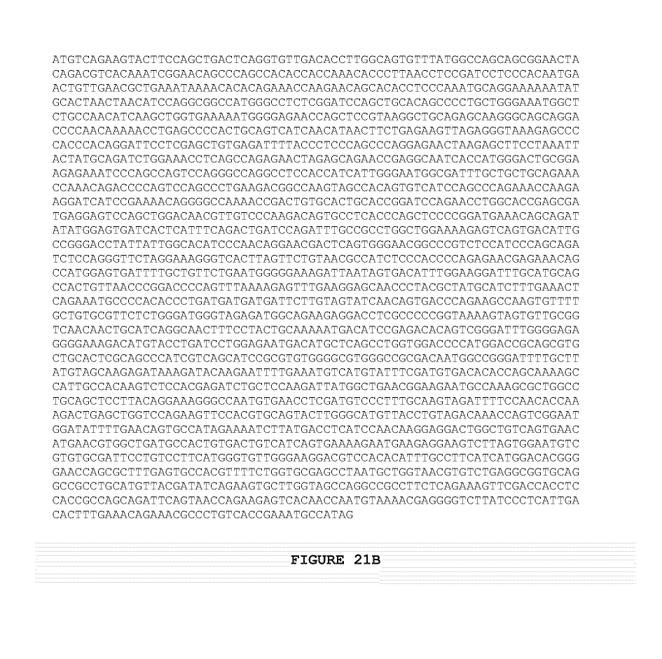

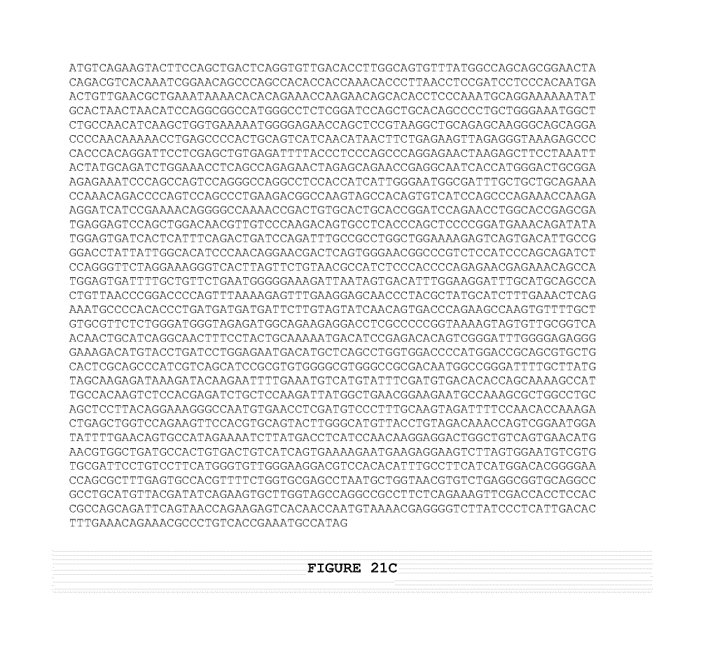

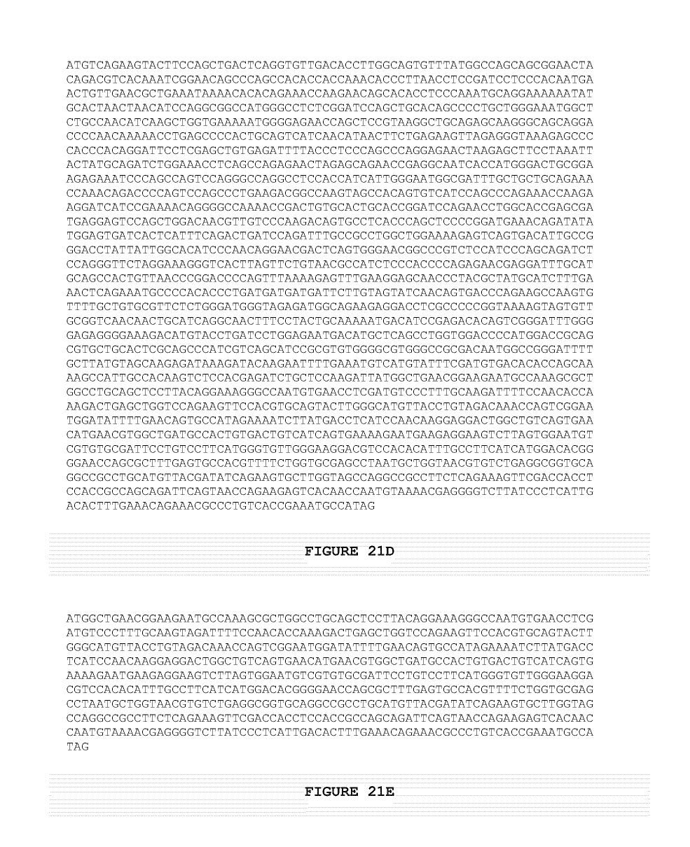

Amyloid beta A4 precursor protein-binding family B member 2, also known as APBB2, is used herein to encompass both human APBB2 as shown in FIG. 21A, including all isoforms such as isoform B in which residues 348-368 and residues 577 are missing and isoform C in which residues 1-548 are missing, as well as functionally equivalent variants thereof such as non-human forms thereof. Transcript sequences for isoforms A-D are shown in FIG. 21B-E.

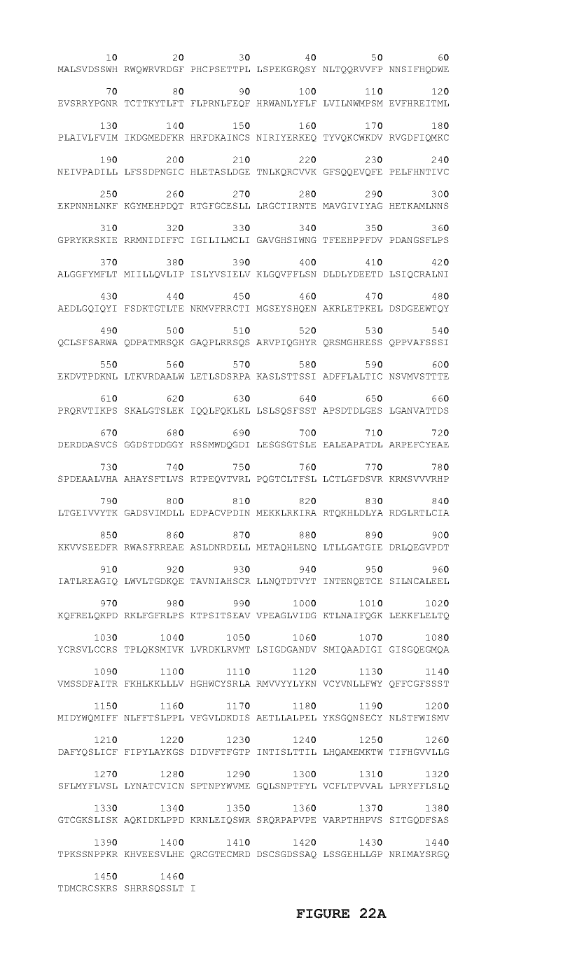

ATPase, class V, type 10B, also known as ATP10B, is used herein to encompass both human ATP10B as shown in FIG. 22A, including all isoforms, such as isoform B and isoform C, and functionally equivalent variants thereof, such as the variant in which residue 271 is arginine, as well as non-human forms thereof. Transcript sequence for ATP10B is shown in FIG. 22B.

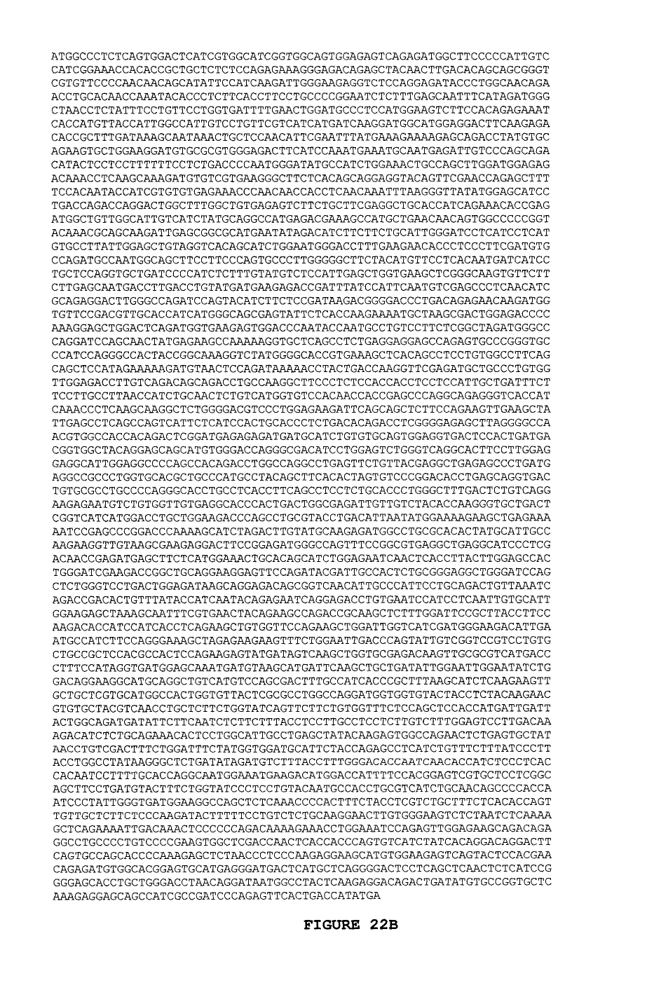

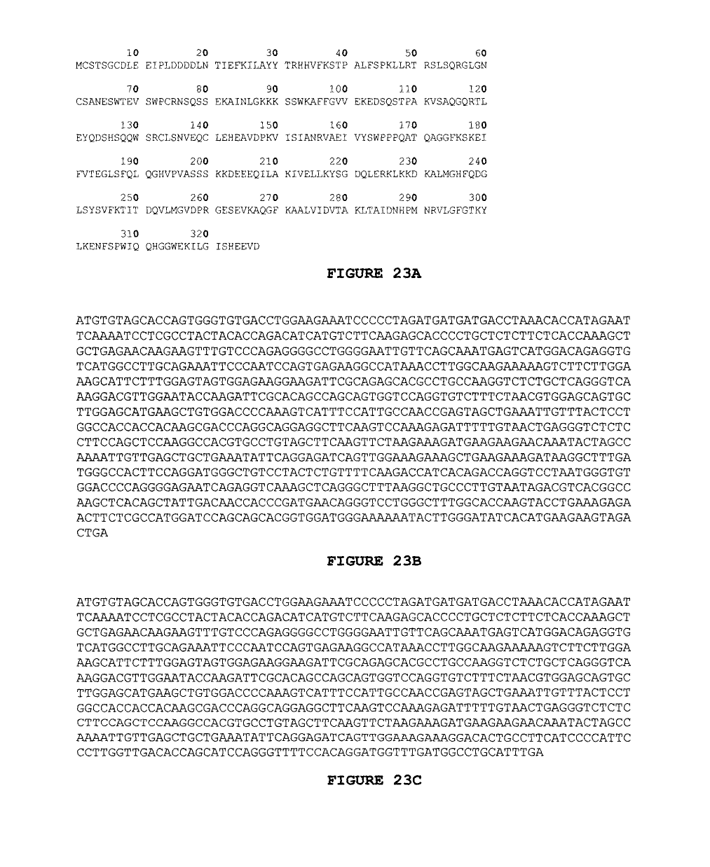

Apoptosis facilitator Bc1-2-like protein 14, or BCL2L14, is used herein to encompass both human BCL2L14 as shown in FIG. 23A, including all isoforms, such as isoform 2 (Uniprot identifier Q9BZR8-2) and isoform 3 (Uniprot identifier Q9BZR8-3), and functionally equivalent variants thereof, for example, modified and non-human forms thereof. Transcript sequences for isoforms 1 and 2 are shown in FIGS. 23B and C.

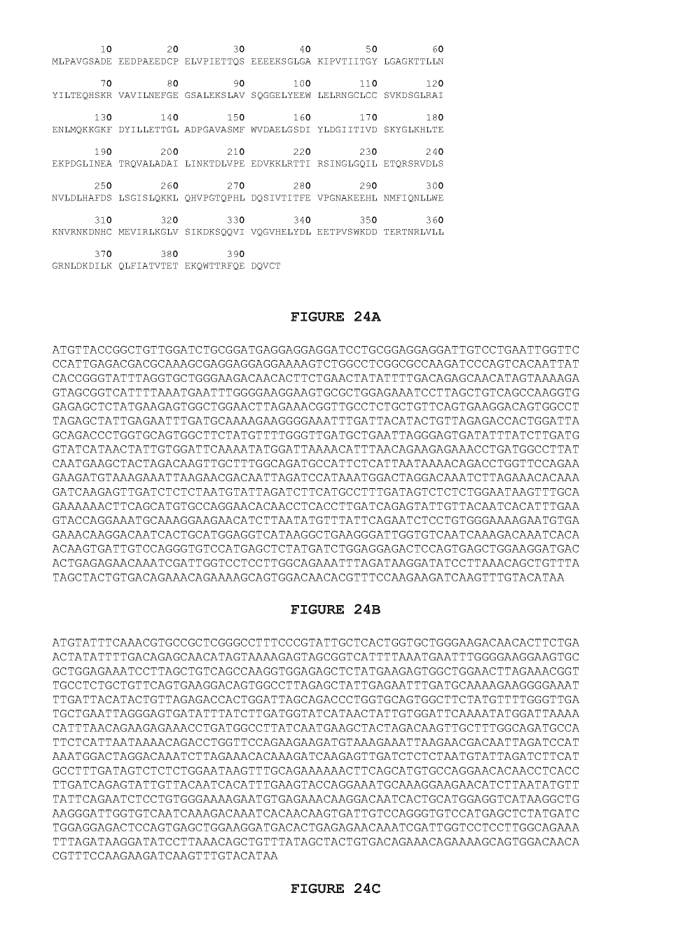

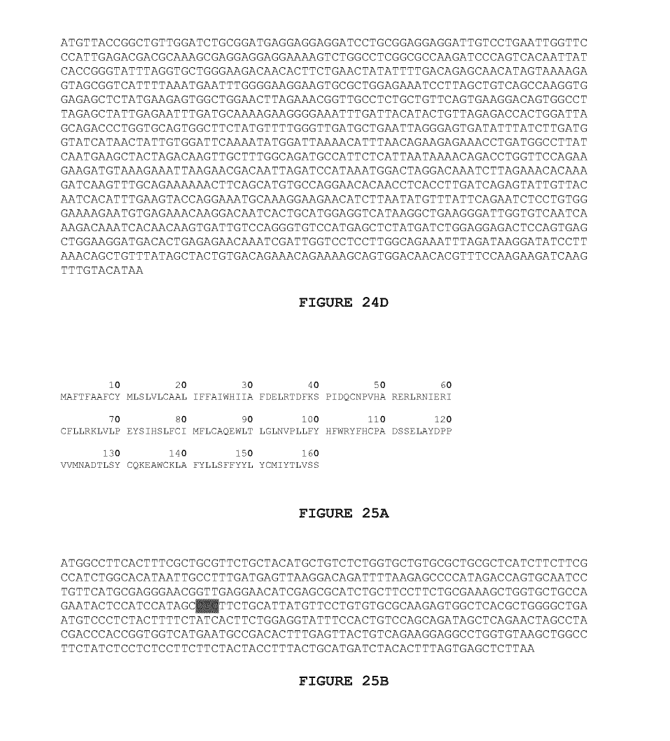

COBW domain-containing protein 1, or CBWD1, is used herein to encompass both human CBWD1 as shown in FIG. 24A, including all isoforms such as isoform 2, isoform 3 in which residues 236-254 are missing and isoform 4 in which residues 114-395 are missing, as well as functionally equivalent variants, such as the variant in which residue 8 is valine, and non-human variants thereof. Transcript sequences for isoforms 1-3 are shown in FIG. 24B-D.

Cornichon homolog 3, also referred to as CNIH3, is used herein to encompass both human CN1H3 as shown in FIG. 25A, including functionally equivalent variants thereof, such as all isoforms and non-human variants thereof. Transcript sequence for CNIH3 is shown in FIG. 25B.

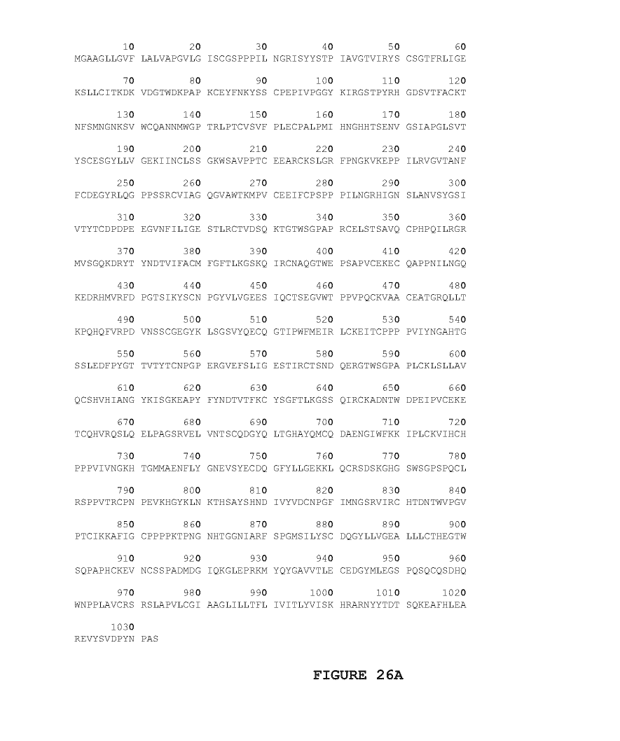

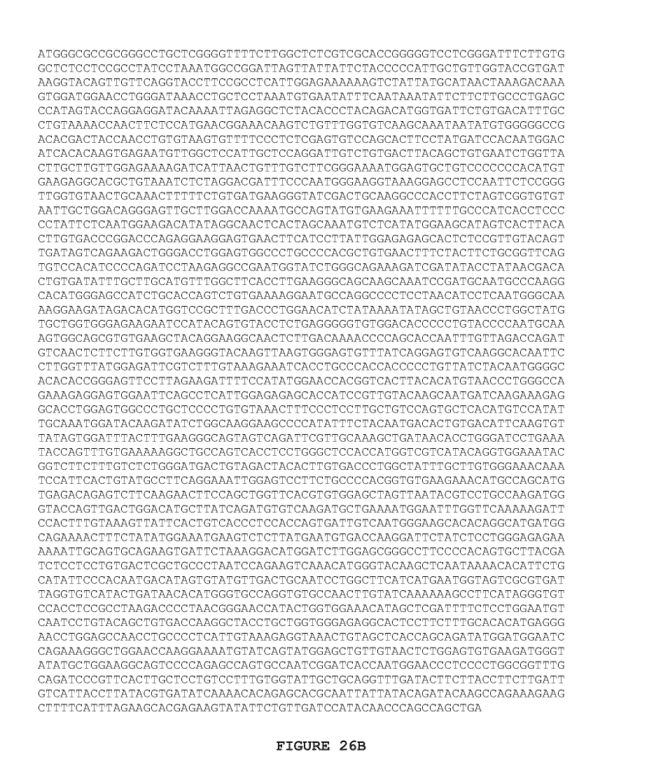

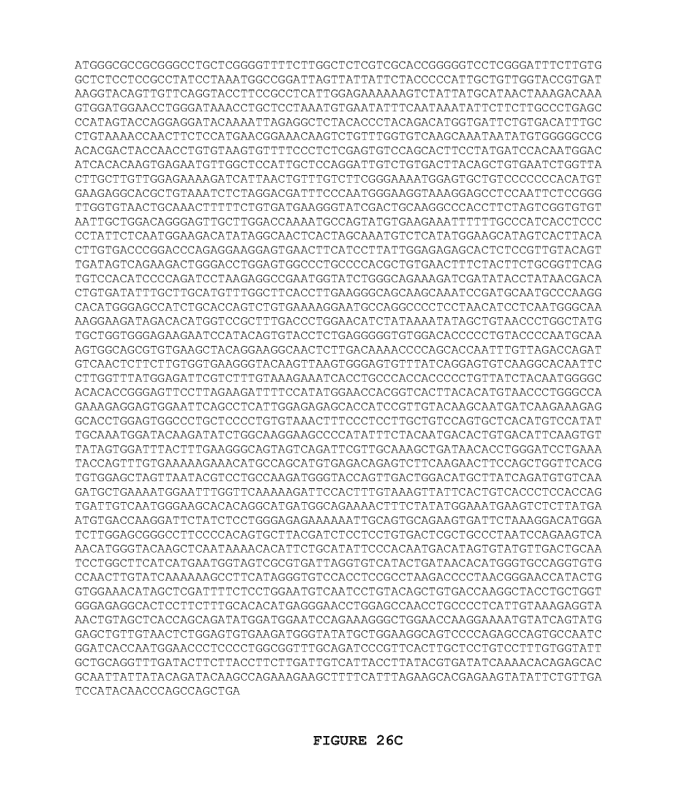

Complement receptor 2, also referred to as CR2, is used herein to encompass both human CR2 as shown in FIG. 26A, including functionally equivalent variants thereof, such as all isoforms, including isoforms 2-4, variants such as the variant in which residues 639 is asparagine, the variant in which residue 993 is valine and the variant in which residue 1003 is glutamic acid, and non-human variants thereof. Transcript sequences for isoforms 1 and 2 are shown in FIGS. 26B and C.

DENN domain-containing protein 3, or DENND3, is used herein to encompass both human DENND3 as shown in FIG. 27A, including functionally equivalent variants thereof, such as isoform 2 in which residues 319-370 are missing and isoforms 3 and 4, variants such as the variant in which residue 143 is asparagine and the variant in which residue 364 is arginine, and non-human variants thereof. Transcript sequence for DENND3 is shown in FIG. 27B.

DHX15 is a putative pre-mRNA-splicing factor ATP-dependent RNA helicase. As used herein, DHX15 is meant to encompass human DHX15 as shown in FIG. 28A, including functionally equivalent variants thereof, such as isoforms thereof, variants and non-human variants thereof. Transcript sequence for DHX15 is shown in FIG. 28B.

Eukaryotic translation initiation factor 3 subunit H, also referred to as EIF3H is a protein that in humans is encoded by the EIF3H gene. As used herein, EIF3H is meant to encompass human EIF3H as shown in FIG. 29A, including functionally equivalent variants thereof, such as isoforms thereof, naturally occurring variants and non-human variants thereof. Transcript sequence for EIF3H is shown in FIG. 29B.

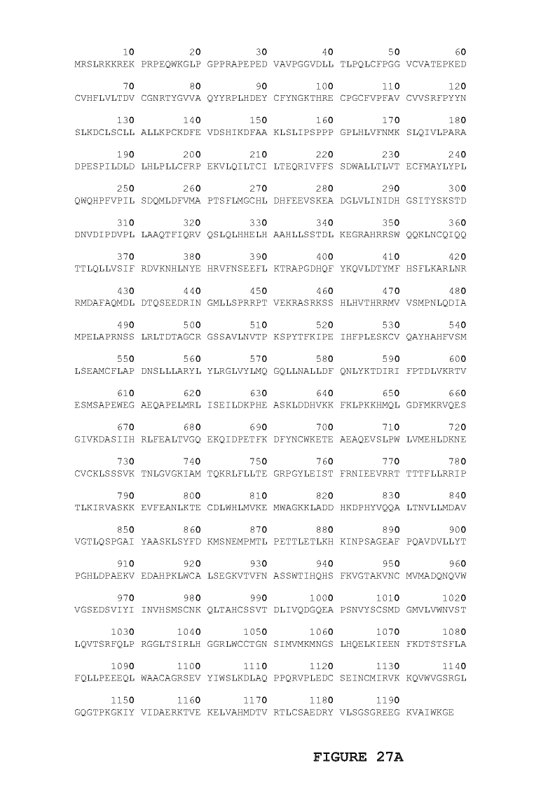

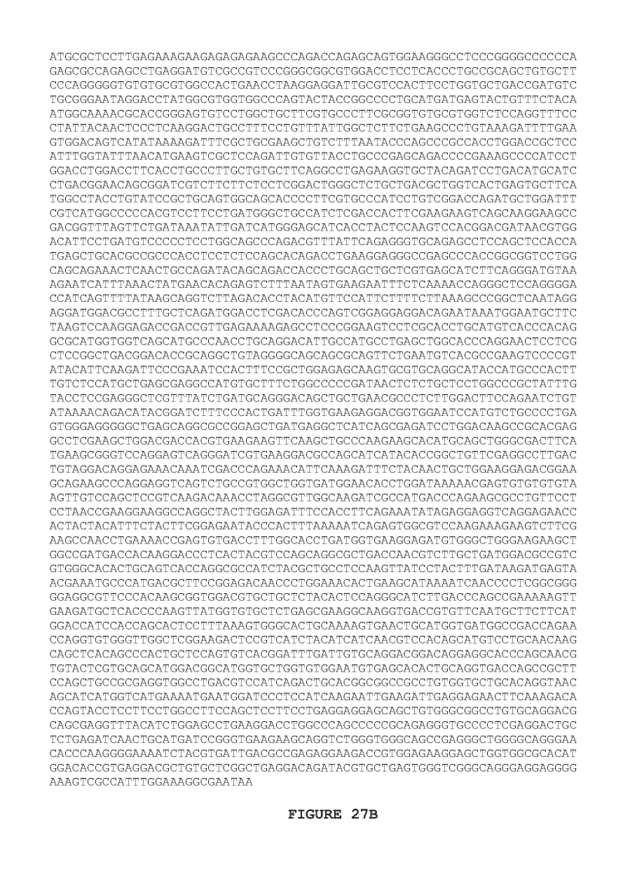

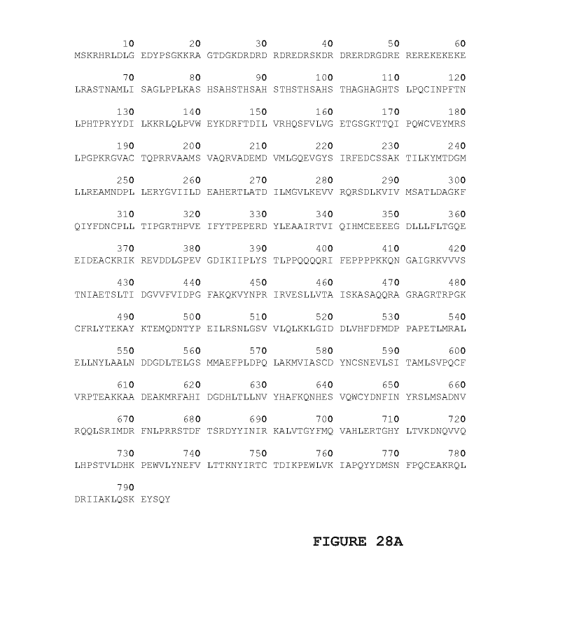

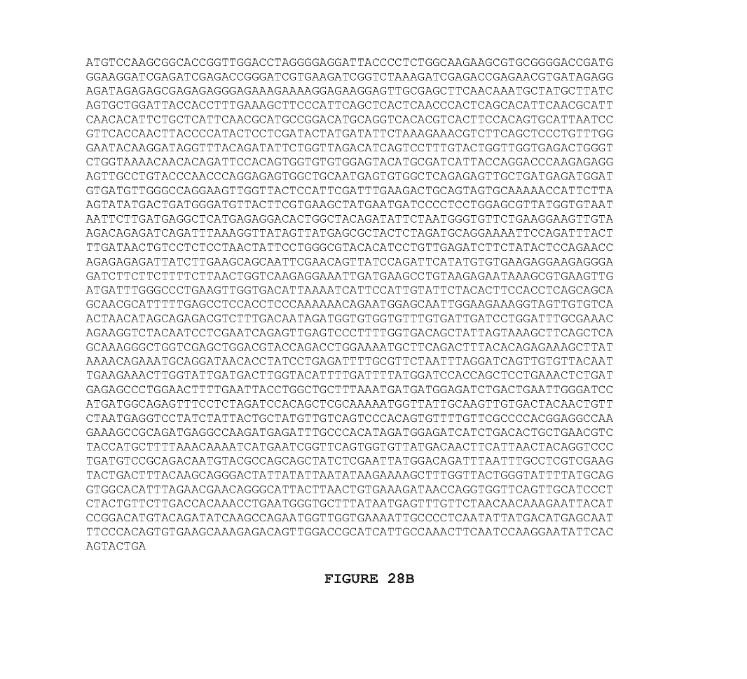

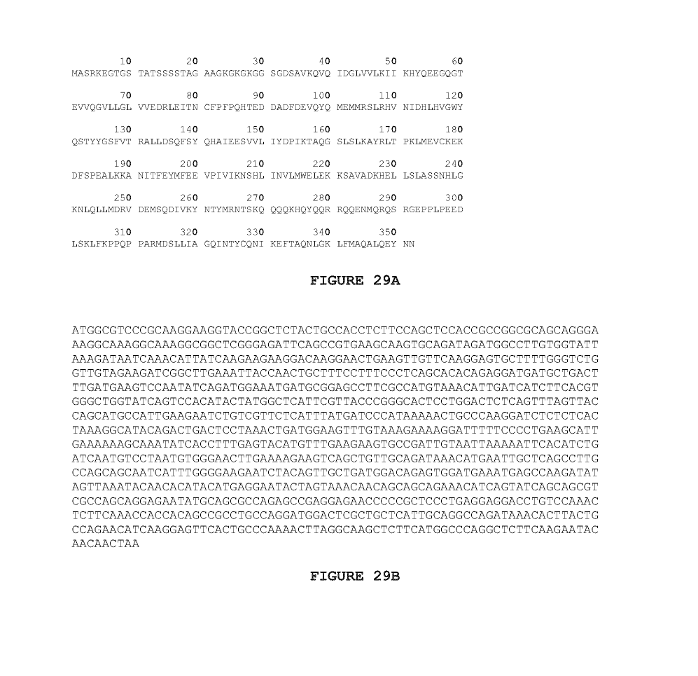

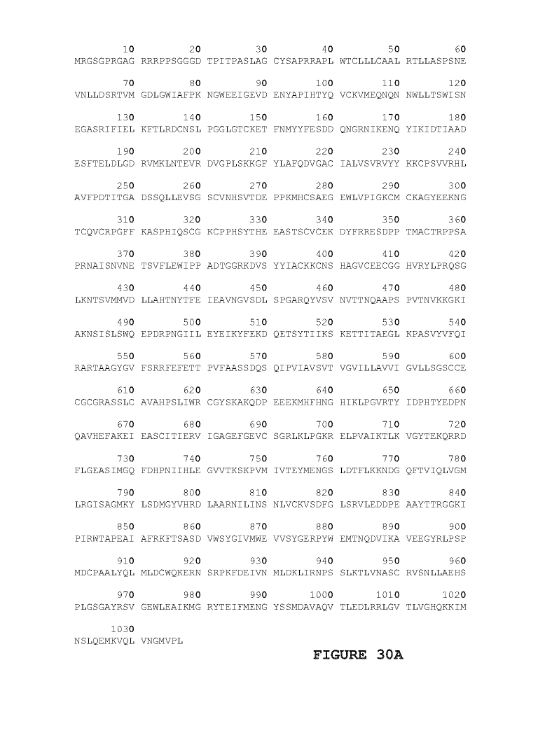

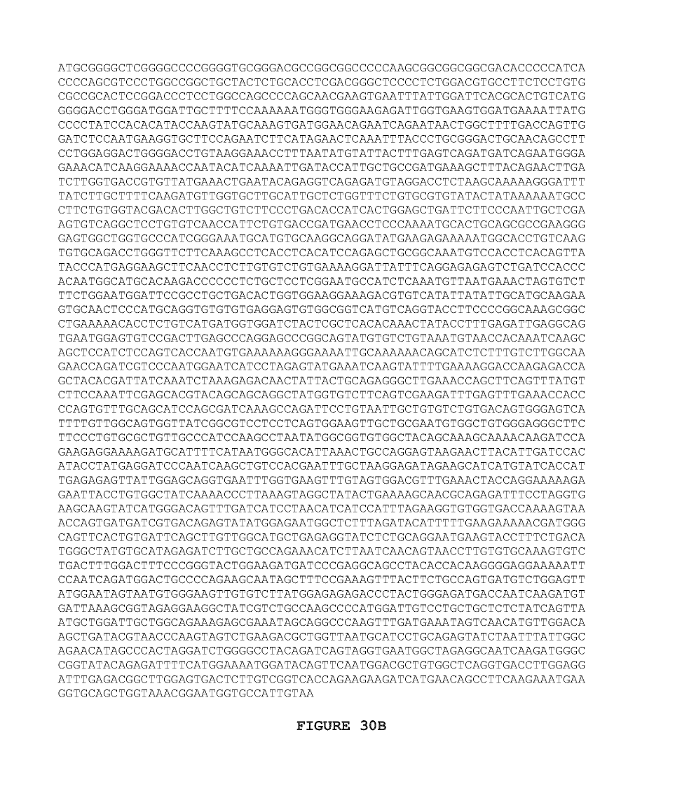

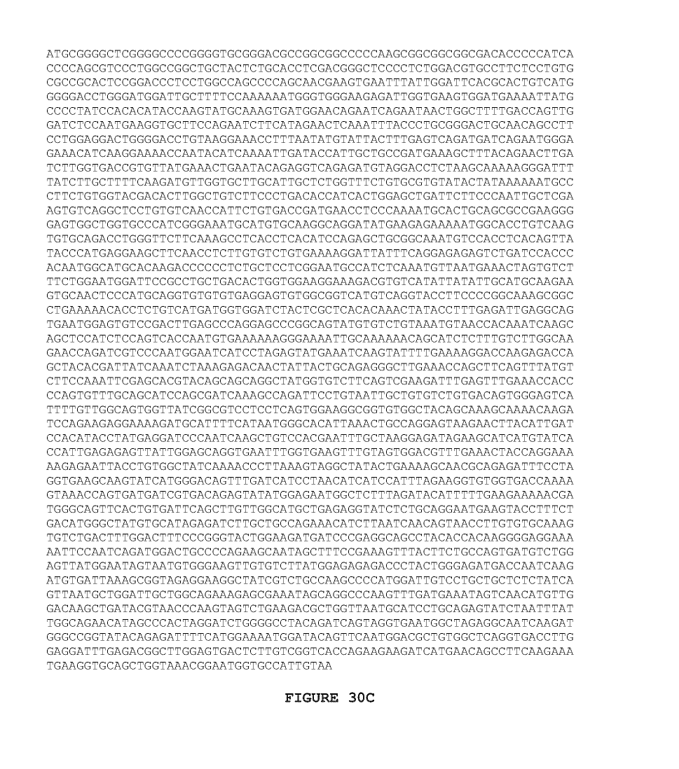

EPH receptor A5 (ephrin type-A receptor 5), or EPHA5, is a receptor tyrosine kinase. The term “EPHA5” is used herein to encompass both human EPHA5 as shown in FIG. 30A, including functionally equivalent variants thereof, such as all isoforms, including isoform 2 in which residues 597-619 of isoform are replaced with arginine and isoform in which residues 1-69 are missing and residue 563 is replaced by serine-valine, and variants including the variant in which residue 81 is threonine, the variant in which residue 231 is alanine and the variant in which residue 503 is lysine, and non-human variants thereof. Transcript sequences for isoforms A and B are shown in FIGS. 30B and C.

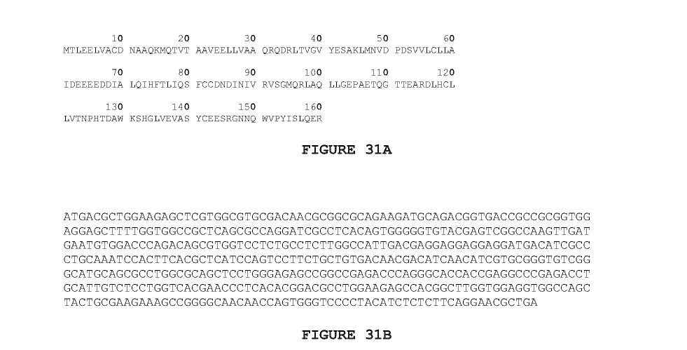

GADD45B or Growth arrest and DNA-damage-inducible, beta, refers herein to human GADD45B as shown in FIG. 31A, including functionally equivalent variants thereof, such as all isoforms thereof, naturally occurring variants thereof, and non-human variants thereof. Transcript sequence for GADD45B is shown in FIG. 31B.

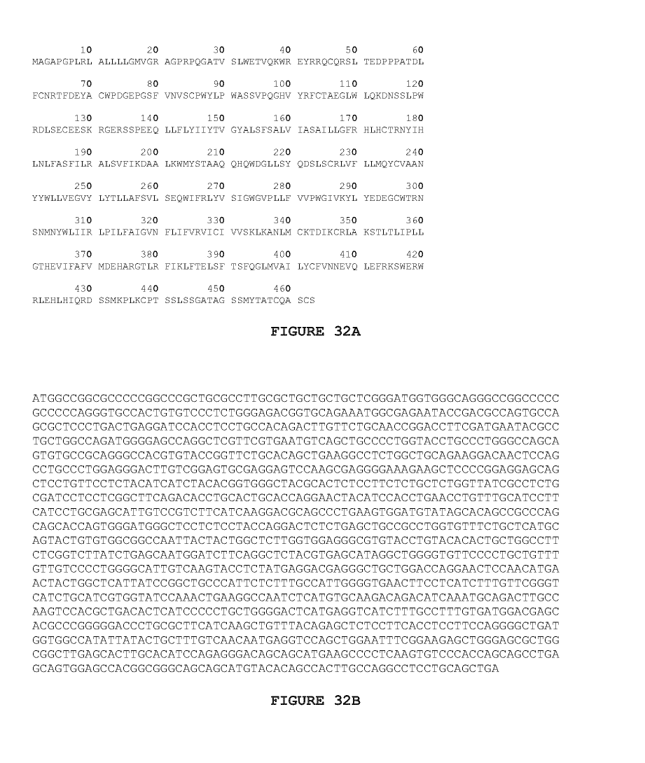

Glucagon-like peptide 1 receptor (GLP1R) refers herein to human GADD45B as shown in FIG. 32A, including functionally equivalent variants thereof, such as all isoforms, and variants including the variant in which residue 20 is lysine, the variant in which residue 44 is histidine and the variant in which residue 333 is cysteine, and non-human variants thereof. Transcript sequence for GLP1R is shown in FIG. 32B.

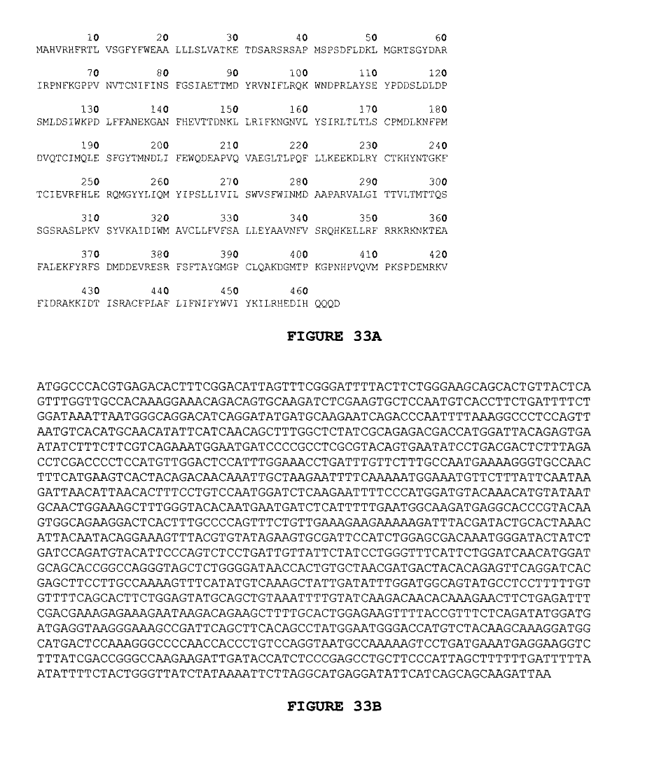

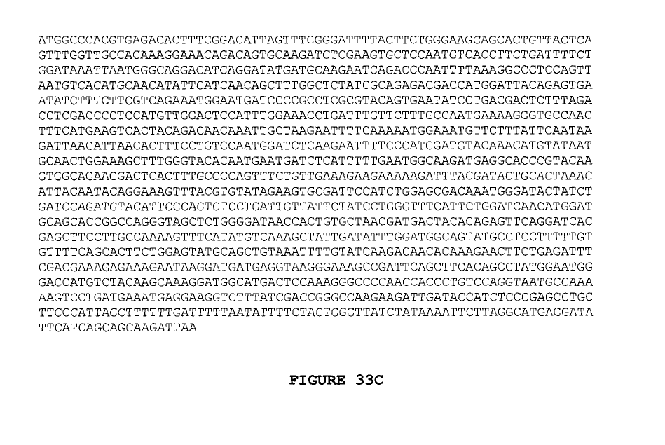

Glycine receptor subunit alpha-3, also known as GLRA3, refers herein to human GLRA3 as shown in FIG. 33A, including functionally equivalent variants thereof, such as all isoforms including isoform alpha-3K in which residues 358-372 are missing, naturally occurring variants, and non-human variants thereof. Transcript sequences for GLRA3 isoforms A and B are shown in FIGS. 33B and C.

HEXIM1, also referred to as Hexamethylene bis-acetamide-inducible protein 1, is meant to encompass human HEXIM1 as shown in FIG. 34A, including functionally equivalent variants thereof, such as all isoforms including isoform alpha-3K in which residues 358-372 are missing, naturally occurring variants, and non-human variants thereof. Transcript sequence for HEXIM1 is shown in FIG. 34B.

HIST1H3J is a gene that encodes the Histone H3.1 protein, and is meant to encompass the gene that encodes the human protein as shown in FIG. 35A, as well as functionally equivalent proteins thereof, including isoforms or naturally occurring variants thereof, as well as non-human variants. Transcript sequence for HIST1H3J is shown in FIG. 35B.

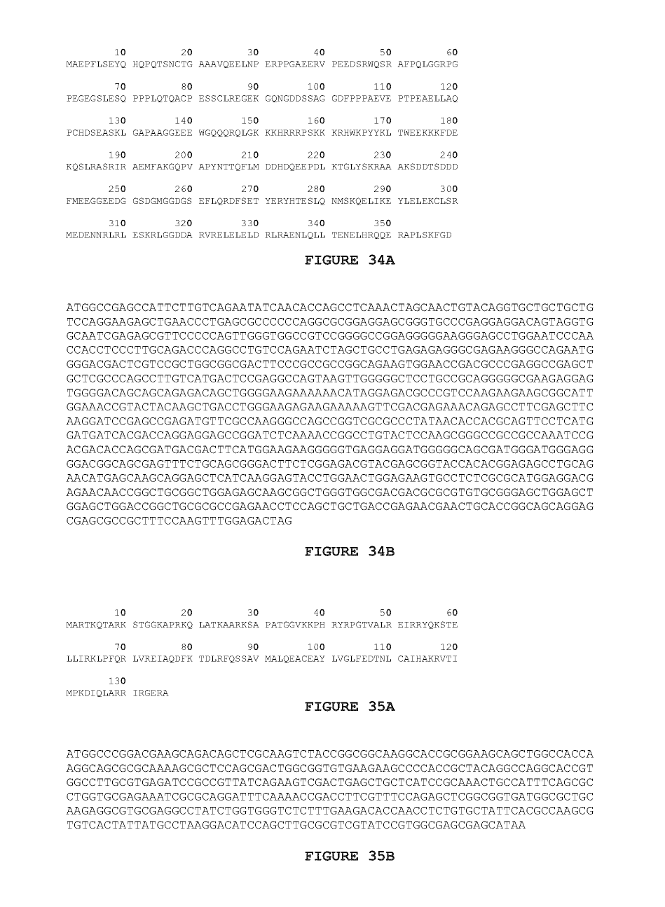

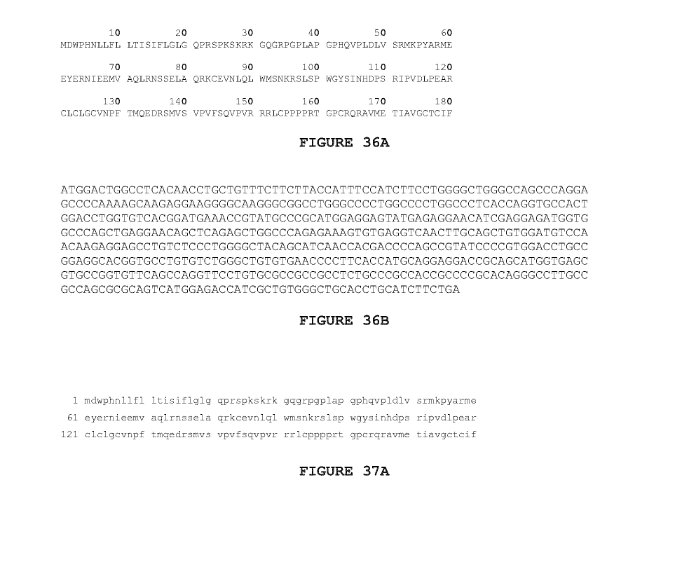

IL17B interleukin 17B, or IL17B, refers to human IL17B, as shown in FIG. 36A, including functionally equivalent isoforms and variants thereof, as well as non-human variants thereof. Transcript sequence for IL17B is shown in FIG. 36B.

IQ motif containing with AAA domain 1 (IQCA1), refers to human IQCA1, as shown in FIG. 37A, including functionally equivalent isoforms and variants thereof, as well as non-human variants thereof. Transcript sequence for IQCA1 is shown in FIG. 37B.

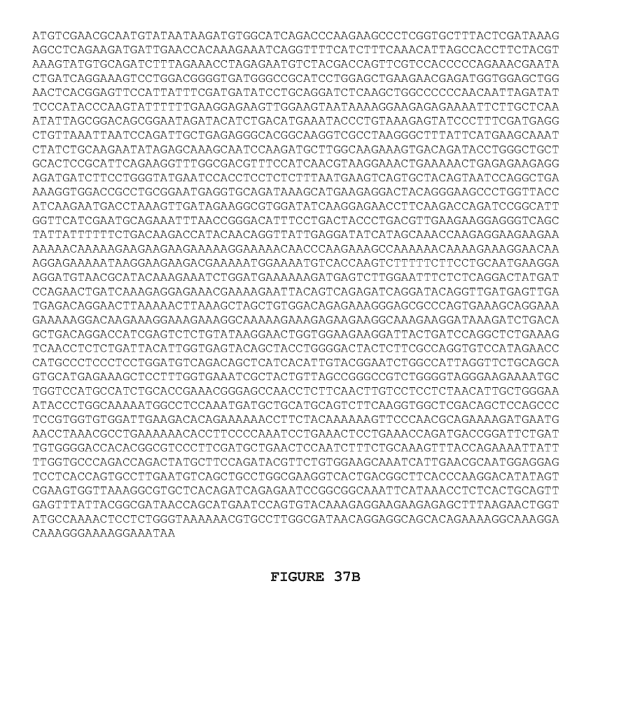

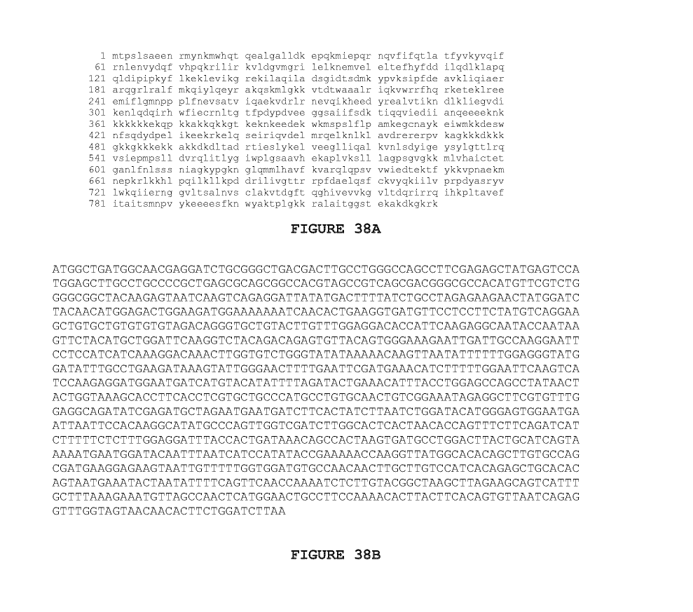

Kelch domain containing 2 (KLHDC2) refers to human KLHDC2, as shown in FIG. 38A, including functionally equivalent isoforms and variants thereof, as well as non-human variants thereof. Transcript sequence for KLHDC2 is shown in FIG. 38B.

Lectin, mannose-binding, 1 like (LMAN1L) refers to human LMAN1L, as shown in FIG. 39A, including functionally equivalent isoforms and variants thereof, such as the variant in which residue 105 is glutamic acid and the variant in which the residue at position 517 is serine, as well as non-human variants thereof. Transcript sequence for LMAN1L is shown in FIG. 39B.

LOC642131 refers to a protein, including human LOC642131 as shown in FIG. 40A, and functionally equivalent isoforms including variants thereof, as well as non-human variants thereof. Transcript sequence for LOC642131 is shown in FIG. 40B.

Leucine rich repeat containing 37, member A4, or LRRC37A4, refers to human LRRC37A4, as shown in FIG. 41A, including functionally equivalent isoforms and variants thereof, as well as non-human variants thereof. Transcript sequence for LRRC37A4 is shown in FIG. 41B.

Mitochondrial ribosomal protein L46, or MRPL46, refers to human LRRC37A4, as shown in FIG. 42A, including functionally equivalent isoforms and variants thereof, as well as non-human variants thereof. Transcript sequence for MRPL46 is shown in FIG. 42B.

N-myc oncogene, or MYCNOS, encompasses the human gene that encodes the N-cym human protein as shown in FIG. 43A, as well as related genes that encode functionally equivalent isoforms, variants and non-human equivalent proteins. Transcript sequence for MYCNOS is shown in FIG. 43B.

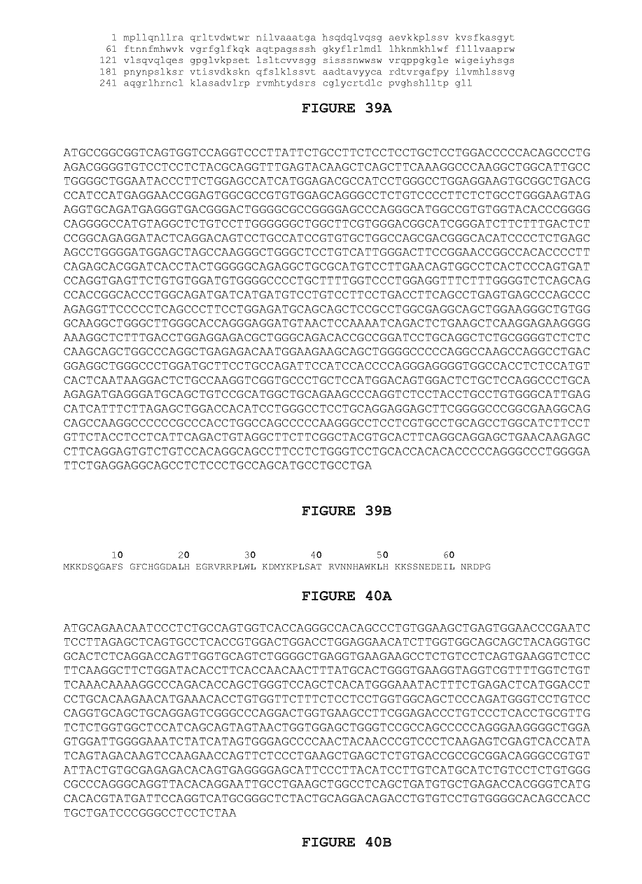

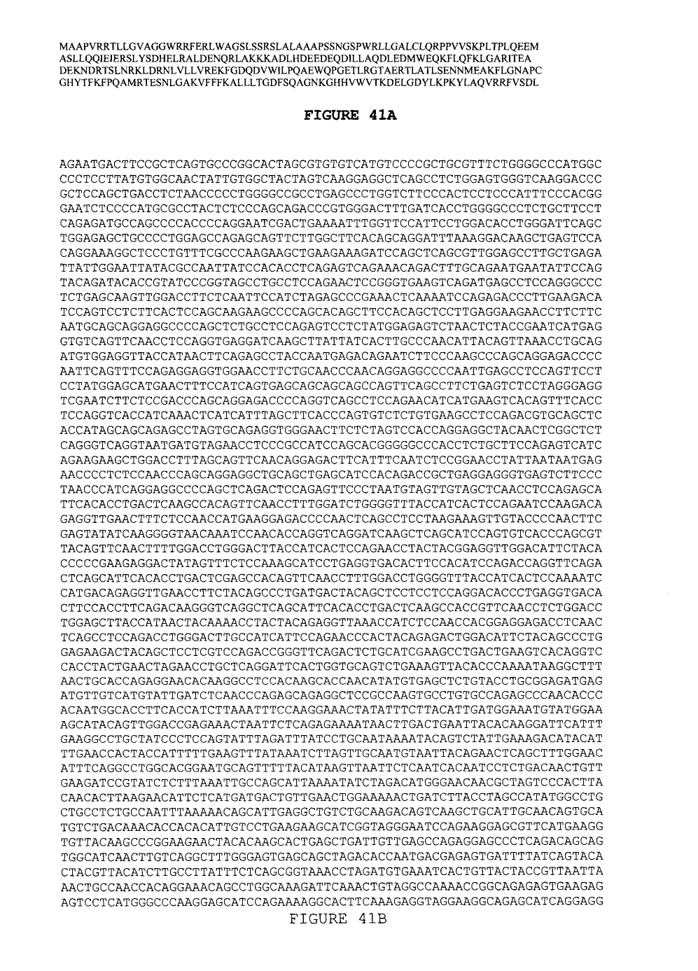

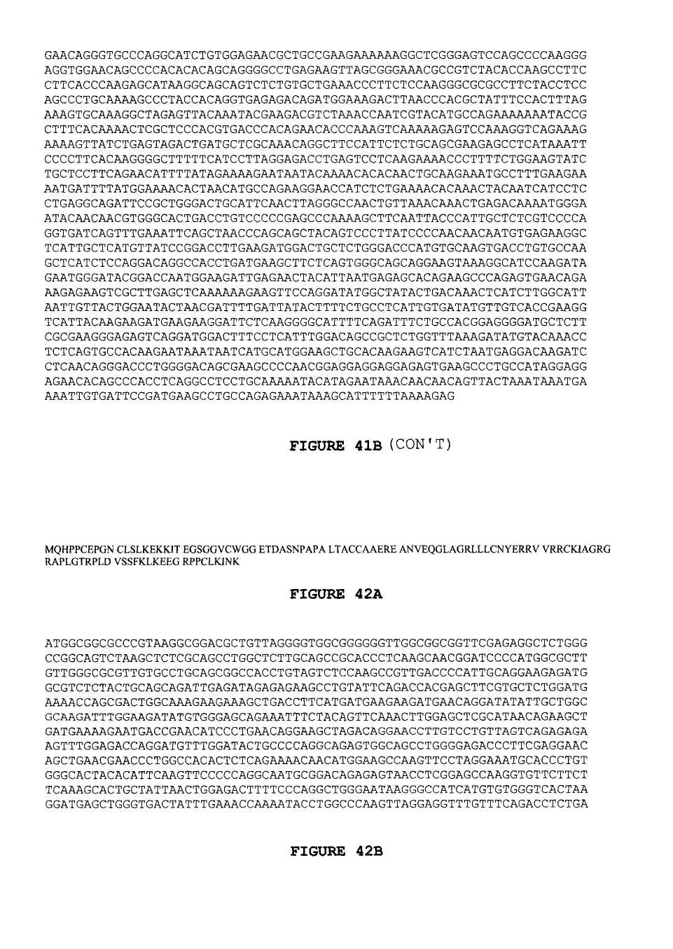

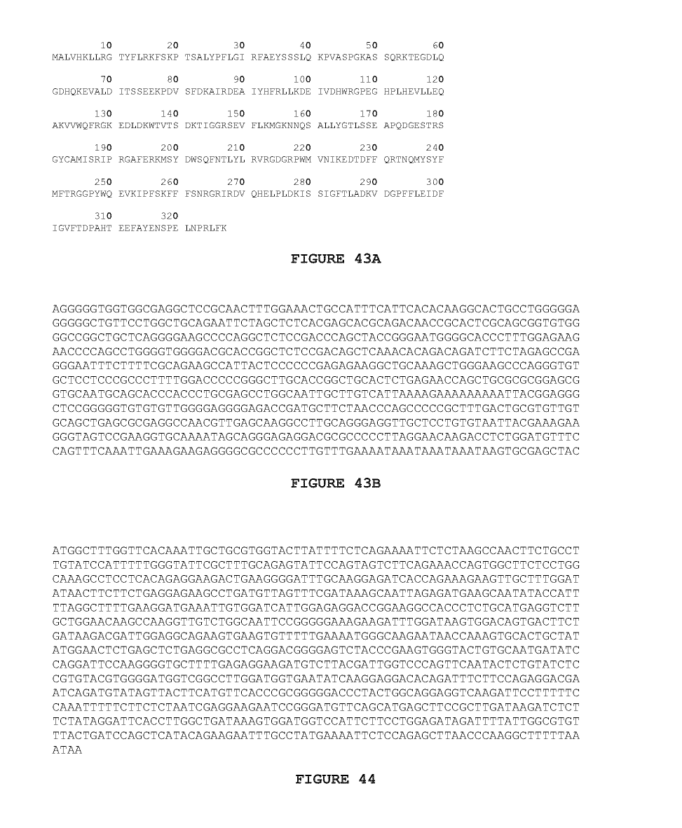

NADH dehydrogenase (ubiquinone) complex I, assembly factor 1, or NDUFAF1, is used herein to refer to human NDUFAF1 as shown in FIG. 44A, including functionally equivalent isoforms and variants thereof, such as the variant in which the residue at position 9 is histidine and the variant in which the residue at position 314 is glycine, as well as non-human variants thereof. Transcript sequence for NDUFAF1 is shown in FIG. 44B.

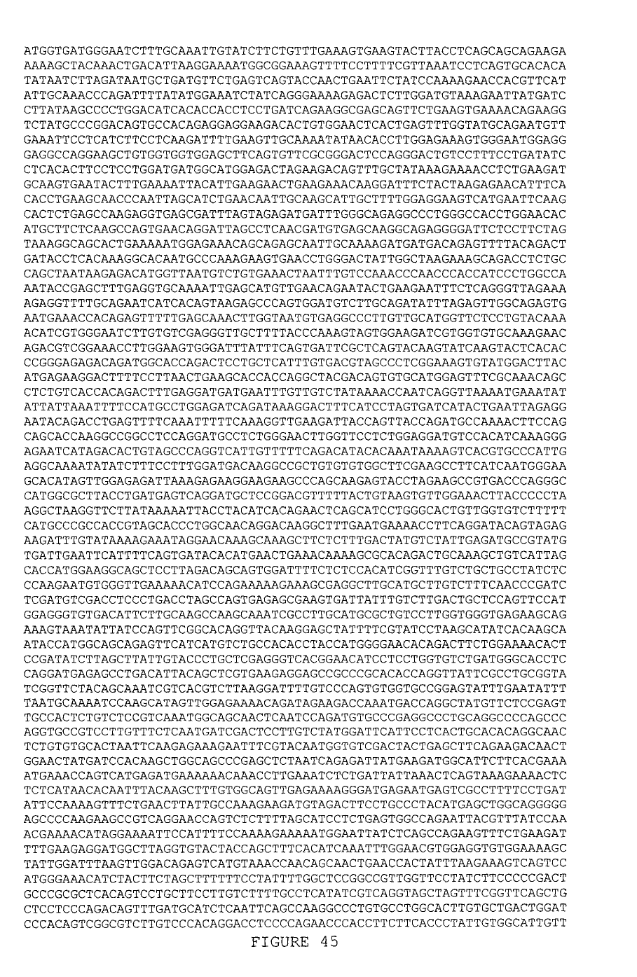

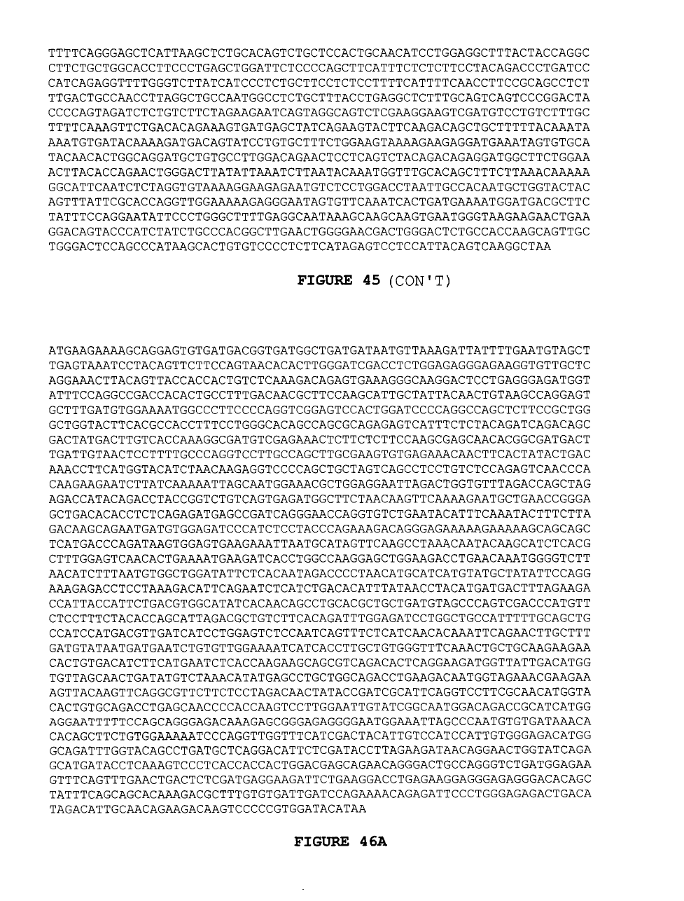

Poly [ADP-ribose] polymerase 4, or PARP4, is used herein to encompass human PARP4, the mRNA transcript for which is shown in FIG. 45A, as well as functionally equivalent isoforms and variants thereof, and non-human variants thereof. Transcript sequence for PARP4 is shown in FIG. 45B.

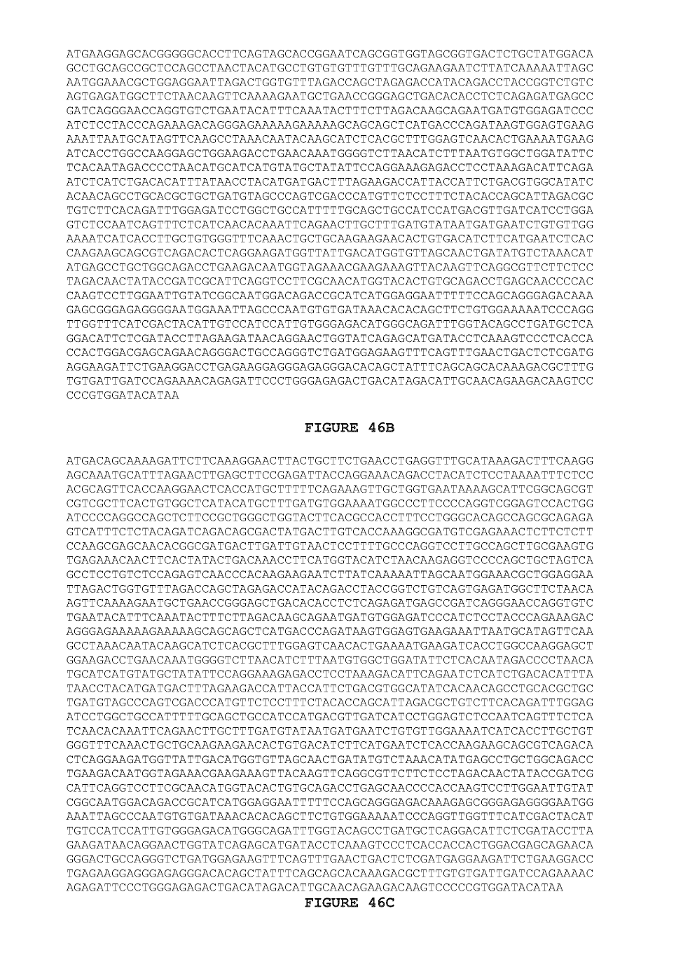

cAMP-specific 3′,5′-cyclic phosphodiesterase 4B, or PDE4B, is used herein to encompass human PDE4B, the mRNA transcript for which is shown in FIG. 46A, as well as functionally equivalent isoforms thereof, e.g. isoforms 2 and 3, the mRNA transcript for which is shown in FIGS. 46B and C, variants thereof, and non-human variants thereof.

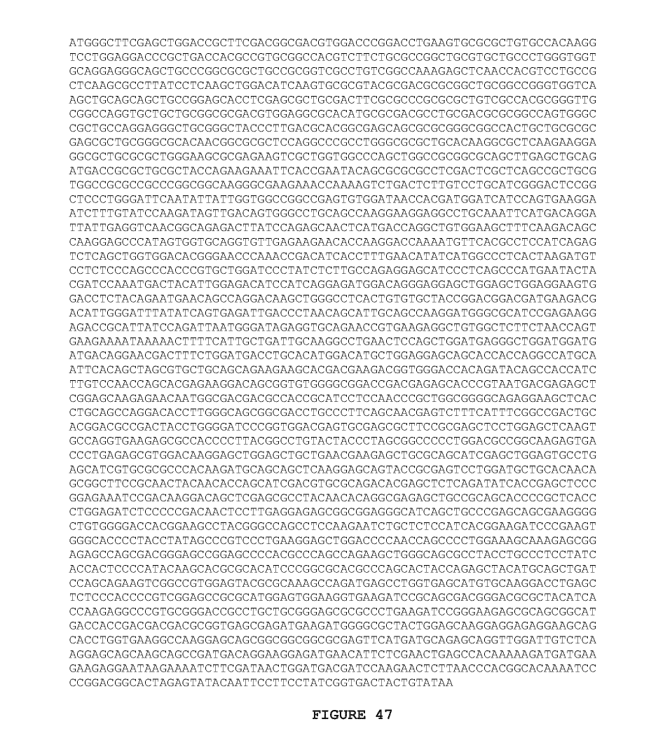

PDZ domain-containing RING finger protein 3, or PDZRN3, is used herein to encompass human PDZRN3, the mRNA transcript for which is shown in FIG. 47, as well as functionally equivalent isoforms thereof, variants thereof, and non-human variants thereof.

Prefoldin subunit 5, or PFDN5, is used herein to encompass human PFDN5, alpha isoform, the mRNA transcript for which is shown in FIG. 48A, as well as functionally equivalent isoforms thereof, e.g. isoform gamma, the mRNA transcript for which is shown in FIG. 48B, variants thereof, and non-human variants thereof.

Pregnancy specific beta-1-glycoprotein 11, or PSG11, is used herein to encompass human PSG11, the mRNA transcript for which is shown in FIG. 49A, as well as functionally equivalent isoforms thereof, e.g. isoform 2, the mRNA transcript for which is shown in FIG. 49B, variants thereof, and non-human variants thereof.

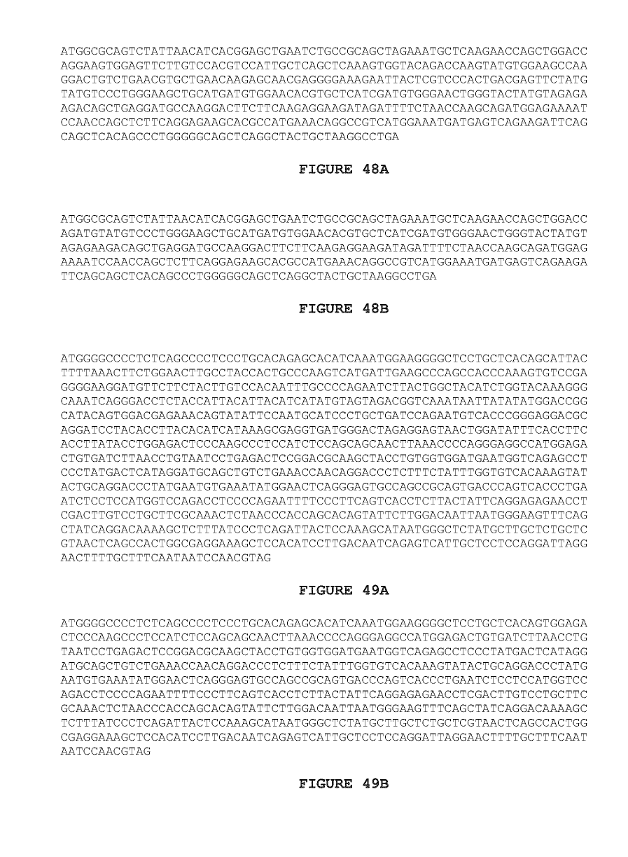

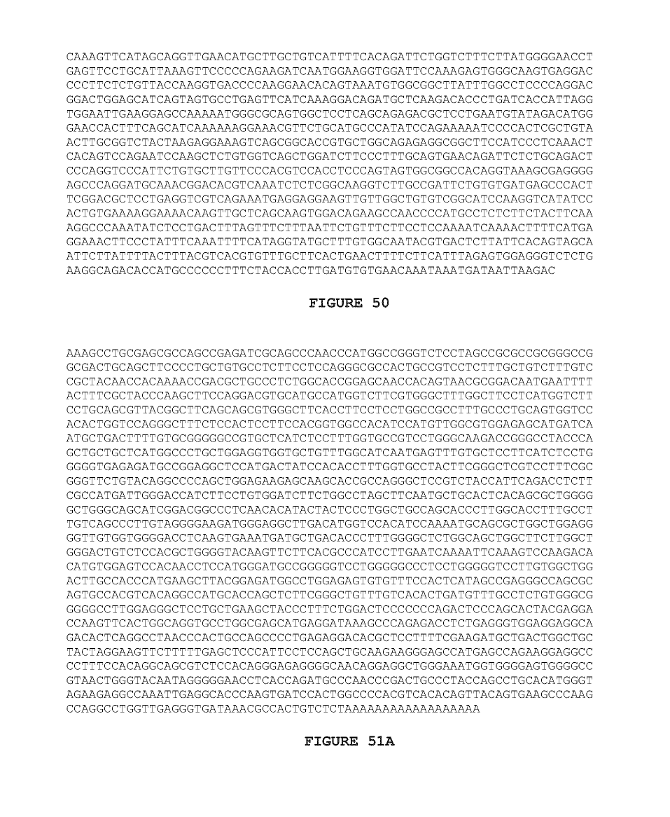

Ret finger protein-like 3, or RFPL3S, is used herein to encompass human RFPL3S, the mRNA transcript for which is shown in FIG. 50, as well as functionally equivalent isoforms thereof, variants thereof, and non-human variants thereof.

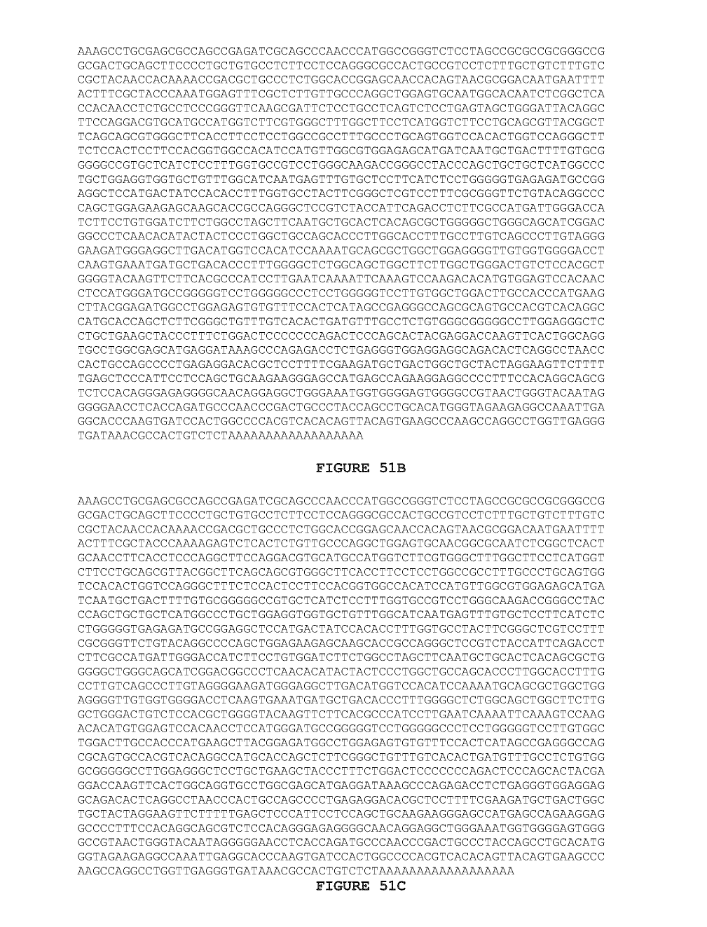

Rh family, B glycoprotein, or RHBG, is used herein to encompass human RHBG, the mRNA transcript for which is shown in FIG. 51A, as well as functionally equivalent isoforms thereof, e.g. isoforms B and C, the mRNA transcripts for which is shown in FIGS. 51B and C, variants thereof, and non-human variants thereof.

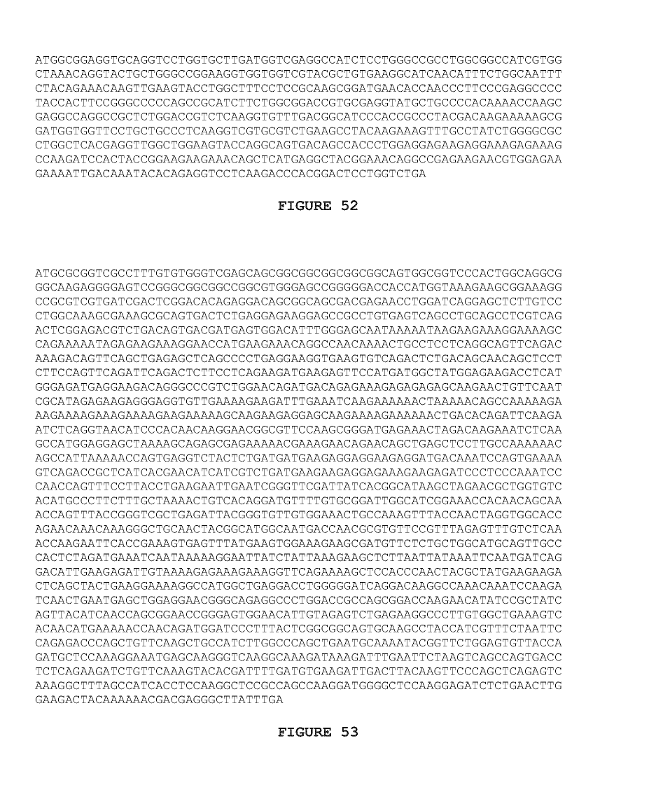

Ribosomal protein L13a, 60 s, also referred to as RPL13A, is used herein to encompass human RPL13A, the mRNA transcript for which is shown in FIG. 52, as well as functionally equivalent isoforms thereof, variants thereof, and non-human variants thereof.

Paf1/RNA polymerase II complex component, homolog, or RTF1, is used herein to encompass human RTF1, the mRNA transcript for which is shown in FIG. 53, as well as functionally equivalent isoforms thereof, variants thereof, and non-human variants thereof.

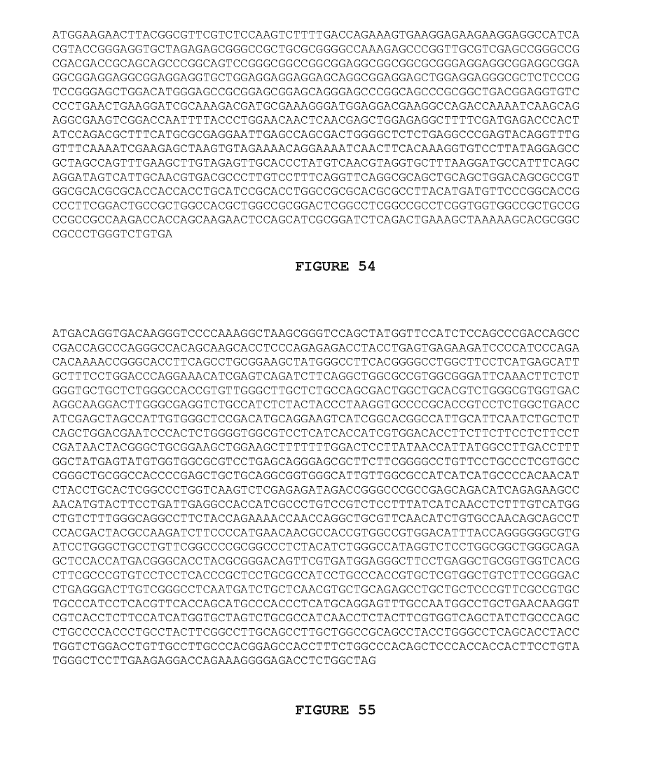

Short stature homeobox 2, or SHOX2, is used herein to encompass human SHOX2, the mRNA transcript for which is shown in FIG. 54, as well as functionally equivalent isoforms thereof, variants thereof, and non-human variants thereof.

Natural resistance-associated macrophage protein 1, or SLC11A1, is used herein to encompass human SLC11A1, the mRNA transcript for which is shown in FIG. 55, as well as functionally equivalent isoforms thereof, variants thereof, and non-human variants thereof.

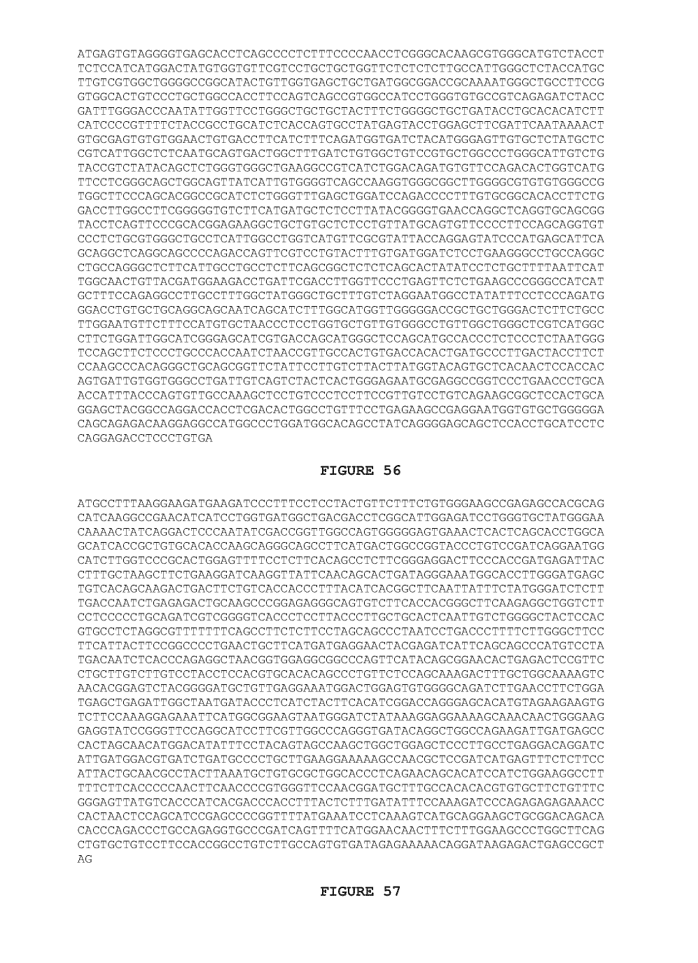

Sodium-dependent multivitamin transporter, or SLC5A6, is used herein to encompass human SLC5A6, the mRNA transcript for which is shown in FIG. 56, as well as functionally equivalent isoforms thereof, variants thereof, and non-human variants thereof.

Steroid sulphatase, or STS, is used herein to encompass human STS, the mRNA transcript for which is shown in FIG. 57, as well as functionally equivalent isoforms thereof, variants thereof, and non-human variants thereof.

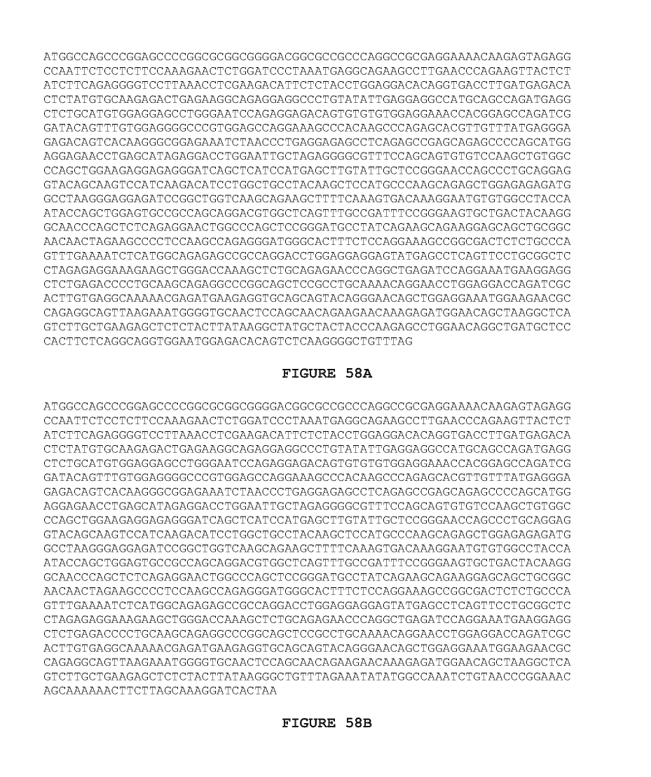

Syncoilin, intermediate filament protein, or SYNC1, is used herein to encompass human SYNC1, the mRNA transcript for which is shown in FIG. 58A, as well as functionally equivalent isoforms thereof, e.g. isoform 2, the mRNA transcript for which is shown in FIG. 58B, variants thereof, and non-human variants thereof.

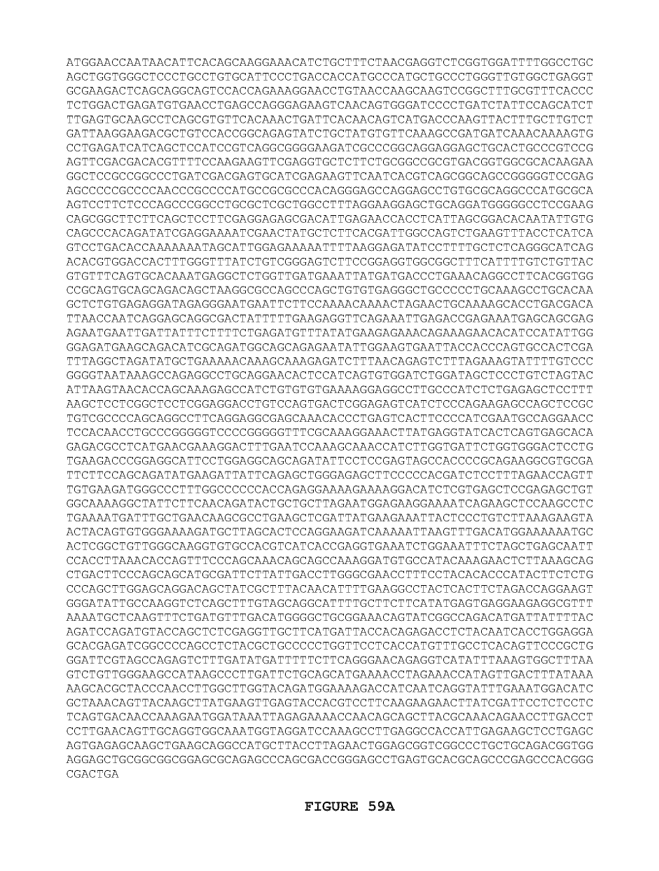

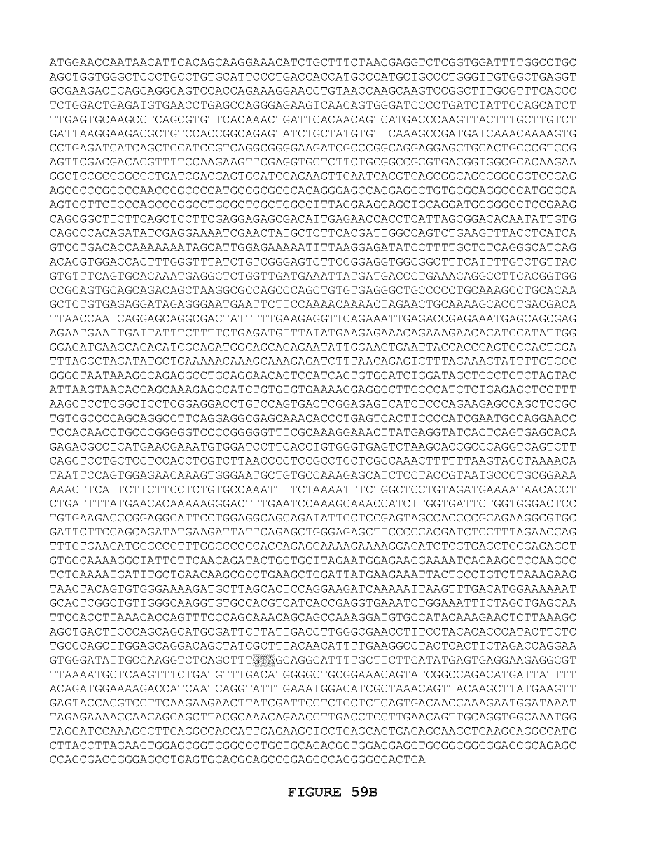

TBC1 domain family member 1, or TBC1D1, is used herein to encompass human TBC1D1, the mRNA transcript for which is shown in FIG. 59A, as well as functionally equivalent isoforms thereof, e.g. isoform 2, the mRNA transcript for which is shown in FIG. 59B, variants thereof, and non-human variants thereof.

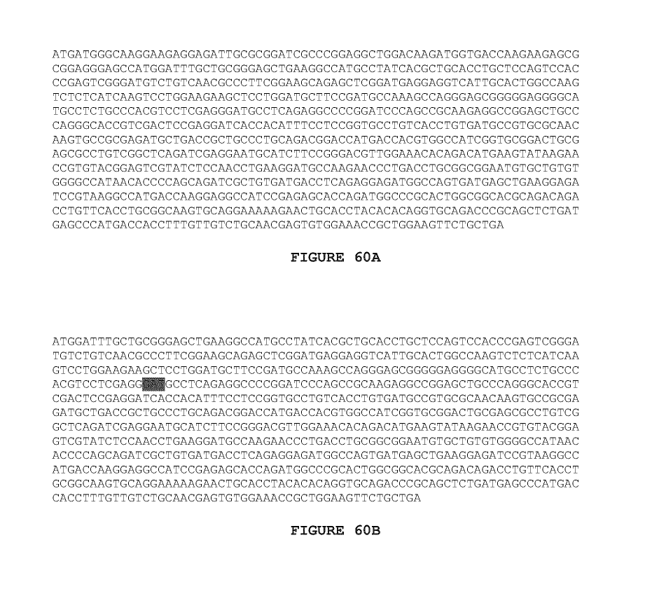

Transcription elongation factor A protein 2, or TCEA2, is used herein to encompass human TCEA2, the mRNA transcript for which is shown in FIG. 60A, as well as functionally equivalent isoforms thereof, e.g. isoform 2, the mRNA transcript for which is shown in FIG. 60B, variants thereof, and non-human variants thereof.

In a first step of the method, a biological sample is obtained from a mammal with breast cancer. The term “biological sample” is meant to encompass any mammalian sample that may contain nucleic acid encoding the target genes or that may contain the proteins encoded by the target genes. Suitable biological samples include, for example, blood, serum, plasma, urine, biopsied tumor tissue or pleural effusions. The sample is obtained from the mammal in a manner well-established in the art. The term “mammal” is used herein to refer to both human and non-human mammals including domestic animals, e.g. cats, dogs and the like, livestock and undomesticated animals.

Once a suitable biological sample is obtained, it is analyzed to determine the expression level or concentration of each of the biomarkers in the sample. As one of skill in the art will appreciate, the expression level of each biomarker may be determined using one of several techniques established in the art, including methods of quantifying nucleic acid encoding a target biomarker, such as PCR-based techniques, microarrays, the Nanospring nCounter gene expression system using color-coded probe pairs, and Northern or Southern blotting techniques, and/or methods of quantifying protein biomarkers, such as immunological assay, western blotting, or mass spectrometry.

In one embodiment, the expression level of protein biomarkers in a biological sample from a mammal may be determined based on the levels of nucleic acid (i.e. DNA or mRNA transcript) encoding the target protein biomarkers in the biological sample. Methods of determining DNA or mRNA levels are known in the art, and include, for example, PCR-based techniques (such as RT-PCR), microarrays, the Nanospring nCounter gene expression system using color-coded probe pairs and Northern or Southern blotting techniques which generally include the application of gel electrophoresis to isolate the target nucleic acid, followed by hybridization with specific labeled probes. Probes for use in these methods can be readily designed based on the known sequences of genes encoding the protein biomarkers, as well as the known amino acid sequence of the target biomarkers. Suitable labels for use are well-known, and include, for example, fluorescent, chemiluminescent and radioactive labels.

A preferred assay method to measure biomarker transcript abundance includes using the NanoString nCounter gene expression system. The system utilizes a pair of probes, namely, a capture probe and a reporter probe, each comprising a 35- to 50-base sequence complementary to the biomarker transcript. The capture probe additionally includes a short common sequence coupled to an immobilization tag, e.g. an affinity tag that allows the complex to be immobilized for data collection. The reporter probe additionally includes a detectable signal or label, e.g. is coupled to a color-coded tag. Following hybridization, excess probes are removed from the sample, and hybridized probe/target complexes are aligned and immobilized via the affinity or other tag in a cartridge. The samples are then analyzed, for example using a digital analyzer or other processor adapted for this purpose. Generally, the color-coded tag on each transcript is counted and tabulated for each target transcript to yield the expression level of each transcript on the sample.

In other embodiments, the expression level of protein biomarkers in a sample may be measured by immunoassay using an antibody specific to the target biomarker. The antibody is bound to the biomarker and bound antibody is quantified by measuring a detectable marker which may be linked to the antibody or other component of the assay, or which may be generated during the assay. Detectable markers may include radioactive, fluorescent, phosphorescent and luminescent (e.g. chemiluminescent or bioluminescent) compounds, dyes, particles such as colloidal gold and enzyme labels.

The term “antibody” is used herein to refer to monoclonal or polyclonal antibodies, or antigen-binding fragments thereof, e.g. an antibody fragment that retains specific binding affinity for the target biomarker. Antibodies to the target biomarkers are generally commercially available. For example, kits including antibody to destrin (Abnova, Origene and Genway), antibody to GPR27 (Novus Biologicals and Lifespan BioSciences, Inc.) and antibody LCP1 (Lifespan BioSciences, Inc. and Origene) are readily available. As one of skill in the art will appreciate, antibodies to the target biomarkers may also be raised using techniques conventional in the art. For example, antibodies may be made by injecting a host animal, e.g. a mouse or rabbit, with the antigen (target biomarker), and then isolating antibody from a biological sample taken from the host animal.

Different types of immunoassay may be used to determine expression level of target biomarkers, including indirect immunoassay in which the biomarker is non-specifically immobilized on a surface; sandwich immunoassay in which the biomarker is specifically immobilized on a surface by linkage to a capture antibody bound to the surface; competitive binding immunoassay in which a sample is first combined with a known quantity of biomarker antibody to bind biomarker in the sample, and then the sample is exposed to immobilized biomarker which competes with the sample to bind any unbound antibody. To the immobilized biomarker/antibody is added a detectably-labeled secondary antibody that detects the amount of immobilized primary antibody, thereby revealing the inverse of the amount of biomarker in the sample.

A preferred immunoassay for use to determine expression levels of protein biomarkers is an ELISA (Enzyme Linked ImmunoSorbent Assay) or Enzyme ImmunoAssay (EIA). To determine the level or concentration of the biomarker using ELISA, the biomarker to be analyzed is generally immobilized, for example, on a solid adherent support, such as a microtiter plate, polystyrene beads, nitrocellulose, cellulose acetate, glass fibers and other suitable porous polymers, which is pretreated with an appropriate ligand for the target biomarker, and then complexed with a specific reactant or ligand such as an antibody which is itself linked (either before or following formation of the complex) to an indicator, such as an enzyme. Detection may then be accomplished by incubating this enzyme-complex with a substrate for the enzyme that yields a detectable product. The indicator may be linked directly to the reactant (e.g. antibody) or may be linked via another entity, such as a secondary antibody that recognizes the first or primary antibody. Alternatively, the linker may be a protein such as streptavidin if the primary antibody is biotin-labeled. Examples of suitable enzymes for use as an indicator include, but are not limited to, horseradish peroxidase (HRP), alkaline phosphatase (AP), B-galactosidase, acetylcholinesterase and catalase. A large selection of substrates is available for performing the ELISA with these indicator enzymes. As one of skill in the art will appreciate, the substrate will vary with the enzyme utilized. Useful substrates also depend on the level of detection required and the detection instrumentation used, e.g. spectrophotometer, fluorometer or luminometer. Substrates for HRP include 3,3′,5,5′-Tetramethylbenzidine (TMB), 3,3′-Diaminobenzidine (DAB) and 2,2′-azino-bis(3-ethylbenzothiazoline-6-sulphonic acid) (ABTS). Substrates for AP include para-Nitrophenylphosphates. Substrates for β-galactosidase include β-galactosides; the substrate for acetylcholinesterase is acetylcholine, and the substrate for catalase is hydrogen peroxide.

As will be appreciated by one of skill in the art, assay methods which target the activity of a biomarker may also be utilized to determine the level of a biomarker in a sample. In this regard, suitable assays for each target biomarker are readily available to the skilled person.

The expression level of each biomarker in a given sample may be analyzed individually or together using, for example, biochip array technology. Generally, biochip arrays provide a means to simultaneously determine the level of multiple biomarkers in a given sample. These arrays may utilize ELISA technology and, thus, the biochip may be modified to incorporate capture antibodies at pre-defined sites on the surface.

Once the expression level of each signature biomarker in a biological sample of a mammal has been determined, these expression levels are compared to control expression levels, i.e. the expression level of one or more housekeeping genes. The term “housekeeping genes” as used herein is meant to refer to genes that encode protein products that are not connected to, involved in or required for processes specific to cancer cells, and thus, exhibit a fixed expression level in cancerous and non-cancerous cells. Examples of suitable housekeeping genes include, but are not limited to, genes encoding ACTB (Beta-actin), GAPDH (Glyceraldehyde 3-phosphate dehydrogenase), RPLP0 (60 S acidic ribosomal protein P0), GUSB (beta-glucuronidase), and TFRC (transferring receptor 1). In a comparison of the expression levels of target biomarkers to housekeeping genes, a determination of an increase in transcript abundance or expression of certain biomarkers and a decrease in transcript abundance or expression of other biomarkers has been determined to be indicative of prognosis in the mammal. For example, in one embodiment, a determination of a decrease in expression of DSTN, TDRD3, RGS4, MYO1E, RPL3, Hypothetical FLJ13769 and ANP32C, and an increase in expression of MC2R, DKFZp434L092, GPR27, HPS5 and LCP1 is indicative of a positive prognosis, e.g. a high probability of survival, for example, a greater than 50% probability of survival, for an extended period of time, e.g. at least about 5 years. Preferably, a positive prognosis indicates a probability of survival of at least about 60%, such as 70%, 75%, 80%, 85%, 90% or 95% probability of survival for at least about 5 years.

The level of expression that would be considered to represent increased or decreased expression of a target biomarker in accordance with the present method is determined relative to the expression of one or more housekeeping genes. Generally, a reproduceable statistically significant increase or decrease in the expression of a biomarker, for example, an increase or decrease of a least about 5%, e.g. at least about 10%, 15%, 20% or 25%, in comparison to the expression of a housekeeping gene, is considered to be increased or decreased expression that is relevant with respect to prognosis. As one of skill in the art will appreciate, the difference in the level of biomarker expression as compared to expression of the housekeeping gene(s) may vary contingent on the methodology employed to quantify and analyse nucleic acid and/or protein expression.

In another embodiment, in addition to a determination of expression of the base biomarkers DSTN, TDRD3, RGS4, MYO1E, RPL3, Hypothetical FLJ13769, ANP32C, MC2R, DKFZp434L092, GPR27, HPS5 and LCP1, a determination of the expression of one or more biomarkers selected from the group consisting of ADAM22, ATP10B, BCL2L14, CR2, DENND3, EPHA5, GLP1R, GLRA3, GPR27, HIST1H3J, HPS5, IQCA1, LCP1, LMAN1L, LOC642131, LRRC37A4, MC2R, MYCNOS, PARP4, PDE4B, PSG11, RFPL3S, RHBG, SLC11A1, SLC5A6 and STS, ANP32C, ANXA2P1, APBB2_CBWD1, CNIH3 DHX15, DSTN, EIF3H, GADD45B, HEXIM1, IL17B, KLHDC2, MRPL46, MYO1E, NDUFAF1, PDZRN3, PFDN5, RGS4, RPL13A, RTF1, SHOX2, SYNC, TBC1D1, and TCEA2 may be incorporated into the present method of prognosis. A determination of an increase in expression of ADAM22, ATP10B, BCL2L14, CR2, DENND3, EPHA5, GLP1R, GLRA3, GPR27, HIST1H3J, HPS5, IQCA1, LCP1, LMAN1L, LOC642131, LRRC37A4, MC2R, MYCNOS, PARP4, PDE4B, PSG11, RFPL3S, RHBG, SLC11A1, SLC5A6 or STS, or a decrease in expression of ANP32C, ANXA2P1, APBB2_CBWD1, CNIH3 DHX15, DSTN, EIF3H, GADD45B, HEXIM1, IL17B, KLHDC2, MRPL46, MYO1E, NDUFAF1, PDZRN3, PFDN5, RGS4, RPL13A, RTF1, SHOX2, SYNC, TBC1D1 or TCEA2, in addition to the prognostic expression signature of the base biomarkers, would be indicative of a positive prognosis.

In another embodiment, determination of the expression of at least 10, and preferably 11, of the base biomarkers DSTN, TDRD3, RGS4, MYO1E, RPL3, Hypothetical FLJ13769, ANP32C, MC2R, DKFZp434L092, GPR27, HPS5 and LCP1, alone with a determination of the expression of one or more biomarkers selected from the group consisting of ADAM22, ATP10B, BCL2L14, CR2, DENND3, EPHA5, GLP1R, GLRA3, GPR27, HIST1H3J, HPS5, IQCA1, LCP1, LMAN1L, LOC642131, LRRC37A4, MC2R, MYCNOS, PARP4, PDE4B, PSG11, RFPL3S, RHBG, SLC11A1, SLC5A6 and STS, ANP32C, ANXA2P1, APBB2 CBWD1, CNIH3 DHX15, DSTN, EIF3H, GADD45B, HEXIM1, IL17B, KLHDC2, MRPL46, MYO1E, NDUFAF1, PDZRN3, PFDN5, RGS4, RPL13A, RTF1, SHOX2, SYNC, TBC1D1, and TCEA2 is used to provided a prognosis for a mammal with BLBC or a molecularly similar cancer. For example, the method may be conducted without determining the expression level of one or two of TDRD3, RPL3, Hypothetical protein FLJ13769 and DKFZp434L092.

Thus, the prognostic method may include the determination of the expression of 10 or more of the base biomarkers and one or more additional biomarkers as identified, and may include a determination of expression of all of such biomarkers.

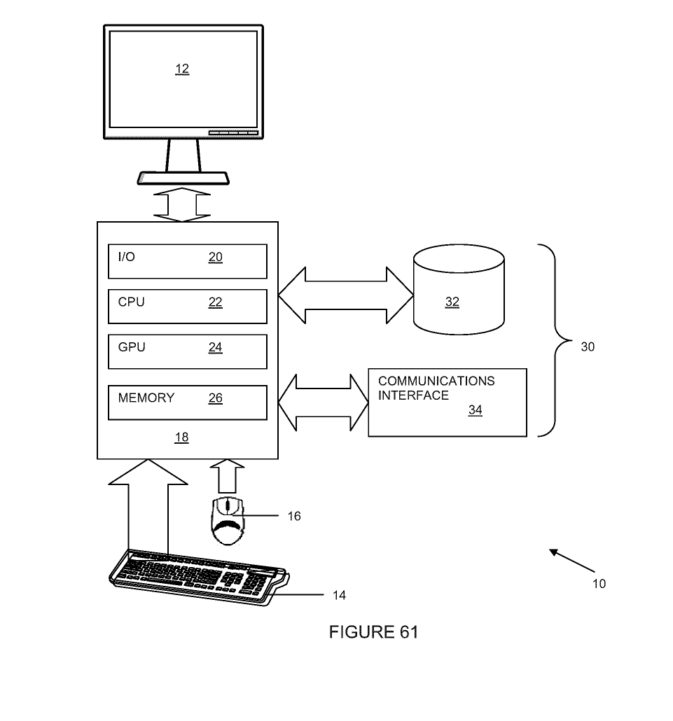

The methods described herein, or one or more steps thereof, may be implemented in whole or in part, using any suitable processing device, including any suitable computer or microprocessor-based system, such as a desktop or laptop computer or a mobile wireless telecommunication computing device, such as a smartphone or tablet computer, which may receive the electroencephalogram signals. The computer or microprocessor-based system may be coupled directly to instrumentation utilized to identify nucleic acid or protein abundance, e.g. Nanostring nCounter instrumentation or other instrumentation utilized in the present method, with a wired or wireless connection, or may obtain data from a separate storage medium or network connection such as the Internet. An illustrative computer system in respect of which the methods herein described may be implemented is presented as a block diagram in FIG. 61. The illustrative computer system is denoted generally by reference numeral 10 and includes a display 12, input devices in the form of keyboard 14 and pointing device 16, computer 18 and external devices 30. While pointing device is depicted as a mouse, it will be appreciated that other types of pointing device may also be used.

The computer may contain one or more processors or microprocessors, such as a central processing unit (CPU) 22. The CPU performs arithmetic calculations and control functions to execute software stored in an internal memory 26, preferably random access memory (RAM) and/or read only memory (ROM), and possibly additional memory 32. The additional memory may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces, removable memory chips such as EPROM or PROM, emerging storage media, such as holographic storage, or similar storage media as known in the art. This additional memory may be physically internal to the computer, external, or both. The computer system may also include other similar means for allowing computer programs or other instructions to be loaded. Such means can include, for example, a communications interface 34 which allows software and data to be transferred between the computer system and external systems and networks. Examples of communications interface include a modem, a network interface such as an Ethernet card, a wireless communication interface, or a serial or parallel communications port. Software and data transferred via communications interface are in the form of signals which can be electronic, acoustic, electromagnetic, optical or other signals capable of being received by communications interface. Multiple interfaces, of course, may be provided on a single computer system.

Input and output to and from the computer is administered by the input/output (I/O) interface 20. This I/O interface administers control of the display, keyboard, external devices and other such components of the computer system. The computer will generally include a graphical processing unit (GPU) 24 useful for computational purposes as an adjunct to, or instead of, the CPU 22, for mathematical calculations.

The various components of the computer system are coupled to one another either directly or by coupling to suitable buses.

The use of the present biomarker signature is particularly applicable in methods of prognosis for mammals with basal-like breast cancer (BLBC), and molecularly similar cancers, i.e. cancers which exhibit the same or a similar gene expression profile, including the Estrogen Receptor (ER) negative breast cancer, HER2-overexpressing (ERBB2+) breast cancer, as well as cancers that arise in tissues other than the breast including, such as those that arise in the bladder, colon, kidney, liver, lung, including small cell lung cancer, esophagus, gall-bladder, ovary (e.g. serous ovarian cancer), pancreas, stomach, cervix, thyroid, prostate, and skin, including squamous cell carcinoma, e.g. lung squamous carcinoma; hematopoietic tumors of lymphoid lineage including leukaemia, acute lymphocytic leukaemia, acute lymphoblastic leukaemia, B-cell lymphoma, T-cell-lymphoma, Hodgkin's lymphoma, non-Hodgkin's lymphoma, hairy cell lymphoma and Burkitt's lymphoma; hematopoietic tumors of myeloid lineage, including acute and chronic myelogenous leukemias, myelodysplastic syndrome and promyelocytic leukaemia; tumors of mesenchymal origin, including fibrosarcoma and rhabdomyosarcoma; tumors of the central and peripheral nervous system, including astrocytoma neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma.

The present prognostic method advantageously permits identification of patient prognosis at the time of cancer diagnosis. This allows subsequent treatment protocols to be tailored to the specific needs of the patient. For example, for patients with a positive prognosis, e.g. greater than 50% probability of survival for an extended period of time, aggressive therapeutic regimens may be avoided. On the other hand, for patients with a negative prognosis, e.g. less than 50% probability of long-term survival, an aggressive therapeutic regimen may be more appropriately implemented.

In another aspect of the invention, an article of manufacture is provided that is useful to practice the present prognostic method. The article of manufacture comprises a biomarker-specific reactant for one or more of the biomarkers, DSTN, TDRD3, RGS4, MYO1E, RPL3, Hypothetical FLJ13769, ANP32C, MC2R, DKFZp434L092, GPR27, HPS5 and LCP1, or nucleic acid encoding a biomarker. The article of manufacture will also include a specific reactant for one or more housekeeping genes or proteins. Reactants will be suitable to determine the expression level of the biomarker or housekeeping nucleic acid or protein in a biological sample from the mammal. Suitable reactants may include, for example, antibodies that specifically bind to the biomarker, or a nucleic acid probe directed against a portion of the gene/mRNA encoding a biomarker. The reactants may or may not be associated with an indicator that is measurable to indicate the expression level of the target biomarker(s). Suitable indicators will depend on the reactant for use to detect biomarker expression level. Antibody reactants may be associated with enzyme labels such as horseradish peroxidase (HRP) and alkaline phosphatase (AP), with or without suitable substrates, or with labeled or unlabeled secondary antibody.

The article of manufacture may additionally include a microtitre plate or other support surface, to conduct the assays, and the support surface may modified to include bound reactant for one or more of the biomarkers, or a non-specific binding material useful to conduct an assay such as an indirect assay.

The packaging of the article of manufacture will generally indicate that a determination in a biological sample of a decreased expression level of DSTN, TDRD3, RGS4, MYO1E, RPL3, Hypothetical FLJ13769 and ANP32C and an increased expression level of MC2R, DKFZp434L092, GPR27, HPS5 and LCP1 in comparison to a control expression level, e.g. the expression level of one or more housekeeping genes, is indicative of a prognosis for the mammal of greater than 50% survival for an extended period of time.

Embodiments of the invention are described in the following specific examples which are not to be construed as limiting.

### Example 1

To identify genes whose expression might be associated with the clinical outcome of BLBC, a large collection of human breast tumor gene expression data for which clinical data was also available (n=995) was compiled as follows.

### Collecting Microarray Data

Gene expression profiles of 5 independent external datasets were analyzed. These were obtained using Affymetrix HG-U133A GeneChips arrays, which have been deposited in the Gene Expression Omnibus (GEO); accession numbers GSE1456, GSE2034, GSE3494, GSE6532, and GSE7390. Together these datasets provided expression profiles of 1,077 human breast tumor samples. All gene expression profiles were normalized with frozen Robust Multi-Array Analysis (fRMA), a procedure that allows one to pre-process microarrays individually or in small batches and to then combine the data into a single comparable dataset for further analyses. To remove batch effect from the combined dataset, the ComBat method, which uses an Empirical Bayes method to adjust for potential batch effects in the dataset, and computed Pearson correlation coefficients for pair-wise comparisons of samples using 68 house-keeping probe sets. Samples exhibiting correlations higher than 0.95 were selected for further classification. The latter filtering method yielded a dataset comprising 995 human breast tumor samples.

### Tumor Classification

Each of the selected 995 samples described above, were classified as basal-like, HER2+, Luminal A, Luminal B, claudin-low or normal-like by assigning it to a cluster representing the subtype to which it had the highest Pearson correlation (as described in Perou et al. Nature 406, 747-752 (2000). The correlation was computed using the subset of 1,500 averaged and median-centered ‘intrinsic’ genes common to both the present dataset (Affymetrix Human Genome U133A Array) and the dataset used by Parker et al. J Clin Oncol 27, 1160-1167 (2009) (Stanford Microarray). For robustness, only tumors exhibiting a correlation higher than 0.3 with any of the molecular subtypes were used for further analysis. This led to the classification of 137 breast tumors into the basal-like molecular subtype yielding a group of 134 tumors with useable clinical follow-up data. These 134 patients with basal breast tumors were randomly separated, approximately ⅔ (n=85) were taken for signature training purposes (training set), and the remaining ⅓ (n=49) was used as an independent validation set.

### Binary Regression

Identification of the prognostic signature was completed using the Bayesian binary regression algorithm BinReg ver2.0. In most cases, disease free survival (DFS) was used as the relevant clinical variable, however, in some cases only distant metastasis free survival (DMFS) was available within a patient's clinical annotation. In these cases, DMFS was counted as DFS. Five year DFS was used as the clinical endpoint for these studies.

### Training Signature

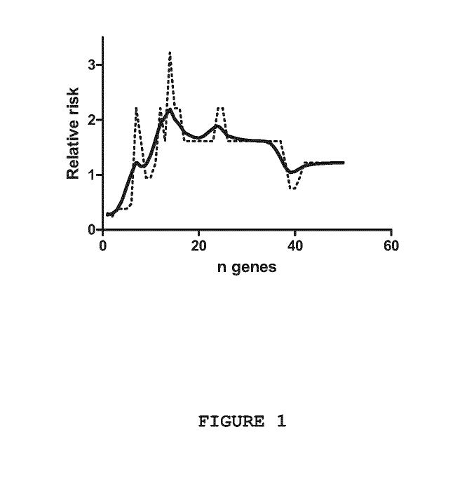

Starting with a single probe set signature, signatures were iteratively generated by gradually adding probe sets and testing the resulting signature using leave-one-out cross-validation. In this fashion, multiple signatures were generated comprising n probe sets, where n=1, 2, 3 . . . , 50. For each discrete value of n, this technique assigned a probability to every patient within the training set that indicated the likelihood of a patient experiencing disease relapse. To establish a probability cut-point, where patients with higher probability are assigned into the poor prognosis category and patients with lower probability are assigned into the good prognosis category, a tertile method as described in Haibe-Kains et al. (Bioinformatics 24, 2200-2208 (2008)) was used. Good prognosis was assigned to patients whose probability score fell in the lowest ⅓ of all probability scores, whereas poor prognosis was assigned to patients whose score fell into the higher ⅔ of probability scores. To determine which n-element signature had optimal performance, the relative risk of relapse for each signature was compared (FIG. 1, dotted line, relative risk; black line: LOWESS (LOcally WEighted Scatterplot Smoothing) curve fitted to relative risk data, n=14 identifies optimal signature length). In this fashion, a 14-probe-set (each gene represented by 1 probe set, while RPL3 is represented by 3 probe sets) henceforth called Basal 14 signature, which separated patients into good and poor outcome groups (Table 1) was determined

TABLE 1
Features comprising the optimal 14-gene signature

| | Affymtetrix | |
|---|---|---|
| Correlation | Probe | Description |
| + | 201022_s_at | destrin (actin depolymerizing factor), |
| | | DSTN |
| + | 203072_at | myosin IE, MYO1E |
| + | 208089_s_at | tudor domain containing 3, TDRD3 |
| + | 204338_s_at | regulator of G-protein signaling 4, RGS4 |
| + | 220719_at | hypothetical protein FLJ13769, FLJ13769 |
| + | 212039_x_at | ribosomal protein L3, RPL3 |
| + | 211073_x_at | ribosomal protein L3, RPL3 |
| + | 201217_x_at | ribosomal protein L3, RPL3 |
| + | 208538_at | acidic (leucine-rich) nuclear |
| | | phosphoprotein 32 family, member C, |
| | | ANP32C |
| − | 217434_at | melanocortin 2 receptor |
| | | (adrenocorticotropic hormone), MC2R |
| − | 216143_at | MRNA; cDNA DKFZp434L092 (from |
| | | clone DKFZp434L092), --- |
| − | 221306_at | G protein-coupled receptor 27, GPR27 |
| − | 204544_at | Hermansky-Pudlak syndrome 5, HPS5 |
| − | 208885_at | lymphocyte cytosolic protein 1 |
| | | (L-plastin), LCP1 |

### Assessment of Signature Performance

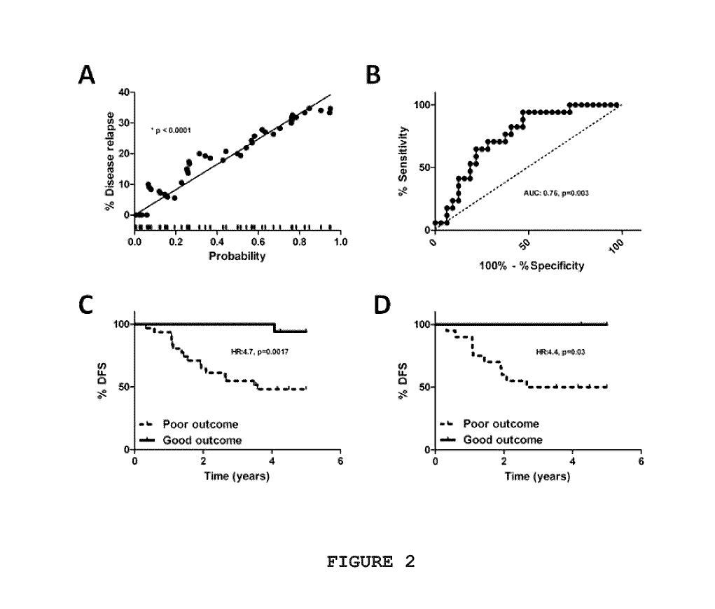

Validation of a gene signature using an independent data set is a more accurate measurement of its prognostic value than using cross-validation on a training data set. Therefore, the 14-probe signature identified above (Basal-14 signature) was tested on an independent cohort of patients with BLBC (n=49). To learn whether the probability of disease relapse predicted by the Basal-14 signature could be used as a continuous predictor of disease relapse, the proportion of patients who had experienced disease relapse was calculated while increasing the cut-off (decreasing stringency) for assigning a patient into the good outcome group. Indeed, the proportion of patients experiencing disease relapse increased in an approximate linear fashion as the probability assigned for disease relapse by the Basal-14 signature increased (FIG. 2A). To assess the predictive accuracy of the Basal-14 signature, a receiver-operator characteristic (ROC) curve analysis was conducted. An AUC (Area Under Curve) value of 0.5 indicates predictive performance which is no better than chance, whereas values greater than 0.5 indicate true predictive capacity. The Basal-14 signature produced an AUC that was statistically significantly higher than 0.5 (AUC: 0.76, p=0.003, FIG. 2B). Taken together, these data demonstrate the capacity for the Basal-14 signature to identify BLBC patients at high risk for disease relapse.

To visualize survival differences between groups of patients that were predicted to have either high or low risk for disease relapse, patients were stratified from the validation cohort into good and poor outcome groups using tertiles, and Kaplan-Meier survival analysis were completed. Patients whose predicted probability for disease relapse fell within the lowest tertile of predicted probabilities were stratified into the good outcome group, whereas those whose predicted probabilities fell within the upper two tertiles were stratified into the poor outcome group. The Kaplan-Meier estimate for the proportion of patients in the low-risk group who did not experience a disease relapse at 5 years (94%) was significantly greater than the proportion in the poor outcome category (48%) (Table 2, FIG. 2C, HR: 4.7 [CI95: 1.8-12.3], p=0.0017).

TABLE 2
Survival characteristics of the 49 patient validation cohort.
Validation cohort (n = 49)

| Risk | # | % | % Disease free |
|---|---|---|---|
| Category | Patients | Patient | survival (5 yr) |
| Low | 16 | 33 | 94 |
| High | 33 | 67 | 48 |

The capacity of the 14-probe signature to predict the outcome of patients who had not received adjuvant chemotherapy was also tested (e.g. for use to identify patients who could be spared aggressive chemotherapy). This allowed testing of the relationship between the Basal-14 signature and the natural progression of BLBCs without having adjuvant chemotherapy as a potentially confounding variable. 26 patients within the 49 patient validation cohort met this criterion (patients from GSE7390 & GSE2034). The predictive capacity of the Basal-14 signature was re-tested on these 26 chemotherapy naïve patients and a statistically significant difference was observed in the survival of patients who were predicted to have either good or poor outcome (FIG. 2D, HR: 4.4 [CI95: 1.1-16.7], p=0.03, Table 3).

TABLE 3
Survival characteristics of the 26 patient chemo-naïve validation cohort
Chemo-naïve validation cohort (n = 26)

| Risk | # | % | % Disease free |
|---|---|---|---|
| Category | Patients | Patients | survival (5 yr) |
| Low | 6 | 23 | 100 |
| High | 20 | 77 | 50 |

The proportion of patients in the chemotherapy naïve validation cohort who were predicted to have good survival and were free of disease at 5 years was 100%, whereas among those patients who were predicted to have poor survival, only 50% were disease free after 5 years. Taken together, these findings demonstrate the capacity of the 14-gene signature to identify patients who have excellent long-term survival even when patients did not receive aggressive adjuvant chemotherapy.

### Example 2

### Comparison of the Basal-14 Signature with Other Multigene Predictors

Previous studies have reported that many published multigene predictors fail to accurately identify high and low risk patients among patients with ER-negative breast cancer. As the majority of BLBCs are ER-negative, it was tested whether or not multiple previously described multigene predictors were prognostic in the context of BLBC. To this end, the association of the Genomic Grade Index 5, NKI-70 signature, Recurrence score, CSR/Wound response signature, Triple-negative signature, MS-14 signature, as well as the Basal-14 signature was measured in the 49 patient validation cohorts. For cross platform comparisons with other gene signatures, signature elements were mapped by Unigene IDs to Affymetrix HG-U133A GeneChip arrays for testing in the 49 patient validation set. The expression values for each gene were transformed such that the mean was 0 and the standard deviation was 1. A signature index was calculated for each patient as follows: where x is the transformed expression, n is the number of genes that could be mapped to the Affymetrix HG-U133 arrays, P is the set of probes with reported positive correlation to poor outcome, and N is the set of probes with reported positive correlation to good outcome. For each signature, Kaplan-Meier survival analysis using tertiles were completed to dichotomize the validation cohort into good and poor outcome groups, or generating ROC curves.

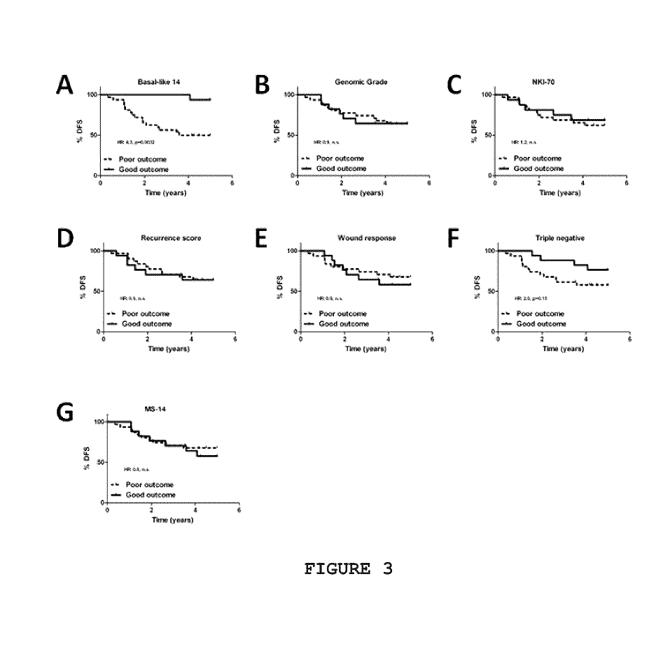

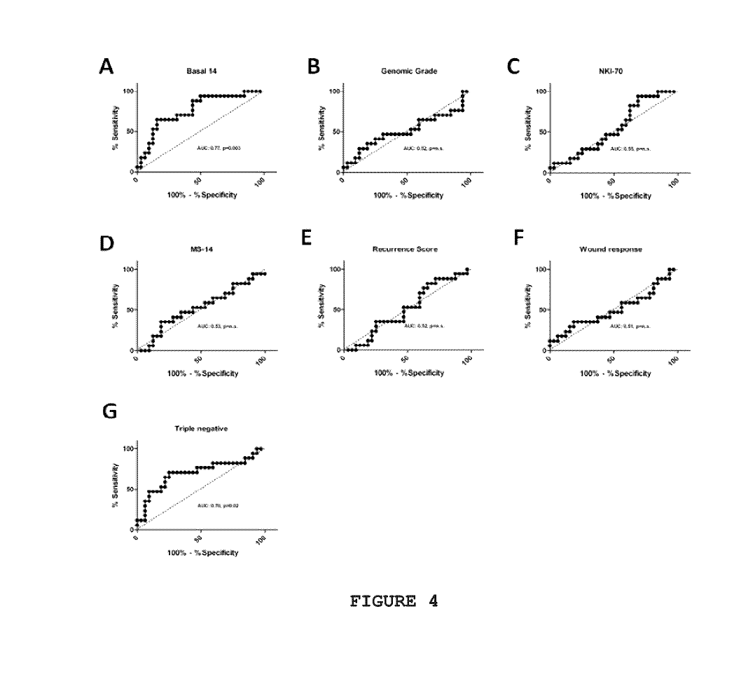

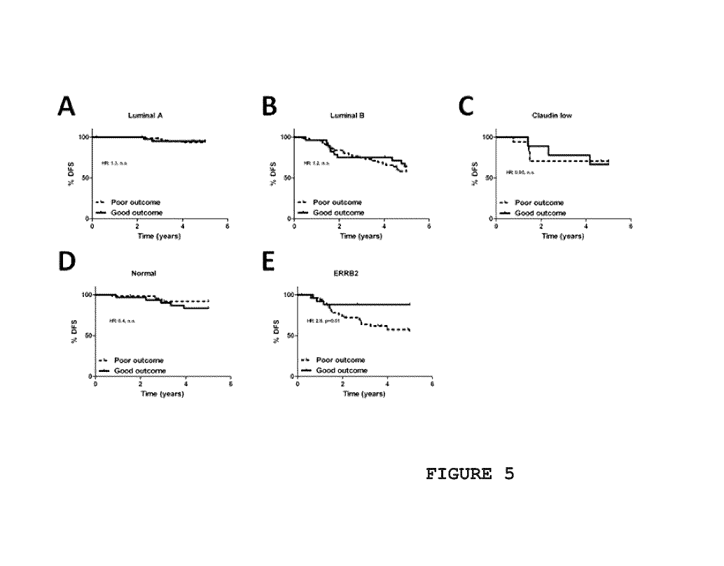

Interestingly, other than the Basal-14 signature (FIG. 3A, HR: 4.3 [CI95: 1.6-11.4], p=0.0032, none of the other signatures identified patient groups with statistically significant differences in survival (Kaplan-Meier: FIG. 3A-F. ROC: FIG. 5A-F). These findings highlight the poor capacity of previously reported multigene outcome predictors to identify patients with BLBC at high and low risk of diseases relapse. However, it should be noted that the triple negative signature trended towards significance in the Kaplan-Meier analysis (FIG. 3F, HR: 2.0 [CI95: 0.8-5.4], p=0.15) and was statistically significant in the ROC curve analysis. This is likely because the triple negative signature was developed on breast tumors histopathologically classified as triple negative, which comprises a sub-group that overlaps with the basal-like molecular subtype. Together, these findings underscore the need for prognostic multigene signatures, such as the Basal 14 signature, for guiding therapy choice for breast cancer patients.

### Performance of Basal-14 Signature in Other Molecular Subtypes of Breast Cancer

Previous studies have demonstrated that biological processes that can be linked to breast cancer patient outcome vary among the different molecular subtypes of breast cancer. In this regard, it was tested whether or not the Basal-14 signature could be used to identify high and low risk patients among the other molecular subtypes of breast cancer, or whether its capacity to stratify patients into high and low risks groups was limited to patients with BLBCs. The Basal-14 signature showed no capacity to identify patients at high and low risk for disease relapse among the luminal A (HR: 1.3, p=n.s.), luminal B (HR: 1.2, p=n.s.), claudin low (HR: 1.0, p=n.s.) and normal (HR: 0.4, p=n.s.) molecular subtypes of breast cancer (FIG. 5A-E). Unexpectedly, the Basal-14 signature was also prognostic in the ERBB2 or HER2 overexpressing molecular subtype (HR: 2.8 [CI95: 1.3-6.5], p=0.01). These data suggest that similar biological processes may govern patient outcome in both the basal-like and ERBB2 molecular subtypes of breast cancer. Taken with previous findings, it appears that transcripts whose expression may be informative for patient prognosis vary between the different molecular subtypes of breast cancer. For example, it appears that signatures that are prognostic in ER-positive breast tumors, such as the Reccurrence score (OncotypeDX®) and the Genomic Grade Index, fail to stratify BLBCs into good and poor outcome groups, whereas the Basal-14 signature is prognostic in basal-like and ERBB2-overexpressing breast cancer, but fails to identify patients in the ER-positive luminal subtypes of breast cancer.

### Example 3

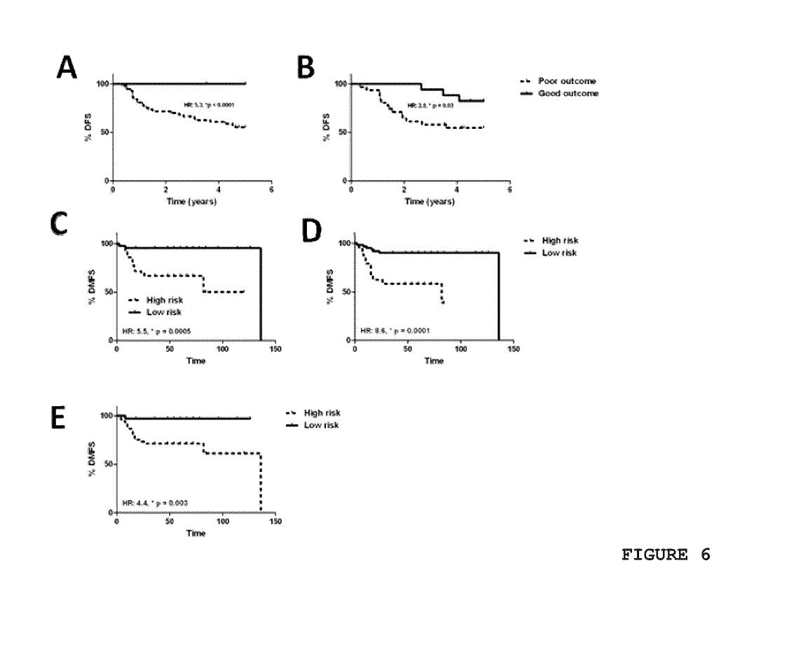

### Identification and Validation of a 50-Gene Signature for Basal-Like Breast Cancer Patients

Whereas the experiments detailed above describe a 14-gene signature for BLBC prognosis, these data were derived using microarray technology which is generally not amenable for use in clinical pathology labs that analyze patient breast tumors. To overcome this limitation, a prognostic signature for BLBC using a NanoString nCounter Gene Expression System was developed. To this end, the top 50 prognostic candidate genes were identified from the microarray experiments, as well as 5 housekeeping genes, and a NanoString nCounter codeset of probes were prepared for each gene to carry forward into the development of a gene expression-based prognostic test (Table 4). The performance of these 50 genes is shown in FIG. 6 (A&B, Training and validation cohorts)

TABLE 4
50 BLBC prognostic genes

| Correlation (poor prognosis) | Gene ID |
|---|---|
| − | ADAM22 |
| − | ATP10B |
| − | BCL2L14 |
| − | CR2 |
| − | DENND3 |
| − | EPHA5 |
| − | GLP1R |
| − | GLRA3 |
| − | GPR27 |
| − | HIST1H3J |
| − | HPS5 |
| − | IQCA |
| − | LCP1 |
| − | LMAN1L |
| − | LOC642131 |
| − | LRRC37A4 |
| − | MC2R |
| − | MYCNOS |
| − | PARP4 |
| − | PDE4B |
| − | PSG11 |
| − | RFPL3S |
| − | RHBG |
| − | SLC11A1 |
| − | SLC5A6 |
| − | STS |
| + | ANP32C |
| + | ANXA2P1 |
| + | APBB2 |
| + | CBWD1 |
| + | CNIH3 |
| + | DHX15 |
| + | DSTN |
| + | EIF3H |
| + | GADD45B |
| + | HEXIM1 |
| + | IL17B |
| + | KLHDC2 |
| + | MRPL46 |
| + | MYO1E |
| + | NDUFAF1 |
| + | PDZRN3 |
| + | PFDN5 |
| + | RGS4 |
| + | RPL13A |
| + | RTF1 |
| + | SHOX2 |
| + | SYNC1 |
| + | TBC1D1 |
| + | TCEA2 |

The capacity of these genes to discriminate among good and poor outcome patients from an independent retrospective cohort of BLBC patients (n=86), using RNA extracted from formalin fixed paraffin embedded archival samples, and using Nanostring nCounter CodeSets (Table 5) to quantify the relative abundance of the transcript counterparts of the 50 BLBC prognostic genes identified from microarray experiments (Table 5), was tested. Examples of stratification of these patients into high (identified as “+” above) and low (identified as “−” above) risk groups are provided in FIG. 6 (C-E).

TABLE 5
Nanostring CodeSets for the 50 BLBC prognostic genes

| Type | Gene | Target Sequence |
|---|---|---|
| Prognostic | GLP1R | GGAACTCCAACATGAACTACTGGCTCATTATCCGGCTGCCCATTCTCTTTGGCATTGGGGTGAACTTCCTCATC |
| | | TTTGTTCGGGTCATCTGCATCGTGGT |
| Prognostic | LCP1 | CCAGGGGGGACAATATGTGCCAATCAATAGCACCCCTACTCACATACACACACACCTAGCCAGCTGTCAAGGGC |
| | | AGAATGAATCTATGCTGGATAAGAAA |
| Prognostic | PDE4B | TCACAGATGATTCTTCTGAATGCTCCCGAACTACTGACTTTGAAGAGGTAGCCTCCTGCCTGCCATTAAGCAGG |
| | | AATGTCATGTTCCAGTTCATTACAAA |
| Prognostic | STS | GACCCAGCTGTAGTGAGGTTGCAGTGATTGAGTAGGATTGGCCTGCTTCAAAGCAGAGGTTTCTCATGGGAATA |
| | | TGCTTATTAAACTCCCACTGGTGCAG |
| Prognostic | MYCNOS | AGCGGTGCAATGCAGCACCCACCCTGCGAGCCTGGCAATTGCTTGTCATTAAAAGAAAAAAAAATTACGGAGGG |
| | | CTCCGGGGGTGTGTGTTGGGGAGGGG |
| Prognostic | BCL2L14 | GGTCTCGTTCGTCTCCAGCTCATAAAATGTAGCAGCATCATCCTTGACAGTGATGTTTTTCAGGCCCTCCATTG |
| | | AGAACCTGAGGAAATCTGTAAAGATA |
| Prognostic | DENND3 | GCATGATGAGTACTGTTTCTACAATGGCAAAACGCACCGGGAGTGTCCTGGCTGCTTCGTGCCCTTCGCGGTGT |
| | | GCGTGGTCTCCAGGTTTCCCTATTAC |
| Prognostic | GPR27 | GCGCCGCCTCCTCGTGCTGGAAGAATTCAAGACGGAGAAGAGGCTGTGCAAGATGTTCTACGCCGTCACGCTGC |
| | | TCTTCCTGCTCCTCTGGGGGCCCTAC |
| Prognostic | LOC642131 | CCTGAAGCTGGCCTCAGCTGATGTGCTGAGACCACGGGTCATGCACACGTATGATTCCAGGTCATGCGGGCTCT |
| | | ACTGCAGGACAGACCTGTGTCCTGTG |
| Prognostic | LRRC37A4 | CAAGCCCTGTCTTTTTCCCAAGCCCTCAAGCACACGCATGAGTGTTCATCCCGACTTGGTAGGGGGCTTTTCAC |
| | | CCTTACAAGATGGCAAAAGATTCACA |
| Prognostic | PSG11 | ACTCAGCCACTGGCGAGGAAAGCTCCACATCCTTGACAATCAGAGTCATTGCTCCTCCAGGATTAGGAACTTTT |
| | | GCTTTCAATAATCCAACGTAGCAGCC |
| Prognostic | ADAM22 | AGTTTGCAGTAATGAGCTGAAGTGTGTGTGTAACAGACACTGGATAGGTTCTGATTGCAACACTTACTTCCCTC |
| | | ACAATGATGATGCAAAGACTGGTATC |
| Prognostic | ATP10B | ACCTGCAAGTTGATTAGAACTGCCTTTCTTCCCAGGCTTGACATAGGTATTAAGTCAAAATTACATGAAACCCA |
| | | GTGGTAAAAAAGCCTCTGAAAGCTGT |
| Prognostic | CR2 | GGTGTCAAGCAAATAATATGTGGGGGCCGACACGACTACCAACCTGTGTAAGTGTTTTCCCTCTCGAGTGTCCA |
| | | GCACTTCCTATGATCCACAATGGACA |
| Prognostic | HPS5 | CTGATATATTGTGCTCGCCCAGGCTCTAGGATGTGGGAAGTGAACTTTGATGGAGAAGTTATAAGTACACATCA |
| | | GTTCAAGAAACTCCTCTCGTTGCCAC |
| Prognostic | HIST1H3J | CTAAGGACATCCAGCTTGCGCGTCGTATCCGTGGCGAGCGAGCATAATCCCCTGCTCTATCTTGGGTTTCTTAA |
| | | TTGCTTCCAAGCTTCCAAAGGCTCTT |
| Prognostic | IQCA1 | CAATGTCGAACGCAATGTATAATAAGATGTGGCATCAGACCCAAGAAGCCCTCGGTGCTTTACTCGATAAAGAG |
| | | CCTCAGAAGATGATTGAACCACAAAG |
| Prognostic | LMAN1L | GCCTGCAGCCTGGCATCTTCCTGTTCTACCTCCTCATTCAGACTGTAGGCTTCTTCGGCTACGTGCACTTCAGG |
| | | CAGGAGCTGAACAAGAGCCTTCAGGA |
| Prognostic | MC2R | GATCGTCCTGCTGGCTGTGTTCAAGAATAAGAATCTCCAGGCACCCATGTACTTTTTCATCTGTAGCTTGGCCA |
| | | TATCTGATATGCTGGGCAGCCTATAT |
| Prognostic | PARP4 | CATGGTTAATGTCTGTGAAACTAATTTGTCCAAACCCAACCCACCATCCCTGGCCAAATACCGAGCTTTGAGGT |
| | | GCAAAATTGAGCATGTTGAACAGAAT |
| Prognostic | SLC11A1 | GTTTCCTAGCGCAGCCATGTGATTACCCTCTGGGTCTCAGTGTCCTCATCTGTAAAATGGAGACGCCACCACCC |
| | | TTGCCATGGAGGTTAAGCACTTTAAC |
| Prognostic | SLC5A6 | CATTGCCTGCCTCTTCAGCGGCTCTCTCAGCACTATATCCTCTGCTTTTAATTCATTGGCAACTGTTACGATGG |
| | | AAGACCTGATTCGACCTTGGTTCCCT |
| Prognostic | EPHA5 | TACGTAACCCAAGTAGTCTGAAGACGCTGGTTAATGCATCCTGCAGAGTATCTAATTTATTGGCAGAACATAGC |
| | | CCACTAGGATCTGGGGCCTACAGATC |
| Prognostic | GLRA3 | ACCACCCTGTCCAGGTAATGCCAAAAAGTCCTGATGAAATGAGGAAGGTCTTTATCGACCGGGCCAAGAAGATT |
| | | GATACCATCTCCCGAGCCTGCTTCCC |
| Prognostic | RFPL3S | CCCCTTCTCTGTTACCAAGGTGACCCCAAGGAACACAGTAAATGTGGCGGCTTATTTGGCCTCCCCAGGACGGA |
| | | CTGGAGCATCAGTAGTGCCTGAGTTC |
| Prognostic | RHBG | GCAAGCACCGCCAGGGCTCCGTCTACCATTCAGACCTCTTCGCCATGATTGGGACCATCTTCCTGTGGATCTTC |
| | | TGGCCTAGCTTCAATGCTGCACTCAC |
| Prognostic | HEXIM1 | GGAGCGAGCGCCGCTTTCCAAGTTTGGAGACTAGACTGAAACTTTTTTGGGGGAGGGGGCAAAGGGGACTTTTT |
| | | ACAGTGATGGAATGTAACATTATATA |
| Prognostic | RPL13A | AGTCCAGGTGCCACAGGCAGCCCTGGGACATAGGAAGCTGGGAGCAAGGAAAGGGTCTTAGTCACTGCCTCCCG |
| | | AAGTTGCTTGAAAGCACTCGGAGAAT |
| Prognostic | GADD45B | TGTGGACCCAGACAGCGTGGTCCTCTGCCTCTTGGCCATTGACGAGGAGGAGGAGGATGACATCGCCCTGCAAA |
| | | TCCACTTCACGCTCATCCAGTCCTTC |
| Prognostic | MYO1E | TCACACCAGGTACTTAAAGATGTGCTCTGCTTTTTTCCAACTACGGAGTGTCACTGCTTTCTAGGTCAGTCCCT |
| | | GCAGACTCTTCTCAACTCTTTCCCTA |
| Prognostic | PDZRN3 | AAATCTTCGATAACTGGATGACGATCCAAGAACTCTTAACCCACGGCACAAAATCCCCGGACGGCACTAGAGTA |
| | | TACAATTCCTTCCTATCGGTGACTAC |
| Prognostic | ANZA2P1 | AGCGTCCAGAAATGGTGCTCCCCATGCTTCCAGCTAACAGGTCTAGAAAACCCGCTTGTGACTAGCAGTCCCTG |
| | | TGGCTGTTCCTGTGAGGATGACGTTA |
| Prognostic | CBWD1 | TATTCAGAGGCTGCTCTGCTGAGAAGATGAACAAATTTCTTGTCCAAAACAATGTATTTCAAACGTGCCGCTCG |
| | | GGCCTTTCCCGTATTGCTCACTGGTG |
| Prognostic | DHX15 | CTCTCCTAACTATTCCTGGGCGTACACATCCTGTTGAGATCTTCTATACTCCAGAACCAGAGAGAGATTATCTT |
| | | GAAGCAGCAATTCGAACAGTTATCCA |
| Prognostic | DSTN | AGTAGCCCTGCACCTGCCAGTGAGCTCGCCATTCACTGATTGGAAGAGTGACCTGGCATCTTGGAAATCATTGT |
| | | GTGTCTTCAGGAGAATGTGCAGTGTC |
| Prognostic | EIF3H | TGAAGTCCAATATCAGATGGAAATGATGCGGAGCCTTCGCCATGTAAACATTGATCATCTTCACGTGGGCTGGT |
| | | ATCAGTCCACATACTATGGCTCATTC |
| Prognostic | IL17B | ACCAGGTGCCACTGGACCTGGTGTCACGGATGAAACCGTATGCCCGCATGGAGGAGTATGAGAGGAACATCGAG |
| | | GAGATGGTGGCCCAGCTGAGGAACAG |
| Prognostic | KLHDC2 | TTTGGAGGACACCATTCAAGAGGCAATACCAATAAGTTCTACATGCTGGATTCAAGGTCTACAGACAGAGTGTT |
| | | ACAGTGGGAAAGAATTGATTGCCAAG |
| Prognostic | RGS4 | CACTGACAGCCTCCACCTTGAGCACTATTCTAAGGAGCAAATACCTTAGCTCCCTTGAGCTGGTTTTCTCTGAT |
| | | GGCACTTTTGAGCTCCTAAGCTGCCA |
| Prognostic | SHOX2 | CGCTGCTTCTCCGTTACCCCTTTGAGACCTCGGGAGCCGGCCCTCTTCCCGCCTCACTGACCATCCCTCGTCCC |
| | | CTATCGCATCTTGGACTCGGAAAGCC |
| Prognostic | TBC1D1 | TTGTGCAGCCCACAGATATCGAGGAAAATCGAACTATGCTCTTCACGATTGGCCAGTCTGAAGTTTACCTCATC |
| | | AGTCCTGACACCAAAAAAATAGCATT |
| Prognostic | ANP32C | GACTACGGAGAAAACGTGTTCAAGCTTCTCCTGCAACTCACATATCTCGACAGCTGTTACTGGGACCACAAGGA |
| | | GGCCCCTTCACTCAGATATTGAGGACC |
| Prognostic | APBB2 | AAATTCCTTACGCAGTGGTATTCATGATGGTGCCCTATCTAAGTCCAGGACTGTTTTCCTACAGCGTGCCTCAA |
| | | AAGTGTTGTAGAGGGCAGGATTCTAC |
| Prognostic | MRPL46 | GGATGAAAACCAGCGACTGGCAAAGAAGAAAGCTGACCTTCATGATGAAGAAGATGAACAGGATATATTGCTGG |
| | | CGCAAGATTTGGAAGATATGTGGGAG |
| Prognostic | NDUFAF1 | CCGAAGTGGGTACTGTGCAATGATATCCAGGATTCCAAGGGGTGCTTTTGAGAGGAAGATGTCTTACGATTGGT |
| | | CCCAGTTCAATACTCTGTATCTCCGT |
| Prognostic | PFDN5 | TCCAACCAGCTCTTCAGGAGAAGCACGCCATGAAACAGGCCGTCATGGAAATGATGAGTCAGAAGATTCAGCAG |
| | | CTCACAGCCCTGGGGGCAGCTCAGGC |
| Prognostic | RTF1 | CTCCACGAGAGACTAATCTAAAAGCTCTGCTTTGTACTTCCTCACCCTGCTTTCGTACAAGGAAGGGGGACGAT |
| | | GGGAAATCATGGACTTGTAAGTTGTA |
| Prognostic | SYNC | TCTCCTCTTCCAAAGAACTCTGGATCCCTAAATGAGGCAGAAGCCTTGAACCCAGAAGTTACTCTATCTTCAGA |
| | | GGGGTCCTTAAACCTCGAAGACATTC |
| Prognostic | TCEA2 | CGGCTCAGATCGAGGAATGCATCTTCCGGGACGTTGGAAACACAGACATGAAGTATAAGAACCGTGTACGGAGT |
| | | CGTATCTCCAACCTGAAGGATGCCAA |
| Prognostic | CNIH3 | AGCTGGCCTTCTATCTCCTCTCCTTCTTCTACTACCTTTACTGCATGATCTACACTTTAGTGAGCTCTTAACGC |
| | | AAAGACCATGCACATCATCAGAGACT |
| Housekeeping | GAPDH | TCCTCCTGTTCGACAGTCAGCCGCATCTTCTTTTGCGTCGCCAGCCGAGCCACATCGCTCAGACACCATGGGGA |
| | | AGGTGAAGGTCGGAGTCAACGGATTT |
| Housekeeping | ACTB | TGCAGAAGGAGATCACTGCCCTGGCACCCAGCACAATGAAGATCAAGATCATTGCTCCTCCTGAGCGCAAGTAC |
| | | TCCGTGTGGATCGGCGGCTCCATCCT |
| Housekeeping | GUSB | CGGTCGTGATGTGGTCTGTGGCCAACGAGCCTGCGTCCCACCTAGAATCTGCTGGCTACTACTTGAAGATGGTG |
| | | ATCGCTCACACCAAATCCTTGGACCC |
| Housekeeping | RPLP0 | CGAAATGTTTCATTGTGGGAGCAGACAATGTGGGCTCCAAGCAGATGCAGCAGATCCGCATGTCCCTTCGCGGG |
| | | AAGGCTGTGGTGCTGATGGGCAAGAA |
| Housekeeping | TFRC | CAGTTTCCACCATCTCGGTCATCAGGATTGCCTAATATACCTGTCCAGACAATCTCCAGAGCTGCTGCAGAAAA |
| | | GCTGTTTGGGAATATGGAAGGAGACT |

1. A method of prognosis for a mammal with BLBC, ERBB2 breast cancer or a molecularly similar cancer comprising:
i) determining in a biological sample obtained from the mammal the expression level of at least each biomarker of the group DSTN, TDRD3, RGS4, MYO1E, RPL3, Hypothetical FLJ13769, ANP32C, MC2R, DKFZp434L092, GPR27, HPS5 and LCP1;
ii) comparing the expression level of each biomarker with the expression level of a housekeeping gene; and
iii) rendering a prognosis for the mammal of a greater than 50% survival for an extended period of time when the expression level of DSTN, TDRD3, RGS4, MYO1E, RPL3, Hypothetical FLJ13769 and ANP32C is decreased in comparison to the expression of the housekeeping gene, and the expression level of MC2R, DKFZp434L092, GPR27, HPS5 and LCP1 is increased in comparison to the expression of the housekeeping gene.

2. The method in claim 1, wherein the molecularly similar cancer is a cancer of the bladder, colon, kidney, liver, lung, esophagus, gall-bladder, ovary, pancreas, stomach, cervix, thyroid, prostate or skin.

3. The method of claim 1, wherein biomarker expression levels is determined by measuring the expression level of biomarker nucleic acid in the sample.

4. The method of claim 3, wherein the level of biomarker nucleic acid in the sample is determined using nucleic acid probes that hybridize to the biomarker nucleic acid.

5. The method of claim 1, wherein biomarker expression level is determined by measuring biomarker activity.

6. The method of claim 1, wherein the housekeeping gene is one or more of ACTB, GAPDH, RPLP0, GUSB, and TFRC.

7. The method of claim 1, wherein the prognosis is rendered when the expression level of DSTN, TDRD3, RGS4, MYO1E, RPL3(1), RPL3(2), RPL3(3), Hypothetical FLJ13769 and ANP32C is decreased by at least 5% in comparison to the expression of the housekeeping gene, and the expression level of MC2R, DKFZp434L092, GPR27, HPS5 and LCP1 is increased by at least about 5% in comparison to the expression of the housekeeping gene.

8. The method of claim 1, including a determination of the expression of one or more additional biomarkers selected from the group consisting of ADAM22, ATP10B, BCL2L14, CR2, DENND3, EPHA5, GLP1R, GLRA3, GPR27, HIST1H3J, HPS5, IQCA1, LCP1, LMAN1L, LOC642131, LRRC37A4, MC2R, MYCNOS, PARP4, PDE4B, PSG11, RFPL3S, RHBG, SLC11A1, SLC5A6 and STS, ANP32C, ANXA2P1, APBB2_CBWD1, CNIH3 DHX15, DSTN, EIF3H, GADD45B, HEXIM1, IL17B, KLHDC2, MRPL46, MYO1E, NDUFAF1, PDZRN3, PFDN5, RGS4, RPL13A, RTF1, SHOX2, SYNC, TBC1D1, and TCEA2, and comparison of the expression of the additional biomarkers to the expression of a housekeeping gene, wherein an increase in expression of ADAM22, ATP10B, BCL2L14, CR2, DENND3, EPHA5, GLP1R, GLRA3, GPR27, HIST1H3J, HPS5, IQCA1, LCP1, LMAN1L, LOC642131, LRRC37A4, MC2R, MYCNOS, PARP4, PDE4B, PSG11, RFPL3S, RHBG, SLC11A1, SLC5A6 or STS, or a decrease in expression of ANP32C, ANXA2P1, APBB2_CBWD1, CNIH3 DHX15, DSTN, EIF3H, GADD45B, HEXIM1, IL17B, KLHDC2, MRPL46, MYO1E, NDUFAF1, PDZRN3, PFDN5, RGS4, RPL13A, RTF1, SHOX2, SYNC, TBC1D1 or TCEA2, is indicative of a positive prognosis.

9. An article of manufacture for use in a method of prognosis in a mammal as defined in claim 1, comprising packaging and a biomarker-specific reactant for each biomarker or nucleic acid encoding the biomarker of the group, DSTN, TDRD3, RGS4, MYO1E, RPL3(1), RPL3(2), RPL3(3), Hypothetical FLJ13769, ANP32C, MC2R, DKFZp434L092, GPR27, HPS5 and LCP1, wherein the reactant is suitable to determine the expression level of the biomarker in a biological sample from the mammal, and wherein the packaging indicates that a determination in the sample of a decreased level of DSTN, TDRD3, RGS4, MYO1E, RPL3(1), RPL3(2), RPL3(3), Hypothetical FLJ13769 and ANP32C and an increased level of MC2R, DKFZp434L092, GPR27, HPS5 and LCP1 in comparison to the expression level of a housekeeping gene is indicative of a prognosis for the mammal of greater than 50% survival for an extended period of time.

10. The article of claim 9, additionally comprising a reactant suitable to detect the expression level of one or more housekeeping genes selected from the group of ACTB, GAPDH, RPLP0, GUSB, and TFRC.

11. The article of claim 10, additionally comprising a biomarker-specific reactant to detect one or more biomarkers selected from the group consisting of ADAM22, ATP10B, BCL2L14, CR2, DENND3, EPHA5, GLP1R, GLRA3, GPR27, HIST1H3J, HPS5, IQCA1, LCP1, LMAN1L, LOC642131, LRRC37A4, MC2R, MYCNOS, PARP4, PDE4B, PSG11, RFPL3S, RHBG, SLC11A1, SLC5A6 and STS, ANP32C, ANXA2P1, APBB2_CBWD1, CNIH3 DHX15, DSTN, EIF3H, GADD45B, HEXIM1, IL17B, KLHDC2, MRPL46, MYO1E, NDUFAF1, PDZRN3, PFDN5, RGS4, RPL13A, RTF1, SHOX2, SYNC, TBC1D1, and TCEA2.

12. The article of claim 9, wherein the biomarker-specific reactant is a nucleic acid probe.

13. The article of claim 12, wherein the biomarker-specific reactant comprises first and second nucleic acid probes for each biomarker.

14. The article of claim 13, wherein, the first probe is labeled with an detectable label, and the second probe is labeled with an immobilization tag.


SEQUENCE LISTING (182 sequences)

SEQ ID NO: 1 - protein, 165 aa, Homo sapiens
MASGVQVADEVCRIFYDMKVRKCSTPEEIKKRKKAVIFCLSADKKCIIVEEGKEILVGDVGVTITDPFKHFVGMLPEKDCRYALYDASFETKESRKEELMFFLWAPELAPLKSKMIYASSKDAIKKKFQGIKHECQANGPEDLNRACIAEKLGGSLIVAFEGCPV

SEQ ID NO: 2 - protein, 148 aa, Homo sapiens
MKVRKCSTPEEIKKRKKAVIFCLSADKKCIIVEEGKEILVGDVGVTITDPFKHFVGMLPEKDCRYALYDASFETKESRKEELMFFLWAPELAPLKSKMIYASSKDAIKKKFQGIKHECQANGPEDLNRACIAEKLGGSLIVAFEGCPV

SEQ ID NO: 3 - DNA, 498 bp, Homo sapiens
atggcctcaggagtgcaagtagctgatgaagtatgtcgcattttttatgacatgaaagttcgtaaatgctccacaccagaagaaatcaagaaaagaaagaaggctgtcattttttgtctcagtgcagacaaaaagtgcatcattgtagaagaaggcaaagagatcttggttggagatgttggtgtaaccataactgatcctttcaagcattttgtgggaatgcttcctgaaaaagattgtcgctatgctttgtatgatgcaagctttgaaacaaaagaatccagaaaagaagagttgatgttttttttgtgggcaccagaactagcacctctgaaaagtaaaatgatctatgcaagctccaaggatgcaattaaaaagaaatttcaaggcataaaacatgaatgtcaagcaaatggaccagaagatctcaatcgggcttgtattgctgaaaagttaggtggatccttaattgtagcctttgaaggatgccctgtgtag

SEQ ID NO: 4 - DNA, 447 bp, Homo sapiens
atgaaagttcgtaaatgctccacaccagaagaaatcaagaaaagaaagaaggctgtcattttttgtctcagtgcagacaaaaagtgcatcattgtagaagaaggcaaagagatcttggttggagatgttggtgtaaccataactgatcctttcaagcattttgtgggaatgcttcctgaaaaagattgtcgctatgctttgtatgatgcaagctttgaaacaaaagaatccagaaaagaagagttgatgttttttttgtgggcaccagaactagcacctctgaaaagtaaaatgatctatgcaagctccaaggatgcaattaaaaagaaatttcaaggcataaaacatgaatgtcaagcaaatggaccagaagatctcaatcgggcttgtattgctgaaaagttaggtggatccttaattgtagcctttgaaggatgccctgtgtag

SEQ ID NO: 5 - protein, 651 aa, Homo sapiens
MLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQKCVSHVQVDSRELDRRKTLQVTMPVKPTNDNDEFEKQRTAAIAEVAKSKETKTFGGGGGGARSNLNMNAAGNRNREVLQKEKSTKSEGKHEGVYRELVDEKALKHITEMGFSKEASRQALMDNGNNLEAALNVLLTSNKQKPVMGPPLRGRGKGRGRIRSEDEEDLGNARPSAPSTLFDFLESKMGTLNVEEPKSQPQQLHQGQYRSSNTEQNGVKDNNHLRHPPRNDTRQPRNEKPPRFQRDSQNSKSVLEGSGLPRNRGSERPSTSSVSEVWAEDRIKCDRPYSRYDRTKDTSYPLGSQHSDGAFKKRDNSMQSRSGKGPSFAEAKENPLPQGSVDYNNQKRGKRESQTSIPDYFYDRKSQTINNEAFSGIKIEKHFNVNTDYQNPVRSNSFIGVPNGEVEMPLKGRRIGPIKPAGPVTAVPCDDKIFYNSGPKRRSGPIKPEKILESSIPMEYAKMWKPGDECFALYWEDNKFYRAEVEALHSSGMTAVVKFIDYGNYEEVLLSNIKPIQTEAWEEEGTYDQTLEFRRGGDGQPRRSTRPTQQFYQPPRARN

SEQ ID NO: 6 - protein, 744 aa, Homo sapiens
MAQVAGAALSQAGWYLSDEGIEACTSSPDKVNVNDIILIALNTDLRTIGKKFLPSDINSGKVEKLEGPCVLQIQKIRNVAAPKDNEESQAAPRMLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQKCVSHVQVDSRELDRRKTLQVTMPVKPTNDNDEFEKQRTAAIAEVAKSKETKTFGGGGGGARSNLNMNAAGNRNREVLQKEKSTKSEGKHEGVYRELVDEKALKHITEMGFSKEASRQALMDNGNNLEAALNVLLTSNKQKPVMGPPLRGRGKGRGRIRSEDEEDLGNARPSAPSTLFDFLESKMGTLNVEEPKSQPQQLHQGQYRSSNTEQNGVKDNNHLRHPPRNDTRQPRNEKPPRFQRDSQNSKSVLEGSGLPRNRGSERPSTSSVSEVWAEDRIKCDRPYSRYDRTKDTSYPLGSQHSDGAFKKRDNSMQSRSGKGPSFAEAKENPLPQGSVDYNNQKRGKRESQTSIPDYFYDRKSQTINNEAFSGIKIEKHFNVNTDYQNPVRSNSFIGVPNGEVEMPLKGRRIGPIKPAGPVTAVPCDDKIFYNSGPKRRSGPIKPEKILESSIPMEYAKMWKPGDECFALYWEDNKFYRAEVEALHSSGMTAVVKFIDYGNYEEVLLSNIKPIQTEAWEEEGTYDQTLEFRRGGDGQPRRSTRPTQQFYQPPRARN

SEQ ID NO: 7 - DNA, 2235 bp, Homo sapiens
atggcccaggtggccggcgcggcgttgtcccaggcgggttggtatctttcagatgaaggcattgaagcttgcacaagctctccagacaaagtcaatgtaaatgacatcatcctgattgctctcaatacagatctgagaacaattggcaagaaattcctccccagtgacatcaatagtggaaaggtagaaaagctcgaaggtccatgtgttttgcaaattcaaaaaattcgcaatgttgctgcaccaaaggataatgaagaatctcaggctgcaccaaggatgctgcgattacagatgactgatggtcatataagttgcacagcagtagaatttagttatatgtcaaaaataagcctgaacacaccacctggaactaaagttaagctctcaggcattgttgacataaaaaatggattcctgctcttgaatgactctaacaccacagttcttggtggtgaagtggaacaccttattgagaaatgggagttacagagaagcttatcaaaacacaatagaagcaatattggaactgaaggtggaccaccgccttttgtgccttttggacagaagtgtgtatctcatgtccaagtggatagcagagaacttgatcgaagaaaaacattgcaagttacaatgcctgtcaaacctacaaatgataatgatgaatttgaaaagcaaaggacggctgctattgctgaagttgcaaagagcaaggaaaccaagacatttggaggaggtggtggtggtgctagaagtaatctcaatatgaatgctgctggtaaccgaaatagggaagttttacagaaagaaaagtcaaccaaatcagagggaaaacatgaaggtgtctatagagaactggttgatgagaaagctctgaagcacataacggaaatgggcttcagtaaggaagcatcgaggcaagctcttatggataatggcaacaacttagaagcagcactgaacgtacttcttacaagcaataaacagaaacctgttatgggtcctcctctgagaggtagaggaaaaggcagggggcgaataagatctgaagatgaagaggacctgggaaatgcaaggccatcagcaccaagcacattatttgatttcttggaatctaaaatgggaactttgaatgtggaagaacctaaatcacagccacagcagcttcatcagggacaatacagatcatcaaatactgagcaaaatggagtaaaagataataatcatctgagacatcctcctcgaaatgataccaggcagccaagaaatgaaaaaccgcctcgttttcaaagagactcccaaaattcaaagtcagttttagaaggcagtggattacctagaaatagaggttctgaaagaccaagtacttcttcagtatctgaagtatgggctgaagacagaatcaaatgtgatagaccgtattctagatatgacagaactaaagatacttcatatcctttaggttctcagcatagtgatggtgcttttaaaaaaagagataactctatgcaaagcagatcaggaaaaggtccctcctttgcagaggcaaaagaaaatccacttcctcaaggatctgtagattataataatcaaaaacgtggaaaaagagaaagccaaacatctattcctgattatttttatgacaggaaatcacaaacaataaataatgaagctttcagtggtataaaaattgaaaaacattttaatgtaaatactgattatcagaatccagttcgaagtaatagtttcattggtgttccaaatggagaagtagaaatgccactgaaaggaagacgaataggacctattaagccagcaggacctgtcacagctgtaccctgtgatgataaaatattttacaatagtgggcccaaacgaagatctgggccaattaagccagaaaaaatactagaatcatctattcctatggagtatgcaaaaatgtggaaacctggagatgaatgttttgcactttattgggaagacaacaagttttaccgggcagaagttgaagccctccattcttcgggtatgacagcagttgttaaattcattgactacggaaactatgaagaggtgctactgagcaatatcaagcccattcaaacagaggcatgggaggaagaaggcacctacgatcaaactctggagttccgtaggggaggtgatggccagccaagacgatccactcggccaacccaacagttttaccaaccaccccgggctcggaactaa

SEQ ID NO: 8 - DNA, 1956 bp, Homo sapiens
atgctgcgattacagatgactgatggtcatataagttgcacagcagtagaatttagttatatgtcaaaaataagcctgaacacaccacctggaactaaagttaagctctcaggcattgttgacataaaaaatggattcctgctcttgaatgactctaacaccacagttcttggtggtgaagtggaacaccttattgagaaatgggagttacagagaagcttatcaaaacacaatagaagcaatattggaactgaaggtggaccaccgccttttgtgccttttggacagaagtgtgtatctcatgtccaagtggatagcagagaacttgatcgaagaaaaacattgcaagttacaatgcctgtcaaacctacaaatgataatgatgaatttgaaaagcaaaggacggctgctattgctgaagttgcaaagagcaaggaaaccaagacatttggaggaggtggtggtggtgctagaagtaatctcaatatgaatgctgctggtaaccgaaatagggaagttttacagaaagaaaagtcaaccaaatcagagggaaaacatgaaggtgtctatagagaactggttgatgagaaagctctgaagcacataacggaaatgggcttcagtaaggaagcatcgaggcaagctcttatggataatggcaacaacttagaagcagcactgaacgtacttcttacaagcaataaacagaaacctgttatgggtcctcctctgagaggtagaggaaaaggcagggggcgaataagatctgaagatgaagaggacctgggaaatgcaaggccatcagcaccaagcacattatttgatttcttggaatctaaaatgggaactttgaatgtggaagaacctaaatcacagccacagcagcttcatcagggacaatacagatcatcaaatactgagcaaaatggagtaaaagataataatcatctgagacatcctcctcgaaatgataccaggcagccaagaaatgaaaaaccgcctcgttttcaaagagactcccaaaattcaaagtcagttttagaaggcagtggattacctagaaatagaggttctgaaagaccaagtacttcttcagtatctgaagtatgggctgaagacagaatcaaatgtgatagaccgtattctagatatgacagaactaaagatacttcatatcctttaggttctcagcatagtgatggtgcttttaaaaaaagagataactctatgcaaagcagatcaggaaaaggtccctcctttgcagaggcaaaagaaaatccacttcctcaaggatctgtagattataataatcaaaaacgtggaaaaagagaaagccaaacatctattcctgattatttttatgacaggaaatcacaaacaataaataatgaagctttcagtggtataaaaattgaaaaacattttaatgtaaatactgattatcagaatccagttcgaagtaatagtttcattggtgttccaaatggagaagtagaaatgccactgaaaggaagacgaataggacctattaagccagcaggacctgtcacagctgtaccctgtgatgataaaatattttacaatagtgggcccaaacgaagatctgggccaattaagccagaaaaaatactagaatcatctattcctatggagtatgcaaaaatgtggaaacctggagatgaatgttttgcactttattgggaagacaacaagttttaccgggcagaagttgaagccctccattcttcgggtatgacagcagttgttaaattcattgactacggaaactatgaagaggtgctactgagcaatatcaagcccattcaaacagaggcatgggaggaagaaggcacctacgatcaaactctggagttccgtaggggaggtgatggccagccaagacgatccactcggccaacccaacagttttaccaaccaccccgggctcggaactaa

SEQ ID NO: 9 - protein, 205 aa, Homo sapiens
MCKGLAGLPASCLRSAKDMKHRLGFLLQKSDSCEHNSSHNKKDKVVICQRVSQEEVKKWAESLENLISHECGLAAFKAFLKSEYSEENIDFWISCEEYKKIKSPSKLSPKAKKIYNEFISVQATKEVNLDSCTREETSRNMLEPTITCFDEAQKKIFNLMEKDSYRRFLKSRFYLDLVNPSSCGAEKQKGAKSSADCASLVPQCA

SEQ ID NO: 10 - protein, 205 aa, Homo sapiens
MCKGLAGLPASCLRSAKDMKHRLGFLLQKSDSCEHNSSHNKKDKVVICQRVSQEEVKKWAESLENLISHECGLAAFKAFYSEENIDFWISCEEYKKIKSPSKLSPKAKKIYNEFISVQATKEVNLDSCTREETSRNMLELKSEPTITCFDEAQKKIFNLMEKDSYRRFLKSRSYLDLVNPSSCGAEKQKGAKSSADCASLVPQCA

SEQ ID NO: 11 - protein, 302 aa, Homo sapiens
MYNMMLLIQKRKGIGSQLLRAGEAEGDRGAGTAERSSDWLDGRSWAIKETPTGLAGRRSEDSDNIFTGEEAKYAQSRSHSSSCRISFLLANSKLLNKMCKGLAGLPASCLRSAKDMKHRLGFLLQKSDSCEHNSSHNKKDKVVICQRVSQEEVKKWAESLENLISHECGLAAFKAFLKSEYSEENIDFWISCEEYKKIKSPSKLSPKAKKIYNEFISVQATKEVNLDSCTREETSRNMLEPTITCFDEAQKKIFNLMEKDSYRRFLKSRFYLDLVNPSSCGAEKQKGAKSSADCASLVPQCA

SEQ ID NO: 12 - protein, 93 aa, Homo sapiens
MCKGLAGLPASCLRSAKDMKHRLGFLLQKSDSCEHNSSHNKKDKVVICQRVSQEEVKKWAESLENLISHECEPGFLHQGRDKPEHARAYNNLL

SEQ ID NO: 13 - protein, 187 aa, Homo sapiens
MKHRLGFLLQKSDSCEHNSSHNKKDKVVICQRVSQEEVKKWAESLENLISHECGLAAFKAFLKSEYSEENIDFWISCEEYKKIKSPSKLSPKAKKIYNEFISVQATKEVNLDSCTREETSRNMLEPTITCFDEAQKKIFNLMEKDSYRRFLKSRFYLDLVNPSSCGAEKQKGAKSSADCASLVPQCA

SEQ ID NO: 14 - DNA, 909 bp, Homo sapiens
atgtataatatgatgcttctaatccaaaagaggaaaggcattgggagtcagctcctaagggctggagaggcagagggagacagaggagctggtactgcagagcggtcgtctgattggctggacggtcgtagctgggctataaaagagacccctacaggcttagcaggaagacgctcagaggattctgacaatatctttaccggagaagaggcaaagtacgctcaaagccgaagccacagctcctcctgccgcatttctttcctgcttgcgaattccaagctgttaaataagatgtgcaaagggcttgcaggtctgccggcttcttgcttgaggagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaa

SEQ ID NO: 15 - DNA, 618 bp, Homo sapiens
atgtgcaaagggcttgcaggtctgccggcttcttgcttgaggagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaa

SEQ ID NO: 16 - DNA, 564 bp, Homo sapiens
atgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaa

SEQ ID NO: 17 - DNA, 282 bp, Homo sapiens
atgtgcaaagggcttgcaggtctgccggcttcttgcttgaggagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgaacctggattcttgcaccagggaagagacaagccggaacatgctagagcctacaataacctgctttga

SEQ ID NO: 18 - protein, 1108 aa, Homo sapiens
MGSKGVYQYHWQSHNVKHSGVDDMVLLSKITENSIVENLKKRYMDDYIFTYIGSVLISVNPFKQMPYFGEKEIEMYQGAAQYENPPHIYALADNMYRNMIIDRENQCVIISGESGAGKTVAAKYIMSYISRVSGGGTKVQHVKDIILQSNPLLEAFGNAKTVRNNNSSRFGKYFEIQFSPGGEPDGGKISNFLLEKSRVVMRNPGERSFHIFYQLIEGASAEQKHSLGITSMDYYYYLSLSGSYKVDDIDDRREFQETLHAMNVIGIFAEEQTLVLQIVAGILHLGNISFKEVGNYAAVESEEFLAFPAYLLGINQDRLKEKLTSRQMDSKWGGKSESIHVTLNVEQACYTRDALAKALHARVFDFLVDSINKAMEKDHEEYNIGVLDIYGFEIFQKNGFEQFCINFVNEKLQQIFIELTLKAEQEEYVQEGIRWTPIEYFNNKIVCDLIENKVNPPGIMSILDDVCATMHAVGEGADQTLLQKLQMQIGSHEHFNSWNQGFIIHHYAGKVSYDMDGFCERNRDVLFMDLIELMQSSELPFIKSLFPENLQADKKGRPTTAGSKIKKQANDLVSTLMKCTPHYIRCIKPNETKKPRDWEESRVKHQVEYLGLKENIRVRRAGYAYRRIFQKFLQRYAILTKATWPSWQGEEKQGVLHLLQSVNMDSDQFQLGRSKVFIKAPESLFLLEEMRERKYDGYARVIQKSWRKFVARKKYVQMREEASDLLLNKKERRRNSINRNFIGDYIGMEEHPELQQFVGKREKIDFADTVTKYDRRFKGVKRDLLLTPKCLYLIGREKVKQGPDKGLVKEVLKRKIEIERILSVSLSTMQDDIFILHEQEYDSLLESVFKTEFLSLLAKRYEEKTQKQLPLKFSNTLELKLKKENWGPWSAGGSRQVQFHQGFGDLAVLKPSNKVLQVSIGPGLPKNSRPTRRNTTQNTGYSSGTQNANYPVRAAPPPPGYHQNGVIRNQYVPYPHAPGSQRSNQKSLYTSMARPPLPRQQSTSSDRVSQTPESLDFLKVPDQGAAGVRRQTTSRPPPAGGRPKPQPKPKPQVPQCKALYAYDAQDTDELSFNANDIIDIIKEDPSGWWTGRLRGKQGLFPNNYVTKI

SEQ ID NO: 19 - DNA, 3327 bp, Homo sapiens
atgggaagcaaaggtgtctaccagtaccactggcaaagccacaatgtcaagcacagtggtgtggacgacatggtgctactgtccaagatcacagagaactccatcgtggagaatctgaagaagagatacatggatgactacatttttacatatataggatctgtattaatctcagtcaaccctttcaagcagatgccatattttggggaaaaggaaattgaaatgtaccaaggagcggcacagtatgaaaacccaccacatatctatgcccttgcagataatatgtacagaaacatgatcattgacagagagaaccagtgcgtcattatcagtggtgaaagtggtgctggaaaaacagtggctgccaaatatatcatgagctacatctccagagtgtctggaggagggaccaaagtccagcacgtgaaggacattatcctgcagtccaacccgctgctggaggccttcgggaacgccaagaccgtccggaacaacaactccagccgatttggaaaatactttgaaatccagttcagtccaggtggggaaccagatggtggaaagatctccaacttccttctggaaaaatctagggtggtgatgaggaacccaggagagcggagttttcacatattttaccagctcatcgagggcgcctctgcagagcagaaacacagccttggcatcaccagcatggactattattactacctgagcctctcgggctcatacaaggttgatgacattgacgacaggcgggagtttcaggaaactctgcacgccatgaatgtgattgggatctttgcagaagagcaaacgctggtgttgcagatagtggcgggtattctccacctgggaaacatcagcttcaaagaagttggcaactacgcggctgtggagagtgaagagtttttagcttttcctgcatatctgctagggataaaccaggaccggttgaaagaaaagctaacaagccggcagatggatagcaagtggggaggcaaatccgaatccatccacgtgaccctcaacgtagagcaggcctgttacacccgggatgcgctcgccaaggccctgcacgcccgggtctttgatttcttggtagattccatcaataaagccatggagaaagaccatgaagaatacaacattggcgtcctagacatctatggctttgaaatattccagaaaaatggctttgaacagttttgtatcaattttgttaatgaaaaactgcagcagatttttattgaactgacattaaaggcagaacaggaagaatatgttcaagagggaataagatggacacccattgagtactttaataataaaatcgtatgtgacctcatagagaacaaagtgaaccctcctggcatcatgagcatcctggatgacgtgtgcgccacgatgcatgcggtgggtgagggggcagatcagacgctgctccagaaacttcagatgcagattgggagtcatgagcacttcaacagttggaaccaaggcttcatcattcatcattatgctgggaaggtatcctatgacatggatggcttttgtgaaaggaaccgggatgtgctttttatggatctcatcgagcttatgcagagcagcgagctgcctttcataaagtctttatttccggaaaatctgcaggctgacaagaaagggcgcccaactactgccggaagcaaaataaagaaacaagccaatgaccttgtgagcaccctgatgaaatgtacgccccactacattcgctgcatcaagccaaacgaaaccaagaagcccagagactgggaggaaagcagggtaaagcatcaagtcgaatatttgggtctgaaagagaacattcgagtgagaagagctggctatgcctatcggcgcatcttccaaaaattcctacagaggtatgccattctgaccaaagccacctggccttcttggcagggagaggagaagcaaggcgtcctgcacctgctgcagtcggtcaacatggacagcgaccagttccagctggggaggagtaaagtgttcatcaaagcccccgagtctctatttcttttagaagagatgagagagagaaagtatgatgggtatgctcgagtgatacagaaatcatggaggaaattcgtggcccggaagaaatacgttcaaatgagagaagaagcctcagacctcttattgaacaagaaggagagaaggagaaacagtattaacaggaactttataggggattatattgggatggaagagcacccagaactccagcagttcgtgggcaagagggagaagattgatttcgcagacacagtcaccaagtatgacaggaggttcaagggtgtaaagcgagacctgctccttaccccaaagtgcttgtacttaatcggacgagaaaaagtcaaacagggcccagacaagggcctggtgaaagaagtcctgaagcggaaaatcgagatagaacggatcttgtctgtgtccctcagtactatgcaggatgacatttttattctccatgagcaagagtatgacagtttgcttgaatctgtcttcaaaactgaattcctaagcctcttagcaaagcgttacgaggagaagacccagaagcaactacctctgaaattcagcaatacgcttgaactgaagttgaaaaaggaaaactggggcccctggagtgcagggggctcccggcaagtgcagttccaccaagggtttggggacctggctgtcctcaagcccagtaacaaagtgctgcaggtcagcatcggacctggactgcccaagaactcccgtcctaccagaaggaacactacccaaaatacaggttattccagtgggactcaaaatgccaactacccagtgagagctgcccctcctcccccaggataccatcagaacggagtcatcagaaaccagtatgtgccatatccccatgctcctggaagccagaggtccaatcagaaaagcctgtacacctccatggcccgcccgcccttgcctcggcagcagtctaccagttcagaccgagtgtcacagacgccagagagcctggatttcctcaaggtcccggaccagggagctgcaggggtcaggagacaaacaaccagtcggcctcccccagcagggggcagacccaagccccagcccaagcccaagcctcaggtgccacagtgcaaggctttgtatgcctatgacgctcaggacacagacgaactcagctttaatgccaatgacattattgatattatcaaagaagatccttctggctggtggacgggtcgactacgaggcaagcagggcctgttccccaacaactatgtgaccaagatctga

SEQ ID NO: 20 - DNA, 432 bp, Homo sapiens
atggtgccagactggagtccttttattaaaattaactgccctgctcagctttctgctgggccaccccagagccaatccttggttcttgggcccaaggctggacccaggggttgcaggaaacagtctgtagcatccaagtggggcctgtcgtacccactccagtgtgtaggtgcagaacgctctttgggggatttctctgctgggccaccttactccagggatccctcagttttcaaaacaaagcaagagggcaaggaagaatggagaaacagctcagtgttgactctcttccccctggtgagtgctgtgctggggcctctgtgcacatcataccacttcccccttgaatcagccccacaaggcagggtgagagatgaggactcagggtgcaaggaggtctcacagcttggaaatggatcaggacagctctga

SEQ ID NO: 21 - DNA, 5243 bp, Homo sapiens
gcagttaagtatttgtttaatcttgctttgtcttttcaaacagcgatttagtaatcctgtttgaggctgcagtgtggcaatgctttccagaggatggagtcctttttgtttgttttgaaaaaatagagatggggtttcactatgttgcctaggctggtctcatactcggcctcgagccatcctcctgcctgggcctcccaaagtgttgggattacaggcgtgagccatggtgccagactggagtccttttattaaaattaactgccctgctcagctttctgctgggccaccccagagccaatccttggttcttgggcccaaggctggacccaggggttgcaggaaacagtctgtagcatccaagtggggcctgtcgtacccactccagtgtgtaggtgcagaacgctctttgggggatttctctgctgggccaccttactccagggatccctcagttttcaaaacaaagcaagagggcaaggaagaatggagaaacagctcagtgttgactctcttccccctggtgagtgctgtgctggggcctctgtgcacatcataccacttcccccttgaatcagccccacaaggcagggtgagagatgaggactcagggtgcaaggaggtctcacagcttggaaatggatcaggacagctctgattctccaaggccaaggtcttctctatatcatgaggcagcccaaaaatgtgtttctacacatattctgtgtagaaactcaggccaggctcctatatgctctgggtgacagcgggaaggtgctttccagtgtgtatcagtgtggcccatctgggagtcataatgtcattgcctcttccaaaatatcagcaaagctgaccctagtgcttgtagtgctcaaatgcacctctctgcctttgtgccatagggaaatggatttggtcctgggggtggtgataccaacttctgtctcaattccatgaccctcactcagcacccccaccccaggcctgtgcagaggaggaataaccgtccttgagaaccatccagccctaggtggagaaattagatttatggacttaatcagttcttcttccctgtcagtggtggtgagaatgacatgggggaaattgataagcctcagtcatgttttcctgcgagacaaggcagctttggtagctggtgggactgacgccttcttcacctgtgtcctctgcctctgagtgactataaattgcgaaccccatgaggtggcacctgcagccacagatcgcttaggcaccttgcacagggggccacctaatgaactggctgcaggacggcaatcctgccgtgaagccaggcacctcgggaggggcaaggtggcagaaccgagagagcagatgctccttgctgtttctgtaaggccaccagtggctgggtggcctggccctggtagcaaggtggtaaccgtgtgagtggctgaatcctgcaccacttttcctgagcctccttcctcctggattcctgcaactatattctcactacatggctcaccaccaattcgttctctctgccccaaggaggctggacaagaggctcaggtcagtgcgggtacgcaggaacctatgtctgtaaaatgaaaatgcctgccttcaagggctgtcttgaggttgaaaggagatggtctacatcaagggttaggaagaccacaatgtggctgtgccggccacctccaggctggcttcagtctcccatccccatagtcatacccctgtgaagtcccttcccatactgaatcgggctacctgtgtatcttatagggtgttggaaatgacagattgtgaattccaaggccccataaaagacagcgtgggctgggcgtggtggctcacgcctgtaatcccagcactttgggaggccgagccaagtggatcacttgaggtcaggagttctagaccagcctggccagcatggtgaaaacctatctctactaaaaatacaaaaaaaaaaatttagccaggtgtgttggcacctgtgcctgtagtcccagatacttgggaggctgagccaggagaatcacttgaacctgagaggtggaggtttcagtgagccgagattgtgccactgcactccagctgggtggcagagactctgtctctaaacaaagaaaatgtggtttctgctgtcctttctcttggatcactttctctgggaaaccagccaccatcttgtgaggacactcaagctctctggaaaggttcagaaatgtggtgaggaagtgagacagacctcttgccaaaaaccatgtactaacttatcagtcacgtacatgagttgacttagaaatgactctatcagcctcagccaaaccttcagatgactgcagccctggtcaacatcttgactgtgaccccatggaagaccctgaaacactcagccaagccactctcagcttcctgatccacagaagcagcatgaaataataaatttttcttgttttaaactaagttttgggttaatttgttatacagctgtagataactccaagaataatactccataaatgttgatttcctttctgcggctccactgagtcatctttgttcatgggcaaagctcactgtctctctactggctgcaaaagatgtccatttgttggctctgactcatgcttgtggtcttgttggctcacagcaaggccttttcacagaggatggtggtttgtcacactcagaatccaggtcttacctgctttcctgctccatggaattttgacctgagtttgaaggctgataggggattgatggatgagggagaaagacacagtcagttcttagagcagtagctaatccaggggtttctctggctttgtttgcatgagtagtgatagttggggatgaagcatgggtattcgagatcttttttgtgtgagtatgggaggaaaaattctgttctcttcctcctccatctttattcatctttgtcttccctcggatcagctttaaagacccctgagactctaggactcggtttgcttttttctcaccactgggatccatctcccactagtaggaagaaggatctaagtagttgagctgcataaaggggaagtaaacagccagacaaatgttacattttagagccttgagtgacccagagctttctatcagcttcattgggtgcagggagggtagcgatttctggctgctgtcacctccttgctcatatcaggaagcgctaattttgaggcaaagttcagtgccctctggctcacctagggcccctgttgcctaatttcacgaggccaagagtttgagggtgggttcggggctgcgcagcctggatgccttggtttgcatgcctacagcagctgtgcccgaaagctttttttagagtaaatatgtagcttttgctaggctttttgtgtgccttgttcattcctctatcattttttgtatacctgctatggtgtcaggcactggggatacaaattagccggggactcggacatgtaagcaagtaagcccaacagcagctaacatttatgcagtgctcactgtgtgcttggatgtccagagtgatagagcaggtttagtaagggggacagagggctgagaccagaggcaaggtggagaagggcatttctggaggcagcaatatgagtgcaaaggagacatacacaaggttccctgggggattccaagttgttgctgtcgtggcttcaaatcttactttagggcctactcagatggcatcttgttcatgaagatgtgcctgttttttgttttatttgtccctctccccccaacaaagagtaaactttttttctgaacttctcgaaagtttgctaacatcagctataaaactctctgggcctgggatattttgcggggagcagggaagatctttgattatcattttaacttatttctgttcatttattcaagctttttcttctttcaccaattctcaaaagccccacaccaccctcctgccaaccccacacccctccacttccagtcgctaccttccacccatcattctcacttctaaagccatcaacagaggttttgaacaccactcatgtgccaggtgttgttctaagcacagattcatcagtgagccaaatgaaattccttcctaaaatatgctctttggtggtgctaaatgccaggaagaaaagtaagcaaggggagggaagagagaggtgtggggtggcggggggcgggagcttggataggatggtcagaaaggacctctttgtagaaacttaaataaatttagaaagcaagtcatgtaatatctgggaaagaggaaatagcaggtgcaaagaccctgaggttgaagtgtcctggcaagtttgaggcaagcaaggaaggcagggtggttggaacagaacaagagaagaaacgtggtaggaaatgatggagagtcagggacacgaccttggaggacttgcaggtcatggtaaagacgctggactcgacacgaagtgtgaacagagttcttggaggattctgaggtagagagtagcataatctcatttgtgtttttattctaacatgctcacttgggccaccttgtagaggctagagtgaaaaggagggagactagttagaaattctgtattgctggctgggcgtggtagctcccggcactgtgggaggccaaggccagaggattgcttgagtctagcagtttgagcccagcctaggcaacatagcgagaccctgtctctacaaaaaattttaaaattgcccagggatggtggtgcacacttgtagtcccaactacttgggaggctgaggtgggaggattgcctgagcccaggagtttgaggctccagtgagctgtgattgtgccattgcactccagcctgggtgacagagcgagaccctgtctctaaaaaaaaaagaaaaaaaaagaaattctgtattgcagcagcctaagagagtcattagttaagagatttgtagtaggcattttttagaaagttgcattgagggctaggtacggtggctcatgcctataatcccagcactttgggaggccaaggtgggtagatcatttgaggtcaggagcctggccaacatagcgaaaccccgtctctactaaaaaaattagccgggcggtagtggcatgcgcctgtaatcccagctactcgggaggctgaggcaggagaattgcttgagcctgggaggtggaggttgccagtgagtcgagatcgcgccactgcactccagcagtctgggcgacagagtgagacgctgtttcaaaaaaaaaaaag

SEQ ID NO: 22 - protein, 403 aa, Homo sapiens
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAFTKYCKKWQDEDGKKQLEKDFSSMKKYCQVIRVIAHTQMRLLPLRQKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKRRALEKIDLKFIDTTSKFGHGRFQTMEEKKAFMGPLKKDRIAKEEGA

SEQ ID NO: 23 - protein, 403 aa, Homo sapiens
MSHRKFSAPRHGSLGFLPRKRSSRHRGKVKSFPKDDASKPVHLTAFLGYKAGMTHIVREVDRPGSKVNKKEVVEAVTIVETPPMVVVGIVGYVETPRGLRTFKTVFAEHISDECKRRFYKNWHKSKKKAFTKYCKKWQDDTGKKQLEKDFNSMKKYCQVIRIIAHTQMRLLPLRQKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFIMLKGCVVGTKKRVLTLRKSLLVQTKRRALEKIDLKFIDTTSKFGHGRFQTMEEKKAFMGPLKKDRIAKEEGA

SEQ ID NO: 24 - DNA, 1212 bp, Homo sapiens
atgtctcacagaaagttctccgctcccagacatgggtccctcggcttcctgcctcggaagcgcagcagcaggcatcgtgggaaggtgaagagcttccctaaggatgacccgtccaagccggtccacctcacagccttcctgggatacaaggctggcatgactcacatcgtgcgggaagtcgacaggccgggatccaaggtgaacaagaaggaggtggtggaggctgtgaccattgtagagacaccacccatggtggttgtgggcattgtgggctacgtggaaacccctcgaggcctccggaccttcaagactgtctttgctgagcacatcagtgatgaatgcaagaggcgtttctataagaattggcataaatctaagaagaaggcctttaccaagtactgcaagaaatggcaggatgaggatggcaagaagcagctggagaaggacttcagcagcatgaagaagtactgccaagtcatccgtgtcattgcccacacccagatgcgcctgcttcctctgcgccagaagaaggcccacctgatggagatccaggtgaacggaggcactgtggccgagaagctggactgggcccgcgagaggcttgagcagcaggtacctgtgaaccaagtgtttgggcaggatgagatgatcgacgtcatcggggtgaccaagggcaaaggctacaaaggggtcaccagtcgttggcacaccaagaagctgccccgcaagacccaccgaggcctgcgcaaggtggcctgtattggggcatggcatcctgctcgtgtagccttctctgtggcacgcgctgggcagaaaggctaccatcaccgcactgagatcaacaagaagatttataagattggccagggctaccttatcaaggacggcaagctgatcaagaacaatgcctccactgactatgacctatctgacaagagcatcaaccctctgggtggctttgtccactatggtgaagtgaccaatgactttgtcatgctgaaaggctgtgtggtgggaaccaagaagcgggtgctcaccctccgcaagtccttgctggtgcagacgaagcggcgggctctggagaagattgaccttaagttcattgacaccacctccaagtttggccatggccgcttccagaccatggaggagaagaaagcattcatgggaccactgaagaaagaccgaattgcaaaggaagaaggagcttaa

SEQ ID NO: 25 - protein, 234 aa, Homo sapiens
MEMGRRIHSELRNRAPSDVKELALDNSRSNEGKLEALTDEFEELEFLSKINGGLTSISDLPKLKLRKLELRVSGGLEVLAEKCPNLTHLYLSGNKIKDLSTIEPLKQLENLKSLDLFNCEVTNLNDYGENVFKLLLQLTYLDSCYWDHKEAPYSDIEDHVEGLDDEEEGEHEEEYDEDAQVVEDEEGEEEEEEGEEEDVSGGDEEDEEGYNDGEVDGEEDEEELGEEERGQKRK

SEQ ID NO: 26 - DNA, 705 bp, Homo sapiens
atggagatgggcagacggattcattcagagctgcggaacagggcgccctctgatgtgaaagaacttgccctggacaacagtcggtcgaatgaaggcaaactcgaagccctcacagatgaatttgaagaactggaattcttaagtaaaatcaacggaggcctcacctcaatctcagacttaccaaagttaaagttgagaaagcttgaactaagagtctcagggggcctggaagtattggcagaaaagtgtccaaacctcacgcatctatatttaagtggcaacaaaattaaagacctcagcacaatagagccactgaaacagttagaaaacctcaagagcttagaccttttcaattgcgaggtaaccaacctgaacgactacggagaaaacgtgttcaagcttctcctgcaactcacatatctcgacagctgttactgggaccacaaggaggccccttactcagatattgaggaccacgtggagggcctggatgacgaggaggagggtgagcatgaggaggagtatgatgaagatgctcaggtagtggaagatgaggagggcgaggaggaggaggaggaaggtgaagaggaggacgtgagtggaggggacgaggaggatgaagaaggttataacgatggagaggtagatggcgaggaagatgaagaagagcttggtgaagaagaaaggggtcagaagcgaaaatga

SEQ ID NO: 27 - protein, 297 aa, Homo sapiens
MKHIINSYENINNTARNNSDCPRVVLPEEIFFTISIVGVLENLIVLLAVFKNKNLQAPMYFFICSLAISDMLGSLYKILENILIILRNMGYLKPRGSFETTADDIIDSLFVLSLLGSIFSLSVIAADRYITIFHALRYHSIVTMRRTVVVLTVIWTFCTGTGITMVIFSHHVPTVITFTSLFPLMLVFILCLYVHMFLLARSHTRKISTLPRANMKGAITLTILLGVFIFCWAPFVLHVLLMTFCPSNPYCACYMSLFQVNGMLIMCNAVIDPFIYAFRSPELRDAFKKMIFCSRYW

SEQ ID NO: 28 - protein, 296 aa, Homo sapiens
MKHIINSYEHTNDTARNNSDCPDVVLPEEIFFTISVIGILENLIVLLAVIKNKNLQSPMYFFICSLAISDMLGSLYKILENILIMFRNMGYLKPRGSFESTADDIIDCMFILSLLGSIFSLSVIAADRYITIFHALQYHSIVTMRRTIITLTIIWMFCTGSGITMVIFSHHIPTVLTFTSLFPLMLVFILCLYIHMFLLARSHARKISTLPRTNMKGAMTLTILLGVFIFCWAPFVLHVLLMTFCPNNPYCVCYMSLFQVNGMLIMCNAVIDPFIYAFRSPELRDAFKRMLFCNRY

SEQ ID NO: 29 - DNA, 894 bp, Homo sapiens
atgaagcacattatcaactcgtatgaaaacatcaacaacacagcaagaaataattccgactgtcctcgtgtggttttgccggaggagatatttttcacaatttccattgttggagttttggagaatctgatcgtcctgctggctgtgttcaagaataagaatctccaggcacccatgtactttttcatctgtagcttggccatatctgatatgctgggcagcctatataagatcttggaaaatatcctgatcatattgagaaacatgggctatctcaagccacgtggcagttttgaaaccacagccgatgacatcatcgactccctgtttgtcctctccctgcttggctccatcttcagcctgtctgtgattgctgcggaccgctacatcaccatcttccacgcactgcggtaccacagcatcgtgaccatgcgccgcactgtggtggtgcttacggtcatctggacgttctgcacggggactggcatcaccatggtgatcttctcccatcatgtgcccacagtgatcaccttcacgtcgctgttcccgctgatgctggtcttcatcctgtgcctctatgtgcacatgttcctgctggctcgatcccacaccaggaagatctccaccctccccagagccaacatgaaaggggccatcacactgaccatcctgctcggggtcttcatcttctgctgggccccctttgtgcttcatgtcctcttgatgacattctgcccaagtaacccctactgcgcctgctacatgtctctcttccaggtgaacggcatgttgatcatgtgcaatgccgtcattgaccccttcatatatgccttccggagcccagagctcagggacgcattcaaaaagatgatcttctgcagcaggtactggtag

SEQ ID NO: 30 - DNA, 2979 bp, Homo sapiens
atgcacaggtgtacagaccacctctgttgatgcgcgcgcaggagactgtgagcagatgccacactaggtgggtgctggactggagcctggggaagaactagaggggctggaagggcgcagggaggtctgccagggtgaagggacagcctaggggaaacccagctcggaacccacaaggcagggcagctggagcgtgcagtctggtctccccgtgttcaagctctcactgtggggcttcatctagtgcatgacacccctgggagcacaaatgcctgctcctactgataaagaagcagggggagtgagcggctcccaagcagcggcgcaggagcgaaggcaggtctagacatccctgccctttttcccatcacagcagtctctgcagttgctgctgaggtcgagtttccgaattgaggtgctgctttgctccctttccaactcaggctgctctaagctcttccagaaaggggctccagagggaactttgccaggacagttggagaactgccgagacaggcagggttcctggttccagagccccacgagaaaggggtctttgctttgcaccttcctgcagcctagcagcgttcctgcctcagagcaggacgaggcctggggtcttctaggaaagccctttgtacactgtcaaggggatccccacgccaaggccatttacagcctttgttcttccagcctgcggaccctcctgcctagccctgccatgttcgcagcagccagccagccagctctgcctctgcccgaggccagggaccccgaccttagagcctggggtttctaggtaggctgccctgcttgcctgtggtttggacaggagagctgggcttggcacaagtgggtgctggaaagaggcttcaatatgacggactttccgtctctggttaaatcacaataaaccactggaggagagctttgaggctgtggtttgaatcctgggtgccaggagacctttctcccacctcgctttccttgctggaaatgagggtacagtaggtgacccctcaggtccctctgagctaccactctacaaatcactctgctcagattttagctactgccattaaggcacagtgcagagagtttaaaaattgctgcagagtttaaaaattgcctgacatacaaaaattagccaggcgtggtggcgggcgcctgtagccccagctactcgggaggctgaggcaggaggatcacttgaatctgggaggcggaggttgcagtgagccgagcttgcgccactacactccagcttgggtgacagaatgagactttatctccataaataaataatcgcccaacagcaggctgggtctccatacccagcttttccaggttcacctctcgtggctgttttcacatggatagggaagacatgggtcatccttccctcccagactccccccaggacactgcccggccctctagacagcaggtggagtcagcagagggagcagggccttctttttgtggttaatgatctctgtcctaagcaatgccatggccccagactcacatacatctctaataaccagcagaggactgtttcacagaaactgctgtgttccagacaagcagggacaccgatttccctcccacttgccacgggagatgggaagaatgataccagcagcaggaatcctttttctgggtgatcgaaagacaattttcagtttaaaaagcatccttggaatggctgtgggtgaagccttctcctgaggcctgggatgggctgactttagcactttggctttctatgggtctttgaagtgagatggagtgaaaggctgttctactcaggtgtcctcagacttggcacctggctaagtgctgtccctcgcccagatgggaagaggctgcagtgactggccctggagctgatgttggcctatcccccaggatggttttagccacctggctgattgtggaaacttctccacaaaatgagatgaattatactgtcctcaacattctcccaataaatactcagaactactatcagcttaggccaaggcatggtgattatactttcgagaaggatagttaagcagagaatcacaggatattttctaactgacgaccggaggtgtgtgtgtggaaggtatccaggcctgggaagttgacacagggcagtgagatgaggacaggctaaagagccatggtaggttgaccccaagaggccattgtggagaatacaagctgaaatggctcttgccaacgcctgggacaagctattattttatcccaggaatccagaaccagaagaaacaagcaaatgaagtgattttttttttttttttggagtgatggagaacaatggcagttttgttttgtatactgtgagctctcaacagagttggatttatttttctctcatataaaccccatcccccaaacattttttttttttttttagaggtgatggtagcagacagtgcttgtcactaagtcctctcttggggtgggggcagaggtggtgaggagcaccggcttaggggaaaaggcaggcaagtgaggagcaggccgaagcctgggactgtggaggatgttcgctctgtttgcaggctgagctgcggaaggtaaaaactccaggccaaaggattctagaaatatgggcacaggggtcatattttcatgaaaaaataaaaataaaagcacagggtgggccgggcacagtggctcatgcctgtaatcccagcactttgggaggctgaggtggatggctcacctgaggtcaggagttcaagaccagcctggccaacatggtgaaaccccgtctctactaaaaatacaaaaattagccgggcatggtggcacttgcctgtaagctcagctactcaggagcctgaggcaggagaatcgcttgaacctgggaggcggaggttgcagtgagctgagattgtgccactgcactccagcctgggcaacaagagcgaaactgttttaaaaaaaaaaaaaaaaaaaaaa

SEQ ID NO: 31 - protein, 375 aa, Homo sapiens
MANASEPGGSGGGEAAALGLKLATLSLLLCVSLAGNVLFALLIVRERSLHRAPYYLLLDLCLADGLRALACLPAVMLAARRAAAAAGAPPGALGCKLLAFLAALFCFHAAFLLLGVGVTRYLAIAHHRFYAERLAGWPCAAMLVCAAWALALAAAFPPVLDGGGDDEDAPCALEQRPDGAPGALGFLLLLAVVVGATHLVYLRLLFFIHDRRKMRPARLVPAVSHDWTFHGPGATGQAAANWTAGFGRGPTPPALVGIRPAGPGRGARRLLVLEEFKTEKRLCKMFYAVTLLFLLLWGPYVVASYLRVLVRPGAVPQAYLTASVWLTFAQAGINPVVCFLFNRELRDCFRAQFPCCQSPRTTQATHPCDLKGIGL

SEQ ID NO: 32 - protein, 375 aa, Homo sapiens; identical to SEQ ID NO: 31

SEQ ID NO: 33 - DNA, 1128 bp, Homo sapiens
atggcgaacgcgagcgagccgggtggcagcggcggcggcgaggcggccgccctgggcctcaagctggccacgctcagcctgctgctgtgcgtgagcctagcgggcaacgtgctgttcgcgctgctgatcgtgcgggagcgcagcctgcaccgcgccccgtactacctgctgctcgacctgtgcctggccgacgggctgcgcgcgctcgcctgcctcccggccgtcatgctggcggcgcggcgtgcggcggccgcggcgggggcgccgccgggcgcgctgggctgcaagctgctcgccttcctggccgcgctcttctgcttccacgccgccttcctgctgctgggcgtgggcgtcacccgctacctggccatcgcgcaccaccgcttctatgcagagcgcctggccggctggccgtgcgccgccatgctggtgtgcgccgcctgggcgctggcgctggccgcggccttcccgccagtgctggacggcggtggcgacgacgaggacgcgccgtgcgccctggagcagcggcccgacggcgcccccggcgcgctgggcttcctgctgctgctggccgtggtggtgggcgccacgcacctcgtctacctccgcctgctcttcttcatccacgaccgccgcaagatgcggcccgcgcgcctggtgcccgccgtcagccacgactggaccttccacggcccgggcgccaccggccaggcggccgccaactggacggcgggcttcggccgcgggcccacgccgcccgcgcttgtgggcatccggcccgcagggccgggccgcggcgcgcgccgcctcctcgtgctggaagaattcaagacggagaagaggctgtgcaagatgttctacgccgtcacgctgctcttcctgctcctctgggggccctacgtcgtggccagctacctgcgggtcctggtgcggcccggcgccgtcccccaggcctacctgacggcctccgtgtggctgaccttcgcgcaggccggcatcaaccccgtcgtgtgcttcctcttcaacagggagctgagggactgcttcagggcccagttcccctgctgccagagcccccggaccacccaggcgacccatccctgcgacctgaaaggcattggtttatga

SEQ ID NO: 34 - protein, 1129 aa, Homo sapiens
MAFVPVIPESYSHVLAEFESLDPLLSALRLDSSRLKCTSIAVSRKWLALGSSGGGLHLIQKEGWKHRLFLSHREGAISQVACCLHDDDYVAVATSQGLVVVWELNQERRGKPEQMYVSSEHKGRRVTALCWDTAILRVFVGDHAGKVSAIKLNTSKQAKAAAAFVMFPVQTITTVDSCVVQLDYLDGRLLISSLTRSFLCDTEREKFWKIGNKERDGEYGACFFPGRCSGGQQPLIYCARPGSRMWEVNFDGEVISTHQFKKLLSLPPLPVITLRSEPQYDHTAGSSQSLSFPKLLHLSEHCVLTWTERGIYIFIPQNVQVLLWSEVKDIQDVAVCRNELFCLHLNGKVSHLSLISVERCVERLLRRGLWNLAARTCCLFQNSVIASRARKTLTADKLEHLKSQLDHGTYNDLISQLEELILKFEPLDSACSSRRSSISSHESFSILDSGIYRIISSRRGSQSDEDSCSLHSQTLSEDERFKEFTSQQEEDLPDQCCGSHGNEDNVSHAPVMFETDKNETFLPFGIPLPFRSPSPLVSLQAVKESVSSFVRKTTEKIGTLHTSPDLKVRPELRGDEQSCEEDVSSDTCPKEEDTEEEKEVTSPPPEEDRFQELKVATAEAMTKLQDPLVLFESESLRMVLQEWLSHLEKTFAMKDFSGVSDTDNSSMKLNQDVLLVNESKKGILDEDNEKEKRDSLGNEESVDKTACECVRSPRESLDDLFQICSPCAIASGLRNDLAELTTLCLELNVLNSKIKSTSGHVDHTLQQYSPEILACQFLKKYFFLLNLKRAKESIKLSYSNSPSVWDTFIEGLKEMASSNPVYMEMEKGDLPTRLKLLDDEVPFDSPLLVVYATRLYEKFGESALRSLIKFFPSILPSDIIQLCHHHPAEFLAYLDSLVKSRPEDQRSSFLESLLQPESLRLDWLLLAVSLDAPPSTSTMDDEGYPRPHSHLLSWGYSQLILHLIKLPADFITKEKMTDICRSCGFWPGYLILCLELERRREAFTNIVYLNDMSLMEGDNGWIPETVEEWKLLLHLIQSKSTRPAPQESLNGSLSDGPSPINVENVALLLAKAMGPDRAWSLLQECGLALELSEKFTRTCDILRIAEKRQRALIQSMLEKCDRFLWSQQA

SEQ ID NO: 35 - protein, 1126 aa, Homo sapiens
MTFVPVIPEAYSHVLAEFESLDPLLTALRLDSSRLRCTSIAVSRKWLALGSTGGGLNLIQKDGWKQRLFLSHREGAISQIACCSHDDDYVAVATSQGLVVVWELNQERRGKPERIHVSSEHKGRKVTALCWDTAVLRVFVGDHVGKVSAIKLNTLKQAKAAAAFVMFPVQTVLTVDSCVVQLDYLDGRLLVSSLTRSFLCDTEREKFWKIGNKERHGEYGACFFPGRCAGGQQPVIYCARPGSRMWEVNFDGEVLSTHQFKKLLSMPPLPVITARSEPQYDHTVGSSQSLAFPKLLHLSEHCVLTWTEKGIYIFIPQNVQVLLWSEVKDIQDVAVYKNELFCLHFNGKISHLSLLSVERCVERLLRRGLWDLAARTCCLFQNSIITSRARKTLTADKLEHLKSQLDLTACSELISQLDDLILRFEPLESACSSRRSSISSHESFSILDSGIYRIISSRRGSQSDEDSCSLHSQTFSEDERLKEFASHQEEEQPEQGCGANRNEESASHSPVMSEVDKSEAFLPFSIALPFRSPSPLVSLQAVKDSVSSFVRKTTEKIGTLHGSPELKEPFESKDADRAHEEEVSAVTCPLEEDTEEKEIHQPPKEDRLQELTAATAEAMTKLLDPLVLFEPKVLRMVLLEWLSQLEKTFAMKDFPGISNTSSPTVKSNLGAHLLGETEKRVLDEESGEGRRVSLVTEEAGGQITCDPVSNLSEPSADRFRVCSPYAITNSLQRDLAELTTLCLELNVLTSAMESVGGHVDRASQQLSPEILACRFLKKYFFLLDLKRAKESIKLTYDSPCVWDTFVEGLKEMARSNPAYTELEEGDLPTGLQLLDGSVPSDSPLLIAFATRLYDRFGESALRACIKFYPSISPSDIAQLCRHHPAQFLAYLDSLVKSRPEDQWPSFLEFLLQPESLRLEWLLLAVSHDAPPSTSTVDDEGHPRPHSHLLSWGYSQLILLLIKLPADFTTKEKMTDICRSYGFWPGYLTLCLELERRREAFTNIVYLNDISLMEGDNGWIPETLEEWKLLLHLLQTKSTRPAPQESLNGSLSDGPAPINVENVALLLAKAMGPDRAWSLLQECGLALELSEKFTRTCDILRIAERRQRALIQGMLEKCDRFLWSQQA

SEQ ID NO: 36 - DNA, 3390 bp, Homo sapiens
atggcttttgtgccagtgataccagagtcctacagccatgttcttgcagagtttgaatctctggatccattactctcagccctgcggctggactccagtcgtctaaagtgcacgagcatagctgtgtctcggaaatggttggctttgggcagttcaggaggaggactccatctcattcagaaagaaggctggaagcacaggctttttctttcacacagggaaggtgcaatttctcaagtcgcctgttgtttacatgatgatgattatgttgctgtagctaccagtcaaggtcttgtggttgtttgggaattaaatcaagagcgtcgtgggaaaccggaacaaatgtatgtgtcttcagaacacaaaggccgaagagtcacagctctctgctgggatacagctattcttagagtttttgtaggtgatcatgctgggaaggtttctgctatcaaactcaatacttctaaacaagcaaaggcagctgctgcttttgtgatgtttcctgttcagacaatcacaactgttgactcctgtgttgtacagttagattatttggatggaaggctacttatatcttcacttactcgatccttcttgtgtgacactgagagagaaaagttttggaaaattggaaacaaggaaagagatggagaatatggagcttgtttctttcctggaagatgttctgggggccagcaacctctgatatattgtgctcgcccaggctctaggatgtgggaagtgaactttgatggagaagttataagtacacatcagttcaagaaactcctctcgttgccacctctccctgtgattactctcagatcagaacctcagtatgatcatacagctggatcctcccagtctttgtctttccccaaactcttacatcttagtgagcattgtgtgctgacttggacagaaagaggaatttatattttcattcctcagaatgttcaagttcttctttggagtgaagtcaaagatattcaggatgtggctgtctgtaggaatgaattgttctgtttgcacctaaatgggaaagtctcacatctctccctgatatctgtggagcgctgtgtggaacgcctgctaagaagaggcctatggaacttggctgctcgtacatgctgtcttttccaaaattctgtcattgccagcagagcaagaaaaactttgactgcagataaattggagcatttgaaatctcagctggaccatggcacctacaatgatctaatttctcaactggaagaattgatcttaaaatttgaacctttggattcagcttgtagcagtagaagaagctccatttcatcacatgaaagtttcagcatcttggactctggtatttatcgtatcattagtagtagaagaggcagtcagtcagatgaagactcttgctcccttcacagccaaaccctctcagaagatgagagatttaaagaattcacctcacagcaggaagaggacctgccagatcagtgttgtggctcacacggaaatgaagacaatgtttctcatgctccagtgatgtttgagacagataagaatgaaacttttctcccgttcggcattccattaccatttcgttctccatctcctcttgtgtctcttcaggctgtcaaagaaagtgtttctagctttgtgcgtaaaactactgagaagattggcacccttcacacgagccctgatctgaaagtgagaccagagctcaggggtgatgagcaatcatgtgaagaggatgtgagttcagatacctgcccaaaggaggaagacactgaggaggaaaaagaggtaactagtccacctccagaagaagacaggttccaggagcttaaagtagcaacagcagaagcaatgaccaagctacaggaccctctggttttatttgaatccgagtctctgagaatggttttacaggagtggctttcacatttagaaaaaacatttgccatgaaggacttttcaggtgtttcagatactgacaactcatccatgaaattgaaccaggatgtgctattagttaatgaatcaaaaaagggaatattagatgaagataatgaaaaagaaaaaagggactctttaggcaatgaagaatctgttgataaaacagcatgtgaatgtgtaaggagtccaagggagtctttggatgacctgtttcaaatatgttctccatgcgccattgcaagtggtcttcggaacgacctggctgaattgacaacattatgtttggagttgaatgtattgaattctaagatcaaaagcaccagtggacatgtggaccacactttgcaacagtactctcctgaaattctggcttgccagttcctgaagaagtacttttttctcctgaacttgaaaagagcgaaggagagtatcaagcttagttacagtaatagcccttctgtttgggatacttttattgaaggattgaaagaaatggcaagttccaatcctgtgtatatggagatggaaaaaggagatctaccaacaaggttaaagttactagatgacgaggttccttttgatagtccgttgttggttgtttatgctacccggttgtatgaaaagtttggggagtctgctcttcgatccttaatcaagttctttccatccattttgccatcggatatcatacaactttgtcatcatcatcctgctgagtttttggcctatttagacagtctggtgaaatcaaggcctgaagatcagcggtcatcttttcttgagtcccttctgcaaccagagtctttaaggttggattggctgcttttggcagtgtcccttgatgctccaccaagcaccagcacaatggatgatgaaggttatcccaggcctcattcacacttgctttcctggggttacagtcagctgatccttcatctaattaaacttcctgcagattttataaccaaagagaaaatgacagacatctgcaggtcttgtggtttctggcctggatatctaattctctgtttggagctggagagaagaagagaggccttcaccaatattgtgtatctgaatgatatgagcctgatggaaggggacaatggttggatcccagagaccgtggaggaatggaagcttctccttcatctcatacagagcaagagcacgaggccagccccccaggagtcactaaatgggagcctcagtgatgggccttcccccatcaatgtggagaatgtggcacttctgttagctaaggccatgggcccagatcgggcttggtcactgctacaggaatgtggtctggcccttgagttgtcagagaagtttaccagaacctgcgatatcctgaggattgctgagaaaaggcagagggccttgatacaaagcatgcttgaaaaatgcgatcggtttctctggtcccagcaggcctag

SEQ ID NO: 37 - DNA, 3048 bp, Homo sapiens
atgtatgtgtcttcagaacacaaaggccgaagagtcacagctctctgctgggatacagctattcttagagtttttgtaggtgatcatgctgggaaggtttctgctatcaaactcaatacttctaaacaagcaaaggcagctgctgcttttgtgatgtttcctgttcagacaatcacaactgttgactcctgtgttgtacagttagattatttggatggaaggctacttatatcttcacttactcgatccttcttgtgtgacactgagagagaaaagttttggaaaattggaaacaaggaaagagatggagaatatggagcttgtttctttcctggaagatgttctgggggccagcaacctctgatatattgtgctcgcccaggctctaggatgtgggaagtgaactttgatggagaagttataagtacacatcagttcaagaaactcctctcgttgccacctctccctgtgattactctcagatcagaacctcagtatgatcatacagctggatcctcccagtctttgtctttccccaaactcttacatcttagtgagcattgtgtgctgacttggacagaaagaggaatttatattttcattcctcagaatgttcaagttcttctttggagtgaagtcaaagatattcaggatgtggctgtctgtaggaatgaattgttctgtttgcacctaaatgggaaagtctcacatctctccctgatatctgtggagcgctgtgtggaacgcctgctaagaagaggcctatggaacttggctgctcgtacatgctgtcttttccaaaattctgtcattgccagcagagcaagaaaaactttgactgcagataaattggagcatttgaaatctcagctggaccatggcacctacaatgatctaatttctcaactggaagaattgatcttaaaatttgaacctttggattcagcttgtagcagtagaagaagctccatttcatcacatgaaagtttcagcatcttggactctggtatttatcgtatcattagtagtagaagaggcagtcagtcagatgaagactcttgctcccttcacagccaaaccctctcagaagatgagagatttaaagaattcacctcacagcaggaagaggacctgccagatcagtgttgtggctcacacggaaatgaagacaatgtttctcatgctccagtgatgtttgagacagataagaatgaaacttttctcccgttcggcattccattaccatttcgttctccatctcctcttgtgtctcttcaggctgtcaaagaaagtgtttctagctttgtgcgtaaaactactgagaagattggcacccttcacacgagccctgatctgaaagtgagaccagagctcaggggtgatgagcaatcatgtgaagaggatgtgagttcagatacctgcccaaaggaggaagacactgaggaggaaaaagaggtaactagtccacctccagaagaagacaggttccaggagcttaaagtagcaacagcagaagcaatgaccaagctacaggaccctctggttttatttgaatccgagtctctgagaatggttttacaggagtggctttcacatttagaaaaaacatttgccatgaaggacttttcaggtgtttcagatactgacaactcatccatgaaattgaaccaggatgtgctattagttaatgaatcaaaaaagggaatattagatgaagataatgaaaaagaaaaaagggactctttaggcaatgaagaatctgttgataaaacagcatgtgaatgtgtaaggagtccaagggagtctttggatgacctgtttcaaatatgttctccatgcgccattgcaagtggtcttcggaacgacctggctgaattgacaacattatgtttggagttgaatgtattgaattctaagatcaaaagcaccagtggacatgtggaccacactttgcaacagtactctcctgaaattctggcttgccagttcctgaagaagtacttttttctcctgaacttgaaaagagcgaaggagagtatcaagcttagttacagtaatagcccttctgtttgggatacttttattgaaggattgaaagaaatggcaagttccaatcctgtgtatatggagatggaaaaaggagatctaccaacaaggttaaagttactagatgacgaggttccttttgatagtccgttgttggttgtttatgctacccggttgtatgaaaagtttggggagtctgctcttcgatccttaatcaagttctttccatccattttgccatcggatatcatacaactttgtcatcatcatcctgctgagtttttggcctatttagacagtctggtgaaatcaaggcctgaagatcagcggtcatcttttcttgagtcccttctgcaaccagagtctttaaggttggattggctgcttttggcagtgtcccttgatgctccaccaagcaccagcacaatggatgatgaaggttatcccaggcctcattcacacttgctttcctggggttacagtcagctgatccttcatctaattaaacttcctgcagattttataaccaaagagaaaatgacagacatctgcaggtcttgtggtttctggcctggatatctaattctctgtttggagctggagagaagaagagaggccttcaccaatattgtgtatctgaatgatatgagcctgatggaaggggacaatggttggatcccagagaccgtggaggaatggaagcttctccttcatctcatacagagcaagagcacgaggccagccccccaggagtcactaaatgggagcctcagtgatgggccttcccccatcaatgtggagaatgtggcacttctgttagctaaggccatgggcccagatcgggcttggtcactgctacaggaatgtggtctggcccttgagttgtcagagaagtttaccagaacctgcgatatcctgaggattgctgagaaaaggcagagggccttgatacaaagcatgcttgaaaaatgcgatcggtttctctggtcccagcaggcctag

SEQ ID NO: 38 - protein, 627 aa, Homo sapiens
MARGSVSDEEMMELREAFAKVDTDGNGYISFNELNDLFKAACLPLPGYRVREITENLMATGDLDQDGRISFDEFIKIFHGLKSTDVAKTFRKAINKKEGICAIGGTSEQSSVGTQHSYSEEEKYAFVNWINKALENDPDCRHVIPMNPNTNDLFNAVGDGIVLCKMINLSVPDTIDERTINKKKLTPFTIQENLNLALNSASAIGCHVVNIGAEDLKEGKPYLVLGLLWQVIKIGLFADIELSRNEALIALLREGESLEDLMKLSPEELLLRWANYHLENAGCNKIGNFSTDIKDSKAYYHLLEQVAPKGDEEGVPAVVIDMSGLREKDDIQRAECMLQQAERLGCRQFVTATDVVRGNPKLNLAFIANLFNRYPALHKPENQDIDWGALEGETREERTFRNWMNSLGVNPRVNHLYSDLSDALVIFQLYEKIKVPVDWNRVNKPPYPKLGGNMKKLENCNYAVELGKNQAKFSLVGIGGQDLNEGNRTLTLALIWQLMRRYTLNILEEIGGGQKVNDDIIVNWVNETLREAKKSSSISSFKDPKISTSLPVLDLIDAIQPGSINYDLLKTENLNDDEKLNNAKYAISMARKIGARVYALPEDLVEVNPKMVMTVFACLMGKGMKRV

SEQ ID NO: 39 - protein, 627 aa, Homo sapiens
MARGSVSDEEMMELREAFAKVDTDGNGYISCNELNDLFKAACLPLPGYRVREITENLMATGDLDQDGKISFDEFIKVFHGLKSTEVAKTFRKAINKKEGICAIGGTSEQSSVGTQHSYSEEEKYAFVNWINKALENDPDCRHVIPMNPNTDDLFNAVGDGIVLCKMINLSVPDTIDERTINKKKLTPFTIQENLNLALNSASAIGCHVVNIGAEDLKEGKPYLVLGLLWQVIKIGLFADIELSRNEALIALLREGESLEDLMKLSPEELLLRWANYHLENAGCTKITNFSTDIKDSKAYYHLLEQVAPKGDEEGIPAVVIDMSGLREKDDIQRAECMLQQAERLGCRQFVTATDVVRGNPKLNLAFIANLFNKYPALHKPENQDIDWGALEGETREERTFRNWMNSLGVNPRVNHLYSDLSDALVIFQLYEKIKVPVDWNRVNKPPYPKLGGNMKKLENCNYAVDLGKNQAKFSLVGIAGQDLNEGNRTLTLALVWQLMRRYTLNILEDIGGGQKVNDDIIVNWVNTTLKEAQKSSSIASFKDPKISTSLPVLDLIDAIQPGSINYDLLKTENLDDEEKLNNAKYAISMARKIGARVYALPEDLVEVNPKMVMTVFACLMGKGMKRV

SEQ ID NO: 40 - DNA, 1884 bp, Homo sapiens
atggccagaggatcagtgtccgatgaggaaatgatggagctcagagaagcttttgccaaagttgatactgatggcaatggatacatcagcttcaatgagttgaatgacttgttcaaggctgcttgcttgcctttgcctgggtatagagtacgagaaattacagaaaacctgatggctacaggtgatctggaccaagatggaaggatcagctttgatgagtttatcaagattttccatggcctaaaaagcacagatgttgccaagacctttagaaaagcaatcaataagaaggaagggatttgtgcaatcggtggtacttcagagcagtctagcgttggcacccaacactcctattcagaggaagaaaagtatgcctttgtcaactggataaacaaagccctggaaaatgatcctgattgtcggcatgtcatcccaatgaacccaaacacgaatgatctctttaatgctgttggagatggcattgtcctttgtaaaatgatcaacctgtcagtgccagacacaattgatgaaagaacaatcaacaaaaagaagctaacccctttcaccattcaggaaaatctgaacttggctctgaactctgcctcagccatcgggtgccatgtggtcaacataggggctgaggacctgaaggaggggaagccttatctggtcctgggacttctgtggcaagtcatcaagattgggttgtttgctgacattgaactcagcagaaatgaagctctgattgctcttttgagagaaggtgagagcctggaggatttgatgaaactctcccctgaagagctcttgctgaggtgggctaattaccacctggaaaatgcaggctgcaacaaaattggcaacttcagtactgacatcaaggactcaaaagcttattaccacctgcttgagcaggtggctccaaaaggagatgaagaaggtgttcctgctgttgttattgacatgtcaggactgcgggagaaggatgacatccagagggcagaatgcatgctgcagcaggcggagaggctgggctgccggcagtttgtcacagccacagatgttgtccgagggaaccccaagttgaacttggcttttattgccaacctctttaacagataccctgccctgcacaaaccagagaaccaggacattgactggggggctcttgaaggtgagacgagagaagagcggacatttaggaactggatgaactccctgggtgttaaccctcgagtcaatcatttgtacagtgacttatcagatgccctggtcatcttccagctctatgaaaagatcaaagttcctgttgactggaacagagtaaacaaaccgccataccccaaactgggaggcaatatgaagaagcttgagaattgtaactacgcggtagaattggggaagaatcaagcgaagttctccctggttggcatcggtggacaagatctcaatgaaggaaaccgcactctcacactggccttgatttggcagctaatgagaaggtatacactgaatatcctcgaagaaattggtggtggccagaaggtcaatgatgacattattgtcaactgggtgaatgaaacattgagggaagcaaagaaaagttcatccatctctagtttcaaggacccgaagattagtacaagtctgcctgttctggacctcatcgatgccatccaaccaggttccattaactatgaccttctgaagacagaaaatctgaatgatgatgagaaactcaacaatgcaaaatatgccatctctatggcccgaaaaattggagcaagagtgtatgccctgccagaagacctggttgaagtgaaccccaaaatggtcatgaccgtgtttgcctgcctcatggggaaaggaatgaagagggtgtga

SEQ ID NO: 41 - protein, 906 aa, Homo sapiens
MQAAVAVSVPFLLLCVLGTCPPARCGQAGDASLMELEKRKENRFVERQSIVPLRLIYRSGGEDESRHDALDTRVRGDLGGPQLTHVDQASFQVDAFGTSFILDVVLNHDLLSSEYIERHIEHGGKTVEVKGGEHCYYQGHIRGNPDSFVALSTCHGLHGMFYDGNHTYLIEPEENDTTQEDFHFHSVYKSRLFEFSLDDLPSEFQQVNITPSKFILKPRPKRSKRQLRRYPRNVEEETKYIELMIVNDHLMFKKHRLSVVHTNTYAKSVVNMADLIYKDQLKTRIVLVAMETWATDNKFAISENPLITLREFMKYRRDFIKEKSDAVHLFSGSQFESSRSGAAYIGGICSLLKGGGVNEFGKTDLMAVTLAQSLAHNIGIISDKRKLASGECKCEDTWSGCIMGDTGYYLPKKFTQCNIEEYHDFLNSGGGACLFNKPSKLLDPPECGNGFIETGEECDCGTPAECVLEGAECCKKCTLTQDSQCSDGLCCKKCKFQPMGTVCREAVNDCDIRETCSGNSSQCAPNIHKMDGYSCDGVQGICFGGRCKTRDRQCKYIWGQKVTASDKYCYEKLNIEGTEKGNCGKDKDTWIQCNKRDVLCGYLLCTNIGNIPRLGELDGEITSTLVVQQGRTLNCSGGHVKLEEDVDLGYVEDGTPCGPQMMCLEHRCLPVASFNFSTCLSSKEGTICSGNGVCSNELKCVCNRHWIGSDCNTYFPHNDDAKTGITLSGNGVAGTNIIIGIIAGTILVLALILGITAWGYKNYREQRQLPQGDYVKKPGDGDSFYSDIPPGVSTNSASSSKKRSNGLSHSWSERIPDTKHISDICENGRPRSNSWQGNLGGNKKKIRGKRFRPRSNSTETLSPAKSPSSSTGSIASSRKYPYPMPPLPDEDKKVNRQSARLWETSI

SEQ ID NO: 42 - DNA, 2721 bp, Homo sapiens
atgcaggcggcagtggctgtgtccgtgcccttcttgctgctctgtgtcctggggacctgccctccggcgcgctgcggccaggcaggagacgcctcattgatggagctagagaagaggaaggaaaaccgcttcgtggagcgccagagcatcgtgccactgcgcctcatctaccgctcgggcggcgaagacgaaagtcggcacgacgcgctcgacacgcgggtgcggggcgacctcggtggcccgcagttgactcatgttgaccaagcaagcttccaggttgatgcctttggaacgtcattcattctcgatgtcgtgctaaatcatgatttgctgtcctctgaatacatagagagacacattgaacatggaggcaagactgtggaagttaaaggaggagagcactgttactaccagggccatatccgaggaaaccctgactcatttgttgcattgtcaacatgccacggacttcatgggatgttctatgacgggaaccacacatatctcattgagccagaagaaaatgacactactcaagaggatttccattttcattcagtttacaaatccagactgtttgaattttccttggatgatcttccatctgaatttcagcaagtaaacattactccatcaaaatttattttgaagccaagaccaaaaaggagtaaacggcagcttcgtcgatatcctcgtaatgtagaagaagaaaccaaatacattgaactgatgattgtgaatgatcaccttatgtttaaaaaacatcggctttccgttgtacataccaatacctatgcgaaatctgtggtgaacatggcagatttaatatataaagaccaacttaagaccaggatagtattggttgctatggaaacctgggcgactgacaacaagtttgccatatctgaaaatccattgatcaccctacgtgagtttatgaaatacaggagggattttatcaaagagaaaagtgatgcagttcaccttttttcgggaagtcaatttgagagtagccggagcggggcagcttatattggtgggatttgctcgttgctgaaaggaggaggcgtgaatgaatttgggaaaactgatttaatggctgttacacttgcccagtcattagcccataatattggtattatctcagacaaaagaaagttagcaagtggtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtgctttgtggttaccttttgtgtaccaatattggcaatatcccaaggcttggagaactcgatggtgaaatcacatctactttagttgtgcagcaaggaagaacattaaactgcagtggtgggcatgttaagcttgaagaagatgtagatcttggctatgtggaagatgggacaccttgtggtccccaaatgatgtgcttagaacacaggtgtcttcctgtggcttctttcaactttagtacttgcttgagcagtaaagaaggcactatttgctcaggaaatggagtttgcagtaatgagctgaagtgtgtgtgtaacagacactggataggttctgattgcaacacttacttccctcacaatgatgatgcaaagactggtatcactctgtctggcaatggtgttgctggcaccaatatcataataggcataattgctggcaccattttagtgctggccctcatattaggaataactgcgtggggttataaaaactatcgagaacagagacagttaccccagggagattatgtaaaaaagcctggagatggtgactctttttatagcgacattcctcccggagtcagcacaaactcagcatctagttctaagaagaggtcaaatgggctctctcattcttggagtgaaaggattccagacacaaaacatatttcagacatctgtgaaaatgggcgacctcgaagtaactcttggcaaggtaacctgggaggcaacaaaaagaaaatcagaggcaaaagatttagacctcggtctaattcaactgagactttatctcctgccaagtctccttcttcatcaactgggtctattgcctccagcagaaaatacccttacccaatgcctccacttcctgatgaggacaagaaagtgaaccgacaaagtgccaggctatgggagacatccatttaa

SEQ ID NO: 43 - DNA, 2700 bp, Homo sapiens
atgcaggcggcagtggctgtgtccgtgcccttcttgctgctctgtgtcctggggacctgccctccggcgcgctgcggccaggcaggagacgcctcattgatggagctagagaagaggaaggaaaaccgcttcgtggagcgccagagcatcgtgccactgcgcctcatctaccgctcgggcggcgaagacgaaagtcggcacgacgcgctcgacacgcgggtgcggggcgacctcggtggcccgcagttgactcatgttgaccaagcaagcttccaggttgatgcctttggaacgtcattcattctcgatgtcgtgctaaatcatgatttgctgtcctctgaatacatagagagacacattgaacatggaggcaagactgtggaagttaaaggaggagagcactgttactaccagggccatatccgaggaaaccctgactcatttgttgcattgtcaacatgccacggacttcatgggatgttctatgacgggaaccacacatatctcattgagccagaagaaaatgacactactcaagaggatttccattttcattcagtttacaaatccagactgtttgaattttccttggatgatcttccatctgaatttcagcaagtaaacattactccatcaaaatttattttgaagccaagaccaaaaaggagtaaacggcagcttcgtcgatatcctcgtaatgtagaagaagaaaccaaatacattgaactgatgattgtgaatgatcaccttatgtttaaaaaacatcggctttccgttgtacataccaatacctatgcgaaatctgtggtgaacatggcagatttaatatataaagaccaacttaagaccaggatagtattggttgctatggaaacctgggcgactgacaacaagtttgccatatctgaaaatccattgatcaccctacgtgagtttatgaaatacaggagggattttatcaaagagaaaagtgatgcagttcaccttttttcgggaagtcaatttgagagtagccggagcggggcagcttatattggtgggatttgctcgttgctgaaaggaggaggcgtgaatgaatttgggaaaactgatttaatggctgttacacttgcccagtcattagcccataatattggtattatctcagacaaaagaaagttagcaagtggtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtgctttgtggttaccttttgtgtaccaatattggcaatatcccaaggcttggagaactcgatggtgaaatcacatctactttagttgtgcagcaaggaagaacattaaactgcagtggtgggcatgttaagcttgaagaagatgtagatcttggctatgtggaagatgggacaccttgtggtccccaaatgatgtgcttagaacacaggtgtcttcctgtggcttctttcaactttagtacttgcttgagcagtaaagaaggcactatttgctcaggaaatggagtttgcagtaatgagctgaagtgtgtgtgtaacagacactggataggttctgattgcaacacttacttccctcacaatgatgatgcaaagactggtatcactctgtctggcaatggtgttgctggcaccaatatcataataggcataattgctggcaccattttagtgctggccctcatattaggaataactgcgtggggttataaaaactatcgagaacagaggtcaaatgggctctctcattcttggagtgaaaggattccagacacaaaacatatttcagacatctgtgaaaatgggcgacctcgaagtaactcttggcaaggtaacctgggaggcaacaaaaagaaaatcagaggcaaaagatttagacctcggtctaattcaactgagtatttaaacccatggttcaaaagagactataatgtagctaagtgggtagaagatgtgaataaaaacactgaaggaccatactttaggactttatctcctgccaagtctccttcttcatcaactgggtctattgcctccagcagaaaatacccttacccaatgcctccacttcctgatgaggacaagaaagtgaaccgacaaagtgccaggctatgggagacatccatttaa

SEQ ID NO: 44 - DNA, 2613 bp, Homo sapiens
atgcaggcggcagtggctgtgtccgtgcccttcttgctgctctgtgtcctggggacctgccctccggcgcgctgcggccaggcaggagacgcctcattgatggagctagagaagaggaaggaaaaccgcttcgtggagcgccagagcatcgtgccactgcgcctcatctaccgctcgggcggcgaagacgaaagtcggcacgacgcgctcgacacgcgggtgcggggcgacctcggtggcccgcagttgactcatgttgaccaagcaagcttccaggttgatgcctttggaacgtcattcattctcgatgtcgtgctaaatcatgatttgctgtcctctgaatacatagagagacacattgaacatggaggcaagactgtggaagttaaaggaggagagcactgttactaccagggccatatccgaggaaaccctgactcatttgttgcattgtcaacatgccacggacttcatgggatgttctatgacgggaaccacacatatctcattgagccagaagaaaatgacactactcaagaggatttccattttcattcagtttacaaatccagactgtttgaattttccttggatgatcttccatctgaatttcagcaagtaaacattactccatcaaaatttattttgaagccaagaccaaaaaggagtaaacggcagcttcgtcgatatcctcgtaatgtagaagaagaaaccaaatacattgaactgatgattgtgaatgatcaccttatgtttaaaaaacatcggctttccgttgtacataccaatacctatgcgaaatctgtggtgaacatggcagatttaatatataaagaccaacttaagaccaggatagtattggttgctatggaaacctgggcgactgacaacaagtttgccatatctgaaaatccattgatcaccctacgtgagtttatgaaatacaggagggattttatcaaagagaaaagtgatgcagttcaccttttttcgggaagtcaatttgagagtagccggagcggggcagcttatattggtgggatttgctcgttgctgaaaggaggaggcgtgaatgaatttgggaaaactgatttaatggctgttacacttgcccagtcattagcccataatattggtattatctcagacaaaagaaagttagcaagtggtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtgctttgtggttaccttttgtgtaccaatattggcaatatcccaaggcttggagaactcgatggtgaaatcacatctactttagttgtgcagcaaggaagaacattaaactgcagtggtgggcatgttaagcttgaagaagatgtagatcttggctatgtggaagatgggacaccttgtggtccccaaatgatgtgcttagaacacaggtgtcttcctgtggcttctttcaactttagtacttgcttgagcagtaaagaaggcactatttgctcaggaaatggagtttgcagtaatgagctgaagtgtgtgtgtaacagacactggataggttctgattgcaacacttacttccctcacaatgatgatgcaaagactggtatcactctgtctggcaatggtgttgctggcaccaatatcataataggcataattgctggcaccattttagtgctggccctcatattaggaataactgcgtggggttataaaaactatcgagaacagaggtcaaatgggctctctcattcttggagtgaaaggattccagacacaaaacatatttcagacatctgtgaaaatgggcgacctcgaagtaactcttggcaaggtaacctgggaggcaacaaaaagaaaatcagaggcaaaagatttagacctcggtctaattcaactgagactttatctcctgccaagtctccttcttcatcaactgggtctattgcctccagcagaaaatacccttacccaatgcctccacttcctgatgaggacaagaaagtgaaccgacaaagtgccaggctatgggagacatccatttaa

SEQ ID NO: 45 - DNA, 2580 bp, Homo sapiens
atgcaggcggcagtggctgtgtccgtgcccttcttgctgctctgtgtcctggggacctgccctccggcgcgctgcggccaggcaggagacgcctcattgatggagctagagaagaggaaggaaaaccgcttcgtggagcgccagagcatcgtgccactgcgcctcatctaccgctcgggcggcgaagacgaaagtcggcacgacgcgctcgacacgcgggtgcggggcgacctcggtggcccgcagttgactcatgttgaccaagcaagcttccaggttgatgcctttggaacgtcattcattctcgatgtcgtgctaaatcatgatttgctgtcctctgaatacatagagagacacattgaacatggaggcaagactgtggaagttaaaggaggagagcactgttactaccagggccatatccgaggaaaccctgactcatttgttgcattgtcaacatgccacggacttcatgggatgttctatgacgggaaccacacatatctcattgagccagaagaaaatgacactactcaagaggatttccattttcattcagtttacaaatccagactgtttgaattttccttggatgatcttccatctgaatttcagcaagtaaacattactccatcaaaatttattttgaagccaagaccaaaaaggagtaaacggcagcttcgtcgatatcctcgtaatgtagaagaagaaaccaaatacattgaactgatgattgtgaatgatcaccttatgtttaaaaaacatcggctttccgttgtacataccaatacctatgcgaaatctgtggtgaacatggcagatttaatatataaagaccaacttaagaccaggatagtattggttgctatggaaacctgggcgactgacaacaagtttgccatatctgaaaatccattgatcaccctacgtgagtttatgaaatacaggagggattttatcaaagagaaaagtgatgcagttcaccttttttcgggaagtcaatttgagagtagccggagcggggcagcttatattggtgggatttgctcgttgctgaaaggaggaggcgtgaatgaatttgggaaaactgatttaatggctgttacacttgcccagtcattagcccataatattggtattatctcagacaaaagaaagttagcaagtggtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtgctttgtggttaccttttgtgtaccaatattggcaatatcccaaggcttggagaactcgatggtgaaatcacatctactttagttgtgcagcaaggaagaacattaaactgcagtggtgggcatgttaagcttgaagaagatgtagatcttggctatgtggaagatgggacaccttgtggtccccaaatgatgtgcttagaacacaggtgtcttcctgtggcttctttcaactttagtacttgcttgagcagtaaagaaggcactatttgctcaggaaatggagtttgcagtaatgagctgaagtgtgtgtgtaacagacactggataggttctgattgcaacacttacttccctcacaatgatgatgcaaagactggtatcactctgtctggcaatggtgttgctggcaccaatatcataataggcataattgctggcaccattttagtgctggccctcatattaggaataactgcgtggggttataaaaactatcgagaacagagacagttaccccagggagattatgtaaaaaagcctggagatggtgactctttttatagcgacattcctcccggagtcagcacaaactcagcatctagttctaagaagaggtcaaatgggctctctcattcttggagtgaaaggattccagacacaaaacatatttcagacatctgtgaaaatgggcgacctcgaagtaactcttggcaaggtaacctgggaggcaacaaaaagaaaatcagaggcaaaagatttagacctcggtctaattcaactgagtaa

SEQ ID NO: 46 - DNA, 1342 bp, Homo sapiens
cattcggggacgctctcagcgctctgctcacgacccagcttccttcaaaatatctgctgttcatgaaatcctgtgcaagctcagcttggagggtgatcactctacatccccaagtgcatttgggtcagtcaaagcctacaccaaatttgatgctgagcaggatgctttgaacattgaaatggccatcaagaccaaaggtgtggatgagatcaccactgtcaacattttgactaaccacagcaatgcacagagacaggatattgccttcacctaccagagaaggaccaaaaaggaacttgtatcagcacttaagtcagccttatctggccacctagatagtggctttgggcctattaaagacacctgctcagtatgatgcttctgagctaaaatcttccatgaaggggctgggaaccaacaaggactccctcattgagatcatctgctcaagaaccaaccaagagctgcaggaaattaacagagtctacaaggaaatgtacaagactgatctggagaaggacattatttcggacacatctggtgacttctgcaagctgatggttgccctggcaaagggtagaagagcagaggatggctctgtcattgattatgaactgattgaccaagatgcccgggatctctatgacgctggggtgaagaggaaaggaactgaagttcccaagtggatcagcgtcatgaccgagcggagcatgtcccacttccagaaagtatttgataggtacaagagctacagtccttatgacatgttggagagcatcaagaaagaggttaaaggagacctggaaaattctttcctgaacctggtccagtgtattcagaacaagcccctgtatttcgctgaccggctgtacgactccataatgggcatggggactcaagataaggtcctgatcagaatcatggtctcccacaatgaagtggacatgttgaaaattaggtctgaattcaagagaaagtatagcaagtccctgtactactatatccagcaagacactaagggtgctgtacctgtgtggtggagatggctgaagtccgacacagcacgagcgtccagaaatggtgctccccatgcttccagctaacaggtctagaaaacccgcttgtgactagcagtccctgtggctgttcctgtgaggatgacgttagcattacccccaacctcattttagttgcctaagcattgcctggctttcctgtctagtctctcctgtaagccaaagaaatgaacattccaaggaattggaagtgaagtctatgatgtaaacactttgcctcctgtgtactgtgtcataaacggattaataaactgaatttgtacttt

SEQ ID NO: 47 - protein, 758 aa, Homo sapiens
MSEVLPADSGVDTLAVFMASSGTTDVTNRNSPATPPNTLNLRSSHNELLNAEIKHTETKNSTPPKCRKKYALTNIQAAMGLSDPAAQPLLGNGSANIKLVKNGENQLRKAAEQGQQDPNKNLSPTAVINITSEKLEGKEPHPQDSSSCEILPSQPRRTKSFLNYYADLETSARELEQNRGNHHGTAEEKSQPVQGQASTIIGNGDLLLQKPNRPQSSPEDGQVATVSSSPETKKDHPKTGAKTDCALHRIQNLAPSDEESSWTTLSQDSASPSSPDETDIWSDHSFQTDPDLPPGWKRVSDIAGTYYWHIPTGTTQWERPVSIPADLQGSRKGSLSSVTPSPTPENEKQPWSDFAVLNGGKINSDIWKDLHAATVNPDPSLKEFEGATLRYASLKLRNAPHPDDDDSCSINSDPEAKCFAVRSLGWVEMAEEDLAPGKSSVAVNNCIRQLSYCKNDIRDTVGIWGEGKDMYLILENDMLSLVDPMDRSVLHSQPIVSIRVWGVGRDNGRDFAYVARDKDTRILKCHVFRCDTPAKAIATSLHEICSKIMAERKNAKALACSSLQERANVNLDVPLQVDFPTPKTELVQKFHVQYLGMLPVDKPVGMDILNSAIENLMTSSNKEDWLSVNMNVADATVTVISEKNEEEVLVECRVRFLSFMGVGKDVHTFAFIMDTGNQRFECHVFWCEPNAGNVSEAVQAACMLRYQKCLVARPPSQKVRPPPPPADSVTRRVTTNVKRGVLSLIDTLKQKRPVTEMP

SEQ ID NO: 48 - DNA, 2280 bp, Homo sapiens
atgtcagaagtacttccagctgactcaggtgttgacaccttggcagtgtttatggccagcagcggaactacagacgtcacaaatcggaacagcccagccacaccaccaaacacccttaacctccgatcctcccacaatgaactgttgaacgctgaaataaaacacacagaaaccaagaacagcacacctcccaaatgcaggaaaaaatatgcactaactaacatccaggcggccatgggcctctcggatccagctgcacagcccctgctgggaaatggctctgccaacatcaagctggtgaaaaatggggagaaccagctccgtaaggctgcagagcaagggcagcaggaccccaacaaaaacctgagccccactgcagtcatcaacataacttctgagaagttagagggtaaagagccccacccacaggattcctcgagctgtgagattttaccctcccagcccaggagaactaagagcttcctaaattactatgcagatctggaaacctcagccagagaactagagcagaaccgaggcaatcaccatgggactgcggaagagaaatcccagccagtccagggccaggcctccaccatcattgggaatggcgatttgctgctgcagaaaccaaacagaccccagtccagccctgaagacggccaagtagccacagtgtcatccagcccagaaaccaagaaggatcatccgaaaacaggggccaaaaccgactgtgcactgcaccggatccagaacctggcaccgagcgatgaggagtccagctggacaacgttgtcccaagacagtgcctcacccagctccccggatgaaacagcagatatatggagtgatcactcatttcagactgatccagatttgccgcctggctggaaaagagtcagtgacattgccgggacctattattggcacatcccaacaggaacgactcagtgggaacggcccgtctccatcccagcagatctccagggttctaggaaagggtcacttagttctgtaacgccatctcccaccccagagaacgagaaacagccatggagtgattttgctgttctgaatgggggaaagattaatagtgacatttggaaggatttgcatgcagccactgttaacccggaccccagtttaaaagagtttgaaggagcaaccctacgctatgcatctttgaaactcagaaatgccccacaccctgatgatgatgattcttgtagtatcaacagtgacccagaagccaagtgttttgctgtgcgttctctgggatgggtagagatggcagaagaggacctcgcccccggtaaaagtagtgttgcggtcaacaactgcatcaggcaactttcctactgcaaaaatgacatccgagacacagtcgggatttggggagaggggaaagacatgtacctgatcctggagaatgacatgctcagcctggtggaccccatggaccgcagcgtgctgcactcgcagcccatcgtcagcatccgcgtgtggggcgtgggccgcgacaatggccgggattttgcttatgtagcaagagataaagatacaagaattttgaaatgtcatgtatttcgatgtgacacaccagcaaaagccattgccacaagtctccacgagatctgctccaagattatggctgaacggaagaatgccaaagcgctggcctgcagctccttacaggaaagggccaatgtgaacctcgatgtccctttgcaagtagattttccaacaccaaagactgagctggtccagaagttccacgtgcagtacttgggcatgttacctgtagacaaaccagtcggaatggatattttgaacagtgccatagaaaatcttatgacctcatccaacaaggaggactggctgtcagtgaacatgaacgtggctgatgccactgtgactgtcatcagtgaaaagaatgaagaggaagtcttagtggaatgtcgtgtgcgattcctgtccttcatgggtgttgggaaggacgtccacacatttgccttcatcatggacacggggaaccagcgctttgagtgccacgttttctggtgcgagcctaatgctggtaacgtgtctgaggcggtgcaggccgcctgcatgttacgatatcagaagtgcttggtagccaggccgccttctcagaaagttcgaccacctccaccgccagcagattcagtaaccagaagagtcacaaccaatgtaaaacgaggggtcttatccctcattgacactttgaaacagaaacgccctgtcaccgaaatgccatag

SEQ ID NO: 49 - DNA, 2277 bp, Homo sapiens
atgtcagaagtacttccagctgactcaggtgttgacaccttggcagtgtttatggccagcagcggaactacagacgtcacaaatcggaacagcccagccacaccaccaaacacccttaacctccgatcctcccacaatgaactgttgaacgctgaaataaaacacacagaaaccaagaacagcacacctcccaaatgcaggaaaaaatatgcactaactaacatccaggcggccatgggcctctcggatccagctgcacagcccctgctgggaaatggctctgccaacatcaagctggtgaaaaatggggagaaccagctccgtaaggctgcagagcaagggcagcaggaccccaacaaaaacctgagccccactgcagtcatcaacataacttctgagaagttagagggtaaagagccccacccacaggattcctcgagctgtgagattttaccctcccagcccaggagaactaagagcttcctaaattactatgcagatctggaaacctcagccagagaactagagcagaaccgaggcaatcaccatgggactgcggaagagaaatcccagccagtccagggccaggcctccaccatcattgggaatggcgatttgctgctgcagaaaccaaacagaccccagtccagccctgaagacggccaagtagccacagtgtcatccagcccagaaaccaagaaggatcatccgaaaacaggggccaaaaccgactgtgcactgcaccggatccagaacctggcaccgagcgatgaggagtccagctggacaacgttgtcccaagacagtgcctcacccagctccccggatgaaacagatatatggagtgatcactcatttcagactgatccagatttgccgcctggctggaaaagagtcagtgacattgccgggacctattattggcacatcccaacaggaacgactcagtgggaacggcccgtctccatcccagcagatctccagggttctaggaaagggtcacttagttctgtaacgccatctcccaccccagagaacgagaaacagccatggagtgattttgctgttctgaatgggggaaagattaatagtgacatttggaaggatttgcatgcagccactgttaacccggaccccagtttaaaagagtttgaaggagcaaccctacgctatgcatctttgaaactcagaaatgccccacaccctgatgatgatgattcttgtagtatcaacagtgacccagaagccaagtgttttgctgtgcgttctctgggatgggtagagatggcagaagaggacctcgcccccggtaaaagtagtgttgcggtcaacaactgcatcaggcaactttcctactgcaaaaatgacatccgagacacagtcgggatttggggagaggggaaagacatgtacctgatcctggagaatgacatgctcagcctggtggaccccatggaccgcagcgtgctgcactcgcagcccatcgtcagcatccgcgtgtggggcgtgggccgcgacaatggccgggattttgcttatgtagcaagagataaagatacaagaattttgaaatgtcatgtatttcgatgtgacacaccagcaaaagccattgccacaagtctccacgagatctgctccaagattatggctgaacggaagaatgccaaagcgctggcctgcagctccttacaggaaagggccaatgtgaacctcgatgtccctttgcaagtagattttccaacaccaaagactgagctggtccagaagttccacgtgcagtacttgggcatgttacctgtagacaaaccagtcggaatggatattttgaacagtgccatagaaaatcttatgacctcatccaacaaggaggactggctgtcagtgaacatgaacgtggctgatgccactgtgactgtcatcagtgaaaagaatgaagaggaagtcttagtggaatgtcgtgtgcgattcctgtccttcatgggtgttgggaaggacgtccacacatttgccttcatcatggacacggggaaccagcgctttgagtgccacgttttctggtgcgagcctaatgctggtaacgtgtctgaggcggtgcaggccgcctgcatgttacgatatcagaagtgcttggtagccaggccgccttctcagaaagttcgaccacctccaccgccagcagattcagtaaccagaagagtcacaaccaatgtaaaacgaggggtcttatccctcattgacactttgaaacagaaacgccctgtcaccgaaatgccatag

SEQ ID NO: 50 - DNA, 2211 bp, Homo sapiens
atgtcagaagtacttccagctgactcaggtgttgacaccttggcagtgtttatggccagcagcggaactacagacgtcacaaatcggaacagcccagccacaccaccaaacacccttaacctccgatcctcccacaatgaactgttgaacgctgaaataaaacacacagaaaccaagaacagcacacctcccaaatgcaggaaaaaatatgcactaactaacatccaggcggccatgggcctctcggatccagctgcacagcccctgctgggaaatggctctgccaacatcaagctggtgaaaaatggggagaaccagctccgtaaggctgcagagcaagggcagcaggaccccaacaaaaacctgagccccactgcagtcatcaacataacttctgagaagttagagggtaaagagccccacccacaggattcctcgagctgtgagattttaccctcccagcccaggagaactaagagcttcctaaattactatgcagatctggaaacctcagccagagaactagagcagaaccgaggcaatcaccatgggactgcggaagagaaatcccagccagtccagggccaggcctccaccatcattgggaatggcgatttgctgctgcagaaaccaaacagaccccagtccagccctgaagacggccaagtagccacagtgtcatccagcccagaaaccaagaaggatcatccgaaaacaggggccaaaaccgactgtgcactgcaccggatccagaacctggcaccgagcgatgaggagtccagctggacaacgttgtcccaagacagtgcctcacccagctccccggatgaaacagatatatggagtgatcactcatttcagactgatccagatttgccgcctggctggaaaagagtcagtgacattgccgggacctattattggcacatcccaacaggaacgactcagtgggaacggcccgtctccatcccagcagatctccagggttctaggaaagggtcacttagttctgtaacgccatctcccaccccagagaacgaggatttgcatgcagccactgttaacccggaccccagtttaaaagagtttgaaggagcaaccctacgctatgcatctttgaaactcagaaatgccccacaccctgatgatgatgattcttgtagtatcaacagtgacccagaagccaagtgttttgctgtgcgttctctgggatgggtagagatggcagaagaggacctcgcccccggtaaaagtagtgttgcggtcaacaactgcatcaggcaactttcctactgcaaaaatgacatccgagacacagtcgggatttggggagaggggaaagacatgtacctgatcctggagaatgacatgctcagcctggtggaccccatggaccgcagcgtgctgcactcgcagcccatcgtcagcatccgcgtgtggggcgtgggccgcgacaatggccgggattttgcttatgtagcaagagataaagatacaagaattttgaaatgtcatgtatttcgatgtgacacaccagcaaaagccattgccacaagtctccacgagatctgctccaagattatggctgaacggaagaatgccaaagcgctggcctgcagctccttacaggaaagggccaatgtgaacctcgatgtccctttgcaagattttccaacaccaaagactgagctggtccagaagttccacgtgcagtacttgggcatgttacctgtagacaaaccagtcggaatggatattttgaacagtgccatagaaaatcttatgacctcatccaacaaggaggactggctgtcagtgaacatgaacgtggctgatgccactgtgactgtcatcagtgaaaagaatgaagaggaagtcttagtggaatgtcgtgtgcgattcctgtccttcatgggtgttgggaaggacgtccacacatttgccttcatcatggacacggggaaccagcgctttgagtgccacgttttctggtgcgagcctaatgctggtaacgtgtctgaggcggtgcaggccgcctgcatgttacgatatcagaagtgcttggtagccaggccgccttctcagaaagttcgaccacctccaccgccagcagattcagtaaccagaagagtcacaaccaatgtaaaacgaggggtcttatccctcattgacactttgaaacagaaacgccctgtcaccgaaatgccatag

SEQ ID NO: 51 - DNA, 633 bp, Homo sapiens
atggctgaacggaagaatgccaaagcgctggcctgcagctccttacaggaaagggccaatgtgaacctcgatgtccctttgcaagtagattttccaacaccaaagactgagctggtccagaagttccacgtgcagtacttgggcatgttacctgtagacaaaccagtcggaatggatattttgaacagtgccatagaaaatcttatgacctcatccaacaaggaggactggctgtcagtgaacatgaacgtggctgatgccactgtgactgtcatcagtgaaaagaatgaagaggaagtcttagtggaatgtcgtgtgcgattcctgtccttcatgggtgttgggaaggacgtccacacatttgccttcatcatggacacggggaaccagcgctttgagtgccacgttttctggtgcgagcctaatgctggtaacgtgtctgaggcggtgcaggccgcctgcatgttacgatatcagaagtgcttggtagccaggccgccttctcagaaagttcgaccacctccaccgccagcagattcagtaaccagaagagtcacaaccaatgtaaaacgaggggtcttatccctcattgacactttgaaacagaaacgccctgtcaccgaaatgccatag

SEQ ID NO: 52 - protein, 1461 aa, Homo sapiens
MALSVDSSWHRWQWRVRDGFPHCPSETTPLLSPEKGRQSYNLTQQRVVFPNNSIFHQDWEEVSRRYPGNRTCTTKYTLFTFLPRNLFEQFHRWANLYFLFLVILNWMPSMEVFHREITMLPLAIVLFVIMIKDGMEDFKRHRFDKAINCSNIRIYERKEQTYVQKCWKDVRVGDFIQMKCNEIVPADILLLFSSDPNGICHLETASLDGETNLKQRCVVKGFSQQEVQFEPELFHNTIVCEKPNNHLNKFKGYMEHPDQTRTGFGCESLLLRGCTIRNTEMAVGIVIYAGHETKAMLNNSGPRYKRSKIERRMNIDIFFCIGILILMCLIGAVGHSIWNGTFEEHPPFDVPDANGSFLPSALGGFYMFLTMIILLQVLIPISLYVSIELVKLGQVFFLSNDLDLYDEETDLSIQCRALNIAEDLGQIQYIFSDKTGTLTENKMVFRRCTIMGSEYSHQENAKRLETPKELDSDGEEWTQYQCLSFSARWAQDPATMRSQKGAQPLRRSQSARVPIQGHYRQRSMGHRESSQPPVAFSSSIEKDVTPDKNLLTKVRDAALWLETLSDSRPAKASLSTTSSIADFFLALTICNSVMVSTTTEPRQRVTIKPSSKALGTSLEKIQQLFQKLKLLSLSQSFSSTAPSDTDLGESLGANVATTDSDERDDASVCSGGDSTDDGGYRSSMWDQGDILESGSGTSLEEALEAPATDLARPEFCYEAESPDEAALVHAAHAYSFTLVSRTPEQVTVRLPQGTCLTFSLLCTLGFDSVRKRMSVVVRHPLTGEIVVYTKGADSVIMDLLEDPACVPDINMEKKLRKIRARTQKHLDLYARDGLRTLCIAKKVVSEEDFRRWASFRREAEASLDNRDELLMETAQHLENQLTLLGATGIEDRLQEGVPDTIATLREAGIQLWVLTGDKQETAVNIAHSCRLLNQTDTVYTINTENQETCESILNCALEELKQFRELQKPDRKLFGFRLPSKTPSITSEAVVPEAGLVIDGKTLNAIFQGKLEKKFLELTQYCRSVLCCRSTPLQKSMIVKLVRDKLRVMTLSIGDGANDVSMIQAADIGIGISGQEGMQAVMSSDFAITRFKHLKKLLLVHGHWCYSRLARMVVYYLYKNVCYVNLLFWYQFFCGFSSSTMIDYWQMIFFNLFFTSLPPLVFGVLDKDISAETLLALPELYKSGQNSECYNLSTFWISMVDAFYQSLICFFIPYLAYKGSDIDVFTFGTPINTISLTTILLHQAMEMKTWTIFHGVVLLGSFLMYFLVSLLYNATCVICNSPTNPYWVMEGQLSNPTFYLVCFLTPVVALLPRYFFLSLQGTCGKSLISKAQKIDKLPPDKRNLEIQSWRSRQRPAPVPEVARPTHHPVSSITGQDFSASTPKSSNPPKRKHVEESVLHEQRCGTECMRDDSCSGDSSAQLSSGEHLLGPNRIMAYSRGQTDMCRCSKRSSHRRSQSSLTI

SEQ ID NO: 53 - DNA, 4386 bp, Homo sapiens
atggccctctcagtggactcatcgtggcatcggtggcagtggagagtcagagatggcttcccccattgtccatcggaaaccacaccgctgctctctccagagaaagggagacagagctacaacttgacacagcagcgggtcgtgttccccaacaacagcatattccatcaagattgggaagaggtctccaggagataccctggcaacagaacctgcacaaccaaatacaccctcttcaccttcctgccccggaatctctttgagcaatttcatagatgggctaacctctatttcctgttcctggtgattttgaactggatgccctccatggaagtcttccacagagaaatcaccatgttaccattggccattgtcctgttcgtcatcatgatcaaggatggcatggaggacttcaagagacaccgctttgataaagcaataaactgctccaacattcgaatttatgaaagaaaagagcagacctatgtgcagaagtgctggaaggatgtgcgcgtgggagacttcatccaaatgaaatgcaatgagattgtcccagcagacatactcctccttttttcctctgaccccaatgggatatgccatctggaaactgccagcttggatggagagacaaacctcaagcaaagatgtgtcgtgaagggcttctcacagcaggaggtacagttcgaaccagagcttttccacaataccatcgtgtgtgagaaacccaacaaccacctcaacaaatttaagggttatatggagcatcctgaccagaccaggactggctttggctgtgagagtcttctgcttcgaggctgcaccatcagaaacaccgagatggctgttggcattgtcatctatgcaggccatgagacgaaagccatgctgaacaacagtggcccccggtacaaacgcagcaagattgagcggcgcatgaatatagacatcttcttctgcattgggatcctcatcctcatgtgccttattggagctgtaggtcacagcatctggaatgggacctttgaagaacaccctcccttcgatgtgccagatgccaatggcagcttccttcccagtgcccttgggggcttctacatgttcctcacaatgatcatcctgctccaggtgctgatccccatctctttgtatgtctccattgagctggtgaagctcgggcaagtgttcttcttgagcaatgaccttgacctgtatgatgaagagaccgatttatccattcaatgtcgagccctcaacatcgcagaggacttgggccagatccagtacatcttctccgataagacggggaccctgacagagaacaagatggtgttccgacgttgcaccatcatgggcagcgagtattctcaccaagaaaatgctaagcgactggagaccccaaaggagctggactcagatggtgaagagtggacccaataccaatgcctgtccttctcggctagatgggcccaggatccagcaactatgagaagccaaaaaggtgctcagcctctgaggaggagccagagtgcccgggtgcccatccagggccactaccggcaaaggtctatggggcaccgtgaaagctcacagcctcctgtggccttcagcagctccatagaaaaagatgtaactccagataaaaacctactgaccaaggttcgagatgctgccctgtggttggagaccttgtcagacagcagacctgccaaggcttccctctccaccacctcctccattgctgatttcttccttgccttaaccatctgcaactctgtcatggtgtccacaaccaccgagcccaggcagagggtcaccatcaaaccctcaagcaaggctctggggacgtccctggagaagattcagcagctcttccagaagttgaagctattgagcctcagccagtcattctcatccactgcaccctctgacacagacctcggggagagcttaggggccaacgtggccaccacagactcggatgagagagatgatgcatctgtgtgcagtggaggtgactccactgatgacggtggctacaggagcagcatgtgggaccagggcgacatcctggagtctgggtcaggcacttccttggaggaggcattggaggccccagccacagacctggccaggcctgagttctgttacgaggctgagagccctgatgaggccgccctggtgcacgctgcccatgcctacagcttcacactagtgtcccggacacctgagcaggtgactgtgcgcctgccccagggcacctgcctcaccttcagcctcctctgcaccctgggctttgactctgtcaggaagagaatgtctgtggttgtgaggcacccactgactggcgagattgttgtctacaccaagggtgctgactcggtcatcatggacctgctggaagacccagcctgcgtacctgacattaatatggaaaagaagctgagaaaaatccgagcccggacccaaaagcatctagacttgtatgcaagagatggcctgcgcacactatgcattgccaagaaggttgtaagcgaagaggacttccggagatgggccagtttccggcgtgaggctgaggcatccctcgacaaccgagatgagcttctcatggaaactgcacagcatctggagaatcaactcaccttacttggagccactgggatcgaagaccggctgcaggaaggagttccagatacgattgccactctgcgggaggctgggatccagctctgggtcctgactggagataagcaggagacagcggtcaacattgcccattcctgcagactgttaaatcagaccgacactgtttataccatcaatacagagaatcaggagacctgtgaatccatcctcaattgtgcattggaagagctaaagcaatttcgtgaactacagaagccagaccgcaagctctttggattccgcttaccttccaagacaccatccatcacctcagaagctgtggttccagaagctggattggtcatcgatgggaagacattgaatgccatcttccagggaaagctagagaagaagtttctggaattgacccagtattgtcggtccgtcctgtgctgccgctccacgccactccagaagagtatgatagtcaagctggtgcgagacaagttgcgcgtcatgaccctttccataggtgatggagcaaatgatgtaagcatgattcaagctgctgatattggaattggaatatctggacaggaaggcatgcaggctgtcatgtccagcgactttgccatcacccgctttaagcatctcaagaagttgctgctcgtgcatggccactggtgttactcgcgcctggccaggatggtggtgtactacctctacaagaacgtgtgctacgtcaacctgctcttctggtatcagttcttctgtggtttctccagctccaccatgattgattactggcagatgatattcttcaatctcttctttacctccttgcctcctcttgtctttggagtccttgacaaagacatctctgcagaaacactcctggcattgcctgagctatacaagagtggccagaactctgagtgctataacctgtcgactttctggatttctatggtggatgcattctaccagagcctcatctgtttctttatcccttacctggcctataagggctctgatatagatgtctttacctttgggacaccaatcaacaccatctccctcaccacaatccttttgcaccaggcaatggaaatgaagacatggaccattttccacggagtcgtgctcctcggcagcttcctgatgtactttctggtatccctcctgtacaatgccacctgcgtcatctgcaacagccccaccaatccctattgggtgatggaaggccagctctcaaaccccactttctacctcgtctgctttctcacaccagttgttgctcttctcccaagatactttttcctgtctctgcaaggaacttgtgggaagtctctaatctcaaaagctcagaaaattgacaaactccccccagacaaaagaaacctggaaatccagagttggagaagcagacagaggcctgcccctgtccccgaagtggctcgaccaactcaccacccagtgtcatctatcacaggacaggacttcagtgccagcaccccaaagagctctaaccctcccaagaggaagcatgtggaagagtcagtactccacgaacagagatgtggcacggagtgcatgagggatgactcatgctcaggggactcctcagctcaactctcatccggggagcacctgctgggacctaacaggataatggcctactcaagaggacagactgatatgtgccggtgctcaaagaggagcagccatcgccgatcccagagttcactgaccatatga

SEQ ID NO: 54 - protein, 327 aa, Homo sapiens
MCSTSGCDLEEIPLDDDDLNTIEFKILAYYTRHHVFKSTPALFSPKLLRTRSLSQRGLGNCSANESWTEVSWPCRNSQSSEKAINLGKKKSSWKAFFGVVEKEDSQSTPAKVSAQGQRTLEYQDSHSQQWSRCLSNVEQCLEHEAVDPKVISIANRVAEIVYSWPPPQATQAGGFKSKEIFVTEGLSFQLQGHVPVASSSKKDEEEQILAKIVELLKYSGDQLERKLKKDKALMGHFQDGLSYSVFKTITDQVLMGVDPRGESEVKAQGFKAALVIDVTAKLTAIDNHPMNRVLGFGTKYLKENFSPWIQQHGGWEKILGISHEEVD

SEQ ID NO: 55 - DNA, 984 bp, Homo sapiens
atgtgtagcaccagtgggtgtgacctggaagaaatccccctagatgatgatgacctaaacaccatagaattcaaaatcctcgcctactacaccagacatcatgtcttcaagagcacccctgctctcttctcaccaaagctgctgagaacaagaagtttgtcccagaggggcctggggaattgttcagcaaatgagtcatggacagaggtgtcatggccttgcagaaattcccaatccagtgagaaggccataaaccttggcaagaaaaagtcttcttggaaagcattctttggagtagtggagaaggaagattcgcagagcacgcctgccaaggtctctgctcagggtcaaaggacgttggaataccaagattcgcacagccagcagtggtccaggtgtctttctaacgtggagcagtgcttggagcatgaagctgtggaccccaaagtcatttccattgccaaccgagtagctgaaattgtttactcctggccaccaccacaagcgacccaggcaggaggcttcaagtccaaagagatttttgtaactgagggtctctccttccagctccaaggccacgtgcctgtagcttcaagttctaagaaagatgaagaagaacaaatactagccaaaattgttgagctgctgaaatattcaggagatcagttggaaagaaagctgaagaaagataaggctttgatgggccacttccaggatgggctgtcctactctgttttcaagaccatcacagaccaggtcctaatgggtgtggaccccaggggagaatcagaggtcaaagctcagggctttaaggctgcccttgtaatagacgtcacggccaagctcacagctattgacaaccacccgatgaacagggtcctgggctttggcaccaagtacctgaaagagaacttctcgccatggatccagcagcacggtggatgggaaaaaatacttgggatatcacatgaagaagtagactga

SEQ ID NO: 56 - DNA, 759 bp, Homo sapiens
atgtgtagcaccagtgggtgtgacctggaagaaatccccctagatgatgatgacctaaacaccatagaattcaaaatcctcgcctactacaccagacatcatgtcttcaagagcacccctgctctcttctcaccaaagctgctgagaacaagaagtttgtcccagaggggcctggggaattgttcagcaaatgagtcatggacagaggtgtcatggccttgcagaaattcccaatccagtgagaaggccataaaccttggcaagaaaaagtcttcttggaaagcattctttggagtagtggagaaggaagattcgcagagcacgcctgccaaggtctctgctcagggtcaaaggacgttggaataccaagattcgcacagccagcagtggtccaggtgtctttctaacgtggagcagtgcttggagcatgaagctgtggaccccaaagtcatttccattgccaaccgagtagctgaaattgtttactcctggccaccaccacaagcgacccaggcaggaggcttcaagtccaaagagatttttgtaactgagggtctctccttccagctccaaggccacgtgcctgtagcttcaagttctaagaaagatgaagaagaacaaatactagccaaaattgttgagctgctgaaatattcaggagatcagttggaaagaaaggacactgccttcatccccattcccttggttgacaccagcatccagggttttccacaggatggtttgatggcctgcatttga

SEQ ID NO: 57 - protein, 395 aa, Homo sapiens
MLPAVGSADEEEDPAEEDCPELVPIETTQSEEEEKSGLGAKIPVTIITGYLGAGKTTLLNYILTEQHSKRVAVILNEFGEGSALEKSLAVSQGGELYEEWLELRNGCLCCSVKDSGLRAIENLMQKKGKFDYILLETTGLADPGAVASMFWVDAELGSDIYLDGIITIVDSKYGLKHLTEEKPDGLINEATRQVALADAILINKTDLVPEEDVKKLRTTIRSINGLGQILETQRSRVDLSNVLDLHAFDSLSGISLQKKLQHVPGTQPHLDQSIVTITFEVPGNAKEEHLNMFIQNLLWEKNVRNKDNHCMEVIRLKGLVSIKDKSQQVIVQGVHELYDLEETPVSWKDDTERTNRLVLLGRNLDKDILKQLFIATVTETEKQWTTRFQEDQVCT

SEQ ID NO: 58 - DNA, 1188 bp, Homo sapiens
atgttaccggctgttggatctgcggatgaggaggaggatcctgcggaggaggattgtcctgaattggttcccattgagacgacgcaaagcgaggaggaggaaaagtctggcctcggcgccaagatcccagtcacaattatcaccgggtatttaggtgctgggaagacaacacttctgaactatattttgacagagcaacatagtaaaagagtagcggtcattttaaatgaatttggggaaggaagtgcgctggagaaatccttagctgtcagccaaggtggagagctctatgaagagtggctggaacttagaaacggttgcctctgctgttcagtgaaggacagtggccttagagctattgagaatttgatgcaaaagaaggggaaatttgattacatactgttagagaccactggattagcagaccctggtgcagtggcttctatgttttgggttgatgctgaattagggagtgatatttatcttgatggtatcataactattgtggattcaaaatatggattaaaacatttaacagaagagaaacctgatggccttatcaatgaagctactagacaagttgctttggcagatgccattctcattaataaaacagacctggttccagaagaagatgtaaagaaattaagaacgacaattagatccataaatggactaggacaaatcttagaaacacaaagatcaagagttgatctctctaatgtattagatcttcatgcctttgatagtctctctggaataagtttgcagaaaaaacttcagcatgtgccaggaacacaacctcaccttgatcagagtattgttacaatcacatttgaagtaccaggaaatgcaaaggaagaacatcttaatatgtttattcagaatctcctgtgggaaaagaatgtgagaaacaaggacaatcactgcatggaggtcataaggctgaagggattggtgtcaatcaaagacaaatcacaacaagtgattgtccagggtgtccatgagctctatgatctggaggagactccagtgagctggaaggatgacactgagagaacaaatcgattggtcctccttggcagaaatttagataaggatatccttaaacagctgtttatagctactgtgacagaaacagaaaagcagtggacaacacgtttccaagaagatcaagtttgtacataa

SEQ ID NO: 59 - DNA, 1080 bp, Homo sapiens
atgtatttcaaacgtgccgctcgggcctttcccgtattgctcactggtgctgggaagacaacacttctgaactatattttgacagagcaacatagtaaaagagtagcggtcattttaaatgaatttggggaaggaagtgcgctggagaaatccttagctgtcagccaaggtggagagctctatgaagagtggctggaacttagaaacggttgcctctgctgttcagtgaaggacagtggccttagagctattgagaatttgatgcaaaagaaggggaaatttgattacatactgttagagaccactggattagcagaccctggtgcagtggcttctatgttttgggttgatgctgaattagggagtgatatttatcttgatggtatcataactattgtggattcaaaatatggattaaaacatttaacagaagagaaacctgatggccttatcaatgaagctactagacaagttgctttggcagatgccattctcattaataaaacagacctggttccagaagaagatgtaaagaaattaagaacgacaattagatccataaatggactaggacaaatcttagaaacacaaagatcaagagttgatctctctaatgtattagatcttcatgcctttgatagtctctctggaataagtttgcagaaaaaacttcagcatgtgccaggaacacaacctcaccttgatcagagtattgttacaatcacatttgaagtaccaggaaatgcaaaggaagaacatcttaatatgtttattcagaatctcctgtgggaaaagaatgtgagaaacaaggacaatcactgcatggaggtcataaggctgaagggattggtgtcaatcaaagacaaatcacaacaagtgattgtccagggtgtccatgagctctatgatctggaggagactccagtgagctggaaggatgacactgagagaacaaatcgattggtcctccttggcagaaatttagataaggatatccttaaacagctgtttatagctactgtgacagaaacagaaaagcagtggacaacacgtttccaagaagatcaagtttgtacataa

SEQ ID NO: 60 - DNA, 1131 bp, Homo sapiens
atgttaccggctgttggatctgcggatgaggaggaggatcctgcggaggaggattgtcctgaattggttcccattgagacgacgcaaagcgaggaggaggaaaagtctggcctcggcgccaagatcccagtcacaattatcaccgggtatttaggtgctgggaagacaacacttctgaactatattttgacagagcaacatagtaaaagagtagcggtcattttaaatgaatttggggaaggaagtgcgctggagaaatccttagctgtcagccaaggtggagagctctatgaagagtggctggaacttagaaacggttgcctctgctgttcagtgaaggacagtggccttagagctattgagaatttgatgcaaaagaaggggaaatttgattacatactgttagagaccactggattagcagaccctggtgcagtggcttctatgttttgggttgatgctgaattagggagtgatatttatcttgatggtatcataactattgtggattcaaaatatggattaaaacatttaacagaagagaaacctgatggccttatcaatgaagctactagacaagttgctttggcagatgccattctcattaataaaacagacctggttccagaagaagatgtaaagaaattaagaacgacaattagatccataaatggactaggacaaatcttagaaacacaaagatcaagtttgcagaaaaaacttcagcatgtgccaggaacacaacctcaccttgatcagagtattgttacaatcacatttgaagtaccaggaaatgcaaaggaagaacatcttaatatgtttattcagaatctcctgtgggaaaagaatgtgagaaacaaggacaatcactgcatggaggtcataaggctgaagggattggtgtcaatcaaagacaaatcacaacaagtgattgtccagggtgtccatgagctctatgatctggaggagactccagtgagctggaaggatgacactgagagaacaaatcgattggtcctccttggcagaaatttagataaggatatccttaaacagctgtttatagctactgtgacagaaacagaaaagcagtggacaacacgtttccaagaagatcaagtttgtacataa

SEQ ID NO: 61 - protein, 160 aa, Homo sapiens
MAFTFAAFCYMLSLVLCAALIFFAIWHIIAFDELRTDFKSPIDQCNPVHARERLRNIERICFLLRKLVLPEYSIHSLFCIMFLCAQEWLTLGLNVPLLFYHFWRYFHCPADSSELAYDPPVVMNADTLSYCQKEAWCKLAFYLLSFFYYLYCMIYTLVSS

SEQ ID NO: 62 - DNA, 483 bp, Homo sapiens
atggccttcactttcgctgcgttctgctacatgctgtctctggtgctgtgcgctgcgctcatcttcttcgccatctggcacataattgcctttgatgagttaaggacagattttaagagccccatagaccagtgcaatcctgttcatgcgagggaacggttgaggaacatcgagcgcatctgcttccttctgcgaaagctggtgctgccagaatactccatccatagcctcttctgcattatgttcctgtgtgcgcaagagtggctcacgctggggctgaatgtccctctacttttctatcacttctggaggtatttccactgtccagcagatagctcagaactagcctacgacccaccggtggtcatgaatgccgacactttgagttactgtcagaaggaggcctggtgtaagctggccttctatctcctctccttcttctactacctttactgcatgatctacactttagtgagctcttaa

SEQ ID NO: 63 - protein, 1033 aa, Homo sapiens
MGAAGLLGVFLALVAPGVLGISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFCPSPPPILNGRHIGNSLANVSYGSIVTYTCDPDPEEGVNFILIGESTLRCTVDSQKTGTWSGPAPRCELSTSAVQCPHPQILRGRMVSGQKDRYTYNDTVIFACMFGFTLKGSKQIRCNAQGTWEPSAPVCEKECQAPPNILNGQKEDRHMVRFDPGTSIKYSCNPGYVLVGEESIQCTSEGVWTPPVPQCKVAACEATGRQLLTKPQHQFVRPDVNSSCGEGYKLSGSVYQECQGTIPWFMEIRLCKEITCPPPPVIYNGAHTGSSLEDFPYGTTVTYTCNPGPERGVEFSLIGESTIRCTSNDQERGTWSGPAPLCKLSLLAVQCSHVHIANGYKISGKEAPYFYNDTVTFKCYSGFTLKGSSQIRCKADNTWDPEIPVCEKETCQHVRQSLQELPAGSRVELVNTSCQDGYQLTGHAYQMCQDAENGIWFKKIPLCKVIHCHPPPVIVNGKHTGMMAENFLYGNEVSYECDQGFYLLGEKKLQCRSDSKGHGSWSGPSPQCLRSPPVTRCPNPEVKHGYKLNKTHSAYSHNDIVYVDCNPGFIMNGSRVIRCHTDNTWVPGVPTCIKKAFIGCPPPPKTPNGNHTGGNIARFSPGMSILYSCDQGYLLVGEALLLCTHEGTWSQPAPHCKEVNCSSPADMDGIQKGLEPRKMYQYGAVVTLECEDGYMLEGSPQSQCQSDHQWNPPLAVCRSRSLAPVLCGIAAGLILLTFLIVITLYVISKHRARNYYTDTSQKEAFHLEAREVYSVDPYNPAS

SEQ ID NO: 64 - DNA, 3279 bp, Homo sapiens
atgggcgccgcgggcctgctcggggttttcttggctctcgtcgcaccgggggtcctcgggatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaattttttgcccatcacctccccctattctcaatggaagacatataggcaactcactagcaaatgtctcatatggaagcatagtcacttacacttgtgacccggacccagaggaaggagtgaacttcatccttattggagagagcactctccgttgtacagttgatagtcagaagactgggacctggagtggccctgccccacgctgtgaactttctacttctgcggttcagtgtccacatccccagatcctaagaggccgaatggtatctgggcagaaagatcgatatacctataacgacactgtgatatttgcttgcatgtttggcttcaccttgaagggcagcaagcaaatccgatgcaatgcccaaggcacatgggagccatctgcaccagtctgtgaaaaggaatgccaggcccctcctaacatcctcaatgggcaaaaggaagatagacacatggtccgctttgaccctggaacatctataaaatatagctgtaaccctggctatgtgctggtgggagaagaatccatacagtgtacctctgagggggtgtggacaccccctgtaccccaatgcaaagtggcagcgtgtgaagctacaggaaggcaactcttgacaaaaccccagcaccaatttgttagaccagatgtcaactcttcttgtggtgaagggtacaagttaagtgggagtgtttatcaggagtgtcaaggcacaattccttggtttatggagattcgtctttgtaaagaaatcacctgcccaccaccccctgttatctacaatggggcacacaccgggagttccttagaagattttccatatggaaccacggtcacttacacatgtaaccctgggccagaaagaggagtggaattcagcctcattggagagagcaccatccgttgtacaagcaatgatcaagaaagaggcacctggagtggccctgctcccctgtgtaaactttccctccttgctgtccagtgctcacatgtccatattgcaaatggatacaagatatctggcaaggaagccccatatttctacaatgacactgtgacattcaagtgttatagtggatttactttgaagggcagtagtcagattcgttgcaaagctgataacacctgggatcctgaaataccagtttgtgaaaaaggctgccagtcacctcctgggctccaccatggtcgtcatacaggtggaaatacggtcttctttgtctctgggatgactgtagactacacttgtgaccctggctatttgcttgtgggaaacaaatccattcactgtatgccttcaggaaattggagtccttctgccccacggtgtgaagaaacatgccagcatgtgagacagagtcttcaagaacttccagctggttcacgtgtggagctagttaatacgtcctgccaagatgggtaccagttgactggacatgcttatcagatgtgtcaagatgctgaaaatggaatttggttcaaaaagattccactttgtaaagttattcactgtcaccctccaccagtgattgtcaatgggaagcacacaggcatgatggcagaaaactttctatatggaaatgaagtctcttatgaatgtgaccaaggattctatctcctgggagagaaaaaattgcagtgcagaagtgattctaaaggacatggatcttggagcgggccttccccacagtgcttacgatctcctcctgtgactcgctgccctaatccagaagtcaaacatgggtacaagctcaataaaacacattctgcatattcccacaatgacatagtgtatgttgactgcaatcctggcttcatcatgaatggtagtcgcgtgattaggtgtcatactgataacacatgggtgccaggtgtgccaacttgtatcaaaaaagccttcatagggtgtccacctccgcctaagacccctaacgggaaccatactggtggaaacatagctcgattttctcctggaatgtcaatcctgtacagctgtgaccaaggctacctgctggtgggagaggcactccttctttgcacacatgagggaacctggagccaacctgcccctcattgtaaagaggtaaactgtagctcaccagcagatatggatggaatccagaaagggctggaaccaaggaaaatgtatcagtatggagctgttgtaactctggagtgtgaagatgggtatatgctggaaggcagtccccagagccagtgccaatcggatcaccaatggaaccctcccctggcggtttgcagatcccgttcacttgctcctgtcctttgtggtattgctgcaggtttgatacttcttaccttcttgattgtcattaccttatacgtgatatcaaaacacagagcacgcaattattatacagatacaagccagaaagaagcttttcatttagaagcacgagaagtatattctgttgatccatacaacccagccagctga

SEQ ID NO: 65 - DNA, 3102 bp, Homo sapiens
atgggcgccgcgggcctgctcggggttttcttggctctcgtcgcaccgggggtcctcgggatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaattttttgcccatcacctccccctattctcaatggaagacatataggcaactcactagcaaatgtctcatatggaagcatagtcacttacacttgtgacccggacccagaggaaggagtgaacttcatccttattggagagagcactctccgttgtacagttgatagtcagaagactgggacctggagtggccctgccccacgctgtgaactttctacttctgcggttcagtgtccacatccccagatcctaagaggccgaatggtatctgggcagaaagatcgatatacctataacgacactgtgatatttgcttgcatgtttggcttcaccttgaagggcagcaagcaaatccgatgcaatgcccaaggcacatgggagccatctgcaccagtctgtgaaaaggaatgccaggcccctcctaacatcctcaatgggcaaaaggaagatagacacatggtccgctttgaccctggaacatctataaaatatagctgtaaccctggctatgtgctggtgggagaagaatccatacagtgtacctctgagggggtgtggacaccccctgtaccccaatgcaaagtggcagcgtgtgaagctacaggaaggcaactcttgacaaaaccccagcaccaatttgttagaccagatgtcaactcttcttgtggtgaagggtacaagttaagtgggagtgtttatcaggagtgtcaaggcacaattccttggtttatggagattcgtctttgtaaagaaatcacctgcccaccaccccctgttatctacaatggggcacacaccgggagttccttagaagattttccatatggaaccacggtcacttacacatgtaaccctgggccagaaagaggagtggaattcagcctcattggagagagcaccatccgttgtacaagcaatgatcaagaaagaggcacctggagtggccctgctcccctgtgtaaactttccctccttgctgtccagtgctcacatgtccatattgcaaatggatacaagatatctggcaaggaagccccatatttctacaatgacactgtgacattcaagtgttatagtggatttactttgaagggcagtagtcagattcgttgcaaagctgataacacctgggatcctgaaataccagtttgtgaaaaagaaacatgccagcatgtgagacagagtcttcaagaacttccagctggttcacgtgtggagctagttaatacgtcctgccaagatgggtaccagttgactggacatgcttatcagatgtgtcaagatgctgaaaatggaatttggttcaaaaagattccactttgtaaagttattcactgtcaccctccaccagtgattgtcaatgggaagcacacaggcatgatggcagaaaactttctatatggaaatgaagtctcttatgaatgtgaccaaggattctatctcctgggagagaaaaaattgcagtgcagaagtgattctaaaggacatggatcttggagcgggccttccccacagtgcttacgatctcctcctgtgactcgctgccctaatccagaagtcaaacatgggtacaagctcaataaaacacattctgcatattcccacaatgacatagtgtatgttgactgcaatcctggcttcatcatgaatggtagtcgcgtgattaggtgtcatactgataacacatgggtgccaggtgtgccaacttgtatcaaaaaagccttcatagggtgtccacctccgcctaagacccctaacgggaaccatactggtggaaacatagctcgattttctcctggaatgtcaatcctgtacagctgtgaccaaggctacctgctggtgggagaggcactccttctttgcacacatgagggaacctggagccaacctgcccctcattgtaaagaggtaaactgtagctcaccagcagatatggatggaatccagaaagggctggaaccaaggaaaatgtatcagtatggagctgttgtaactctggagtgtgaagatgggtatatgctggaaggcagtccccagagccagtgccaatcggatcaccaatggaaccctcccctggcggtttgcagatcccgttcacttgctcctgtcctttgtggtattgctgcaggtttgatacttcttaccttcttgattgtcattaccttatacgtgatatcaaaacacagagcacgcaattattatacagatacaagccagaaagaagcttttcatttagaagcacgagaagtatattctgttgatccatacaacccagccagctga

SEQ ID NO: 66 - protein, 1198 aa, Homo sapiens
MRSLRKKREKPRPEQWKGLPGPPRAPEPEDVAVPGGVDLLTLPQLCFPGGVCVATEPKEDCVHFLVLTDVCGNRTYGVVAQYYRPLHDEYCFYNGKTHRECPGCFVPFAVCVVSRFPYYNSLKDCLSCLLALLKPCKDFEVDSHIKDFAAKLSLIPSPPPGPLHLVFNMKSLQIVLPARADPESPILDLDLHLPLLCFRPEKVLQILTCILTEQRIVFFSSDWALLTLVTECFMAYLYPLQWQHPFVPILSDQMLDFVMAPTSFLMGCHLDHFEEVSKEADGLVLINIDHGSITYSKSTDDNVDIPDVPLLAAQTFIQRVQSLQLHHELHAAHLLSSTDLKEGRAHRRSWQQKLNCQIQQTTLQLLVSIFRDVKNHLNYEHRVFNSEEFLKTRAPGDHQFYKQVLDTYMFHSFLKARLNRRMDAFAQMDLDTQSEEDRINGMLLSPRRPTVEKRASRKSSHLHVTHRRMVVSMPNLQDIAMPELAPRNSSLRLTDTAGCRGSSAVLNVTPKSPYTFKIPEIHFPLESKCVQAYHAHFVSMLSEAMCFLAPDNSLLLARYLYLRGLVYLMQGQLLNALLDFQNLYKTDIRIFPTDLVKRTVESMSAPEWEGAEQAPELMRLISEILDKPHEASKLDDHVKKFKLPKKHMQLGDFMKRVQESGIVKDASIIHRLFEALTVGQEKQIDPETFKDFYNCWKETEAEAQEVSLPWLVMEHLDKNECVCKLSSSVKTNLGVGKIAMTQKRLFLLTEGRPGYLEISTFRNIEEVRRTTTTFLLRRIPTLKIRVASKKEVFEANLKTECDLWHLMVKEMWAGKKLADDHKDPHYVQQALTNVLLMDAVVGTLQSPGAIYAASKLSYFDKMSNEMPMTLPETTLETLKHKINPSAGEAFPQAVDVLLYTPGHLDPAEKVEDAHPKLWCALSEGKVTVFNASSWTIHQHSFKVGTAKVNCMVMADQNQVWVGSEDSVIYIINVHSMSCNKQLTAHCSSVTDLIVQDGQEAPSNVYSCSMDGMVLVWNVSTLQVTSRFQLPRGGLTSIRLHGGRLWCCTGNSIMVMKMNGSLHQELKIEENFKDTSTSFLAFQLLPEEEQLWAACAGRSEVYIWSLKDLAQPPQRVPLEDCSEINCMIRVKKQVWVGSRGLGQGTPKGKIYVIDAERKTVEKELVAHMDTVRTLCSAEDRYVLSGSGREEGKVAIWKGE

SEQ ID NO: 67 - DNA, 3597 bp, Homo sapiens
atgcgctccttgagaaagaagagagagaagcccagaccagagcagtggaagggcctcccggggccccccagagcgccagagcctgaggatgtcgccgtcccgggcggcgtggacctcctcaccctgccgcagctgtgcttcccagggggtgtgtgcgtggccactgaacctaaggaggattgcgtccacttcctggtgctgaccgatgtctgcgggaataggacctatggcgtggtggcccagtactaccggcccctgcatgatgagtactgtttctacaatggcaaaacgcaccgggagtgtcctggctgcttcgtgcccttcgcggtgtgcgtggtctccaggtttccctattacaactccctcaaggactgcctttcctgtttattggctcttctgaagccctgtaaagattttgaagtggacagtcatataaaagatttcgctgcgaagctgtctttaatacccagcccgccacctggaccgctccatttggtatttaacatgaagtcgctccagattgtgttacctgcccgagcagaccccgaaagccccatcctggacctggaccttcacctgcccttgctgtgcttcaggcctgagaaggtgctacagatcctgacatgcatcctgacggaacagcggatcgtcttcttctcctcggactgggctctgctgacgctggtcactgagtgcttcatggcctacctgtatccgctgcagtggcagcaccccttcgtgcccatcctgtcggaccagatgctggatttcgtcatggcccccacgtccttcctgatgggctgccatctcgaccacttcgaagaagtcagcaaggaagccgacggtttagttctgataaatattgatcatgggagcatcacctactccaagtccacggacgataacgtggacattcctgatgtccccctcctggcagcccagacgtttattcagagggtgcagagcctccagctccaccatgagctgcacgccgcccacctcctctccagcacagacctgaaggagggccgagcccaccggcggtcctggcagcagaaactcaactgccagatacagcagaccaccctgcagctgctcgtgagcatcttcagggatgtaaagaatcatttaaactatgaacacagagtctttaatagtgaagaatttctcaaaaccagggctccaggggaccatcagttttataagcaggtcttagacacctacatgttccattcttttcttaaagcccggctcaataggaggatggacgcctttgctcagatggacctcgacacccagtcggaggaggacagaataaatggaatgcttctaagtccaaggagaccgaccgttgagaaaagagcctcccggaagtcctcgcacctgcatgtcacccacaggcgcatggtggtcagcatgcccaacctgcaggacattgccatgcctgagctggcacccaggaactcctcgctccggctgacggacaccgcaggctgtaggggcagcagcgcagttctgaatgtcacgccgaagtccccgtatacattcaagattcccgaaatccactttccgctggagagcaagtgcgtgcaggcataccatgcccactttgtctccatgctgagcgaggccatgtgctttctggcccccgataactctctgctcctggcccgctatttgtacctccgagggctcgtttatctgatgcagggacagctgctgaacgccctcttggacttccagaatctgtataaaacagacatacggatctttcccactgatttggtgaagaggacggtggaatccatgtctgcccctgagtgggagggggctgagcaggcgccggagctgatgaggctcatcagcgagatcctggacaagccgcacgaggcctcgaagctggacgaccacgtgaagaagttcaagctgcccaagaagcacatgcagctgggcgacttcatgaagcgggtccaggagtcagggatcgtgaaggacgccagcatcatacaccggctgttcgaggccttgactgtaggacaggagaaacaaatcgacccagaaacattcaaagatttctacaactgctggaaggagacggaagcagaagcccaggaggtcagtctgccgtggctggtgatggaacacctggataaaaacgagtgtgtgtgtaagttgtccagctccgtcaagacaaacctaggcgttggcaagatcgccatgacccagaagcgcctgttcctcctaaccgaaggaaggccaggctacttggagatttccaccttcagaaatatagaggaggtcaggagaaccactactacatttctacttcggagaatacccactttaaaaatcagagtggcgtccaagaaagaagtcttcgaagccaacctgaaaaccgagtgtgacctttggcacctgatggtgaaggagatgtgggctgggaagaagctggccgatgaccacaaggaccctcactacgtccagcaggcgctgaccaacgtcttgctgatggacgccgtcgtgggcacactgcagtcaccaggcgccatctacgctgcctccaagttatcctactttgataagatgagtaacgaaatgcccatgacgcttccggagacaaccctggaaacactgaagcataaaatcaacccctcggcgggggaggcgttcccacaagcggtggacgtgctgctctacactccagggcatcttgacccagccgaaaaagttgaagatgctcaccccaagttatggtgtgctctgagcgaaggcaaggtgaccgtgttcaatgcttcttcatggaccatccaccagcactcctttaaagtgggcactgcaaaagtgaactgcatggtgatggccgaccagaaccaggtgtgggttggctcggaagactccgtcatctacatcatcaacgtccacagcatgtcctgcaacaagcagctcacagcccactgctccagtgtcacggatttgattgtgcaggacggacaggaggcacccagcaacgtgtactcgtgcagcatggacggcatggtgctggtgtggaatgtgagcacactgcaggtgaccagccgcttccagctgccgcgaggtggcctgacgtccatcagactgcacggcggccgcctgtggtgctgcacaggtaacagcatcatggtcatgaaaatgaatggatccctccatcaagaattgaagattgaggagaacttcaaagacaccagtacctccttcctggccttccagctccttcctgaggaggagcagctgtgggcggcctgtgcaggacgcagcgaggtttacatctggagcctgaaggacctggcccagcccccgcagagggtgcccctcgaggactgctctgagatcaactgcatgatccgggtgaagaagcaggtctgggtgggcagccgagggctggggcagggaacacccaaggggaaaatctacgtgattgacgccgagaggaagaccgtggagaaggagctggtggcgcacatggacaccgtgaggacgctgtgctcggctgaggacagatacgtgctgagtgggtcgggcagggaggaggggaaagtcgccatttggaaaggcgaataa

SEQ ID NO: 68 - protein, 795 aa, Homo sapiens
MSKRHRLDLGEDYPSGKKRAGTDGKDRDRDRDREDRSKDRDRERDRGDREREREKEKEKELRASTNAMLISAGLPPLKASHSAHSTHSAHSTHSTHSAHSTHAGHAGHTSLPQCINPFTNLPHTPRYYDILKKRLQLPVWEYKDRFTDILVRHQSFVLVGETGSGKTTQIPQWCVEYMRSLPGPKRGVACTQPRRVAAMSVAQRVADEMDVMLGQEVGYSIRFEDCSSAKTILKYMTDGMLLREAMNDPLLERYGVIILDEAHERTLATDILMGVLKEVVRQRSDLKVIVMSATLDAGKFQIYFDNCPLLTIPGRTHPVEIFYTPEPERDYLEAAIRTVIQIHMCEEEEGDLLLFLTGQEEIDEACKRIKREVDDLGPEVGDIKIIPLYSTLPPQQQQRIFEPPPPKKQNGAIGRKVVVSTNIAETSLTIDGVVFVIDPGFAKQKVYNPRIRVESLLVTAISKASAQQRAGRAGRTRPGKCFRLYTEKAYKTEMQDNTYPEILRSNLGSVVLQLKKLGIDDLVHFDFMDPPAPETLMRALELLNYLAALNDDGDLTELGSMMAEFPLDPQLAKMVIASCDYNCSNEVLSITAMLSVPQCFVRPTEAKKAADEAKMRFAHIDGDHLTLLNVYHAFKQNHESVQWCYDNFINYRSLMSADNVRQQLSRIMDRFNLPRRSTDFTSRDYYINIRKALVTGYFMQVAHLERTGHYLTVKDNQVVQLHPSTVLDHKPEWVLYNEFVLTTKNYIRTCTDIKPEWLVKIAPQYYDMSNFPQCEAKRQLDRIIAKLQSKEYSQY

SEQ ID NO: 69 - DNA, 2388 bp, Homo sapiens
atgtccaagcggcaccggttggacctaggggaggattacccctctggcaagaagcgtgcggggaccgatgggaaggatcgagatcgagaccgggatcgtgaagatcggtctaaagatcgagaccgagaacgtgatagaggagatagagagcgagagagggagaaagaaaaggagaaggagttgcgagcttcaacaaatgctatgcttatcagtgctggattaccacctttgaaagcttcccattcagctcactcaacccactcagcacattcaacgcattcaacacattctgctcattcaacgcatgccggacatgcaggtcacacgtcacttccacagtgcattaatccgttcaccaacttaccccatactcctcgatactatgatattctaaagaaacgtcttcagctccctgtttgggaatacaaggataggtttacagatattctggttagacatcagtcctttgtactggttggtgagactgggtctggtaaaacaacacagattccacagtggtgtgtggagtacatgcgatcattaccaggacccaagagaggagttgcctgtacccaacccaggagagtggctgcaatgagtgtggctcagagagttgctgatgagatggatgtgatgttgggccaggaagttggttactccattcgatttgaagactgcagtagtgcaaaaaccattcttaagtatatgactgatgggatgttacttcgtgaagctatgaatgatcccctcctggagcgttatggtgtaataattcttgatgaggctcatgagaggacactggctacagatattctaatgggtgttctgaaggaagttgtaagacagagatcagatttaaaggttatagttatgagcgctactctagatgcaggaaaattccagatttactttgataactgtcctctcctaactattcctgggcgtacacatcctgttgagatcttctatactccagaaccagagagagattatcttgaagcagcaattcgaacagttatccagattcatatgtgtgaagaggaagagggagatcttcttcttttcttaactggtcaagaggaaattgatgaagcctgtaagagaataaagcgtgaagttgatgatttgggccctgaagttggtgacattaaaatcattccattgtattctacacttccacctcagcagcagcaacgcatttttgagcctccacctcccaaaaaacagaatggagcaattggaagaaaggtagttgtgtcaactaacatagcagagacgtctttgacaatagatggtgtggtgtttgtgattgatcctggatttgcgaaacagaaggtctacaatcctcgaatcagagttgagtcccttttggtgacagctattagtaaagcttcagctcagcaaagggctggtcgagctggacgtaccagacctggaaaatgcttcagactttacacagagaaagcttataaaacagaaatgcaggataacacctatcctgagattttgcgttctaatttaggatcagttgtgttacaattgaagaaacttggtattgatgacttggtacattttgattttatggatccaccagctcctgaaactctgatgagagccctggaacttttgaattacctggctgctttaaatgatgatggagatctgactgaattgggatccatgatggcagagtttcctctagatccacagctcgcaaaaatggttattgcaagttgtgactacaactgttctaatgaggtcctatctattactgctatgttgtcagtcccacagtgttttgttcgccccacggaggccaagaaagccgcagatgaggccaagatgagatttgcccacatagatggagatcatctgacactgctgaacgtctaccatgcttttaaacaaaatcatgaatcggttcagtggtgttatgacaacttcattaactacaggtccctgatgtccgcagacaatgtacgccagcagctatctcgaattatggacagatttaatttgcctcgtcgaagtactgactttacaagcagggactattatattaatataagaaaagctttggttactgggtattttatgcaggtggcacatttagaacgaacagggcattacttaactgtgaaagataaccaggtggttcagttgcatccctctactgttcttgaccacaaacctgaatgggtgctttataatgagtttgttctaacaacaaagaattacatccggacatgtacagatatcaagccagaatggttggtgaaaattgcccctcaatattatgacatgagcaatttcccacagtgtgaagcaaagagacagttggaccgcatcattgccaaacttcaatccaaggaatattcacagtactga

SEQ ID NO: 70 - protein, 352 aa, Homo sapiens
MASRKEGTGSTATSSSSTAGAAGKGKGKGGSGDSAVKQVQIDGLVVLKIIKHYQEEGQGTEVVQGVLLGLVVEDRLEITNCFPFPQHTEDDADFDEVQYQMEMMRSLRHVNIDHLHVGWYQSTYYGSFVTRALLDSQFSYQHAIEESVVLIYDPIKTAQGSLSLKAYRLTPKLMEVCKEKDFSPEALKKANITFEYMFEEVPIVIKNSHLINVLMWELEKKSAVADKHELLSLASSNHLGKNLQLLMDRVDEMSQDIVKYNTYMRNTSKQQQQKHQYQQRRQQENMQRQSRGEPPLPEEDLSKLFKPPQPPARMDSLLIAGQINTYCQNIKEFTAQNLGKLFMAQALQEYNN

SEQ ID NO: 71 - DNA, 1059 bp, Homo sapiens
atggcgtcccgcaaggaaggtaccggctctactgccacctcttccagctccaccgccggcgcagcagggaaaggcaaaggcaaaggcggctcgggagattcagccgtgaagcaagtgcagatagatggccttgtggtattaaagataatcaaacattatcaagaagaaggacaaggaactgaagttgttcaaggagtgcttttgggtctggttgtagaagatcggcttgaaattaccaactgctttcctttccctcagcacacagaggatgatgctgactttgatgaagtccaatatcagatggaaatgatgcggagccttcgccatgtaaacattgatcatcttcacgtgggctggtatcagtccacatactatggctcattcgttacccgggcactcctggactctcagtttagttaccagcatgccattgaagaatctgtcgttctcatttatgatcccataaaaactgcccaaggatctctctcactaaaggcatacagactgactcctaaactgatggaagtttgtaaagaaaaggatttttcccctgaagcattgaaaaaagcaaatatcacctttgagtacatgtttgaagaagtgccgattgtaattaaaaattcacatctgatcaatgtcctaatgtgggaacttgaaaagaagtcagctgttgcagataaacatgaattgctcagccttgccagcagcaatcatttggggaagaatctacagttgctgatggacagagtggatgaaatgagccaagatatagttaaatacaacacatacatgaggaatactagtaaacaacagcagcagaaacatcagtatcagcagcgtcgccagcaggagaatatgcagcgccagagccgaggagaacccccgctccctgaggaggacctgtccaaactcttcaaaccaccacagccgcctgccaggatggactcgctgctcattgcaggccagataaacacttactgccagaacatcaaggagttcactgcccaaaacttaggcaagctcttcatggcccaggctcttcaagaatacaacaactaa

SEQ ID NO: 72 - protein, 1037 aa, Homo sapiens
MRGSGPRGAGRRRPPSGGGDTPITPASLAGCYSAPRRAPLWTCLLLCAALRTLLASPSNEVNLLDSRTVMGDLGWIAFPKNGWEEIGEVDENYAPIHTYQVCKVMEQNQNNWLLTSWISNEGASRIFIELKFTLRDCNSLPGGLGTCKETFNMYYFESDDQNGRNIKENQYIKIDTIAADESFTELDLGDRVMKLNTEVRDVGPLSKKGFYLAFQDVGACIALVSVRVYYKKCPSVVRHLAVFPDTITGADSSQLLEVSGSCVNHSVTDEPPKMHCSAEGEWLVPIGKCMCKAGYEEKNGTCQVCRPGFFKASPHIQSCGKCPPHSYTHEEASTSCVCEKDYFRRESDPPTMACTRPPSAPRNAISNVNETSVFLEWIPPADTGGRKDVSYYIACKKCNSHAGVCEECGGHVRYLPRQSGLKNTSVMMVDLLAHTNYTFEIEAVNGVSDLSPGARQYVSVNVTTNQAAPSPVTNVKKGKIAKNSISLSWQEPDRPNGIILEYEIKYFEKDQETSYTIIKSKETTITAEGLKPASVYVFQIRARTAAGYGVFSRRFEFETTPVFAASSDQSQIPVIAVSVTVGVILLAVVIGVLLSGSCCECGCGRASSLCAVAHPSLIWRCGYSKAKQDPEEEKMHFHNGHIKLPGVRTYIDPHTYEDPNQAVHEFAKEIEASCITIERVIGAGEFGEVCSGRLKLPGKRELPVAIKTLKVGYTEKQRRDFLGEASIMGQFDHPNIIHLEGVVTKSKPVMIVTEYMENGSLDTFLKKNDGQFTVIQLVGMLRGISAGMKYLSDMGYVHRDLAARNILINSNLVCKVSDFGLSRVLEDDPEAAYTTRGGKIPIRWTAPEAIAFRKFTSASDVWSYGIVMWEVVSYGERPYWEMTNQDVIKAVEEGYRLPSPMDCPAALYQLMLDCWQKERNSRPKFDEIVNMLDKLIRNPSSLKTLVNASCRVSNLLAEHSPLGSGAYRSVGEWLEAIKMGRYTEIFMENGYSSMDAVAQVTLEDLRRLGVTLVGHQKKIMNSLQEMKVQLVNGMVPL

SEQ ID NO: 73 - DNA, 3114 bp, Homo sapiens
atgcggggctcggggccccggggtgcgggacgccggcggcccccaagcggcggcggcgacacccccatcaccccagcgtccctggccggctgctactctgcacctcgacgggctcccctctggacgtgccttctcctgtgcgccgcactccggaccctcctggccagccccagcaacgaagtgaatttattggattcacgcactgtcatgggggacctgggatggattgcttttccaaaaaatgggtgggaagagattggtgaagtggatgaaaattatgcccctatccacacataccaagtatgcaaagtgatggaacagaatcagaataactggcttttgaccagttggatctccaatgaaggtgcttccagaatcttcatagaactcaaatttaccctgcgggactgcaacagccttcctggaggactggggacctgtaaggaaacctttaatatgtattactttgagtcagatgatcagaatgggagaaacatcaaggaaaaccaatacatcaaaattgataccattgctgccgatgaaagctttacagaacttgatcttggtgaccgtgttatgaaactgaatacagaggtcagagatgtaggacctctaagcaaaaagggattttatcttgcttttcaagatgttggtgcttgcattgctctggtttctgtgcgtgtatactataaaaaatgcccttctgtggtacgacacttggctgtcttccctgacaccatcactggagctgattcttcccaattgctcgaagtgtcaggctcctgtgtcaaccattctgtgaccgatgaacctcccaaaatgcactgcagcgccgaaggggagtggctggtgcccatcgggaaatgcatgtgcaaggcaggatatgaagagaaaaatggcacctgtcaagtgtgcagacctgggttcttcaaagcctcacctcacatccagagctgcggcaaatgtccacctcacagttatacccatgaggaagcttcaacctcttgtgtctgtgaaaaggattatttcaggagagagtctgatccacccacaatggcatgcacaagacccccctctgctcctcggaatgccatctcaaatgttaatgaaactagtgtctttctggaatggattccgcctgctgacactggtggaaggaaagacgtgtcatattatattgcatgcaagaagtgcaactcccatgcaggtgtgtgtgaggagtgtggcggtcatgtcaggtaccttccccggcaaagcggcctgaaaaacacctctgtcatgatggtggatctactcgctcacacaaactatacctttgagattgaggcagtgaatggagtgtccgacttgagcccaggagcccggcagtatgtgtctgtaaatgtaaccacaaatcaagcagctccatctccagtcaccaatgtgaaaaaagggaaaattgcaaaaaacagcatctctttgtcttggcaagaaccagatcgtcccaatggaatcatcctagagtatgaaatcaagtattttgaaaaggaccaagagaccagctacacgattatcaaatctaaagagacaactattactgcagagggcttgaaaccagcttcagtttatgtcttccaaattcgagcacgtacagcagcaggctatggtgtcttcagtcgaagatttgagtttgaaaccaccccagtgtttgcagcatccagcgatcaaagccagattcctgtaattgctgtgtctgtgacagtgggagtcattttgttggcagtggttatcggcgtcctcctcagtggaagttgctgcgaatgtggctgtgggagggcttcttccctgtgcgctgttgcccatccaagcctaatatggcggtgtggctacagcaaagcaaaacaagatccagaagaggaaaagatgcattttcataatgggcacattaaactgccaggagtaagaacttacattgatccacatacctatgaggatcccaatcaagctgtccacgaatttgctaaggagatagaagcatcatgtatcaccattgagagagttattggagcaggtgaatttggtgaagtttgtagtggacgtttgaaactaccaggaaaaagagaattacctgtggctatcaaaacccttaaagtaggctatactgaaaagcaacgcagagatttcctaggtgaagcaagtatcatgggacagtttgatcatcctaacatcatccatttagaaggtgtggtgaccaaaagtaaaccagtgatgatcgtgacagagtatatggagaatggctctttagatacatttttgaagaaaaacgatgggcagttcactgtgattcagcttgttggcatgctgagaggtatctctgcaggaatgaagtacctttctgacatgggctatgtgcatagagatcttgctgccagaaacatcttaatcaacagtaaccttgtgtgcaaagtgtctgactttggactttcccgggtactggaagatgatcccgaggcagcctacaccacaaggggaggaaaaattccaatcagatggactgccccagaagcaatagctttccgaaagtttacttctgccagtgatgtctggagttatggaatagtaatgtgggaagttgtgtcttatggagagagaccctactgggagatgaccaatcaagatgtgattaaagcggtagaggaaggctatcgtctgccaagccccatggattgtcctgctgctctctatcagttaatgctggattgctggcagaaagagcgaaatagcaggcccaagtttgatgaaatagtcaacatgttggacaagctgatacgtaacccaagtagtctgaagacgctggttaatgcatcctgcagagtatctaatttattggcagaacatagcccactaggatctggggcctacagatcagtaggtgaatggctagaggcaatcaagatgggccggtatacagagattttcatggaaaatggatacagttcaatggacgctgtggctcaggtgaccttggaggatttgagacggcttggagtgactcttgtcggtcaccagaagaagatcatgaacagccttcaagaaatgaaggtgcagctggtaaacggaatggtgccattgtaa

SEQ ID NO: 74 - DNA, 3048 bp, Homo sapiens
atgcggggctcggggccccggggtgcgggacgccggcggcccccaagcggcggcggcgacacccccatcaccccagcgtccctggccggctgctactctgcacctcgacgggctcccctctggacgtgccttctcctgtgcgccgcactccggaccctcctggccagccccagcaacgaagtgaatttattggattcacgcactgtcatgggggacctgggatggattgcttttccaaaaaatgggtgggaagagattggtgaagtggatgaaaattatgcccctatccacacataccaagtatgcaaagtgatggaacagaatcagaataactggcttttgaccagttggatctccaatgaaggtgcttccagaatcttcatagaactcaaatttaccctgcgggactgcaacagccttcctggaggactggggacctgtaaggaaacctttaatatgtattactttgagtcagatgatcagaatgggagaaacatcaaggaaaaccaatacatcaaaattgataccattgctgccgatgaaagctttacagaacttgatcttggtgaccgtgttatgaaactgaatacagaggtcagagatgtaggacctctaagcaaaaagggattttatcttgcttttcaagatgttggtgcttgcattgctctggtttctgtgcgtgtatactataaaaaatgcccttctgtggtacgacacttggctgtcttccctgacaccatcactggagctgattcttcccaattgctcgaagtgtcaggctcctgtgtcaaccattctgtgaccgatgaacctcccaaaatgcactgcagcgccgaaggggagtggctggtgcccatcgggaaatgcatgtgcaaggcaggatatgaagagaaaaatggcacctgtcaagtgtgcagacctgggttcttcaaagcctcacctcacatccagagctgcggcaaatgtccacctcacagttatacccatgaggaagcttcaacctcttgtgtctgtgaaaaggattatttcaggagagagtctgatccacccacaatggcatgcacaagacccccctctgctcctcggaatgccatctcaaatgttaatgaaactagtgtctttctggaatggattccgcctgctgacactggtggaaggaaagacgtgtcatattatattgcatgcaagaagtgcaactcccatgcaggtgtgtgtgaggagtgtggcggtcatgtcaggtaccttccccggcaaagcggcctgaaaaacacctctgtcatgatggtggatctactcgctcacacaaactatacctttgagattgaggcagtgaatggagtgtccgacttgagcccaggagcccggcagtatgtgtctgtaaatgtaaccacaaatcaagcagctccatctccagtcaccaatgtgaaaaaagggaaaattgcaaaaaacagcatctctttgtcttggcaagaaccagatcgtcccaatggaatcatcctagagtatgaaatcaagtattttgaaaaggaccaagagaccagctacacgattatcaaatctaaagagacaactattactgcagagggcttgaaaccagcttcagtttatgtcttccaaattcgagcacgtacagcagcaggctatggtgtcttcagtcgaagatttgagtttgaaaccaccccagtgtttgcagcatccagcgatcaaagccagattcctgtaattgctgtgtctgtgacagtgggagtcattttgttggcagtggttatcggcgtcctcctcagtggaaggcggtgtggctacagcaaagcaaaacaagatccagaagaggaaaagatgcattttcataatgggcacattaaactgccaggagtaagaacttacattgatccacatacctatgaggatcccaatcaagctgtccacgaatttgctaaggagatagaagcatcatgtatcaccattgagagagttattggagcaggtgaatttggtgaagtttgtagtggacgtttgaaactaccaggaaaaagagaattacctgtggctatcaaaacccttaaagtaggctatactgaaaagcaacgcagagatttcctaggtgaagcaagtatcatgggacagtttgatcatcctaacatcatccatttagaaggtgtggtgaccaaaagtaaaccagtgatgatcgtgacagagtatatggagaatggctctttagatacatttttgaagaaaaacgatgggcagttcactgtgattcagcttgttggcatgctgagaggtatctctgcaggaatgaagtacctttctgacatgggctatgtgcatagagatcttgctgccagaaacatcttaatcaacagtaaccttgtgtgcaaagtgtctgactttggactttcccgggtactggaagatgatcccgaggcagcctacaccacaaggggaggaaaaattccaatcagatggactgccccagaagcaatagctttccgaaagtttacttctgccagtgatgtctggagttatggaatagtaatgtgggaagttgtgtcttatggagagagaccctactgggagatgaccaatcaagatgtgattaaagcggtagaggaaggctatcgtctgccaagccccatggattgtcctgctgctctctatcagttaatgctggattgctggcagaaagagcgaaatagcaggcccaagtttgatgaaatagtcaacatgttggacaagctgatacgtaacccaagtagtctgaagacgctggttaatgcatcctgcagagtatctaatttattggcagaacatagcccactaggatctggggcctacagatcagtaggtgaatggctagaggcaatcaagatgggccggtatacagagattttcatggaaaatggatacagttcaatggacgctgtggctcaggtgaccttggaggatttgagacggcttggagtgactcttgtcggtcaccagaagaagatcatgaacagccttcaagaaatgaaggtgcagctggtaaacggaatggtgccattgtaa

SEQ ID NO: 75 - protein, 160 aa, Homo sapiens
MTLEELVACDNAAQKMQTVTAAVEELLVAAQRQDRLTVGVYESAKLMNVDPDSVVLCLLAIDEEEEDDIALQIHFTLIQSFCCDNDINIVRVSGMQRLAQLLGEPAETQGTTEARDLHCLLVTNPHTDAWKSHGLVEVASYCEESRGNNQWVPYISLQER

SEQ ID NO: 76 - DNA, 483 bp, Homo sapiens
atgacgctggaagagctcgtggcgtgcgacaacgcggcgcagaagatgcagacggtgaccgccgcggtggaggagcttttggtggccgctcagcgccaggatcgcctcacagtgggggtgtacgagtcggccaagttgatgaatgtggacccagacagcgtggtcctctgcctcttggccattgacgaggaggaggaggatgacatcgccctgcaaatccacttcacgctcatccagtccttctgctgtgacaacgacatcaacatcgtgcgggtgtcgggcatgcagcgcctggcgcagctcctgggagagccggccgagacccagggcaccaccgaggcccgagacctgcattgtctcctggtcacgaaccctcacacggacgcctggaagagccacggcttggtggaggtggccagctactgcgaagaaagccggggcaacaaccagtgggtcccctacatctctcttcaggaacgctga

SEQ ID NO: 77 - protein, 463 aa, Homo sapiens
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 78 - DNA, 1392 bp, Homo sapiens
atggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggaaagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttatctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttgccattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatattatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagctga

SEQ ID NO: 79 - protein, 464 aa, Homo sapiens
MAHVRHFRTLVSGFYFWEAALLLSLVATKETDSARSRSAPMSPSDFLDKLMGRTSGYDARIRPNFKGPPVNVTCNIFINSFGSIAETTMDYRVNIFLRQKWNDPRLAYSEYPDDSLDLDPSMLDSIWKPDLFFANEKGANFHEVTTDNKLLRIFKNGNVLYSIRLTLTLSCPMDLKNFPMDVQTCIMQLESFGYTMNDLIFEWQDEAPVQVAEGLTLPQFLLKEEKDLRYCTKHYNTGKFTCIEVRFHLERQMGYYLIQMYIPSLLIVILSWVSFWINMDAAPARVALGITTVLTMTTQSSGSRASLPKVSYVKAIDIWMAVCLLFVFSALLEYAAVNFVSRQHKELLRFRRKRKNKTEAFALEKFYRFSDMDDEVRESRFSFTAYGMGPCLQAKDGMTPKGPNHPVQVMPKSPDEMRKVFIDRAKKIDTISRACFPLAFLIFNIFYWVIYKILRHEDIHQQQD

SEQ ID NO: 80 - DNA, 1395 bp, Homo sapiens
atggcccacgtgagacactttcggacattagtttcgggattttacttctgggaagcagcactgttactcagtttggttgccacaaaggaaacagacagtgcaagatctcgaagtgctccaatgtcaccttctgattttctggataaattaatgggcaggacatcaggatatgatgcaagaatcagacccaattttaaaggccctccagttaatgtcacatgcaacatattcatcaacagctttggctctatcgcagagacgaccatggattacagagtgaatatctttcttcgtcagaaatggaatgatccccgcctcgcgtacagtgaatatcctgacgactctttagacctcgacccctccatgttggactccatttggaaacctgatttgttctttgccaatgaaaagggtgccaactttcatgaagtcactacagacaacaaattgctaagaattttcaaaaatggaaatgttctttattcaataagattaacattaacactttcctgtccaatggatctcaagaattttcccatggatgtacaaacatgtataatgcaactggaaagctttgggtacacaatgaatgatctcatttttgaatggcaagatgaggcacccgtacaagtggcagaaggactcactttgccccagtttctgttgaaagaagaaaaagatttacgatactgcactaaacattacaatacaggaaagtttacgtgtatagaagtgcgattccatctggagcgacaaatgggatactatctgatccagatgtacattcccagtctcctgattgttattctatcctgggtttcattctggatcaacatggatgcagcaccggccagggtagctctggggataaccactgtgctaacgatgactacacagagttcaggatcacgagcttccttgccaaaagtttcatatgtcaaagctattgatatttggatggcagtatgcctcctttttgtgttttcagcacttctggagtatgcagctgtaaattttgtatcaagacaacacaaagaacttctgagatttcgacgaaagagaaagaataagacagaagcttttgcactggagaagttttaccgtttctcagatatggatgatgaggtaagggaaagccgattcagcttcacagcctatggaatgggaccatgtctacaagcaaaggatggcatgactccaaagggccccaaccaccctgtccaggtaatgccaaaaagtcctgatgaaatgaggaaggtctttatcgaccgggccaagaagattgataccatctcccgagcctgcttcccattagcttttttgatttttaatattttctactgggttatctataaaattcttaggcatgaggatattcatcagcagcaagattaa

SEQ ID NO: 81 - DNA, 1350 bp, Homo sapiens
atggcccacgtgagacactttcggacattagtttcgggattttacttctgggaagcagcactgttactcagtttggttgccacaaaggaaacagacagtgcaagatctcgaagtgctccaatgtcaccttctgattttctggataaattaatgggcaggacatcaggatatgatgcaagaatcagacccaattttaaaggccctccagttaatgtcacatgcaacatattcatcaacagctttggctctatcgcagagacgaccatggattacagagtgaatatctttcttcgtcagaaatggaatgatccccgcctcgcgtacagtgaatatcctgacgactctttagacctcgacccctccatgttggactccatttggaaacctgatttgttctttgccaatgaaaagggtgccaactttcatgaagtcactacagacaacaaattgctaagaattttcaaaaatggaaatgttctttattcaataagattaacattaacactttcctgtccaatggatctcaagaattttcccatggatgtacaaacatgtataatgcaactggaaagctttgggtacacaatgaatgatctcatttttgaatggcaagatgaggcacccgtacaagtggcagaaggactcactttgccccagtttctgttgaaagaagaaaaagatttacgatactgcactaaacattacaatacaggaaagtttacgtgtatagaagtgcgattccatctggagcgacaaatgggatactatctgatccagatgtacattcccagtctcctgattgttattctatcctgggtttcattctggatcaacatggatgcagcaccggccagggtagctctggggataaccactgtgctaacgatgactacacagagttcaggatcacgagcttccttgccaaaagtttcatatgtcaaagctattgatatttggatggcagtatgcctcctttttgtgttttcagcacttctggagtatgcagctgtaaattttgtatcaagacaacacaaagaacttctgagatttcgacgaaagagaaagaataaggatgatgaggtaagggaaagccgattcagcttcacagcctatggaatgggaccatgtctacaagcaaaggatggcatgactccaaagggccccaaccaccctgtccaggtaatgccaaaaagtcctgatgaaatgaggaaggtctttatcgaccgggccaagaagattgataccatctcccgagcctgcttcccattagcttttttgatttttaatattttctactgggttatctataaaattcttaggcatgaggatattcatcagcagcaagattaa

SEQ ID NO: 82 - protein, 359 aa, Homo sapiens
MAEPFLSEYQHQPQTSNCTGAAAVQEELNPERPPGAEERVPEEDSRWQSRAFPQLGGRPGPEGEGSLESQPPPLQTQACPESSCLREGEKGQNGDDSSAGGDFPPPAEVEPTPEAELLAQPCHDSEASKLGAPAAGGEEEWGQQQRQLGKKKHRRRPSKKKRHWKPYYKLTWEEKKKFDEKQSLRASRIRAEMFAKGQPVAPYNTTQFLMDDHDQEEPDLKTGLYSKRAAAKSDDTSDDDFMEEGGEEDGGSDGMGGDGSEFLQRDFSETYERYHTESLQNMSKQELIKEYLELEKCLSRMEDENNRLRLESKRLGGDDARVRELELELDRLRAENLQLLTENELHRQQERAPLSKFGD

SEQ ID NO: 83 - DNA, 1080 bp, Homo sapiens
atggccgagccattcttgtcagaatatcaacaccagcctcaaactagcaactgtacaggtgctgctgctgtccaggaagagctgaaccctgagcgccccccaggcgcggaggagcgggtgcccgaggaggacagtaggtggcaatcgagagcgttcccccagttgggtggccgtccggggccggagggggaagggagcctggaatcccaaccacctcccttgcagacccaggcctgtccagaatctagctgcctgagagagggcgagaagggccagaatggggacgactcgtccgctggcggcgacttcccgccgccggcagaagtggaaccgacgcccgaggccgagctgctcgcccagccttgtcatgactccgaggccagtaagttgggggctcctgccgcagggggcgaagaggagtggggacagcagcagagacagctggggaagaaaaaacataggagacgcccgtccaagaagaagcggcattggaaaccgtactacaagctgacctgggaagagaagaaaaagttcgacgagaaacagagccttcgagcttcaaggatccgagccgagatgttcgccaagggccagccggtcgcgccctataacaccacgcagttcctcatggatgatcacgaccaggaggagccggatctcaaaaccggcctgtactccaagcgggccgccgccaaatccgacgacaccagcgatgacgacttcatggaagaagggggtgaggaggatgggggcagcgatgggatgggaggggacggcagcgagtttctgcagcgggacttctcggagacgtacgagcggtaccacacggagagcctgcagaacatgagcaagcaggagctcatcaaggagtacctggaactggagaagtgcctctcgcgcatggaggacgagaacaaccggctgcggctggagagcaagcggctgggtggcgacgacgcgcgtgtgcgggagctggagctggagctggaccggctgcgcgccgagaacctccagctgctgaccgagaacgaactgcaccggcagcaggagcgagcgccgctttccaagtttggagactag

SEQ ID NO: 84 - protein, 136 aa, Homo sapiens
MARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGERA

SEQ ID NO: 85 - DNA, 411 bp, Homo sapiens
atggcccggacgaagcagacagctcgcaagtctaccggcggcaaggcaccgcggaagcagctggccaccaaggcagcgcgcaaaagcgctccagcgactggcggtgtgaagaagccccaccgctacaggccaggcaccgtggccttgcgtgagatccgccgttatcagaagtcgactgagctgctcatccgcaaactgccatttcagcgcctggtgcgagaaatcgcgcaggatttcaaaaccgaccttcgtttccagagctcggcggtgatggcgctgcaagaggcgtgcgaggcctatctggtgggtctctttgaagacaccaacctctgtgctattcacgccaagcgtgtcactattatgcctaaggacatccagcttgcgcgtcgtatccgtggcgagcgagcataa

SEQ ID NO: 86 - protein, 180 aa, Homo sapiens
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 87 - DNA, 543 bp, Homo sapiens
atggactggcctcacaacctgctgtttcttcttaccatttccatcttcctggggctgggccagcccaggagccccaaaagcaagaggaaggggcaagggcggcctgggcccctggcccctggccctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctga

SEQ ID NO: 88 - protein, 180 aa, Homo sapiens; identical to SEQ ID NO: 86

SEQ ID NO: 89 - DNA, 2469 bp, Homo sapiens
atgtcgaacgcaatgtataataagatgtggcatcagacccaagaagccctcggtgctttactcgataaagagcctcagaagatgattgaaccacaaagaaatcaggttttcatctttcaaacattagccaccttctacgtaaagtatgtgcagatctttagaaacctagagaatgtctacgaccagttcgtccacccccagaaacgaatactgatcaggaaagtcctggacggggtgatgggccgcatcctggagctgaagaacgagatggtggagctggaactcacggagttccattatttcgatgatatcctgcaggatctcaagctggccccccaacaattagatattcccatacccaagtattttttgaaggagaagttggaagtaataaaaggaagagagaaaattcttgctcaaatattagcggacagcggaatagatacatctgacatgaaataccctgtaaagagtatccctttcgatgaggctgttaaattaatccagattgctgagagggcacggcaaggtcgcctaagggctttattcatgaagcaaatctatctgcaagaatatagagcaaagcaatccaagatgcttggcaagaaagtgacagatacctgggctgctgcactccgcattcagaaggtttggcgacgtttccatcaacgtaaggaaactgaaaaactgagagaagaggagatgatcttcctgggtatgaatccacctcctctctttaatgaagtcagtgctacagtaatccaggctgaaaaggtggaccgcctgcggaatgaggtgcagataaagcatgaagaggactacagggaagccctggttaccatcaagaatgacctaaagttgatagaaggcgtggatatcaaggagaaccttcaagaccagatccggcattggttcatcgaatgcagaaatttaaccgggacatttcctgactaccctgacgttgaagaaggagggtcagctattattttttctgacaagaccatacaacaggttattgaggatatcatagcaaaccaagaggaagaagaaaaaaacaaaaagaagaagaagaaaaaggaaaaacaacccaagaaagccaaaaaacaaaagaaaggaacaaaggagaaaaataaggaagaagacgaaaaatggaaaatgtcaccaagtctttttcttcctgcaatgaaggaaggatgtaacgcatacaaagaaatctggatgaaaaaagatgagtcttggaatttctctcaggactatgatccagaactgatcaaagaggagaaacgaaaagaattacagtcagagatcaggatacaggttgatgagttgatgagacaggaacttaaaaacttaaagctagctgtggacagagaaagggagcgcccagtgaaagcaggaaagaaaaaggacaagaaaggaaagaaaggcaaaaagaaagagaagaaggcaaagaaggataaagatctgacagctgacaggaccatcgagtctctgtataaggaactggtggaagaaggattactgatccaggctctgaaagtcaacctctctgattacattggtgagtacagctacctggggactactcttcgccaggtgtccatagaacccatgccctccctcctggatgtcagacagctcatcacattgtacggaatctggccattaggttctgcagcagtgcatgagaaagctcctttggtgaaatcgctactgttagccgggccgtctggggtagggaagaaaatgctggtccatgccatctgcaccgaaacgggagccaacctcttcaacttgtcctcctctaacattgctgggaaataccctggcaaaaatggcctccaaatgatgctgcatgcagtcttcaaggtggctcgacagctccagccctccgtggtgtggattgaagacacagaaaaaaccttctacaaaaaagttcccaacgcagaaaagatgaatgaacctaaacgcctgaaaaaacaccttccccaaatcctgaaactcctgaaaccagatgaccggattctgattgtggggaccacacggcgtcccttcgatgctgaactccaatctttctgcaaagtttaccagaaaattattttggtgcccagaccagactatgcttccagatacgttctgtggaagcaaatcattgaacgcaatggaggagtcctcaccagtgccttgaatgtcagctgcctggcgaaggtcactgacggcttcacccaaggacatatagtcgaagtggttaaaggcgtgctcacagatcagagaatccggcggcaaattcataaacctctcactgcagttgagtttattacggcgataaccagcatgaatccagtgtacaaagaggaagaagagagctttaagaactggtatgccaaaactcctctgggtaaaaaacgtgccttggcgataacaggaggcagcacagaaaaggcaaaggacaaagggaaaaggaaataa

SEQ ID NO: 90 - protein, 830 aa, Homo sapiens
MTPSLSAEENRMYNKMWHQTQEALGALLDKEPQKMIEPQRNQVFIFQTLATFYVKYVQIFRNLENVYDQFVHPQKRILIRKVLDGVMGRILELKNEMVELELTEFHYFDDILQDLKLAPQQLDIPIPKYFLKEKLEVIKGREKILAQILADSGIDTSDMKYPVKSIPFDEAVKLIQIAERARQGRLRALFMKQIYLQEYRAKQSKMLGKKVTDTWAAALRIQKVWRRFHQRKETEKLREEEMIFLGMNPPPLFNEVSATVIQAEKVDRLRNEVQIKHEEDYREALVTIKNDLKLIEGVDIKENLQDQIRHWFIECRNLTGTFPDYPDVEEGGSAIIFSDKTIQQVIEDIIANQEEEEKNKKKKKKKEKQPKKAKKQKKGTKEKNKEEDEKWKMSPSLFLPAMKEGCNAYKEIWMKKDESWNFSQDYDPELIKEEKRKELQSEIRIQVDELMRQELKNLKLAVDRERERPVKAGKKKDKKKGKKGKKKEKKAKKDKDLTADRTIESLYKELVEEGLLIQALKVNLSDYIGEYSYLGTTLRQVSIEPMPSLLDVRQLITLYGIWPLGSAAVHEKAPLVKSLLLAGPSGVGKKMLVHAICTETGANLFNLSSSNIAGKYPGKNGLQMMLHAVFKVARQLQPSVVWIEDTEKTFYKKVPNAEKMNEPKRLKKHLPQILKLLKPDDRILIVGTTRRPFDAELQSFCKVYQKIILVPRPDYASRYVLWKQIIERNGGVLTSALNVSCLAKVTDGFTQGHIVEVVKGVLTDQRIRRQIHKPLTAVEFITAITSMNPVYKEEEESFKNWYAKTPLGKKRALAITGGSTEKAKDKGKRK

SEQ ID NO: 91 - DNA, 1221 bp, Homo sapiens
atggctgatggcaacgaggatctgcgggctgacgacttgcctgggccagccttcgagagctatgagtccatggagcttgcctgccccgctgagcgcagcggccacgtagccgtcagcgacgggcgccacatgttcgtctggggcggctacaagagtaatcaagtcagaggattatatgacttttatctgcctagagaagaactatggatctacaacatggagactggaagatggaaaaaaatcaacactgaaggtgatgttcctccttctatgtcaggaagctgtgctgtgtgtgtagacagggtgctgtacttgtttggaggacaccattcaagaggcaataccaataagttctacatgctggattcaaggtctacagacagagtgttacagtgggaaagaattgattgccaaggaattcctccatcatcaaaggacaaacttggtgtctgggtatataaaaacaagttaatattttttggagggtatggatatttgcctgaagataaagtattgggaacttttgaattcgatgaaacatctttttggaattcaagtcatccaagaggatggaatgatcatgtacatattttagatactgaaacatttacctggagccagcctataactactggtaaagcaccttcacctcgtgctgcccatgcctgtgcaactgtcggaaatagaggcttcgtgtttggaggcagatatcgagatgctagaatgaatgatcttcactatcttaatctggatacatgggagtggaatgaattaattccacaaggcatatgcccagttggtcgatcttggcactcactaacaccagtttcttcagatcatctttttctctttggaggatttaccactgataaacagccactaagtgatgcctggacttactgcatcagtaaaaatgaatggatacaatttaatcatccatataccgaaaaaccaaggttatggcacacagcttgtgccagcgatgaaggagaagtaattgtttttggtggatgtgccaacaacttgcttgtccatcacagagctgcacacagtaatgaaatactaatattttcagttcaaccaaaatctcttgtacggctaagcttagaagcagtcatttgctttaaagaaatgttagccaactcatggaactgccttccaaaacacttacttcacagtgttaatcagaggtttggtagtaacaacacttctggatcttaa

SEQ ID NO: 92 - protein, 293 aa, Homo sapiens
MPLLQNLLRAQRLTVDWTWRNILVAAATGAHSQDQLVQSGAEVKKPLSSVKVSFKASGYTFTNNFMHWVKVGRFGLFKQKAQTPAGSSSHGKYFLRLMDLLHKNMKHLWFFLLLVAAPRWVLSQVQLQESGPGLVKPSETLSLTCVVSGGSISSSNWWSWVRQPPGKGLEWIGEIYHSGSPNYNPSLKSRVTISVDKSKNQFSLKLSSVTAADTAVYYCARDTVRGAFPYILVMHLSSVGAQGRLHRNCLKLASADVLRPRVMHTYDSRSCGLYCRTDLCPVGHSHLLTPGLL

SEQ ID NO: 93 - DNA, 1581 bp, Homo sapiens
atgccggcggtcagtggtccaggtcccttattctgccttctcctcctgctcctggacccccacagccctgagacggggtgtcctcctctacgcaggtttgagtacaagctcagcttcaaaggcccaaggctggcattgcctggggctggaatacccttctggagccatcatggagacgccatcctgggcctggaggaagtgcggctgacgccatccatgaggaaccggagtggcgccgtgtggagcagggcctctgtccccttctctgcctgggaagtagaggtgcagatgagggtgacgggactggggcgccggggagcccagggcatggccgtgtggtacacccggggcaggggccatgtaggctctgtccttggggggctggcttcgtgggacggcatcgggatcttctttgactctccggcagaggatactcaggacagtcctgccatccgtgtgctggccagcgacgggcacatcccctctgagcagcctggggatggagctagccaagggctgggctcctgtcattgggacttccggaaccggccacaccccttcagagcacggatcacctactgggggcagaggctgcgcatgtccttgaacagtggcctcactcccagtgatccaggtgagttctgtgtggatgtggggcccctgcttttggtccctggaggtttctttggggtctcagcagccaccggcaccctggcagatgatcatgatgtcctgtccttcctgaccttcagcctgagtgagcccagcccagaggttccccctcagcccttcctggagatgcagcagctccgcctggcgaggcagctggaagggctgtgggcaaggctgggcttgggcaccagggaggatgtaactccaaaatcagactctgaagctcaaggagaaggggaaaggctctttgacctggaggagacgctgggcagacaccgccggatcctgcaggctctgcggggtctctccaagcagctggcccaggctgagagacaatggaagaagcagctggggcccccaggccaagccaggcctgacggaggctgggccctggatgcttcctgccagattccatccaccccagggaggggtggccacctctccatgtcactcaataaggactctgccaaggtcggtgccctgctccatggacagtggactctgctccaggccctgcaagagatgagggatgcagctgtccgcatggctgcagaagcccaggtctcctacctgcctgtgggcattgagcatcatttcttagagctggaccacatcctgggcctcctgcaggaggagcttcggggcccggcgaaggcagcagccaaggccccccgcccacctggccagcccccaagggcctcctcgtgcctgcagcctggcatcttcctgttctacctcctcattcagactgtaggcttcttcggctacgtgcacttcaggcaggagctgaacaagagccttcaggagtgtctgtccacaggcagccttcctctgggtcctgcaccacacacccccagggccctggggattctgaggaggcagcctctccctgccagcatgcctgcctga

SEQ ID NO: 94 - protein, 65 aa, Homo sapiens
MKKDSQGAFSGFCHGGDALHEGRVRRPLWLKDMYKPLSATRVNNHAWKLHKKSSNEDEILNRDPG

SEQ ID NO: 95 - DNA, 933 bp, Homo sapiens
atgcagaacaatccctctgccagtggtcaccagggccacagccctgtggaagctgagtggaacccgaatctccttagagctcagtgcctcaccgtggactggacctggaggaacatcttggtggcagcagctacaggtgcgcactctcaggaccagttggtgcagtctggggctgaggtgaagaagcctctgtcctcagtgaaggtctccttcaaggcttctggatacaccttcaccaacaactttatgcactgggtgaaggtaggtcgttttggtctgttcaaacaaaaggcccagacaccagctgggtccagctcacatgggaaatactttctgagactcatggacctcctgcacaagaacatgaaacacctgtggttctttctcctcctggtggcagctcccagatgggtcctgtcccaggtgcagctgcaggagtcgggcccaggactggtgaagccttcggagaccctgtccctcacctgcgttgtctctggtggctccatcagcagtagtaactggtggagctgggtccgccagcccccagggaaggggctggagtggattggggaaatctatcatagtgggagccccaactacaacccgtccctcaagagtcgagtcaccatatcagtagacaagtccaagaaccagttctccctgaagctgagctctgtgaccgccgcggacagggccgtgtattactgtgcgagagacacagtgaggggagcattcccttacatccttgtcatgcatctgtcctctgtgggcgcccagggcaggttacacaggaattgcctgaagctggcctcagctgatgtgctgagaccacgggtcatgcacacgtatgattccaggtcatgcgggctctactgcaggacagacctgtgtcctgtggggcacagccacctgctgatcccgggcctcctctaa

SEQ ID NO: 96 - protein, 279 aa, Homo sapiens [sequence omitted]

SEQ ID NO: 97 - DNA, 5233 bp, Homo sapiens
agaatgacttccgctcagtgcccggcactagcgtgtgtcatgtccccgctgcgtttctggggcccatggcccctccttatgtggcaactattgtggctactagtcaaggaggctcagcctctggagtgggtcaaggacccgctccagctgacctctaaccccctggggccgcctgagccctggtcttcccactcctcccatttcccacgggaatctccccatgcgcctactctcccagcagacccgtgggactttgatcacctggggccctctgcttcctcagagatgccagccccaccccaggaatcgactgaaaatttggttccattcctggacacctgggattcagctggagagctgcccctggagccagagcagttcttggcttcacagcaggatttaaaggacaagctgagtccacaggaaaggctccctgtttcgcccaagaagctgaagaaagatccagctcagcgttggagccttgctgagattattggaattatacgccaattatccacacctcagagtcagaaacagactttgcagaatgaatattccagtacagatacaccgtatcccggtagcctgcctccagaactccgggtgaagtcagatgagcctccagggccctctgagcaagttggaccttctcaattccatctagagcccgaaactcaaaatccagagacccttgaagacatccagtcctcttcactccagcaagaagccccagcacagcttccacagctccttgaggaagaaccttcttcaatgcagcaggaggccccagctctgcctccagagtcctctatggagagtctaactctaccgaatcatgaggtgtcagttcaacctccaggtgaggatcaagcttattatcacttgcccaacattacagttaaacctgcagatgtggaggttaccataacttcagagcctaccaatgagacagaatcttcccaagcccagcaggagaccccaattcagtttccagaggaggtggaaccttctgcaacccaacaggaggccccaattgagcctccagttcctcctatggagcatgaactttccatcagtgagcagcagcagccagttcagccttctgagtctcctagggaggtcgaatcttctccgacccagcaggagaccccaggtcagcctccagaacatcatgaagtcacagtttcacctccaggtcaccatcaaactcatcatttagcttcacccagtgtctctgtgaagcctccagacgtgcagctcaccatagcagcagagcctagtgcagaggtgggaacttctctagtccaccaggaggctacaactcggctctcagggtcaggtaatgatgtagaacctcccgccatccagcacgggggcccacctctgcttccagagtcatcagaagaagctggacctttagcagttcaacaggagacttcatttcaatctccggaacctattaataatgagaacccctctccaacccagcaggaggctgcagctgagcatccacagaccgctgaggagggtgagtcttccctaacccatcaggaggccccagctcagactccagagttccctaatgtagttgtagctcaacctccagagcattcacacctgactcaagccacagttcaacctttggatctggggtttaccatcactccagaatccaagacagaggttgaactttctccaaccatgaaggagaccccaactcagcctcctaagaaagttgtaccccaacttcgagtatatcaaggggtaacaaatccaacaccaggtcaggatcaagctcagcatccagtgtcacccagcgttacagttcaacttttggacctgggacttaccatcactccagaacctactacggaggttggacattctacacccccgaagaggactatagtttctccaaagcatcctgaggtgacacttccacatccagaccaggttcagactcagcattcacacctgactcgagccacagttcaacctttggacctggggtttaccatcactccaaaatccatgacagaggttgaaccttctacagccctgatgactacagctcctcctccaggacaccctgaggtgacacttccaccttcagacaagggtcaggctcagcattcacacctgactcaagccaccgttcaacctctggacctggagcttaccataactacaaaacctactacagaggttaaaccatctccaaccacggaggagacctcaactcagcctccagacctgggacttgccatcattccagaacccactacagagactggacattctacagccctggagaagactacagctcctcgtccagaccgggttcagactctgcatcgaagcctgactgaagtcacaggtccacctactgaactagaacctgctcaggattcactggtgcagtctgaaagttacacccaaaataaggctttaactgcaccagaggaacacaaggcctccacaagcaccaacatatgtgagctctgtacctgcggagatgagatgttgtcatgtattgatctcaacccagagcagaggctccgccaagtgcctgtgccagagcccaacacccacaatggcaccttcaccatcttaaatttccaaggaaactatatttcttacattgatggaaatgtatggaaagcatacagttggaccgagaaactaattctcagagaaaataacttgactgaattacacaaggattcatttgaaggcctgctatccctccagtatttagatttatcctgcaataaaatacagtctattgaaagacatacatttgaaccactaccatttttgaagtttataaatcttagttgcaatgtaattacagaactcagctttggaacatttcaggcctggcacggaatgcagtttttacataagttaattctcaatcacaatcctctgacaactgttgaagatccgtatctctttaaattgccagcattaaaatatctagacatgggaacaacgctagtcccacttacaacacttaagaacattctcatgatgactgttgaactggaaaaactgatcttacctagccatatggcctgctgcctctgccaatttaaaaacagcattgaggctgtctgcaagacagtcaagctgcattgcaacagtgcatgtctgacaaacaccacacattgtcctgaagaagcatcggtagggaatccagaaggagcgttcatgaaggtgttacaagcccggaagaactacacaagcactgagctgattgttgagccagaggagccctcagacagcagtggcatcaacttgtcaggctttgggagtgagcagctagacaccaatgacgagagtgattttatcagtacactacgttacatcttgccttatttctcagcggtaaacctagatgtgaaatcactgttactaccgttaattaaactgccaaccacaggaaacagcctggcaaagattcaaactgtaggccaaaaccggcagagagtgaagagagtcctcatgggcccaaggagcatccagaaaaggcacttcaaagaggtaggaaggcagagcatcaggagggaacagggtgcccaggcatctgtggagaacgctgccgaagaaaaaaggctcgggagtccagccccaagggaggtggaacagccccacacacagcaggggcctgagaagttagcgggaaacgccgtctacaccaagccttccttcacccaagagcataaggcagcagtctctgtgctgaaacccttctccaagggcgcgccttctacctccagccctgcaaaagccctaccacaggtgagagacagatggaaagacttaacccacgctatttccactttagaaagtgcaaaggctagagttacaaatacgaagacgtctaaaccaatcgtacatgccagaaaaaaataccgctttcacaaaactcgctcccacgtgacccacagaacacccaaagtcaaaaagagtccaaaggtcagaaagaaaagttatctgagtagactgatgctcgcaaacaggcttccattctctgcagcgaagagcctcataaattccccttcacaaggggctttttcatccttaggagacctgagtcctcaagaaaacccttttctggaagtatctgctccttcagaacattttatagaaaagaataatacaaaacacacaactgcaagaaatgcctttgaagaaaatgattttatggaaaacactaacatgccagaaggaaccatctctgaaaacacaaactacaatcatcctcctgaggcagattccgctgggactgcattcaacttagggccaactgttaaacaaactgagacaaaatgggaatacaacaacgtgggcactgacctgtcccccgagcccaaaagcttcaattacccattgctctcgtccccaggtgatcagtttgaaattcagctaacccagcagctacagtcccttatccccaacaacaatgtgagaaggctcattgctcatgttatccggaccttgaagatggactgctctgggacccatgtgcaagtgacctgtgccaagctcatctccaggacaggccacctgatgaagcttctcagtgggcagcaggaagtaaaggcatccaagatagaatgggatacggaccaatggaagattgagaactacattaatgagagcacagaagcccagagtgaacagaaagagaagtcgcttgagctcaaaaaagaagttccaggatatggctatactgacaaactcatcttggcattaattgttactggaatactaacgattttgattatacttttctgcctcattgtgatatgttgtcaccgaaggtcattacaagaagatgaagaaggattctcaaggggcattttcagatttctgccacggaggggatgctcttcgcgaagggagagtcaggatggactttcctcatttggacagccgctctggtttaaagatatgtacaaacctctcagtgccacaagaataaataatcatgcatggaagctgcacaagaagtcatctaatgaggacaagatcctcaacagggaccctggggacagcgaagccccaacggaggaggaggagagtgaagccctgccataggaggagaacacagcccacctcaggcctcctgcaaaaatacatagaataaacaacaacagttactaaataaatgaaaattgtgattccgatgaagcctgccagagaaataaagcattttttaaaagag

SEQ ID NO: 98 - protein, 109 aa, Homo sapiens
MQHPPCEPGNCLSLKEKKITEGSGGVCWGGETDASNPAPALTACCAAEREANVEQGLAGRLLLCNYERRVVRRCKIAGRGRAPLGTRPLDVSSFKLKEEGRPPCLKINK

SEQ ID NO: 99 - DNA, 840 bp, Homo sapiens
atggcggcgcccgtaaggcggacgctgttaggggtggcggggggttggcggcggttcgagaggctctgggccggcagtctaagctctcgcagcctggctcttgcagccgcaccctcaagcaacggatccccatggcgcttgttgggcgcgttgtgcctgcagcggccacctgtagtctccaagccgttgaccccattgcaggaagagatggcgtctctactgcagcagattgagatagagagaagcctgtattcagaccacgagcttcgtgctctggatgaaaaccagcgactggcaaagaagaaagctgaccttcatgatgaagaagatgaacaggatatattgctggcgcaagatttggaagatatgtgggagcagaaatttctacagttcaaacttggagctcgcataacagaagctgatgaaaagaatgaccgaacatccctgaacaggaagctagacaggaaccttgtcctgttagtcagagagaagtttggagaccaggatgtttggatactgccccaggcagagtggcagcctggggagacccttcgaggaacagctgaacgaaccctggccacactctcagaaaacaacatggaagccaagttcctaggaaatgcaccctgtgggcactacacattcaagttcccccaggcaatgcggacagagagtaacctcggagccaaggtgttcttcttcaaagcactgctattaactggagacttttcccaggctgggaataagggccatcatgtgtgggtcactaaggatgagctgggtgactatttgaaaccaaaatacctggcccaagttaggaggtttgtttcagacctctga

SEQ ID NO: 100 - protein, 327 aa, Homo sapiens
MALVHKLLRGTYFLRKFSKPTSALYPFLGIRFAEYSSSLQKPVASPGKASSQRKTEGDLQGDHQKEVALDITSSEEKPDVSFDKAIRDEAIYHFRLLKDEIVDHWRGPEGHPLHEVLLEQAKVVWQFRGKEDLDKWTVTSDKTIGGRSEVFLKMGKNNQSALLYGTLSSEAPQDGESTRSGYCAMISRIPRGAFERKMSYDWSQFNTLYLRVRGDGRPWMVNIKEDTDFFQRTNQMYSYFMFTRGGPYWQEVKIPFSKFFFSNRGRIRDVQHELPLDKISSIGFTLADKVDGPFFLEIDFIGVFTDPAHTEEFAYENSPELNPRLFK

SEQ ID NO: 101 - DNA, 770 bp, Homo sapiens
agggggtggtggcgaggctccgcaactttggaaactgccatttcattcacacaaggcactgcctgggggagggggctgttcctggctgcagaattctagctctcacgagcacgcagacaaccgcactcgcagcggtgtggggccggctgctcaggggaagccccaggctctccgacccagctaccgggaatggggcaccctttggagaagaaccccagcctggggtggggacgcaccggctctccgacagctcaaacacagacagatcttctagagccgagggaatttcttttcgcagaagccattactccccccgagagaaggctgcaaagctgggaagcccagggtgtgctcctcccgcccttttggacccccgggcttgcaccggctgcactctgagaaccagctgcgcgcggagcggtgcaatgcagcacccaccctgcgagcctggcaattgcttgtcattaaaagaaaaaaaaattacggagggctccgggggtgtgtgttggggaggggagaccgatgcttctaacccagcccccgctttgactgcgtgttgtgcagctgagcgcgaggccaacgttgagcaaggccttgcagggaggttgctcctgtgtaattacgaaagaagggtagtccgaaggtgcaaaatagcagggagaggacgcgcccccttaggaacaagacctctggatgtttccagtttcaaattgaaagaagaggggcgccccccttgtttgaaaataaataaataaataagtgcgagctac

SEQ ID NO: 102 - DNA, 984 bp, Homo sapiens
atggctttggttcacaaattgctgcgtggtacttattttctcagaaaattctctaagccaacttctgccttgtatccatttttgggtattcgctttgcagagtattccagtagtcttcagaaaccagtggcttctcctggcaaagcctcctcacagaggaagactgaaggggatttgcaaggagatcaccagaaagaagttgctttggatataacttcttctgaggagaagcctgatgttagtttcgataaagcaattagagatgaagcaatataccattttaggcttttgaaggatgaaattgtggatcattggagaggaccggaaggccaccctctgcatgaggtcttgctggaacaagccaaggttgtctggcaattccgggggaaagaagatttggataagtggacagtgacttctgataagacgattggaggcagaagtgaagtgtttttgaaaatgggcaagaataaccaaagtgcactgctatatggaactctgagctctgaggcgcctcaggacggggagtctacccgaagtgggtactgtgcaatgatatccaggattccaaggggtgcttttgagaggaagatgtcttacgattggtcccagttcaatactctgtatctccgtgtacgtggggatggtcggccttggatggtgaatatcaaggaggacacagatttcttccagaggacgaatcagatgtatagttacttcatgttcacccgcgggggaccctactggcaggaggtcaagattcctttttccaaatttttcttctctaatcgaggaagaatccgggatgttcagcatgagcttccgcttgataagatctcttctataggattcaccttggctgataaagtggatggtccattcttcctggagatagattttattggcgtgtttactgatccagctcatacagaagaatttgcctatgaaaattctccagagcttaacccaaggctttttaaataa

SEQ ID NO: 103 - DNA, 5175 bp, Homo sapiens
atggtgatgggaatctttgcaaattgtatcttctgtttgaaagtgaagtacttacctcagcagcagaagaaaaagctacaaactgacattaaggaaaatggcggaaagttttccttttcgttaaatcctcagtgcacacatataatcttagataatgctgatgttctgagtcagtaccaactgaattctatccaaaagaaccacgttcatattgcaaacccagattttatatggaaatctatcagggaaaagagactcttggatgtaaagaattatgatccttataagcccctggacatcacaccacctcctgatcagaaggcgagcagttctgaagtgaaaacagaaggtctatgcccggacagtgccacagaggaggaagacactgtggaactcactgagtttggtatgcagaatgttgaaattcctcatcttcctcaagattttgaagttgcaaaatataacaccttggagaaagtgggaatggagggaggccaggaagctgtggtggtggagcttcagtgttcgcgggactccagggactgtcctttcctgatatcctcacacttcctcctggatgatggcatggagactagaagacagtttgctataaagaaaacctctgaagatgcaagtgaatactttgaaaattacattgaagaactgaagaaacaaggatttctactaagagaacatttcacacctgaagcaacccaattagcatctgaacaattgcaagcattgcttttggaggaagtcatgaattcaagcactctgagccaagaggtgagcgatttagtagagatgatttgggcagaggccctgggccacctggaacacatgcttctcaagccagtgaacaggattagcctcaacgatgtgagcaaggcagaggggattctccttctagtaaaggcagcactgaaaaatggagaaacagcagagcaattgcaaaagatgatgacagagttttacagactgatacctcacaaaggcacaatgcccaaagaagtgaacctgggactattggctaagaaagcagacctctgccagctaataagagacatggttaatgtctgtgaaactaatttgtccaaacccaacccaccatccctggccaaataccgagctttgaggtgcaaaattgagcatgttgaacagaatactgaagaatttctcagggttagaaaagaggttttgcagaatcatcacagtaagagcccagtggatgtcttgcagatatttagagttggcagagtgaatgaaaccacagagtttttgagcaaacttggtaatgtgaggcccttgttgcatggttctcctgtacaaaacatcgtgggaatcttgtgtcgagggttgcttttacccaaagtagtggaagatcgtggtgtgcaaagaacagacgtcggaaaccttggaagtgggatttatttcagtgattcgctcagtacaagtatcaagtactcacacccgggagagacagatggcaccagactcctgctcatttgtgacgtagccctcggaaagtgtatggacttacatgagaaggacttttccttaactgaagcaccaccaggctacgacagtgtgcatggagtttcgcaaacagcctctgtcaccacagactttgaggatgatgaatttgttgtctataaaaccaatcaggttaaaatgaaatatattattaaattttccatgcctggagatcagataaaggactttcatcctagtgatcatactgaattagaggaatacagacctgagttttcaaatttttcaaaggttgaagattaccagttaccagatgccaaaacttccagcagcaccaaggccggcctccaggatgcctctgggaacttggttcctctggaggatgtccacatcaaagggagaatcatagacactgtagcccaggtcattgtttttcagacatacacaaataaaagtcacgtgcccattgaggcaaaatatatctttcctttggatgacaaggccgctgtgtgtggcttcgaagccttcatcaatgggaagcacatagttggagagattaaagagaaggaagaagcccagcaagagtacctagaagccgtgacccagggccatggcgcttacctgatgagtcaggatgctccggacgtttttactgtaagtgttggaaacttaccccctaaggctaaggttcttataaaaattacctacatcacagaactcagcatcctgggcactgttggtgtctttttcatgcccgccaccgtagcaccctggcaacaggacaaggctttgaatgaaaaccttcaggatacagtagagaagatttgtataaaagaaataggaacaaagcaaagcttctctttgactatgtctattgagatgccgtatgtgattgaattcattttcagtgatacacatgaactgaaacaaaagcgcacagactgcaaagctgtcattagcaccatggaaggcagctccttagacagcagtggattttctctccacatcggtttgtctgctgcctatctcccaagaatgtgggttgaaaaacatccagaaaaagaaagcgaggcttgcatgcttgtctttcaacccgatctcgatgtcgacctccctgacctagccagtgagagcgaagtgattatttgtcttgactgctccagttccatggagggtgtgacattcttgcaagccaagcaaatcgccttgcatgcgctgtccttggtgggtgagaagcagaaagtaaatattatccagttcggcacaggttacaaggagctattttcgtatcctaagcatatcacaagcaataccatggcagcagagttcatcatgtctgccacacctaccatggggaacacagacttctggaaaacactccgatatcttagcttattgtaccctgctcgagggtcacggaacatcctcctggtgtctgatgggcacctccaggatgagagcctgacattacagctcgtgaagaggagccgcccgcacaccaggttattcgcctgcggtatcggttctacagcaaatcgtcacgtcttaaggattttgtcccagtgtggtgccggagtatttgaatattttaatgcaaaatccaagcatagttggagaaaacagatagaagaccaaatgaccaggctatgttctccgagttgccactctgtctccgtcaaatggcagcaactcaatccagatgtgcccgaggccctgcaggccccagcccaggtgccgtccttgtttctcaatgatcgactccttgtctatggattcattcctcactgcacacaggcaactctgtgtgcactaattcaagagaaagaatttcgtacaatggtgtcgactactgagcttcagaagacaactggaactatgatccacaagctggcagcccgagctctaatcagagattatgaagatggcattcttcacgaaaatgaaaccagtcatgagatgaaaaaacaaaccttgaaatctctgattattaaactcagtaaagaaaactctctcataacacaatttacaagctttgtggcagttgagaaaagggatgagaatgagtcgccttttcctgatattccaaaagtttctgaacttattgccaaagaagatgtagacttcctgccctacatgagctggcagggggagccccaagaagccgtcaggaaccagtctcttttagcatcctctgagtggccagaattacgtttatccaaacgaaaacataggaaaattccattttccaaaagaaaaatggaattatctcagccagaagtttctgaagattttgaagaggatggcttaggtgtactaccagctttcacatcaaatttggaacgtggaggtgtggaaaagctattggatttaagttggacagagtcatgtaaaccaacagcaactgaaccactatttaagaaagtcagtccatgggaaacatctacttctagcttttttcctattttggctccggccgttggttcctatcttcccccgactgcccgcgctcacagtcctgcttccttgtcttttgcctcatatcgtcaggtagctagtttcggttcagctgctcctcccagacagtttgatgcatctcaattcagccaaggccctgtgcctggcacttgtgctgactggatcccacagtcggcgtcttgtcccacaggacctccccagaacccaccttcttcaccctattgtggcattgttttttcagggagctcattaagctctgcacagtctgctccactgcaacatcctggaggctttactaccaggccttctgctggcaccttccctgagctggattctccccagcttcatttctctcttcctacagaccctgatcccatcagaggttttgggtcttatcatccctctgcttcctctccttttcattttcaaccttccgcagcctctttgactgccaaccttaggctgccaatggcctctgctttacctgaggctctttgcagtcagtcccggactaccccagtagatctctgtcttctagaagaatcagtaggcagtctcgaaggaagtcgatgtcctgtctttgcttttcaaagttctgacacagaaagtgatgagctatcagaagtacttcaagacagctgctttttacaaataaaatgtgatacaaaagatgacagtatcctgtgctttctggaagtaaaagaagaggatgaaatagtgtgcatacaacactggcaggatgctgtgccttggacagaactcctcagtctacagacagaggatggcttctggaaacttacaccagaactgggacttatattaaatcttaatacaaatggtttgcacagctttcttaaacaaaaaggcattcaatctctaggtgtaaaaggaagagaatgtctcctggacctaattgccacaatgctggtactacagtttattcgcaccaggttggaaaaagagggaatagtgttcaaatcactgatgaaaatggatgacgcttctatttccaggaatattccctgggcttttgaggcaataaagcaagcaagtgaatgggtaagaagaactgaaggacagtacccatctatctgcccacggcttgaactggggaacgactgggactctgccaccaagcagttgctgggactccagcccataagcactgtgtcccctcttcatagagtcctccattacagtcaaggctaa

SEQ ID NO: 104 - DNA, 2211 bp, Homo sapiens
atgaagaaaagcaggagtgtgatgacggtgatggctgatgataatgttaaagattattttgaatgtagcttgagtaaatcctacagttcttccagtaacacacttgggatcgacctctggagagggagaaggtgttgctcaggaaacttacagttaccaccactgtctcaaagacagagtgaaagggcaaggactcctgagggagatggtatttccaggccgaccacactgcctttgacaacgcttccaagcattgctattacaactgtaagccaggagtgctttgatgtggaaaatggcccttccccaggtcggagtccactggatccccaggccagctcttccgctgggctggtacttcacgccacctttcctgggcacagccagcgcagagagtcatttctctacagatcagacagcgactatgacttgtcaccaaaggcgatgtcgagaaactcttctcttccaagcgagcaacacggcgatgacttgattgtaactccttttgcccaggtccttgccagcttgcgaagtgtgagaaacaacttcactatactgacaaaccttcatggtacatctaacaagaggtccccagctgctagtcagcctcctgtctccagagtcaacccacaagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataa

SEQ ID NO: 105 - DNA, 1695 bp, Homo sapiens
atgaaggagcacgggggcaccttcagtagcaccggaatcagcggtggtagcggtgactctgctatggacagcctgcagccgctccagcctaactacatgcctgtgtgtttgtttgcagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataa

SEQ ID NO: 106 - DNA, 2166 bp, Homo sapiens
atgacagcaaaagattcttcaaaggaacttactgcttctgaacctgaggtttgcataaagactttcaaggagcaaatgcatttagaacttgagcttccgagattaccaggaaacagacctacatctcctaaaatttctccacgcagttcaccaaggaactcaccatgctttttcagaaagttgctggtgaataaaagcattcggcagcgtcgtcgcttcactgtggctcatacatgctttgatgtggaaaatggcccttccccaggtcggagtccactggatccccaggccagctcttccgctgggctggtacttcacgccacctttcctgggcacagccagcgcagagagtcatttctctacagatcagacagcgactatgacttgtcaccaaaggcgatgtcgagaaactcttctcttccaagcgagcaacacggcgatgacttgattgtaactccttttgcccaggtccttgccagcttgcgaagtgtgagaaacaacttcactatactgacaaaccttcatggtacatctaacaagaggtccccagctgctagtcagcctcctgtctccagagtcaacccacaagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataa

SEQ ID NO: 107 - DNA, 3201 bp, Homo sapiens
atgggcttcgagctggaccgcttcgacggcgacgtggacccggacctgaagtgcgcgctgtgccacaaggtcctggaggacccgctgaccacgccgtgcggccacgtcttctgcgccggctgcgtgctgccctgggtggtgcaggagggcagctgcccggcgcgctgccgcggtcgcctgtcggccaaagagctcaaccacgtcctgccgctcaagcgccttatcctcaagctggacatcaagtgcgcgtacgcgacgcgcggctgcggccgggtggtcaagctgcagcagctgccggagcacctcgagcgctgcgacttcgcgcccgcgcgctgtcgccacgcgggttgcggccaggtgctgctgcggcgcgacgtggaggcgcacatgcgcgacgcctgcgacgcgcggccagtgggccgctgccaggagggctgcgggctacccttgacgcacggcgagcagcgcgcgggcggccactgctgcgcgcgagcgctgcgggcgcacaacggcgcgctccaggcccgcctgggcgcgctgcacaaggcgctcaagaaggaggcgctgcgcgctgggaagcgcgagaagtcgctggtggcccagctggccgcggcgcagcttgagctgcagatgaccgcgctgcgctaccagaagaaattcaccgaatacagcgcgcgcctcgactcgctcagccgctgcgtggccgcgccgcccggcggcaagggcgaagaaaccaaaagtctgactcttgtcctgcatcgggactccggctccctgggattcaatattattggtggccggccgagtgtggataaccacgatggatcatccagtgaaggaatctttgtatccaagatagttgacagtgggcctgcagccaaggaaggaggcctgcaaattcatgacaggattattgaggtcaacggcagagacttatccagagcaactcatgaccaggctgtggaagctttcaagacagccaaggagcccatagtggtgcaggtgttgagaagaacaccaaggaccaaaatgttcacgcctccatcagagtctcagctggtggacacgggaacccaaaccgacatcacctttgaacatatcatggccctcactaagatgtcctctcccagcccacccgtgctggatccctatctcttgccagaggagcatccctcagcccatgaatactacgatccaaatgactacattggagacatccatcaggagatggacagggaggagctggagctggaggaagtggacctctacagaatgaacagccaggacaagctgggcctcactgtgtgctaccggacggacgatgaagacgacattgggatttatatcagtgagattgaccctaacagcattgcagccaaggatgggcgcatccgagaaggagaccgcattatccagattaatgggatagaggtgcagaaccgtgaagaggctgtggctcttctaaccagtgaagaaaataaaaacttttcattgctgattgcaaggcctgaactccagctggatgagggctggatggatgatgacaggaacgactttctggatgacctgcacatggacatgctggaggagcagcaccaccaggccatgcaattcacagctagcgtgctgcagcagaagaagcacgacgaagacggtgggaccacagatacagccaccatcttgtccaaccagcacgagaaggacagcggtgtggggcggaccgacgagagcacccgtaatgacgagagctcggagcaagagaacaatggcgacgacgccaccgcatcctccaacccgctggcggggcagaggaagctcacctgcagccaggacaccttgggcagcggcgacctgcccttcagcaacgagtctttcatttcggccgactgcacggacgccgactacctggggatcccggtggacgagtgcgagcgcttccgcgagctcctggagctcaagtgccaggtgaagagcgccaccccttacggcctgtactaccctagcggccccctggacgccggcaagagtgaccctgagagcgtggacaaggagctggagctgctgaacgaagagctgcgcagcatcgagctggagtgcctgagcatcgtgcgcgcccacaagatgcagcagctcaaggagcagtaccgcgagtcctggatgctgcacaacagcggcttccgcaactacaacaccagcatcgacgtgcgcagacacgagctctcagatatcaccgagctcccggagaaatccgacaaggacagctcgagcgcctacaacacaggcgagagctgccgcagcaccccgctcaccctggagatctcccccgacaactccttgaggagagcggcggagggcatcagctgcccgagcagcgaaggggctgtggggaccacggaagcctacgggccagcctccaagaatctgctctccatcacggaagatcccgaagtgggcacccctacctatagcccgtccctgaaggagctggaccccaaccagcccctggaaagcaaagagcggagagccagcgacgggagccggagccccacgcccagccagaagctgggcagcgcctacctgccctcctatcaccactccccatacaagcacgcgcacatcccggcgcacgcccagcactaccagagctacatgcagctgatccagcagaagtcggccgtggagtacgcgcaaagccagatgagcctggtgagcatgtgcaaggacctgagctctcccaccccgtcggagccgcgcatggagtggaaggtgaagatccgcagcgacgggacgcgctacatcaccaagaggcccgtgcgggaccgcctgctgcgggagcgcgccctgaagatccgggaagagcgcagcggcatgaccaccgacgacgacgcggtgagcgagatgaagatggggcgctactggagcaaggaggagaggaagcagcacctggtgaaggccaaggagcagcggcggcggcgcgagttcatgatgcagagcaggttggattgtctcaaggagcagcaagcagccgatgacaggaaggagatgaacattctcgaactgagccacaaaaagatgatgaagaagaggaataagaaaatcttcgataactggatgacgatccaagaactcttaacccacggcacaaaatccccggacggcactagagtatacaattccttcctatcggtgactactgtataa

SEQ ID NO: 108 - DNA, 465 bp, Homo sapiens
atggcgcagtctattaacatcacggagctgaatctgccgcagctagaaatgctcaagaaccagctggaccaggaagtggagttcttgtccacgtccattgctcagctcaaagtggtacagaccaagtatgtggaagccaaggactgtctgaacgtgctgaacaagagcaacgaggggaaagaattactcgtcccactgacgagttctatgtatgtccctgggaagctgcatgatgtggaacacgtgctcatcgatgtgggaactgggtactatgtagagaagacagctgaggatgccaaggacttcttcaagaggaagatagattttctaaccaagcagatggagaaaatccaaccagctcttcaggagaagcacgccatgaaacaggccgtcatggaaatgatgagtcagaagattcagcagctcacagccctgggggcagctcaggctactgctaaggcctga

SEQ ID NO: 109 - DNA, 330 bp, Homo sapiens
atggcgcagtctattaacatcacggagctgaatctgccgcagctagaaatgctcaagaaccagctggaccagatgtatgtccctgggaagctgcatgatgtggaacacgtgctcatcgatgtgggaactgggtactatgtagagaagacagctgaggatgccaaggacttcttcaagaggaagatagattttctaaccaagcagatggagaaaatccaaccagctcttcaggagaagcacgccatgaaacaggccgtcatggaaatgatgagtcagaagattcagcagctcacagccctgggggcagctcaggctactgctaaggcctga

SEQ ID NO: 110 - DNA, 1008 bp, Homo sapiens
atggggcccctctcagcccctccctgcacagagcacatcaaatggaaggggctcctgctcacagcattacttttaaacttctggaacttgcctaccactgcccaagtcatgattgaagcccagccacccaaagtgtccgaggggaaggatgttcttctacttgtccacaatttgccccagaatcttactggctacatctggtacaaagggcaaatcagggacctctaccattacattacatcatatgtagtagacggtcaaataattatatatggaccggcatacagtggacgagaaacagtatattccaatgcatccctgctgatccagaatgtcacccgggaggacgcaggatcctacaccttacacatcataaagcgaggtgatgggactagaggagtaactggatatttcaccttcaccttatacctggagactcccaagccctccatctccagcagcaacttaaaccccagggaggccatggagactgtgatcttaacctgtaatcctgagactccggacgcaagctacctgtggtggatgaatggtcagagcctccctatgactcataggatgcagctgtctgaaaccaacaggaccctctttctatttggtgtcacaaagtatactgcaggaccctatgaatgtgaaatatggaactcagggagtgccagccgcagtgacccagtcaccctgaatctcctccatggtccagacctccccagaattttcccttcagtcacctcttactattcaggagagaacctcgacttgtcctgcttcgcaaactctaacccaccagcacagtattcttggacaattaatgggaagtttcagctatcaggacaaaagctctttatccctcagattactccaaagcataatgggctctatgcttgctctgctcgtaactcagccactggcgaggaaagctccacatccttgacaatcagagtcattgctcctccaggattaggaacttttgctttcaataatccaacgtag

SEQ ID NO: 111 - DNA, 642 bp, Homo sapiens
atggggcccctctcagcccctccctgcacagagcacatcaaatggaaggggctcctgctcacagtggagactcccaagccctccatctccagcagcaacttaaaccccagggaggccatggagactgtgatcttaacctgtaatcctgagactccggacgcaagctacctgtggtggatgaatggtcagagcctccctatgactcataggatgcagctgtctgaaaccaacaggaccctctttctatttggtgtcacaaagtatactgcaggaccctatgaatgtgaaatatggaactcagggagtgccagccgcagtgacccagtcaccctgaatctcctccatggtccagacctccccagaattttcccttcagtcacctcttactattcaggagagaacctcgacttgtcctgcttcgcaaactctaacccaccagcacagtattcttggacaattaatgggaagtttcagctatcaggacaaaagctctttatccctcagattactccaaagcataatgggctctatgcttgctctgctcgtaactcagccactggcgaggaaagctccacatccttgacaatcagagtcattgctcctccaggattaggaacttttgctttcaataatccaacgtag

SEQ ID NO: 112 - DNA, 1115 bp, Homo sapiens
caaagttcatagcaggttgaacatgcttgctgtcattttcacagattctggtctttcttatggggaacctgagttcctgcattaaagttcccccagaagatcaatggaaggtggattccaaagagtgggcaagtgaggaccccttctctgttaccaaggtgaccccaaggaacacagtaaatgtggcggcttatttggcctccccaggacggactggagcatcagtagtgcctgagttcatcaaaggacagatgctcaagacaccctgatcaccattaggtggaattgaaggagccaaaaatgggcgcagtggctcctcagcagagacgctcctgaatgtatagacatgggaaccactttcagcatcaaaaaaggaaacgttctgcatgcccatatccagaaaaatccccactcgctgtaacttgcggtctactaagaggaaagtcagcggcaccgtgctggcagagaggcggcttccatccctcaaactcacagtccagaatccaagctctgtggtcagctggatcttccctttgcagtgaacagattctctgcagactcccaggtcccattctgtgcttgttcccacgtccacctcccagtagtggcggccacaggtaaagcgaggggagcccaggatgcaaacggacacgtcaaatctctcggcaaggtcttgccgattctgtgtgatgagcccacttcggacgctcctgaggtcgtcagaaatgaggaggaagttgttggctgtgtcggcatccaaggtcatatccactgtgaaaaggaaaacaagttgctcagcaagtggacagaagccaaccccatgcctctcttctacttcaaaggcccaaatatctcctgactttagtttctttaattctgtttcttcctccaaaatcaaaacttttcatgaggaaacttccctatttcaaattttcataggtatgctttgtggcaatacgtgactcttattcacagtagcaattcttattttactttacgtcacgtgtttgcttcactgaacttttcttcatttagagtggagggtctctgaaggcagacaccatgcccccctttctaccaccttgatgtgtgaacaaataaatgataattaagac

SEQ ID NO: 113 - DNA, 1805 bp, Homo sapiens
aaagcctgcgagcgccagccgagatcgcagcccaacccatggccgggtctcctagccgcgccgcgggccggcgactgcagcttcccctgctgtgcctcttcctccagggcgccactgccgtcctctttgctgtctttgtccgctacaaccacaaaaccgacgctgccctctggcaccggagcaaccacagtaacgcggacaatgaattttactttcgctacccaagcttccaggacgtgcatgccatggtcttcgtgggctttggcttcctcatggtcttcctgcagcgttacggcttcagcagcgtgggcttcaccttcctcctggccgcctttgccctgcagtggtccacactggtccagggctttctccactccttccacggtggccacatccatgttggcgtggagagcatgatcaatgctgacttttgtgcgggggccgtgctcatctcctttggtgccgtcctgggcaagaccgggcctacccagctgctgctcatggccctgctggaggtggtgctgtttggcatcaatgagtttgtgctccttcatctcctgggggtgagagatgccggaggctccatgactatccacacctttggtgcctacttcgggctcgtcctttcgcgggttctgtacaggccccagctggagaagagcaagcaccgccagggctccgtctaccattcagacctcttcgccatgattgggaccatcttcctgtggatcttctggcctagcttcaatgctgcactcacagcgctgggggctgggcagcatcggacggccctcaacacatactactccctggctgccagcacccttggcacctttgccttgtcagcccttgtaggggaagatgggaggcttgacatggtccacatccaaaatgcagcgctggctggaggggttgtggtggggacctcaagtgaaatgatgctgacaccctttggggctctggcagctggcttcttggctgggactgtctccacgctggggtacaagttcttcacgcccatccttgaatcaaaattcaaagtccaagacacatgtggagtccacaacctccatgggatgccgggggtcctgggggccctcctgggggtccttgtggctggacttgccacccatgaagcttacggagatggcctggagagtgtgtttccactcatagccgagggccagcgcagtgccacgtcacaggccatgcaccagctcttcgggctgtttgtcacactgatgtttgcctctgtgggcgggggccttggagggctcctgctgaagctaccctttctggactcccccccagactcccagcactacgaggaccaagttcactggcaggtgcctggcgagcatgaggataaagcccagagacctctgagggtggaggaggcagacactcaggcctaacccactgccagcccctgagaggacacgctccttttcgaagatgctgactggctgctactaggaagttctttttgagctcccattcctccagctgcaagaagggagccatgagccagaaggaggcccctttccacaggcagcgtctccacagggagaggggcaacaggaggctgggaaatggtggggagtggggccgtaactgggtacaatagggggaacctcaccagatgcccaacccgactgccctaccagcctgcacatgggtagaagaggccaaattgaggcacccaagtgatccactggccccacgtcacacagttacagtgaagcccaagccaggcctggttgagggtgataaacgccactgtctctaaaaaaaaaaaaaaaaaa

SEQ ID NO: 114 - DNA, 1928 bp, Homo sapiens
aaagcctgcgagcgccagccgagatcgcagcccaacccatggccgggtctcctagccgcgccgcgggccggcgactgcagcttcccctgctgtgcctcttcctccagggcgccactgccgtcctctttgctgtctttgtccgctacaaccacaaaaccgacgctgccctctggcaccggagcaaccacagtaacgcggacaatgaattttactttcgctacccaaatggagtttcgctcttgttgcccaggctggagtgcaatggcacaatctcggctcaccacaacctctgcctcccgggttcaagcgattctcctgcctcagtctcctgagtagctgggattacaggcttccaggacgtgcatgccatggtcttcgtgggctttggcttcctcatggtcttcctgcagcgttacggcttcagcagcgtgggcttcaccttcctcctggccgcctttgccctgcagtggtccacactggtccagggctttctccactccttccacggtggccacatccatgttggcgtggagagcatgatcaatgctgacttttgtgcgggggccgtgctcatctcctttggtgccgtcctgggcaagaccgggcctacccagctgctgctcatggccctgctggaggtggtgctgtttggcatcaatgagtttgtgctccttcatctcctgggggtgagagatgccggaggctccatgactatccacacctttggtgcctacttcgggctcgtcctttcgcgggttctgtacaggccccagctggagaagagcaagcaccgccagggctccgtctaccattcagacctcttcgccatgattgggaccatcttcctgtggatcttctggcctagcttcaatgctgcactcacagcgctgggggctgggcagcatcggacggccctcaacacatactactccctggctgccagcacccttggcacctttgccttgtcagcccttgtaggggaagatgggaggcttgacatggtccacatccaaaatgcagcgctggctggaggggttgtggtggggacctcaagtgaaatgatgctgacaccctttggggctctggcagctggcttcttggctgggactgtctccacgctggggtacaagttcttcacgcccatccttgaatcaaaattcaaagtccaagacacatgtggagtccacaacctccatgggatgccgggggtcctgggggccctcctgggggtccttgtggctggacttgccacccatgaagcttacggagatggcctggagagtgtgtttccactcatagccgagggccagcgcagtgccacgtcacaggccatgcaccagctcttcgggctgtttgtcacactgatgtttgcctctgtgggcgggggccttggagggctcctgctgaagctaccctttctggactcccccccagactcccagcactacgaggaccaagttcactggcaggtgcctggcgagcatgaggataaagcccagagacctctgagggtggaggaggcagacactcaggcctaacccactgccagcccctgagaggacacgctccttttcgaagatgctgactggctgctactaggaagttctttttgagctcccattcctccagctgcaagaagggagccatgagccagaaggaggcccctttccacaggcagcgtctccacagggagaggggcaacaggaggctgggaaatggtggggagtggggccgtaactgggtacaatagggggaacctcaccagatgcccaacccgactgccctaccagcctgcacatgggtagaagaggccaaattgaggcacccaagtgatccactggccccacgtcacacagttacagtgaagcccaagccaggcctggttgagggtgataaacgccactgtctctaaaaaaaaaaaaaaaaaa

SEQ ID NO: 115 - DNA, 1878 bp, Homo sapiens
aaagcctgcgagcgccagccgagatcgcagcccaacccatggccgggtctcctagccgcgccgcgggccggcgactgcagcttcccctgctgtgcctcttcctccagggcgccactgccgtcctctttgctgtctttgtccgctacaaccacaaaaccgacgctgccctctggcaccggagcaaccacagtaacgcggacaatgaattttactttcgctacccaaaagagtctcactctgttgcccaggctggagtgcaacggcgcaatctcggctcactgcaaccttcacctcccaggcttccaggacgtgcatgccatggtcttcgtgggctttggcttcctcatggtcttcctgcagcgttacggcttcagcagcgtgggcttcaccttcctcctggccgcctttgccctgcagtggtccacactggtccagggctttctccactccttccacggtggccacatccatgttggcgtggagagcatgatcaatgctgacttttgtgcgggggccgtgctcatctcctttggtgccgtcctgggcaagaccgggcctacccagctgctgctcatggccctgctggaggtggtgctgtttggcatcaatgagtttgtgctccttcatctcctgggggtgagagatgccggaggctccatgactatccacacctttggtgcctacttcgggctcgtcctttcgcgggttctgtacaggccccagctggagaagagcaagcaccgccagggctccgtctaccattcagacctcttcgccatgattgggaccatcttcctgtggatcttctggcctagcttcaatgctgcactcacagcgctgggggctgggcagcatcggacggccctcaacacatactactccctggctgccagcacccttggcacctttgccttgtcagcccttgtaggggaagatgggaggcttgacatggtccacatccaaaatgcagcgctggctggaggggttgtggtggggacctcaagtgaaatgatgctgacaccctttggggctctggcagctggcttcttggctgggactgtctccacgctggggtacaagttcttcacgcccatccttgaatcaaaattcaaagtccaagacacatgtggagtccacaacctccatgggatgccgggggtcctgggggccctcctgggggtccttgtggctggacttgccacccatgaagcttacggagatggcctggagagtgtgtttccactcatagccgagggccagcgcagtgccacgtcacaggccatgcaccagctcttcgggctgtttgtcacactgatgtttgcctctgtgggcgggggccttggagggctcctgctgaagctaccctttctggactcccccccagactcccagcactacgaggaccaagttcactggcaggtgcctggcgagcatgaggataaagcccagagacctctgagggtggaggaggcagacactcaggcctaacccactgccagcccctgagaggacacgctccttttcgaagatgctgactggctgctactaggaagttctttttgagctcccattcctccagctgcaagaagggagccatgagccagaaggaggcccctttccacaggcagcgtctccacagggagaggggcaacaggaggctgggaaatggtggggagtggggccgtaactgggtacaatagggggaacctcaccagatgcccaacccgactgccctaccagcctgcacatgggtagaagaggccaaattgaggcacccaagtgatccactggccccacgtcacacagttacagtgaagcccaagccaggcctggttgagggtgataaacgccactgtctctaaaaaaaaaaaaaaaaaa

SEQ ID NO: 116 - DNA, 612 bp, Homo sapiens
atggcggaggtgcaggtcctggtgcttgatggtcgaggccatctcctgggccgcctggcggccatcgtggctaaacaggtactgctgggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctga

SEQ ID NO: 117 - DNA, 2133 bp, Homo sapiens
atgcgcggtcgcctttgtgtgggtcgagcagcggcggcggcggcggcagtggcggtcccactggcaggcgggcaagaggggagtccgggcggcggccggcgtgggagccgggggaccaccatggtaaagaagcggaaaggccgcgtcgtgatcgactcggacacagaggacagcggcagcgacgagaacctggatcaggagctcttgtccctggcaaagcgaaagcgcagtgactctgaggagaaggagccgcctgtgagtcagcctgcagcctcgtcagactcggagacgtctgacagtgacgatgagtggacatttgggagcaataaaaataagaagaaaggaaaagccagaaaaatagagaagaaaggaaccatgaagaaacaggccaacaaaactgcctcctcaggcagttcagacaaagacagttcagctgagagctcagcccctgaggaaggtgaagtgtcagactctgacagcaacagctcctcttccagttcagattcagactcttcctcagaagatgaagagttccatgatggctatggagaagacctcatgggagatgaggaagacagggcccgtctggaacagatgacagagaaagagagagagcaagaactgttcaatcgcatagagaagagggaggtgttgaaaagaagatttgaaatcaagaaaaaactaaaaacagccaaaaagaaagaaaagaaagaaaagaagaaaaagcaagaagaggagcaagaaaagaaaaaactgacacagattcaagaatctcaggtaacatcccacaacaaggaacggcgttccaagcgggatgagaaactagacaagaaatctcaagccatggaggagctaaaagcagagcgagaaaaacgaaagaacagaacagctgagctccttgccaaaaaacagccattaaaaaccagtgaggtctactctgatgatgaagaggaggaagaggatgacaaatccagtgaaaagtcagaccgctcatcacgaacatcatcgtctgatgaagaagaggagaaagaagagatccctcccaaatcccaaccagtttccttacctgaagaattgaatcgggttcgattatcacggcataagctagaacgctggtgtcacatgcccttctttgctaaaactgtcacaggatgttttgtgcggattggcatcggaaaccacaacagcaaaccagtttaccgggtcgctgagattacgggtgttgtggaaactgccaaagtttaccaactaggtggcaccagaacaaacaaagggctgcaactacggcatggcaatgaccaacgcgtgttccgtttagagtttgtctcaaaccaagaattcaccgaaagtgagtttatgaagtggaaagaagcgatgttctctgctggcatgcagttgcccactctagatgaaatcaataaaaaggaattatctattaaagaagctcttaattataaattcaatgatcaggacattgaagagattgtaaaagagaaagaaaggttcagaaaagctccacccaactacgctatgaagaagactcagctactgaaggaaaaggccatggctgaggacctgggggatcaggacaaggccaaacaaatccaagatcaactgaatgagctggaggaacgggcagaggccctggaccgccagcggaccaagaacatatccgctatcagttacatcaaccagcggaaccgggagtggaacattgtagagtctgagaaggcccttgtggctgaaagtcacaacatgaaaaaccaacagatggatccctttactcggcggcagtgcaagcctaccatcgtttctaattccagagacccagctgttcaagctgccatcttggcccagctgaatgcaaaatacggttctggagtgttaccagatgctccaaaggaaatgagcaagggtcaaggcaaagataaagatttgaattctaagtcagccagtgacctctcagaagatctgttcaaagtacacgattttgatgtgaagattgacttacaagttcccagctcagagtcaaaggctttagccatcacctccaaggctccgccagccaaggatggggctccaaggagatctctgaacttggaagactacaaaaaacgacgagggcttatttga

SEQ ID NO: 118 - DNA, 996 bp, Homo sapiens
atggaagaacttacggcgttcgtctccaagtcttttgaccagaaagtgaaggagaagaaggaggccatcacgtaccgggaggtgctagagagcgggccgctgcgcggggccaaagagcccggttgcgtcgagccgggccgcgacgaccgcagcagcccggcagtccgggcggccggcggaggcggcggcgcgggaggaggcggaggcggaggcggaggaggcggaggaggtgctggaggaggaggagcaggcggaggagctggaggagggcgctctcccgtccgggagctggacatgggagccgcggagcggagcagggagcccggcagcccgcggctgacggaggtgtcccctgaactgaaggatcgcaaagacgatgcgaaagggatggaggacgaaggccagaccaaaatcaagcagaggcgaagtcggaccaattttaccctggaacaactcaacgagctggagaggcttttcgatgagacccactatccagacgctttcatgcgcgaggaattgagccagcgactggggctctctgaggcccgagtacaggtttggtttcaaaatcgaagagctaagtgtagaaaacaggaaaatcaacttcacaaaggtgtccttataggagccgctagccagtttgaagcttgtagagttgcaccctatgtcaacgtaggtgctttaaggatgccatttcagcaggatagtcattgcaacgtgacgcccttgtcctttcaggttcaggcgcagctgcagctggacagcgccgtggcgcacgcgcaccaccacctgcatccgcacctggccgcgcacgcgccttacatgatgttcccggcaccgcccttcggactgccgctggccacgctggccgcggactcggcctcggccgcctcggtggtggccgctgccgccgccgccaagaccaccagcaagaactccagcatcgcggatctcagactgaaagctaaaaagcacgcggccgccctgggtctgtga

SEQ ID NO: 119 - DNA, 1653 bp, Homo sapiens
atgacaggtgacaagggtccccaaaggctaagcgggtccagctatggttccatctccagcccgaccagcccgaccagcccagggccacagcaagcacctcccagagagacctacctgagtgagaagatccccatcccagacacaaaaccgggcaccttcagcctgcggaagctatgggccttcacggggcctggcttcctcatgagcattgctttcctggacccaggaaacatcgagtcagatcttcaggctggcgccgtggcgggattcaaacttctctgggtgctgctctgggccaccgtgttgggcttgctctgccagcgactggctgcacgtctgggcgtggtgacaggcaaggacttgggcgaggtctgccatctctactaccctaaggtgccccgcaccgtcctctggctgaccatcgagctagccattgtgggctccgacatgcaggaagtcatcggcacggccattgcattcaatctgctctcagctggacgaatcccactctggggtggcgtcctcatcaccatcgtggacaccttcttcttcctcttcctcgataactacgggctgcggaagctggaagctttttttggactccttataaccattatggccttgacctttggctatgagtatgtggtggcgcgtcctgagcagggagcgcttcttcggggcctgttcctgccctcgtgcccgggctgcggccaccccgagctgctgcaggcggtgggcattgttggcgccatcatcatgccccacaacatctacctgcactcggccctggtcaagtctcgagagatagaccgggcccgccgagcagacatcagagaagccaacatgtacttcctgattgaggccaccatcgccctgtccgtctcctttatcatcaacctctttgtcatggctgtctttgggcaggccttctaccagaaaaccaaccaggctgcgttcaacatctgtgccaacagcagcctccacgactacgccaagatcttccccatgaacaacgccaccgtggccgtggacatttaccaggggggcgtgatcctgggctgcctgttcggccccgcggccctctacatctgggccataggtctcctggcggctgggcagagctccaccatgacgggcacctacgcgggacagttcgtgatggagggcttcctgaggctgcggtggtcacgcttcgcccgtgtcctcctcacccgctcctgcgccatcctgcccaccgtgctcgtggctgtcttccgggacctgagggacttgtcgggcctcaatgatctgctcaacgtgctgcagagcctgctgctcccgttcgccgtgctgcccatcctcacgttcaccagcatgcccaccctcatgcaggagtttgccaatggcctgctgaacaaggtcgtcacctcttccatcatggtgctagtctgcgccatcaacctctacttcgtggtcagctatctgcccagcctgccccaccctgcctacttcggccttgcagccttgctggccgcagcctacctgggcctcagcacctacctggtctggacctgttgccttgcccacggagccacctttctggcccacagctcccaccaccacttcctgtatgggctccttgaagaggaccagaaaggggagacctctggctag

SEQ ID NO: 120 - DNA, 1908 bp, Homo sapiens
atgagtgtaggggtgagcacctcagcccctctttccccaacctcgggcacaagcgtgggcatgtctaccttctccatcatggactatgtggtgttcgtcctgctgctggttctctctcttgccattgggctctaccatgcttgtcgtggctggggccggcatactgttggtgagctgctgatggcggaccgcaaaatgggctgccttccggtggcactgtccctgctggccaccttccagtcagccgtggccatcctgggtgtgccgtcagagatctaccgatttgggacccaatattggttcctgggctgctgctactttctggggctgctgatacctgcacacatcttcatccccgttttctaccgcctgcatctcaccagtgcctatgagtacctggagcttcgattcaataaaactgtgcgagtgtgtggaactgtgaccttcatctttcagatggtgatctacatgggagttgtgctctatgctccgtcattggctctcaatgcagtgactggctttgatctgtggctgtccgtgctggccctgggcattgtctgtaccgtctatacagctctgggtgggctgaaggccgtcatctggacagatgtgttccagacactggtcatgttcctcgggcagctggcagttatcattgtggggtcagccaaggtgggcggcttggggcgtgtgtgggccgtggcttcccagcacggccgcatctctgggtttgagctggatccagacccctttgtgcggcacaccttctggaccttggccttcgggggtgtcttcatgatgctctccttatacggggtgaaccaggctcaggtgcagcggtacctcagttcccgcacggagaaggctgctgtgctctcctgttatgcagtgttccccttccagcaggtgtccctctgcgtgggctgcctcattggcctggtcatgttcgcgtattaccaggagtatcccatgagcattcagcaggctcaggcagccccagaccagttcgtcctgtactttgtgatggatctcctgaagggcctgccaggcctgccagggctcttcattgcctgcctcttcagcggctctctcagcactatatcctctgcttttaattcattggcaactgttacgatggaagacctgattcgaccttggttccctgagttctctgaagcccgggccatcatgctttccagaggccttgcctttggctatgggctgctttgtctaggaatggcctatatttcctcccagatgggacctgtgctgcaggcagcaatcagcatctttggcatggttgggggaccgctgctgggactcttctgccttggaatgttctttccatgtgctaaccctcctggtgctgttgtgggcctgttggctgggctcgtcatggccttctggattggcatcgggagcatcgtgaccagcatgggctccagcatgccaccctctccctctaatgggtccagcttctccctgcccaccaatctaaccgttgccactgtgaccacactgatgcccttgactaccttctccaagcccacagggctgcagcggttctattccttgtcttacttatggtacagtgctcacaactccaccacagtgattgtggtgggcctgattgtcagtctactcactgggagaatgcgaggccggtccctgaaccctgcaaccatttacccagtgttgccaaagctcctgtccctccttccgttgtcctgtcagaagcggctccactgcaggagctacggccaggaccacctcgacactggcctgtttcctgagaagccgaggaatggtgtgctgggggacagcagagacaaggaggccatggccctggatggcacagcctatcaggggagcagctccacctgcatcctccaggagacctccctgtga

SEQ ID NO: 121 - DNA, 1752 bp, Homo sapiens
atgcctttaaggaagatgaagatccctttcctcctactgttctttctgtgggaagccgagagccacgcagcatcaaggccgaacatcatcctggtgatggctgacgacctcggcattggagatcctgggtgctatgggaacaaaactatcaggactcccaatatcgaccggttggccagtgggggagtgaaactcactcagcacctggcagcatcaccgctgtgcacaccaagcagggcagccttcatgactggccggtaccctgtccgatcaggaatggcatcttggtcccgcactggagttttcctcttcacagcctcttcgggaggacttcccaccgatgagattacctttgctaagcttctgaaggatcaaggttattcaacagcactgatagggaaatggcaccttgggatgagctgtcacagcaagactgacttctgtcaccaccctttacatcacggcttcaattatttctatgggatctctttgaccaatctgagagactgcaagcccggagagggcagtgtcttcaccacgggcttcaagaggctggtcttcctccccctgcagatcgtcggggtcaccctccttacccttgctgcactcaattgtctggggctactccacgtgcctctaggcgtttttttcagccttctcttcctagcagccctaatcctgacccttttcttgggcttccttcattacttccggcccctgaactgcttcatgatgaggaactacgagatcattcagcagcccatgtcctatgacaatctcacccagaggctaacggtggaggcggcccagttcatacagcggaacactgagactccgttcctgcttgtcttgtcctacctccacgtgcacacagccctgttctccagcaaagactttgctggcaaaagtcaacacggagtctacggggatgctgttgaggaaatggactggagtgtggggcagatcttgaaccttctggatgagctgagattggctaatgataccctcatctacttcacatcggaccagggagcacatgtagaagaagtgtcttccaaaggagaaattcatggcggaagtaatgggatctataaaggaggaaaagcaaacaactgggaaggaggtatccgggttccaggcatccttcgttggcccagggtgatacaggctggccagaagattgatgagcccactagcaacatggacatatttcctacagtagccaagctggctggagctcccttgcctgaggacaggatcattgatggacgtgatctgatgcccctgcttgaaggaaaaagccaacgctccgatcatgagtttctcttccattactgcaacgcctacttaaatgctgtgcgctggcaccctcagaacagcacatccatctggaaggcctttttcttcacccccaacttcaaccccgtgggttccaacggatgctttgccacacacgtgtgcttctgtttcgggagttatgtcacccatcacgacccacctttactctttgatatttccaaagatcccagagagagaaacccactaactccagcatccgagccccggttttatgaaatcctcaaagtcatgcaggaagctgcggacagacacacccagaccctgccagaggtgcccgatcagttttcatggaacaactttctttggaagccctggcttcagctgtgctgtccttccaccggcctgtcttgccagtgtgatagagaaaaacaggataagagactgagccgctag

SEQ ID NO: 122 - DNA, 1449 bp, Homo sapiens
atggccagcccggagccccggcgcggcggggacggcgccgcccaggccgcgaggaaaacaagagtagaggccaattctcctcttccaaagaactctggatccctaaatgaggcagaagccttgaacccagaagttactctatcttcagaggggtccttaaacctcgaagacattctctacctggaggacacaggtgaccttgatgagacactctatgtgcaagagactgagaaggcagaggaggccctgtatattgaggaggccatgcagccagatgaggctctgcatgtggaggagcctgggaatccagaggagacagtgtgtgtggaggaaaccacggagccagatcggatacagtttgtggaggggcccgtggagccaggaaagcccacaagcccagagcacgttgtttatgagggagagacagtcacaagggcggagaaatctaaccctgaggagagcctcagagccgagcagagccccagcatggaggagaacctgagcatagaggacctggaattgctagaggggcgtttccagcagtgtgtccaagctgtggcccagctggaagaggagagggatcagctcatccatgagcttgtattgctccgggaaccagccctgcaggaggtacagcaagtccatcaagacatcctggctgcctacaagctccatgcccaagcagagctggagagagatggcctaagggaggagatccggctggtcaagcagaagcttttcaaagtgacaaaggaatgtgtggcctaccaataccagctggagtgccgccagcaggacgtggctcagtttgccgatttccgggaagtgctgactacaagggcaacccagctctcagaggaactggcccagctccgggatgcctatcagaagcagaaggagcagctgcggcaacaactagaagcccctccaagccagagggatgggcactttctccaggaaagccggcgactctctgcccagtttgaaaatctcatggcagagagccgccaggacctggaggaggagtatgagcctcagttcctgcggctcctagagaggaaagaagctgggaccaaagctctgcagagaacccaggctgagatccaggaaatgaaggaggctctgagacccctgcaagcagaggcccggcagctccgcctgcaaaacaggaacctggaggaccagatcgcacttgtgaggcaaaaacgagatgaagaggtgcagcagtacagggaacagctggaggaaatggaagaacgccagaggcagttaagaaatggggtgcaactccagcaacagaagaacaaagagatggaacagctaaggctcagtcttgctgaagagctctctacttataaggctatgctactacccaagagcctggaacaggctgatgctcccacttctcaggcaggtggaatggagacacagtctcaaggggctgtttag

SEQ ID NO: 123 - DNA, 1431 bp, Homo sapiens
atggccagcccggagccccggcgcggcggggacggcgccgcccaggccgcgaggaaaacaagagtagaggccaattctcctcttccaaagaactctggatccctaaatgaggcagaagccttgaacccagaagttactctatcttcagaggggtccttaaacctcgaagacattctctacctggaggacacaggtgaccttgatgagacactctatgtgcaagagactgagaaggcagaggaggccctgtatattgaggaggccatgcagccagatgaggctctgcatgtggaggagcctgggaatccagaggagacagtgtgtgtggaggaaaccacggagccagatcggatacagtttgtggaggggcccgtggagccaggaaagcccacaagcccagagcacgttgtttatgagggagagacagtcacaagggcggagaaatctaaccctgaggagagcctcagagccgagcagagccccagcatggaggagaacctgagcatagaggacctggaattgctagaggggcgtttccagcagtgtgtccaagctgtggcccagctggaagaggagagggatcagctcatccatgagcttgtattgctccgggaaccagccctgcaggaggtacagcaagtccatcaagacatcctggctgcctacaagctccatgcccaagcagagctggagagagatggcctaagggaggagatccggctggtcaagcagaagcttttcaaagtgacaaaggaatgtgtggcctaccaataccagctggagtgccgccagcaggacgtggctcagtttgccgatttccgggaagtgctgactacaagggcaacccagctctcagaggaactggcccagctccgggatgcctatcagaagcagaaggagcagctgcggcaacaactagaagcccctccaagccagagggatgggcactttctccaggaaagccggcgactctctgcccagtttgaaaatctcatggcagagagccgccaggacctggaggaggagtatgagcctcagttcctgcggctcctagagaggaaagaagctgggaccaaagctctgcagagaacccaggctgagatccaggaaatgaaggaggctctgagacccctgcaagcagaggcccggcagctccgcctgcaaaacaggaacctggaggaccagatcgcacttgtgaggcaaaaacgagatgaagaggtgcagcagtacagggaacagctggaggaaatggaagaacgccagaggcagttaagaaatggggtgcaactccagcaacagaagaacaaagagatggaacagctaaggctcagtcttgctgaagagctctctacttataagggctgtttagaaatatatggccaaatctgtaacccggaaacagcaaaaaacttcttagcaaaggatcactaa

SEQ ID NO: 124 - DNA, 3507 bp, Homo sapiens
atggaaccaataacattcacagcaaggaaacatctgctttctaacgaggtctcggtggattttggcctgcagctggtgggctccctgcctgtgcattccctgaccaccatgcccatgctgccctgggttgtggctgaggtgcgaagactcagcaggcagtccaccagaaaggaacctgtaaccaagcaagtccggctttgcgtttcaccctctggactgagatgtgaacctgagccagggagaagtcaacagtgggatcccctgatctattccagcatctttgagtgcaagcctcagcgtgttcacaaactgattcacaacagtcatgacccaagttactttgcttgtctgattaaggaagacgctgtccaccggcagagtatctgctatgtgttcaaagccgatgatcaaacaaaagtgcctgagatcatcagctccatccgtcaggcggggaagatcgcccggcaggaggagctgcactgcccgtccgagttcgacgacacgttttccaagaagttcgaggtgctcttctgcggccgcgtgacggtggcgcacaagaaggctccgccggccctgatcgacgagtgcatcgagaagttcaatcacgtcagcggcagccgggggtccgagagcccccgccccaacccgccccatgccgcgcccacagggagccaggagcctgtgcgcaggcccatgcgcaagtccttctcccagcccggcctgcgctcgctggcctttaggaaggagctgcaggatgggggcctccgaagcagcggcttcttcagctccttcgaggagagcgacattgagaaccacctcattagcggacacaatattgtgcagcccacagatatcgaggaaaatcgaactatgctcttcacgattggccagtctgaagtttacctcatcagtcctgacaccaaaaaaatagcattggagaaaaattttaaggagatatccttttgctctcagggcatcagacacgtggaccactttgggtttatctgtcgggagtcttccggaggtggcggctttcattttgtctgttacgtgtttcagtgcacaaatgaggctctggttgatgaaattatgatgaccctgaaacaggccttcacggtggccgcagtgcagcagacagctaaggcgccagcccagctgtgtgagggctgccccctgcaaagcctgcacaagctctgtgagaggatagagggaatgaattcttccaaaacaaaactagaactgcaaaagcacctgacgacattaaccaatcaggagcaggcgactatttttgaagaggttcagaaattgagaccgagaaatgagcagcgagagaatgaattgattatttcttttctgagatgtttatatgaagagaaacagaaagaacacatccatattggggagatgaagcagacatcgcagatggcagcagagaatattggaagtgaattaccacccagtgccactcgatttaggctagatatgctgaaaaacaaagcaaagagatctttaacagagtctttagaaagtattttgtcccggggtaataaagccagaggcctgcaggaacactccatcagtgtggatctggatagctccctgtctagtacattaagtaacaccagcaaagagccatctgtgtgtgaaaaggaggccttgcccatctctgagagctcctttaagctcctcggctcctcggaggacctgtccagtgactcggagagtcatctcccagaagagccagctccgctgtcgccccagcaggccttcaggaggcgagcaaacaccctgagtcacttccccatcgaatgccaggaacctccacaacctgcccgggggtccccgggggtttcgcaaaggaaacttatgaggtatcactcagtgagcacagagacgcctcatgaacgaaaggactttgaatccaaagcaaaccatcttggtgattctggtgggactcctgtgaagacccggaggcattcctggaggcagcagatattcctccgagtagccaccccgcagaaggcgtgcgattcttccagcagatatgaagattattcagagctgggagagcttcccccacgatctcctttagaaccagtttgtgaagatgggccctttggccccccaccagaggaaaagaaaaggacatctcgtgagctccgagagctgtggcaaaaggctattcttcaacagatactgctgcttagaatggagaaggaaaatcagaagctccaagcctctgaaaatgatttgctgaacaagcgcctgaagctcgattatgaagaaattactccctgtcttaaagaagtaactacagtgtgggaaaagatgcttagcactccaggaagatcaaaaattaagtttgacatggaaaaaatgcactcggctgttgggcaaggtgtgccacgtcatcaccgaggtgaaatctggaaatttctagctgagcaattccaccttaaacaccagtttcccagcaaacagcagccaaaggatgtgccatacaaagaactcttaaagcagctgacttcccagcagcatgcgattcttattgaccttgggcgaacctttcctacacacccatacttctctgcccagcttggagcaggacagctatcgctttacaacattttgaaggcctactcacttctagaccaggaagtgggatattgccaaggtctcagctttgtagcaggcattttgcttcttcatatgagtgaggaagaggcgtttaaaatgctcaagtttctgatgtttgacatggggctgcggaaacagtatcggccagacatgattattttacagatccagatgtaccagctctcgaggttgcttcatgattaccacagagacctctacaatcacctggaggagcacgagatcggccccagcctctacgctgccccctggttcctcaccatgtttgcctcacagttcccgctgggattcgtagccagagtctttgatatgatttttcttcagggaacagaggtcatatttaaagtggctttaagtctgttgggaagccataagcccttgattctgcagcatgaaaacctagaaaccatagttgactttataaaaagcacgctacccaaccttggcttggtacagatggaaaagaccatcaatcaggtatttgaaatggacatcgctaaacagttacaagcttatgaagttgagtaccacgtccttcaagaagaacttatcgattcctctcctctcagtgacaaccaaagaatggataaattagagaaaaccaacagcagcttacgcaaacagaaccttgacctccttgaacagttgcaggtggcaaatggtaggatccaaagccttgaggccaccattgagaagctcctgagcagtgagagcaagctgaagcaggccatgcttaccttagaactggagcggtcggccctgctgcagacggtggaggagctgcggcggcggagcgcagagcccagcgaccgggagcctgagtgcacgcagcccgagcccacgggcgactga

SEQ ID NO: 125 - DNA, 3480 bp, Homo sapiens
atggaaccaataacattcacagcaaggaaacatctgctttctaacgaggtctcggtggattttggcctgcagctggtgggctccctgcctgtgcattccctgaccaccatgcccatgctgccctgggttgtggctgaggtgcgaagactcagcaggcagtccaccagaaaggaacctgtaaccaagcaagtccggctttgcgtttcaccctctggactgagatgtgaacctgagccagggagaagtcaacagtgggatcccctgatctattccagcatctttgagtgcaagcctcagcgtgttcacaaactgattcacaacagtcatgacccaagttactttgcttgtctgattaaggaagacgctgtccaccggcagagtatctgctatgtgttcaaagccgatgatcaaacaaaagtgcctgagatcatcagctccatccgtcaggcggggaagatcgcccggcaggaggagctgcactgcccgtccgagttcgacgacacgttttccaagaagttcgaggtgctcttctgcggccgcgtgacggtggcgcacaagaaggctccgccggccctgatcgacgagtgcatcgagaagttcaatcacgtcagcggcagccgggggtccgagagcccccgccccaacccgccccatgccgcgcccacagggagccaggagcctgtgcgcaggcccatgcgcaagtccttctcccagcccggcctgcgctcgctggcctttaggaaggagctgcaggatgggggcctccgaagcagcggcttcttcagctccttcgaggagagcgacattgagaaccacctcattagcggacacaatattgtgcagcccacagatatcgaggaaaatcgaactatgctcttcacgattggccagtctgaagtttacctcatcagtcctgacaccaaaaaaatagcattggagaaaaattttaaggagatatccttttgctctcagggcatcagacacgtggaccactttgggtttatctgtcgggagtcttccggaggtggcggctttcattttgtctgttacgtgtttcagtgcacaaatgaggctctggttgatgaaattatgatgaccctgaaacaggccttcacggtggccgcagtgcagcagacagctaaggcgccagcccagctgtgtgagggctgccccctgcaaagcctgcacaagctctgtgagaggatagagggaatgaattcttccaaaacaaaactagaactgcaaaagcacctgacgacattaaccaatcaggagcaggcgactatttttgaagaggttcagaaattgagaccgagaaatgagcagcgagagaatgaattgattatttcttttctgagatgtttatatgaagagaaacagaaagaacacatccatattggggagatgaagcagacatcgcagatggcagcagagaatattggaagtgaattaccacccagtgccactcgatttaggctagatatgctgaaaaacaaagcaaagagatctttaacagagtctttagaaagtattttgtcccggggtaataaagccagaggcctgcaggaacactccatcagtgtggatctggatagctccctgtctagtacattaagtaacaccagcaaagagccatctgtgtgtgaaaaggaggccttgcccatctctgagagctcctttaagctcctcggctcctcggaggacctgtccagtgactcggagagtcatctcccagaagagccagctccgctgtcgccccagcaggccttcaggaggcgagcaaacaccctgagtcacttccccatcgaatgccaggaacctccacaacctgcccgggggtccccgggggtttcgcaaaggaaacttatgaggtatcactcagtgagcacagagacgcctcatgaacgaaatgtggatccttcacctgtgggtgagtctaagcaccgcccaggtcagtcttcagctcctgctcctccacctcgtcttaacccctccgcctcctcgccaaacttttttaagtacctaaaacataattccagtggagaacaaagtgggaatgctgtgccaaagagcatctcctaccgtaatgccctgcggaaaaaacttcattcttcttcctctgtgccaaattttctaaaatttctggctcctgtagatgaaaataacacctctgattttatgaacacaaaaagggactttgaatccaaagcaaaccatcttggtgattctggtgggactcctgtgaagacccggaggcattcctggaggcagcagatattcctccgagtagccaccccgcagaaggcgtgcgattcttccagcagatatgaagattattcagagctgggagagcttcccccacgatctcctttagaaccagtttgtgaagatgggccctttggccccccaccagaggaaaagaaaaggacatctcgtgagctccgagagctgtggcaaaaggctattcttcaacagatactgctgcttagaatggagaaggaaaatcagaagctccaagcctctgaaaatgatttgctgaacaagcgcctgaagctcgattatgaagaaattactccctgtcttaaagaagtaactacagtgtgggaaaagatgcttagcactccaggaagatcaaaaattaagtttgacatggaaaaaatgcactcggctgttgggcaaggtgtgccacgtcatcaccgaggtgaaatctggaaatttctagctgagcaattccaccttaaacaccagtttcccagcaaacagcagccaaaggatgtgccatacaaagaactcttaaagcagctgacttcccagcagcatgcgattcttattgaccttgggcgaacctttcctacacacccatacttctctgcccagcttggagcaggacagctatcgctttacaacattttgaaggcctactcacttctagaccaggaagtgggatattgccaaggtctcagctttgtagcaggcattttgcttcttcatatgagtgaggaagaggcgtttaaaatgctcaagtttctgatgtttgacatggggctgcggaaacagtatcggccagacatgattattttacagatggaaaagaccatcaatcaggtatttgaaatggacatcgctaaacagttacaagcttatgaagttgagtaccacgtccttcaagaagaacttatcgattcctctcctctcagtgacaaccaaagaatggataaattagagaaaaccaacagcagcttacgcaaacagaaccttgacctccttgaacagttgcaggtggcaaatggtaggatccaaagccttgaggccaccattgagaagctcctgagcagtgagagcaagctgaagcaggccatgcttaccttagaactggagcggtcggccctgctgcagacggtggaggagctgcggcggcggagcgcagagcccagcgaccgggagcctgagtgcacgcagcccgagcccacgggcgactga

SEQ ID NO: 126 - DNA, 900 bp, Homo sapiens
atgatgggcaaggaagaggagattgcgcggatcgcccggaggctggacaagatggtgaccaagaagagcgcggagggagccatggatttgctgcgggagctgaaggccatgcctatcacgctgcacctgctccagtccacccgagtcgggatgtctgtcaacgcccttcggaagcagagctcggatgaggaggtcattgcactggccaagtctctcatcaagtcctggaagaagctcctggatgcttccgatgccaaagccagggagcgggggaggggcatgcctctgcccacgtcctcgagggatgcctcagaggccccggatcccagccgcaagaggccggagctgcccagggcaccgtcgactccgaggatcaccacatttcctccggtgcctgtcacctgtgatgccgtgcgcaacaagtgccgcgagatgctgaccgctgccctgcagacggaccatgaccacgtggccatcggtgcggactgcgagcgcctgtcggctcagatcgaggaatgcatcttccgggacgttggaaacacagacatgaagtataagaaccgtgtacggagtcgtatctccaacctgaaggatgccaagaaccctgacctgcggcggaatgtgctgtgtggggccataacaccccagcagatcgctgtgatgacctcagaggagatggccagtgatgagctgaaggagatccgtaaggccatgaccaaggaggccatccgagagcaccagatggcccgcactggcggcacgcagacagacctgttcacctgcggcaagtgcaggaaaaagaactgcacctacacacaggtgcagacccgcagctctgatgagcccatgaccacctttgttgtctgcaacgagtgtggaaaccgctggaagttctgctga

SEQ ID NO: 127 - DNA, 819 bp, Homo sapiens
atggatttgctgcgggagctgaaggccatgcctatcacgctgcacctgctccagtccacccgagtcgggatgtctgtcaacgcccttcggaagcagagctcggatgaggaggtcattgcactggccaagtctctcatcaagtcctggaagaagctcctggatgcttccgatgccaaagccagggagcgggggaggggcatgcctctgcccacgtcctcgagggatgcctcagaggccccggatcccagccgcaagaggccggagctgcccagggcaccgtcgactccgaggatcaccacatttcctccggtgcctgtcacctgtgatgccgtgcgcaacaagtgccgcgagatgctgaccgctgccctgcagacggaccatgaccacgtggccatcggtgcggactgcgagcgcctgtcggctcagatcgaggaatgcatcttccgggacgttggaaacacagacatgaagtataagaaccgtgtacggagtcgtatctccaacctgaaggatgccaagaaccctgacctgcggcggaatgtgctgtgtggggccataacaccccagcagatcgctgtgatgacctcagaggagatggccagtgatgagctgaaggagatccgtaaggccatgaccaaggaggccatccgagagcaccagatggcccgcactggcggcacgcagacagacctgttcacctgcggcaagtgcaggaaaaagaactgcacctacacacaggtgcagacccgcagctctgatgagcccatgaccacctttgttgtctgcaacgagtgtggaaaccgctggaagttctgctga

SEQ ID NO: 128 - DNA, 100 bp, artificial sequence
  probe
ggaactccaacatgaactactggctcattatccggctgcccattctctttggcattggggtgaacttcctcatctttgttcgggtcatctgcatcgtggt

SEQ ID NO: 129 - DNA, 100 bp, Artificial Sequence
  probe
ccaggggggacaatatgtgccaatcaatagcacccctactcacatacacacacacctagccagctgtcaagggcagaatgaatctatgctggataagaaa

SEQ ID NO: 130 - DNA, 100 bp, Artificial Sequence
  probe
tcacagatgattcttctgaatgctcccgaactactgactttgaagaggtagcctcctgcctgccattaagcaggaatgtcatgttccagttcattacaaa

SEQ ID NO: 131 - DNA, 100 bp, Artificial Sequence
  probe
gacccagctgtagtgaggttgcagtgattgagtaggattggcctgcttcaaagcagaggtttctcatgggaatatgcttattaaactcccactggtgcag

SEQ ID NO: 132 - DNA, 100 bp, Artificial Sequence
  probe
agcggtgcaatgcagcacccaccctgcgagcctggcaattgcttgtcattaaaagaaaaaaaaattacggagggctccgggggtgtgtgttggggagggg

SEQ ID NO: 133 - DNA, 100 bp, Artificial Sequence
  probe
ggtctcgttcgtctccagctcataaaatgtagcagcatcatccttgacagtgatgtttttcaggccctccattgagaacctgaggaaatctgtaaagata

SEQ ID NO: 134 - DNA, 100 bp, Artificial Sequence
  probe
gcatgatgagtactgtttctacaatggcaaaacgcaccgggagtgtcctggctgcttcgtgcccttcgcggtgtgcgtggtctccaggtttccctattac

SEQ ID NO: 135 - DNA, 100 bp, Artificial Sequence
  probe
gcgccgcctcctcgtgctggaagaattcaagacggagaagaggctgtgcaagatgttctacgccgtcacgctgctcttcctgctcctctgggggccctac

SEQ ID NO: 136 - DNA, 100 bp, Artificial Sequence
  probe
cctgaagctggcctcagctgatgtgctgagaccacgggtcatgcacacgtatgattccaggtcatgcgggctctactgcaggacagacctgtgtcctgtg

SEQ ID NO: 137 - DNA, 100 bp, Artificial Sequence
  probe
caagccctgtctttttcccaagccctcaagcacacgcatgagtgttcatcccgacttggtagggggcttttcacccttacaagatggcaaaagattcaca

SEQ ID NO: 138 - DNA, 100 bp, Artificial Sequence
  probe
actcagccactggcgaggaaagctccacatccttgacaatcagagtcattgctcctccaggattaggaacttttgctttcaataatccaacgtagcagcc

SEQ ID NO: 139 - DNA, 100 bp, Artificial Sequence
  probe
agtttgcagtaatgagctgaagtgtgtgtgtaacagacactggataggttctgattgcaacacttacttccctcacaatgatgatgcaaagactggtatc

SEQ ID NO: 140 - DNA, 100 bp, Artificial Sequence
  probe
acctgcaagttgattagaactgcctttcttcccaggcttgacataggtattaagtcaaaattacatgaaacccagtggtaaaaaagcctctgaaagctgt

SEQ ID NO: 141 - DNA, 100 bp, Artificial Sequence
  probe
ggtgtcaagcaaataatatgtgggggccgacacgactaccaacctgtgtaagtgttttccctctcgagtgtccagcacttcctatgatccacaatggaca

SEQ ID NO: 142 - DNA, 100 bp, Artificial Sequence
  probe
ctgatatattgtgctcgcccaggctctaggatgtgggaagtgaactttgatggagaagttataagtacacatcagttcaagaaactcctctcgttgccac

SEQ ID NO: 143 - DNA, 100 bp, Artificial Sequence
  probe
ctaaggacatccagcttgcgcgtcgtatccgtggcgagcgagcataatcccctgctctatcttgggtttcttaattgcttccaagcttccaaaggctctt

SEQ ID NO: 144 - DNA, 100 bp, Artificial Sequence
  probe
caatgtcgaacgcaatgtataataagatgtggcatcagacccaagaagccctcggtgctttactcgataaagagcctcagaagatgattgaaccacaaag

SEQ ID NO: 145 - DNA, 100 bp, Artificial Sequence
  probe
gcctgcagcctggcatcttcctgttctacctcctcattcagactgtaggcttcttcggctacgtgcacttcaggcaggagctgaacaagagccttcagga

SEQ ID NO: 146 - DNA, 100 bp, Artificial Sequence
  probe
gatcgtcctgctggctgtgttcaagaataagaatctccaggcacccatgtactttttcatctgtagcttggccatatctgatatgctgggcagcctatat

SEQ ID NO: 147 - DNA, 100 bp, Artificial Sequence
  probe
catggttaatgtctgtgaaactaatttgtccaaacccaacccaccatccctggccaaataccgagctttgaggtgcaaaattgagcatgttgaacagaat

SEQ ID NO: 148 - DNA, 100 bp, Artificial Sequence
  probe
gtttcctagcgcagccatgtgattaccctctgggtctcagtgtcctcatctgtaaaatggagacgccaccacccttgccatggaggttaagcactttaac

SEQ ID NO: 149 - DNA, 100 bp, Artificial Sequence
  probe
cattgcctgcctcttcagcggctctctcagcactatatcctctgcttttaattcattggcaactgttacgatggaagacctgattcgaccttggttccct

SEQ ID NO: 150 - DNA, 100 bp, Artificial Sequence
  probe
tacgtaacccaagtagtctgaagacgctggttaatgcatcctgcagagtatctaatttattggcagaacatagcccactaggatctggggcctacagatc

SEQ ID NO: 151 - DNA, 100 bp, Artificial Sequence
  probe
accaccctgtccaggtaatgccaaaaagtcctgatgaaatgaggaaggtctttatcgaccgggccaagaagattgataccatctcccgagcctgcttccc

SEQ ID NO: 152 - DNA, 100 bp, Artificial Sequence
  probe
ccccttctctgttaccaaggtgaccccaaggaacacagtaaatgtggcggcttatttggcctccccaggacggactggagcatcagtagtgcctgagttc

SEQ ID NO: 153 - DNA, 100 bp, Artificial Sequence
  probe
gcaagcaccgccagggctccgtctaccattcagacctcttcgccatgattgggaccatcttcctgtggatcttctggcctagcttcaatgctgcactcac

SEQ ID NO: 154 - DNA, 100 bp, Artificial Sequence
  probe
ggagcgagcgccgctttccaagtttggagactagactgaaacttttttgggggagggggcaaaggggactttttacagtgatggaatgtaacattatata

SEQ ID NO: 155 - DNA, 100 bp, Artificial Sequence
  probe
agtccaggtgccacaggcagccctgggacataggaagctgggagcaaggaaagggtcttagtcactgcctcccgaagttgcttgaaagcactcggagaat

SEQ ID NO: 156 - DNA, 100 bp, Artificial Sequence
  probe
tgtggacccagacagcgtggtcctctgcctcttggccattgacgaggaggaggaggatgacatcgccctgcaaatccacttcacgctcatccagtccttc

SEQ ID NO: 157 - DNA, 100 bp, Artificial Sequence
  probe
tcacaccaggtacttaaagatgtgctctgcttttttccaactacggagtgtcactgctttctaggtcagtccctgcagactcttctcaactctttcccta

SEQ ID NO: 158 - DNA, 100 bp, Artificial Sequence
  probe
aaatcttcgataactggatgacgatccaagaactcttaacccacggcacaaaatccccggacggcactagagtatacaattccttcctatcggtgactac

SEQ ID NO: 159 - DNA, 100 bp, Artificial Sequence
  probe
agcgtccagaaatggtgctccccatgcttccagctaacaggtctagaaaacccgcttgtgactagcagtccctgtggctgttcctgtgaggatgacgtta

SEQ ID NO: 160 - DNA, 100 bp, Artificial Sequence
  probe
tattcagaggctgctctgctgagaagatgaacaaatttcttgtccaaaacaatgtatttcaaacgtgccgctcgggcctttcccgtattgctcactggtg

SEQ ID NO: 161 - DNA, 100 bp, Artificial Sequence
  probe
ctctcctaactattcctgggcgtacacatcctgttgagatcttctatactccagaaccagagagagattatcttgaagcagcaattcgaacagttatcca

SEQ ID NO: 162 - DNA, 100 bp, Artificial Sequence
  probe
agtagccctgcacctgccagtgagctcgccattcactgattggaagagtgacctggcatcttggaaatcattgtgtgtcttcaggagaatgtgcagtgtc

SEQ ID NO: 163 - DNA, 100 bp, Artificial Sequence
  probe
tgaagtccaatatcagatggaaatgatgcggagccttcgccatgtaaacattgatcatcttcacgtgggctggtatcagtccacatactatggctcattc

SEQ ID NO: 164 - DNA, 100 bp, Artificial Sequence
  probe
accaggtgccactggacctggtgtcacggatgaaaccgtatgcccgcatggaggagtatgagaggaacatcgaggagatggtggcccagctgaggaacag

SEQ ID NO: 165 - DNA, 100 bp, Artificial Sequence
  probe
tttggaggacaccattcaagaggcaataccaataagttctacatgctggattcaaggtctacagacagagtgttacagtgggaaagaattgattgccaag

SEQ ID NO: 166 - DNA, 100 bp, Artificial Sequence
  probe
cactgacagcctccaccttgagcactattctaaggagcaaataccttagctcccttgagctggttttctctgatggcacttttgagctcctaagctgcca

SEQ ID NO: 167 - DNA, 100 bp, Artificial Sequence
  probe
cgctgcttctccgttacccctttgagacctcgggagccggccctcttcccgcctcactgaccatccctcgtcccctatcgcatcttggactcggaaagcc

SEQ ID NO: 168 - DNA, 100 bp, Artificial Sequence
  probe
ttgtgcagcccacagatatcgaggaaaatcgaactatgctcttcacgattggccagtctgaagtttacctcatcagtcctgacaccaaaaaaatagcatt

SEQ ID NO: 169 - DNA, 100 bp, Artificial Sequence
  probe
gactacggagaaaacgtgttcaagcttctcctgcaactcacatatctcgacagctgttactgggaccacaaggaggccccttactcagatattgaggacc

SEQ ID NO: 170 - DNA, 100 bp, Artificial Sequence
  probe
aaattccttacgcagtggtattcatgatggtgccctatctaagtccaggactgttttcctacagcgtgcctcaaaagtgttgtagagggcaggattctac

SEQ ID NO: 171 - DNA, 100 bp, Artificial Sequence
  probe
ggatgaaaaccagcgactggcaaagaagaaagctgaccttcatgatgaagaagatgaacaggatatattgctggcgcaagatttggaagatatgtgggag

SEQ ID NO: 172 - DNA, 100 bp, Artificial Sequence
  probe
ccgaagtgggtactgtgcaatgatatccaggattccaaggggtgcttttgagaggaagatgtcttacgattggtcccagttcaatactctgtatctccgt

SEQ ID NO: 173 - DNA, 100 bp, Artificial Sequence
  probe
tccaaccagctcttcaggagaagcacgccatgaaacaggccgtcatggaaatgatgagtcagaagattcagcagctcacagccctgggggcagctcaggc

SEQ ID NO: 174 - DNA, 100 bp, Artificial Sequence
  probe
ctccacgagagactaatctaaaagctctgctttgtacttcctcaccctgctttcgtacaaggaagggggacgatgggaaatcatggacttgtaagttgta

SEQ ID NO: 175 - DNA, 100 bp, Artificial Sequence
  probe
tctcctcttccaaagaactctggatccctaaatgaggcagaagccttgaacccagaagttactctatcttcagaggggtccttaaacctcgaagacattc

SEQ ID NO: 176 - DNA, 100 bp, Artificial Sequence
  probe
cggctcagatcgaggaatgcatcttccgggacgttggaaacacagacatgaagtataagaaccgtgtacggagtcgtatctccaacctgaaggatgccaa

SEQ ID NO: 177 - DNA, 100 bp, Artificial Sequence
  probe
agctggccttctatctcctctccttcttctactacctttactgcatgatctacactttagtgagctcttaacgcaaagaccatgcacatcatcagagact

SEQ ID NO: 178 - DNA, 100 bp, Artificial Sequence
  probe
tcctcctgttcgacagtcagccgcatcttcttttgcgtcgccagccgagccacatcgctcagacaccatggggaaggtgaaggtcggagtcaacggattt

SEQ ID NO: 179 - DNA, 100 bp, Artificial Sequence
  probe
tgcagaaggagatcactgccctggcacccagcacaatgaagatcaagatcattgctcctcctgagcgcaagtactccgtgtggatcggcggctccatcct

SEQ ID NO: 180 - DNA, 100 bp, Artificial Sequence
  probe
cggtcgtgatgtggtctgtggccaacgagcctgcgtcccacctagaatctgctggctactacttgaagatggtgatcgctcacaccaaatccttggaccc

SEQ ID NO: 181 - DNA, 100 bp, Artificial Sequence
  probe
cgaaatgtttcattgtgggagcagacaatgtgggctccaagcagatgcagcagatccgcatgtcccttcgcgggaaggctgtggtgctgatgggcaagaa

SEQ ID NO: 182 - DNA, 100 bp, Artificial Sequence
  probe
cagtttccaccatctcggtcatcaggattgcctaatatacctgtccagacaatctccagagctgctgcagaaaagctgtttgggaatatggaaggagact